SEQUENCE LISTING (131 sequences)

SEQ ID NO: 1 - protein, 144 aa, Arabidopsis thaliana
MDYRESTGESQSKYKGIRRRKWGKWVSEIRVPGTRDRLWLGSFSTAEGAAVAHDVAFFCLHQPDSLESLNFPHLLNPSLVSRTSPRSIQQAASNAGMAIDAGIVHSTSVNSGCGDTTTYYENGADQVEPLNISVYDYLGGHDHV

SEQ ID NO: 2 - protein, 361 aa, Populus sp.
MAVELMMGYSGDSFATKMQENDVREAATAGIQSVEEVIKLLKQNQLEQQQKQQYYQELSAASSSSNLGTDNIMAVTDMAVNNFKKVISLLGRTTRTGHARFRRAPVACPPQQRMQEPEPEPQQQKQQVQEPVPYVRAINSQPTEQGSAFRVYQPTPIHRLPPLPHNQQQKTLVVTKNGLSDRNEMATTINFSNSPTISAPTSFLSSVTGETDSFQRSTLSGFQFTQPSAGKPPLSSSSLKRKCNSMDDAALKCGSSSSRCHCSKKRKSRIKRVVRVPAISSKMADIPPDDYSWRKYGQKPIKGSPHPRGYYKCSSVRGCPARKHVERALDDSMMLVVTYEGEHNHSHPIDEAPGALVLESS

SEQ ID NO: 3 - protein, 568 aa, Populus sp.
MERDKLFVSEGANTAATIWNSCSFGMEIQANELSCGPEKLVNCFLNPNWDNSLDQSDPFESALSSIVSSPVASGANANANAVPNAGVGGDGFMIRELIGRLGNICNSGDISPQSFVNNNNNSTNTSCYSTPLNSPPKLNLSMMDSQMRGNLPIPGNSVVKHPGLAPFPADFVERAARFSCFGSNNLGGLNKQFGLNESELINRLMPRVEPGKLSRVSSNNSMKVTVSQANVQESNKSSPQDGSLNSDKKFSRQSRPSTSENGDSREESSVSEQVPGGKLSMKSQNDANSRKRKSIPRGKAKETPSSSPSASDVKVAAENDDSKAKRSKSDETNGSDKDTAKEKEEENGNQKQNKNNSKPPEPPKDYIHVRARRGQATDSHSLAERVRREKISERMKFLQDLVPGCNKVTGKAVMLDEIINYVQSLQRQVEFLSMKLSSVNPRMEFNMETLLSKDIFQSRGSMPHSLYPSDASTPAFPYGYQSQQGLALQNGMPSNAETQFSMNPLNAALRRNPSMHLPPLDGFGDPAVLQASAMWEDDLQSVVQMGYGQNHQESFQGSVPSTHMKIEL

SEQ ID NO: 4 - protein, 347 aa, Populus sp.
MEGVEEANRAAVESCHRVITLLSQPQDQVQYRNLMVETGEAVFRFKKVVSLLNTGLGHARVRKLKKLPTPLSQSILLDNPLSSTGHPSKTSQFLQSSSYLESQSIQELGSIAKNCLSLGTPSLELSSNGKNPLQLGQPTPAAPYQFLQQQQLHRLQLQQQQQMKQQAEIMFRKNNSGMSLNFDSSSCTPTMSSTRSFISSLSIDGNVANLEGTAFHLTGAARSSDQSSQQHKRKCSGRGEDGSMKCGSSVRCHCSKKRKHRVKRSIKVPAISNKLADIPPDDYSWRKYGQKPIKGSPHPRGYYKCSSMRGCPARKHVERCLEDPSMLIVTYEGEHNHPRIPAQSTNT

SEQ ID NO: 5 - protein, 301 aa, Populus sp.
MAVDLVRYSKMEDQMAIQEAASAGLESMEHLIFALSNQTRQSHQLDCGEITNFTVAKFKQVISMLNRTGHARFRRGPTSSPSSYPVPVRPVPQEPQKLNLDFVNSKSPPKAESKNDLSLGSQYSKDSLSSGTTTSSFVSSVTADGSVSNGKQGGSSLFGTQARSTGKPPLSSTHRKKCHDHALSARKISSGGSCHCSKRRKSRVKRTIRVPAVSSKLADIPADEYSWRKYGQKPIKGSPYPRGYYKCSSVRGCPAKKHVERAVDDSAMLIVTYEGEHRHSHTPLPEDVTASAAMRHVFHST

SEQ ID NO: 6 - DNA, 1084 bp, Eucalyptus grandis
  source 1..1084: mol_type = genomic DNA
cagaagcccttgcgttgactacagtctgcatttcagggcagttaagaaagagggaaaggaggagagggagagatgtagaaagatggagagtccatcagaaacagaaactgactacagccatttatttataggaagatgaggactctcgagactcgtggcgaagaacgagtcatcaagcacggggcccttcgatgcggaatttttttggaggtcctgaagatgattattcatttccccaccatgtctataaattcttcgaatttgtggcaattcaagttggggaacaaaacacggccagacccaaagaggaaaatgagtttacaacataattcaaccgacatgaaaaattagttcttgcaaatatgatgccatatagatcattcttcatagttaagctactatgtggttgttaacatgatattcgtgttgatacttataaagagaccaatcaaaatcgatccttatccttaaaaagtcaatatattttcggggaggcctattggaatactaataatcatgctgcctaattttgtcataagaatgtgagatctcgatataatcaaaggtcgtgagatcatgacagatgtagataggtaggtttcttaggagcaaagcaaaatatcggggacactgctggccagtaaagcccatgatgcagataaaggaacgaatccctccctccatctttggaagcccaacccacctcagaggccaaaagctacgttgacaggagaatgggagcagagagtcgcatcgttcaacgacgtgtggttggaccgacgaaaacttcaaaagccagggaaaaagaaagagcgcctgtgatcctttacgtaaatacgcgggggggagttctgtggtgcgtttcggttttttttcaacaccgaccctcccagttgtcttccttcctgcctaagatcggttttacttttagacgatcgattagacgatcgatcgctctctctctctctctctctctgtggcctcacgcgcatttccataggcaaccaacaacagcagcagaaagtagagttcctgcggcgatatcgcgcgatccgagtcgcgagctgaggccccgaccccgacg

SEQ ID NO: 7 - DNA, 2000 bp, Eucalyptus grandis
  source 1..2000: mol_type = genomic DNA
aaaaacacggatatgatacaaaatagagagagagagggatctattattctcacttggatgtattgtaagaaatcacaataaatctttcattcttgctagaaggggaagttgatacccccaatctaaacttgttctttttagtccatctattttggtcaattgagtagctttccctctaccaaactctgaaatgttctaaaataatttctcaaaaaaagatttttttatcaaaagaatagaccttgaatatatattaagccatgtcaaaattgcaaatcacagcaattacaattaataatcctaaaacgaaaaatcaagcttgtaacgatgattgctacaaaaaaaattatgaattgcaatttttttggggagttcactaaattatgtaaccttcaagaactgagtaaatctaaaaaaatgttccaaaactctagtagaattacgatagtaacttacacttgctaaataatctaaagaccccaaaagaaataaagttattttagtaagtgaaacgcttcggattctgatagtggaattcccagctataaaatctcaagcacatcccaaacctatagtattaaaaaaattgccaaatcttcgaacttggagaacttttttttctagaaaccgatcaaatttaattttagtaggcacatgcagacatgtgccatttcaccagtaaactaaaaaccaagaaaagattaacacggaatgacaacattctataataaatttgagaaaatcgcagaaaatgtgcaacaaaatcgagagatccaatccattcactagaaaaatgtaatatcgagcgcaattaccaaaaacatcgagttcccatacgcgcgatcacacaagacatatagcaaaaataaagaaaagagattatttccaaaaaaaataaattggatttatacacggcatctagaaagaggccgactcttggatacattaccaaagaagaaattcactcgcaagaccatccatattctgtagaagattctagaaccaggagctcgcgaaaggtagttgccaacttgctcatgtcttgtctgtccgccatcaacctttcatcggcttttgacttttcctcctgaccaaaaaaaccctaattcaattcaaaattctcctccggctgctgggtttaggtactcctaaattcggcgggcaatgccttaagggccacaagtttttcttcagtctcgcacaatttttttttttttttaacaacacataaaataatagtaatttttgcgggcgtctaatataataaagaatagatcaaaattgcttgatcaaaatttactttgcgctggcgcaatatgctcagtcttttgttcttgaattaatcacttcttcgatatgactgtcgctaaaagcgacatacgcgaaacaaattttggtacgtaagatcgatacaaattgttcacaatttcttagaatttctaatatttcttttattttttgcaaagggggaggcaaaaaattaaatgagaaggaaataatgttagacaagcggaaccattgaaaacctcctcctacccaagcccaccggctctcagatatgctccgaatattcccgcttctcgttttccccacgctcgcttcacgcgcgtggctcacaatcctaccctccgcgccggcctccctttcgaattccccgagctcggcgcgtgcccgtgatgggcacgacattcccgaaccagcgggctcccaacagagtcccgctttcaactaaatcagcggctcgaagcatcgttcttctcgctatcatctttagggatttcgtttcgcccaaggcagagagaaaaaagagagaggctgactcgtcgactcacgtgacccggcccgagcggactcaccgttctgactcgcttatttaaagcgtcttactgtccctcaccctgttgtctcaaaccactctcaactcactctcatctctacactgctttttctcgtcttctctcgctagccatttccagaaccctcgtcgcgcgtcgcttgcc

SEQ ID NO: 8 - DNA, 2000 bp, Eucalyptus grandis
  source 1..2000: mol_type = genomic DNA
aaatggatatgactacacaattattttcgagtgcatactttgtccatcaaacaaaaatgcctgaaaatttctttattaatgggctcgacaaggatgctaattgctagtgaaattcggtgatttgctgctgctgctagagacatgttagactcttgtccatgttcgtgcctcttgggcaattttgttgtctaattcgagagagatcacaaaaaaacatggaagtgtgaatctcttctcgtctacttgatatgtagccaaatcacatgaccttaagagcctatctttttcatcaattcgccaatcatacgagggaggttagatgctgattcgcgagccgtaaaaagcccattcatcgtctaactagccatgcctaggtgaatgcaaccgcacaattagctccaaaagcatatatcgttcgtcgaacggaaatgtctaagaatttatcgacactacaaaaaggatcctgattggatgtgttggactcgtgtctatgcgtgtcccctttggacaacttggttgtctaattcgagagaaaatacaagaagccatgaaagtgcagatctcttgcttttccttttagagtacttgatatatggccaaatcacatgatcctagaggccatatctttcatcgtttcgccaattatatgaaggaggttaggcactaattcacaagctgtaaaagcaccagcatgttaaacaagtaaacgaaaaacgtcatacattcaaaaatgtatacacataacgagtatcttggaatagaatactatgttattatccagtttcgatacgacggagaatcgccaagtcatgatcttctcgcgattccattgatcggaacaaggttcgcctatgctccgacagtaaatcctagggagggccacccccacatttctaaatgctaacaagaatcaagaacgagctcgagacaaaaaagatgtcctgtaataaccttagaagtcctgcattgattcgcacacgtcaggcaatttttcattttggatggtaaccaatccatcgaagcttggctcaatgcatacagaatagaaacaagtatggggggcatcgagtattaaatttggaaatgatgagagatgggagaggccatggtgcgagaacgcagttaattcttgctctgcaacacacagtgaaagagagagaaataattcgtccttttcgacccctgaaatggatcgccatttacattttacccccatctctgcaagaaaacagctcccccccagcaagaaccgagaagcgaaagcgacccattattcattacggcgtatgtaaccaagcatcccaacattcaagaaagagagagagaaacggtagtagtggggtggggaggagggggggaaatgtagagagagaaatttcaccacccagagtgtcatagcgcgcagagagagagagagtatagcgttgcaggcgttgttgcagaaccgtggcagatggtcttttaaccttttctctcgaaataattcgctccctctctctctctacccacgtctatctctctcctcattacggttctattctggggtggctctttgcctgggagcagtgagcattatatgctcatcgaaggaggagaaaaaaatgagaacggaaaagcaaaatgcagtgtaccccatctttctctgcaggcagagagagccctgtgtgtgccagagcgatgtgatggaacgagtaaaagtaagctaatctcagacttgaggctgtcataacatctcagagggagaagaagaagaagaagaaagcccaagaatttacttcttggttttttcaaaatgactatcaaagacccaacgaacatgcgcttctctttcttcagctcctccttcctatctctgcaggcatcgtgtcggtgagctcggagtgggctccgaccgggcaagctttgcttccgaagcattcgttcgtaacagagtccaaagttggctcctttgccgcgaattctggagtttgaaaagtgtgtggttggctgggggaacggcc

SEQ ID NO: 9 - DNA, 2500 bp, Eucalyptus grandis
  source 1..2500: mol_type = genomic DNA
ctcacaatccatatgcatcgcgggccattatatcttaaccgaattttagtgcgccctaaaatgaaatcatgcagatattcattcgttaaatcgcacgtcttttcgcaagagcacgtcataaataatgctctagcggatgcaaaagcgtgtgctgttaatgtcgaatcaaaagctgtaaaccacgttcgaggggctaaggcaatgtttttcgcaggaagtcgcagacaggagaagaagagatctaaaattagatactcaagctgtaaatagggtagagagagggggagggagtactgtacgtaggaagtgactggagggtgcacagtgtccctcccggtcatatctatcttaggccctgcgcctctgaaaaaaaagctacccagttttggttatcttctttgctgtcgccttgggaaaactcgttattttattattttacaaaaaaaataaagtatttttttggtacgtaccatctgtttgatatatactgtgctggatttttttttttttctgggtgtggcggtcgatactttggtggatatcgtatgatttctgttttttttttttgtttattcattttctggtccattttcttttgagttgcttttattataacagaactagtgataatgatttctctgatttcattcctgaaactacattgtaaaatcatgtagaacttattagcagacaacattaacatttaaatctggaaaaaaaaatcataacttgtttcttaaaattagaaacaagctggaaggggccatacccgcgtggattatctctccggacattatcataagagttgtatgcccgccaacattgttcattttgaactagccaattgttattgcctcatcataatgttagcaaaaatggaatcggagttatcctgaatgacaacatgatcaaacctacactttggatacacattcctgaaattctaccctctcaaacttgtcacaacataaccatttaaaggagatgctccaaaagcttaattcctcctattttaaaaaaaaagatcgtgctttacatgagattcatcttgaaaatttatgtacaataaacaaaaagaccacccaaaaaagttagaatagaagattcatccacaacgagaagtatgacaagtgtacgagtcattgattttgcttaggttgggtaagaatcgggaattgaatttcttggcaaaacaaagattctcaatgcaattcaaacaatgatcgcaaaagtctgtacgtatatatagagtttgtaaacgtcgatgatttcaatattaaacaaatctcaaatggcaaaaagatgaaagtcgctctaaattagccctatgtgagcaatatttgtggatgatcatttgccccgtgacattaatatgtggcctttttcctactagacgctcaaaatctaataaatatagacttttgaggccacatcaatagtcttcataatttactaacataacagtttttgggctaccgtgagaataataatttgatgcgagtcacaaattattacgaggtacaaattatcgaaagaatggacctcataataatctacaattatttgttatttagttttttatgatctaaaacaaactattattaaagaacatcatccaattattactcattttcttttttattggaaagagtccataatttcccaaataattataatgtaatgtttactgtgacggaaaaatcaaatgctacttattttttggttggctgagagctggaccagaaagaggcagacgctgtaccaatatatgaatgtcattaatgtaaaatagtgcgttgggattctatggagggagggaaggggaggatgtttgtagtttccattaaataaatatagtgtggaactttaatggtatcaggacgacaacgagccaaagaatgtccggaggtcgctgtaggagcacgttttctgggcttatataatgcgcgcgtccgggatttgtgtgcttgtgttaagtgtcgagttttgttggctgttgtctcattaagccaaaaggccaatccataagagaaaggaggaaaaaaaaaggagaaaagcagagagagagagagagagagagagagggagtagggggatttctcgcacattgccttacacagatccccatggcttttttatgcagggatactacccagcattcgcttggagctgagctctttttattctctttgttttagattgaagtgaagggaccatttctctctctctctccccccctatatatgagagcttgagaggctgtgggtgtgaagttgttgggttatattctcggaactagacaacaacaacagggaaaaaaaaaaaaaaaaacccaagtgtgtgtgggtgtgtgttttctctctcagatgcaagaagatttcgaaatgattacttcccttcttgccaatgattgaagcctcgagctcgtgcttgcctcgcaccacaatgagacatgcttaacgaagtaccgactttttaactgaacgaggaggaggagaag

SEQ ID NO: 10 - DNA, 2700 bp, Eucalyptus grandis
  source 1..2700: mol_type = genomic DNA
aatgcaaaatggccgaaaaagagaggagagaaaagaaaaaactcagtcccgtctctcctccgcacgatccccatccccatccgacgcgcctgatctctccccagcgagatcagaagacgctttttacaccgaaacggatcgttctgatacgggggacggggggcaggaccgagcccaagtccccggcacggcaaaagcgaatcgcgagcaaagctcggtgcgtactgcagagaattcggggcacggagcggtgtaaaatatgcaggggagaaatcgcgaaataataaccggaagcaggggagcttacgatatcaaattccctcgtacacgtcagccgccgtggggaccacctccttcttttgtcctcttgctcctgctgccctcgcatgctcctgctctctactctctctctctctctatactctccatccgtgctttgctctctcttgtcacttcctgtcaaagttgcttgctttctctctctagcttagcttggagcttacctactgtactctgcttttcccccccctctctctctctctctcgctctgaacgtttttcagtcatcgagccgagctgaactcctctctctctctctctctgggtgacccttcttcccttttctctgcgcttccgtcgaagtgaaagagggcgccgaggttgtcgtccgagatgcgtcggagaaggacctctctcctgatttcatgagaccgaccagtgagtaagctcgctccgatgactggagcttcctctgacctccctttctttctttttcgggtgctgagcttgtccagcgtcttggccgtcgtcaattttacgctttcgtgtgagctttgcttgctgtctttgtgggtgccgtgcgggactcgattcgtctggtaatcgcgaatgcaggatcgatgctgacttttgagctcccgaaggacggacccttttatctttactagcagaagaccttaaaaagtggaggttctgatttctttttcatatttttctttggctcacggtctcaaatgagtgatcggtttcggttcttggaaagatggcatttctttttcttgaaagaacagcatctggtgaatattttattgttatctcatgcctagaaaggttccttttatttttattttctttttggttctgggaaagggttccagattccattatatctcatcactcggttgcatattcaattgggctctttggtttccctcgggttgcatctgtcttgtaccctgaagaatttggcatccgccgttcttaatgcagattcgttcactttttggagcttctgtttggtctctctttctgcatgacattcttgcattggttggtccccgtggactactcacaattggaaggttcagatgcttttctgggtgcataatttctatcagatgtttgtagagatggactcctttggggggtcatcctcctacatctactttgggggctctagtatgctgtattctgtatggttttaagctttactggaattttaacatcctttcctcttgtggtgtttggttgatgagatgcggatgatagaattccgaactcatcatttagactttcatttgtcgatttaaagactgataatcagaaaggagaattcaacgcaatttcacgttcaattaacttcttgctccacttaaattgcgggtgcagttcaggggccggtttggattctctccttcaatagatttagatgggtaatctctttataatgttttggagattctggaacaagcatctaggagtcaatgcacaaattgcaacactcagcagcatcaatcatagtgttcaccatttgtttggaactgtagatttgacgatagattattgatagggggttttctggattttctgtatagttagatttgcttggaagggacttgtggatgatctagggcggaataaattggtattagatttcaatctctttttgtctgagctcaaattccttgtttcaatgcattttaactatagttatggtactagatttctttctttccttggctgaatgtatgtgaatattgcttccttcatgaaaattgttgaaccagagaaatgcatggaaacatagtgctgcttgcttcgggaattgaacaatatagctggaccagtttggtcgtgtagtgactggaagtttgcctagggtaatatcattttgcttacaaaaccttaacaaaagaatgtgggactcttcattctgttcttggtgattgttctttcagacatagatttcgcttttcagttgttatatttcttggattaggtataattagagaatttgaacatcagcctgctcatgtcctttccttgaaaatgacatatccacccagataccctatgaatttatggaggataagtgacgacctcggctgtaaatgttgttacacaactgttttctctttatgatgctagggagagattgccgcttgttttttcttgcttgcctcactaattggccattgtcttttatagtttcccctaggtctgctttcatttccttggataaacctgtctcctccttgggattcttttcttttctcagaccagttaactcagctaaaatgttgtgtattatattgtgatgtgagaaattacttctaatttgtattatcaccatcttcttctgtagccacactatgaaagatctaagtttgaggcaatgggaagtggtgtctacacctggca

SEQ ID NO: 11 - DNA, 2500 bp, Eucalyptus grandis
  source 1..2500: mol_type = genomic DNA
ataaaaaactcctaagcataggtgcttcgagcatcctaaatgcgagttctcttattgcaagagattgaaaactctcaagcatgagaaattgagattcccaaacaagattcaatgaaaactctcataaaagtaagagcaatcgaagaaagccaaccataattgtatagtttttacctaaattttaggttgaagaagagcgcaatttgagtcgatgtggaaaattgatctccaaaagatgtcttcttggatttggaatatgagataaaactggacatgccacattactaagtgcaactttagatggagtgataacggaatgagaatcaagtaaaaattagtggtgttttatgagacaaaaaaagtttaggttagaataaggacttgacataaagtttaaggtaggattgtgattagaccaaaagttgagcatttttaatgaaaaaaagaaaagattagggtagaattggaacttaactgaaatttgaggaatttttagggtattagggtcacataaaacacatgataattatatcctagaaaaagtacttcaatttttagcttgcatatcatcttttatttagccaattagtgatgtaataagtaaattatcactcgtatgccaaaaaatatcaattataatatatcatcttcacagagaaaatgtaataatgattttatttctctttaataggatcagattagtaataaactcagtcattaaatgcggcatgtatataaaattttgtagttatagtggtcctataaagatatgcaattttttttttctcatttataacgcagttgatttgataaactcgtgataatatgaagcttagattatatattagtaatgtctaatttctgttgcgtattttttcatccctcatcccacatcataaaatttcagtcttctatgttaccaacaaagggtgtaaaagaactcatcgacatcgagttaaaaggttgtttactaacgtttgcatagcaatttctttttatttttgaaagcaaacaaaaacaattattgaaaaacattgtttgggtggttacaaaaaaaaattaattatgtaaaaataaaagaaaaaaaatgacacaaatggttactcttgccaagcctcgatcttttgaccttaaacaatgcaaagcctttactactgggctactatgttaggtggtgcattttagatagaaggatatcacaagatatttgttattccatgtccttcagacagcaattcacacttactgatttgagattttttgtatgcacaacgttcttatgttctgtgaccatatttactattgtcatagtgttgtaactagattatacaattcacttggttttcttcttttatgtcgagcgtgctaatacacaagttcccttacttgattaaaagagcatacataagatcttgaagactgcggagacataaagattttaactttgagggtggttaaagagattacatggaatattttaaatttaaataactatcgatactcacatggtaggttaggaaagacaaataactttctaaataagatagtggcaagcgattttattcaacaagtatcaaatgcaaagtcctcaccgaatctggggtttataatagttgagtcatcttggtttgtccatataaggattctagtcctttacctaataggttttttcaaggtttatgtgatgtgatgggcttgcccatcttgttgaaaacatcaagatttttgctagtaagcgagacagactttacaatagtctctatataactggatcgattaagacccgactaaactaaatgatatataaacgatatatcggaaacctaaagtccaactcaacaattgacgtagatatcaacgtatgatctactccattaatctgatcgtctctagacaagtgactgggtagtatgtgaaaagagatattatccaatcgatgcactttggattaagggttttaaacttacacattgattaaaatattaagaaaatttcgtgaatttgatttataatatgatacgatagactctttattaaatactaacatgacacatttactctccactcatgatttatatcatgctattttgtaagtcaattatataaaatctagagaggccatgcctcagctcttgcctttggtctaacccagccttgaccccacaaaaaaaaaagagcaaaaatctttccgaggaaaaattgcacaaattctacattaaacttttttgcagatatgaacaaatattttttttttctcgaaaatcttccaagacaaaaggcgaggccccttcatctccttcctcacgcaatcgcttggagcgccctctctccctcactccctccctcctctctctctctctctctctctctctctctctccatcccacattcttgaaagagacacatcagaagcgcaggcatgaatctgggcaccggctcgttctctttcctgctccatctataatgagcaaagccttcgctctcttctccgcagaagggc

SEQ ID NO: 12 - DNA, 2101 bp, Eucalyptus grandis
  source 1..2101: mol_type = genomic DNA
caacctcgtggtaatgcaacgaaactagcccaaatatatgttgatagccaatttggagtaactctaattttgtttgcgttggtataagatgcaacaaggtacataaaatccaattccattgaccctgaattgtgtgaggtcttctcatgctggaaagcactgaaaattacaaattcacccccacccagagtcaatacgtagacatacatatgattttacgcaatcattgaagatcaaagtggaataaaataatatttggagggtgatagtgccactgccatggtggtgcggtgctttaaccctggagctctctacacaattctctctctctctctctttcggattatgaaaatgaaagtggggttacgtattgctggttttgaactgtttgggaaattgaagcaagctgttcccaaagaaaaaagcacgccatttattggcaaaataatattaaacatatatctggggcattcagatagagttttctcaacactttgcctattttaaatatttattattttccaattgtatattccacttccattgcctaaagacgacggcattcagttttccccccttctttattcatttatttccccccccccccccttttcccgtcattggactgcgtagaaaagaggcaatttcgagtatagggattggagggatctttctttcgtttttttctttttttttgcttgaattatttttactattttttttaattatttgcttcattccctcgcacctcttcaactttatggaagaaaaaggttgatagaggccaccgaaaagcatggacaccttatcatgaaagcgagaaaaacacgacccgatttgacgggccaacacacacccacaacacaaaataccaaaaaccactcgagcgcctcgcaaaaaattcacacgcttgttccatcatcacattcacatctccatctccaccttctattccctcttggctattctctccaccccctttttcctcggtctctgttgacgagcatgatggagaaatacagagagttttgtcatgtttttgaagaagagattggctccttctgctggcttgcaatgaagaagtggagagctgctgaatggctccatatccttccaatcttccaaaattccacttgattaggatggacaatagtctgtcaaaaagggaaaataaacttatcctgggaaaatcatcgtttcaatcttggccctttaaagttgaatatcaagattattcgcgttatttacaatttggagaagtaaactgagtcaatttgctccaactcgacatgattgtagtccataaacctttaaaggggaaaaatgacatattgcaattgttttgctggtgagaaatcatgatataaagaggataaaattgagttaggcaattgagagcataccaatccatgcaatcaaaagcactttattgaaaaaaaatcaaataaagatatgataataattcatttttctcaataatggaaaaataccttatcaatctcatgaatgagataaacatgagttaaggatctaggtggatgcatctatattaaactctttacggtaggtggtgtagaaaactatgatcccttttatgagcctaatccacacacaaatatgttttttaatcactagccaaactctacaaattaaataccataagaaaaattaaacaataagaaaggcataatttaaagtattataaatttccatgctagtgcatttaatgcaactgaaagagcacctcaaagcaaaatggcaatcataaacagtttctcctttttcctttttgctccagtgcaactttagctttttcatcgtgtgggtaatcaatctccaagtcaacctcgcctcatcatttggtcttgggttcttctgaacaacccaccatgcacctcatttcccactttcccactttcgctctctctctcgctctctctctctctttctctctctccctccctctctccctccctctttttcgttttgtttttgtttttaccttttcaacaggagtgtgtcattccctatataattgcctctctcttttagccggatcttcccctcatgggaagcagaagcatctcgtttcaggcaaagc

SEQ ID NO: 13 - DNA, 1487 bp, Populus sp.
  source 1..1487: mol_type = genomic DNA
tctgcttatgaatggcatggacaacaagctctgcattttcgcccatttgcaccacaaaatcgttgtgtgaagatttttgaagggcaccagcacaactttgaaaagaacttgtgaaaagatagctggtcaccagatggaagcaaggttactagtgcagatcgcatggcttatatatgaatctgtcaatgagtgtgtcttccacctactgaacctatctttggatcatgcagcagcgacaaacagatttatcttggggaaatctgattactattttgtcagtttgcttaaaacgagttgttagtaacagtgaggatattttggggcttgcttgtgagtacccaacagaaagattaaacaggttgtaatgatactatgttacattttacttaactttctcatatcctgctgacatgtttgcagttcttttttgcttgttggcgatccttttcaactgaagtatgtacgcatgaatcaattagctcaaaatgtatgttcgagcaatatgcctggtgaatgtttgttagatacgaaattaagttactgaacttgtactctttgattttgaaacaagtttccgactatgttcattgtcctagcacggtgatattggtacgtcgaattttcaactatttttgtcttgttttcaactatttctgtcttgattttcaaagtctttctgaaaataaaaaacccaaaaatatgttttcagtgtcttgttagctaaatctgttgtacaaccaagcctccccgacttgctttgtgaaaaagaaagatgggagctgggggttttgtgttgattatacaaaaccttacgagataactgatatacaagcaaaaacaccctcatattattgaatctgcgacttctacagttcatttctgaggaagaattaggtgactactttctggactagatatttccgtgttcaatgcgtcgcaacaattgatttgttggctggattatgaacatgataatcacactatagaaagacgacgattatatttcaagctatggacaatgatgttacctgctcgaagatcaatacgcttgaaggccaagatatgatcacaggtatgcaggcagtccagatggctcttgggatatgagcttcaagattgttcttgaaaagaaccaaataaattgtaaaattctctgggaatatttttttgtaaaattatcagcattgctattttttttttcaaattcatttaaatattgctattgggttagtagttcagcgcaattaacttagctggcagtctggcattagcttcaaatcatatcacaacgttaaaagtagccgttgcaactagtcagcccactagcatttgctggtgaccaatcagatcataaaaggaaaactagccgttggcaaaccctctttcaacggtcatctccctgcttcatgatatatatgcttagatatcccagtatccccttcaacaaattatctcaatcttcaaaacccagtccccagttacagcag

SEQ ID NO: 14 - DNA, 1780 bp, Populus sp.
  source 1..1780: mol_type = genomic DNA
gatgcaaaagctggaatgagaatgcgctaaatgcgaaaagacagagagagcgaataaatcgtgctaaaaaaggagtggggtgggtaacgggttgagctagaagaagaaaagagacaagtgcactttaggaggggggcaaccagagcgtagatgataatggttcatgtggaaacaacacacatgagcagttggtgagaacttgaatgaaccctaacagcccaaccaaacccggagccacccttaccgaaccaccacttctaaaagtacaccatgcctttttcttgagcttggttgcacagggtgccaggtggggttgtttcgttttgggtaatcatgcgatagtttaaatacctttgcgataatcatatcaatggtgacttttaagcacatgttaggtgctcggttcttatctaaacatggacatggcaacaagagttaatgctaaaataatatacgtacattacctgtgaatgaatcgtcgctgtcttctgatcatggcttcaaataatatgcagataaacaagtgtcgatttttcagtgaagattttatgaaagtgcccgttctccttaagattacctgtgaatgaatcatccctgtcaggctgatcctgggtggtttttccccatgatgttcggaagatataattatataaatgatggaatttacatgaaataagtttcagtacattcttagcagaaaagcaatatcgacgaagacaaatgatgctgtttaagacaaactggggtaatatcaatttactagtaagagatttgtctgcttttcttaattctcaagaaactttcactaaaatgcacagccatgttaaacaatttacattcaacttaaaaactaaaattgtaggatgggtagctatccaagaattacgctttttgtaaacttaattttgatgggcatgtagttaacaagtatttttcatcgatcaattcaagagccatgtctgcatcataattgtgggagtggaggaggcttttgttgctagggaaggaatgccttcttagttcatggctttggacttcggacaaggagcgcatagaatggggttaccatttttgaaaaaaattacatttgaaccctccaactattatcatgtatgtttaatctacaatcctcgtccgctagaagaagtttgggttcaaagtcatccgctcaatgtaaaccatggagagacagggactaattgaaaatagtatgttagttggagggtctgcatgtattatgtccaaacattctcttattattcctgtatcatctctgagaaattcatccgaaaataataaaacaaaatggccttttttaaaataagaagctgatgcataggataccaaaagcgccttgtccattaggagcgtcagactttgaaaataagaccaataattccctgtaagctatcatctcatcttttattttgtttgaacttgtagacgtaggctttaagcgttccatgatgttcagtcacatgttgctgtctacttgattatggaatttaattcattcggctcataagaagataaaaggattatgacgttgaagaactctggtcactccttacttacggtcacataaaaacgatgcatctttccccaccaaccatcttcaagtgaacccactttcccttgcattaggtaaggagtatgggttaagtcatcttcatgaaattagtcccctagtggagctaattctactcactccatatttactcattccactatataacgccctcaacgaccatcctcaaagcaacccaaacaccttcttctc

SEQ ID NO: 15 - DNA, 2001 bp, Eucalyptus grandis
  source 1..2001: mol_type = genomic DNA
tcaggctctgtggcgggccaacgaccaattctaggttggtcagttattgctctggacaatataataggcgcagatccatggttcatggtcagacatgtcgagcgcatccatgcacgacatgccgtccctatgtttagccaccgcaaagaacatcaataacaattcgtctcgtgactggtgaaggaactatccagctttcgtctctttgaatgcgaattgcgattgcgtgttgttaccccgtccttccttcacgtccttctagattaaactctccttggttcttcgtacgcaagttccgcatacaataataagaatcccccccaaacgaaaggagattcatgaaaaggtacgagcagcaaagtttctctctacccttttaaaaaaatcaggatgtagaagggtcacaactcatcattcctcatatagaccctttctagaaagcaagtgggtggtgtatgtacacacgtctttgagacagattcccacgtaaaagagcgagcgagcagttggccaaatcaaccggacccttcacacaattatgctctccaccaaaccccaagtcatcaccaacccccctctcctccatctctcgtccgatcccatcgaaagtgaccatcattattcatcagtgtgttctcactttctcagagcttggccatgtgggcgttaccccgttttgtcccgtaattccgcttctcggttcgatccccgcaccgccccattgcaggtaaaaatcgatgtccgtgacgacccctttttatctccttttaggtggatggttgcgatcgtggtttggtggaaaaaggggatcaaatcatgctacatgatcctgctgttgacatgtggtagtaatgtcttcccccatttgtctgaactctgatgtcaagatcgtgtaattctatgcttcaaactgaagcgatgagaccctgtgcgtctcccacttatcatgcctctccagaaggattgatccgcttgtcgccacttacctactttggttttgattttcagtttccaacttattggagcgtagcgcgtaattccccaagcaattgatgacaatttaagcaacttcctgaactttgaaaccatgcgagtactgcaagcatgtcattactgtagctcactaatcaaagatataagccaagaaaatctcggttaaaccactcgatctattgttaaggacggccatttccccgacattttaagatgtgggatatgttaaagagtactgtagttattaggacttatcgaaggtatttgtgtccaaaaatgaggaattcgtgtctacttttgagattggtagttctcttttaatgggcattgatatatccgggacataattagcatcggtttcattaacctcgatcagtaatggtgcaatgacatcgcatccctcttatgtgaggagtcccgtttccaagggctcaacagctcaccagtttaatcaatccaacattcccaaaaataatagtggtaattcaaacaaagaaaggtcaccaattcatttaccttgaatgttactttgaggctttgagtagaatttaagtactcgtacaacataacgactaggttgtaattggtgagctttactagcgtcaaattgctctcatggcaatgacgaatgggttggttcttatccgtcaagaaaattagtgacatgattaactttaaagctttgtgaacattctctggactatgatggttatcggtaaatcttttagccgatcgaaatggaccggacatcatcatacaaagggacaaaagaggctaaagaaagagatgagacctatctgttttttgggcctaggggcattactttcgaaaactgcgccatcatgatgtgattaagtagaccaaacattaccaactccggcggccccaattccttcgtaccatctttccttcgtaccatcttgttttatataagatcaaaaacccggacggcattccatcgaaactcaacgattactctctctctctctcaaaagacataaacataag

SEQ ID NO: 16 - DNA, 600 bp, Eucalyptus grandis
  source 1..600: mol_type = genomic DNA
cgacgcaggtcagacgactgagtactgaaagcattggcctatccgtcctcattgaaccaaacaatcaactgatgggtcgggccgctccgagattaagactatttgtcagagaatttttcccaattggtggtccattttcgtcctttaccttattcaaatggaagtctcgagatattcatgtaaaatcaacgaaaaataattttctaaaatgaaaagaaacaagaggaaaatttcgtgtgacatataagcagacgggccccctgtcccctgtcgcactcaaaacacaagatgacaaactcttctcgattagcacccattctttagaggcggttttgtgtccaccgcgtctcttatccggataaaatggctcgagtgcttaatccgtctaagcccacaatttttttttcccattttctcatttaatttacaatttttttccttttttccctaattcgagtggaatttacattttagaatttagaggtcgagtggagctataaatagaggggcacggtttgcaatgtaccctcaagcaactaggagaaagtgataagccttgtgaggtgttgaagacagctttgccccaaggggagatcagtg

SEQ ID NO: 17 - DNA, 1800 bp, Eucalyptus grandis
  source 1..1800: mol_type = genomic DNA
atttgtcagaaagactaaattggtaaatcttcatagggtttaagactaaattggtcaaattgaaaagtttaaaactgaatcagtacccgtataataggcttaattattttttgataattatcttgtcatatcaatcccctaatatcttgaggcaattaaggctttactcacaagatcaaagacaactgaggctttctgaataggaaaattgttcaaaaaaccataaatttattgtgcaatggtcaatttaattctaaatatttcaattttgtaatttagttttaaaccttttgacaacatgataattcaatcataaactttttgattgtgccaatttaattataaacttttaacggattgtcaatttagtcattttggctaattttggctagatatcgttgatatgatggcctagtcaacgctaactgtcctatgtaacatagtagacgtttatgcatacattaatttttaaaaaataaatttaatttttctattttttttttatattttctttttccactttttcaaagggctaatggagttactagccattgaggaagggcagtgaagcccttgctagtggtaggtgagggccacaacctcgccattccttgttgtggtggcgatagcctaggcaagggctcatggcacccttgccagccatagtgagggcttacgagcccttgccatacctagccagccatgacaccctcgctagattcagcgagggccgtgaccttgtggtcgagaaggcttcgcaatccttgcctagggcccaacgagggtcgccgattcttggctagtgaccctttgtaaaaagaggggaaaaaagaaaaaagattactaaaaactttgaataattttttaaacattaaaaattgttcacgtcagtaacgtccaatcaaaattaattggaatgaccaaatagacaatccgttaaaggatttaggacttatttggcaaattattaaaattttaggataaaattggcataattaaatgggttaggattggattagtgaattatttaaaaatttaagattaaattgataaattttaaaaggtttagaattaaattgactgtcatataataaattaataattttttggatatcttttccctttcagatccataaagcgtggtcacaaattttgaactttttgcaatcgatgtcgatccaactcggatccgtttaaaaccgacgcaggtcagacgactgagtactgaaagcattggcctatccgtcctcattgaaccaaacaatcaactgatgggtcgggccgctccgagattaagactatttgtcagagaatttttcccaattggtggtccattttcgtcctttaccttattcaaatggaagtctcgagatattcatgtaaaatcaacgaaaaataattttctaaaatgaaaagaaacaagaggaaaatttcgtgtgacatataagcagacgggccccctgtcccctgtcgcactcaaaacacaagatgacaaactcttctcgattagcacccattctttagaggcggttttgtgtccaccgcgtctcttatccggataaaatggctcgagtgcttaatccgtctaagcccacaatttttttttcccattttctcatttaatttacaatttttttccttttttccctaattcgagtggaatttacattttagaatttagaggtcgagtggagctataaatagaggggcacggtttgcaatgtaccctcaagcaactaggagaaagtgataagccttgtgaggtgttgaagacagctttgccccaaggggagatcagtg

SEQ ID NO: 18 - DNA, 2000 bp, Eucalyptus grandis
  source 1..2000: mol_type = genomic DNA
ggtcacctcctcccgcttgggaggagacccttcatttccttgtaccacgcccttgtgtttcaaatgggaaggcgtcgcaaggatccaagcggggttaccccttgtggccaggcccctagtaggggatccatttttactgagggggatcccctccctctatcttgtctttccaagatgtcaaaaccttttaaggtcttgcttatgaccaaattgtcaccactcttatcccggtctagaggactaacttagatataacagaactgcaaacatgaattcaacgtagatattaatcaagcaagaggctgttccttacatttgaataatataagatacagctgtatttagatttgtgtgaaagaattataattaaaatatatcgagcgccggcaccacctacaatgtggcatgtgatggtcattgccgttcgcatcattcggcaaggatacttataaaaaaccagtgtttccaatcaggtgtttacacgccagtttttgtcctttatacgaaagagcatcgggcaacttgtgtgactagtgatgtaccgtaataacgatcgcgtcaacgaggacagagatcagcaaaaccaaggtgtgcgagagagagttaagcaatttcactgtattcacattcaagttcgcaaatgaaggtttgacttttatgggaatatgccgtggagatgctgaagctcacatggctcttctcggtgtcagtagactttaaatacaacccccgtccaacatttatctacgactctctctctctctctctctctctcttctctcggggtgagcagagtttaaatacaaccccatccatcccctcgttctctccatcccctctttctctccgtcccaacccaaggttggcgacattttcttacaactctccctctccatcccctctttctctccgtcccaacccaaggttggcgacattttcatacaactctctctctctctccatcccctatttctctccgtcccaacccaaggtcggcgacattttcatacaactctctctctctctctctctctctctctctctgccctccattgttgccgcgagagactgaggttagtgttcattgctcttttattgttgcactcgtttgtggtgtgtcttttgcatttgcagacgacgagaaggggcttaagaaatttggttcgtcacgcttgacagttgaaaaaagcaatcttgtatttagccatgttttggttacgtagaattttagcggactctcttgtctaaggagaatgataacccgcacactgtcatagatatagcaacgggattgaattactttgttgttaagcattattcttgtcctatattcagtagagctatgttggtacccgaaacaggaggtgacgtgacaatttcaaatggcgtctgaaattttgtagacaattacatcatctgtaaatgtctctattggtttctatggttgaagttcataatttgtttttctagaattgatgtcgttttaagaattcttatcaactttgatctctcttgaagataaaacgaagaatgatatgacgcctttatgaagtgagatacttaaaggagtaatgagtcgaccggttgccattgaagttgcatcatcaaacaataattggcataattagattaattgaccatctcgaaagaagtacaacagacagcctcacatcataaattaaaagttttgttctgtacccgccaatcgaaatacgatttatacatccaatgatgtggtgttagaatcttgacatgttcttgtgcttaaaaatctataaaatctaatgtaatatcataaactaaaaaatttgaactagggcacgtaaaaacaattttctttaccccgtatttttacttaagatcattgttggtttttattgtctctcttttccctagatatatatatacttccataatcacttaaatataatttgattatctctttcatgggcaggaaaattgttgaactagttggaaggcattgaactccagcttcgatc

SEQ ID NO: 19 - DNA, 2000 bp, Eucalyptus grandis
  source 1..2000: mol_type = genomic DNA
caccacaatgagcaaataaaaacaaaaaaaatgagagaaataagacaccaaatgaggtgaaaaaaatacttccaaattagatggagggaggaggagaaaagaaaaccaaagcccaaaaaagaaaagtaaagcaaagcaagcttttttgaagggatttttttgggagagagacggttaccaatagaaagattaggattttttgttagttagagagttcctcgttagttttgagataaataaacgaaaggaatcccaacgtgaataacaaacattcccctttttaggcagtgatagagcacgtagacagataaaatgcactctaatttttcgctactggctttgaatgtgagggataaaagtgcttaccacttgggatttatttgcacgaatctaactctcaaattccacaagttgagggagaaattgaagcggcccaacccgatggtagtcttctctctttcgtctcccttagaacggttgggctgcccatggagataaagcacagacacatcctcctcttctaggcctaactttttgatactcaccggcgatttaacagcagccatggatctctctctctcctccgacatgggagaaagcacggccgagccagctaacgaaaattccgaggccgtatctcgctaaaggctcgggctggccggaactagcactcggcctgaaagtccttgtccgatcatcctcatgcttaaaaatctttaggttttccatgtagaaccaaatgggcctagacggcccagtagataggttaggctcaaacaggaatcgctagcccaactcacccaactttgcccgatcggacttgggtaggcccaaacaagcgcgaggcatttgccggctgatcatgtttattcttcgctaatgaaaaatatttagctctcaagggaccgtcaattcaggatcgtgaagttacgtgcgctagtaaagttcatcgactagtagtagatgcggatgacaagtaaatctacacaagcgagacataggcaagtcgtttcaacctcctatcccattcataattaatgaaatgagatcttctaaaacgagtcatcgcagtctgttcttctatctactaaccatatgatgtttgctttaataggcaagtcattccctcactctgcatgactttcataaatttttcaccgtctctgtgttacatttctttatcatagagcattaagactagatactgagataggcaaactagataccttcattatgcccccgaagatgaagccgagataaagaaattaacttgacatgaatcgggatggcaaacctctaagaacagtgggggcaaggttgggcaactcgcccggcccgctctacttcattccattccattccattccattaccattccattaccattaccattaccattcccattccccatatcttcatgaaaggttggtggacaatagtggtgcttatctcgatcgtacctttccgctattttattcatgcaaatagcccttcggcgcgtccattctcatgtcacgaggaaaatgacataaggacatgattagggtcgatggtcctttgcttccgcgaattattccctctaaaaccgcatttaataatctcccggaagagcgacagggactcgcttgtctcgttgcacgtaaattactagttaagctggttgtccttatgcccataatcgcccattattacacatttaagaattcatcggcggttggtcggcgcccaaccaccgcgctccactgcatccccggtcccaatgggcggtgccggctgtgccctcctcgtccatctctagctaatatatactcgaattagcctgataaaagattaaggagatggattaaaataagaaagccactattcgaaaggatgcctttagggcgagtatatatgcgcgtcttgttcgccggacttgacgcacaagctcactgcgaagcatagctaagcgagttctctttctaaaagtggttgattctagttctttcgatcgagcaaac

SEQ ID NO: 20 - DNA, 942 bp, Cauliflower Mosaic Virus
  source 1..942: mol_type = genomic DNA
gatctcctttgccccggagatcaccatggacgactttctctatctctacgatctaggaagaaagttcgacggagaaggtgacgataccatgttcaccaccgataatgagaagattagcctcttcaatttcagaaagaatgctgacccacagatggttagagaggcctacgcggcaggtctcatcaagacgatctacccgagtaataatctccaggagatcaaataccttcccaagaaggttaaagatgcagtcaaaagattcaggactaactgcatcaagaacacagagaaagatatatttctcaagatcagaagtactattccagtatggacgattcaaggcttgcttcataaaccaaggcaagtaatagagattggagtctctaagaaagtagttcctactgaatcaaaggccatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggac

SEQ ID NO: 21 - DNA, 435 bp, Arabidopsis thaliana
  source 1..435: mol_type = genomic DNA
atggattacagagaatccaccggtgaaagtcagtcaaagtacaaaggaatccgtcgtcggaaatggggcaaatgggtatcagagattagagttccgggaactcgtgaccgtctctggttaggttcattctcaacagcagaaggtgccgccgtagcacacgacgttgctttcttctgtttacaccaacctgattctttagaatctctcaatttccctcatttgcttaatccttcactcgtttccagaacttctccgagatctatccagcaagctgcttctaacgccggcatggccattgacgccggaatcgtccacagtaccagcgtgaactctggatgcggagatacgacgacgtattacgagaatggagctgatcaagtggagccgttgaatatttcagtgtatgattatctgggcggccacgatcacgtttga

SEQ ID NO: 22 - DNA, 1086 bp, Populus trichocarpa
  source 1..1086: mol_type = genomic DNA
atggctgtggagcttatgatggggtattctggcgatagttttgctacaaaaatgcaagagaatgatgtgagagaagccgcaactgctgggatacaaagcgttgaggaagtcataaaactgctcaaacaaaatcaactggaacagcaacaaaaacaacaatactaccaggagttgtctgcagcctcctcaagttccaatcttggcacggataatatcatggctgttactgatatggccgtgaacaatttcaaaaaggttatttctttactgggtcgtaccacaagaactggccatgctcgatttagaagagctcctgttgcctgccctcctcaacaacgaatgcaagaaccagaaccagaaccgcaacagcaaaaacagcaagttcaagagccagtaccatatgttcgagcaattaattcgcagccaacagagcaaggctctgcttttagagtttatcaaccgaccccaattcatcgtctccctcctttgcctcacaatcagcaacaaaagacactggtggttacgaaaaatggattatcagatcggaatgaaatggctactacgatcaatttctccaattcgccaacaatatctgctcctacttctttcttgtcttctgtaacaggggaaactgatagcttccagcgttctacgctttctgggtttcagtttacccaaccttctgctggtaaaccccctttgtcctcttcttctcttaagagaaagtgtaactccatggatgatgctgctctcaagtgtggctcctcttctagtcgctgccactgctccaagaaaaggaaatcaagaattaaaagggtggttagagttcctgcaattagtagtaagatggctgatatcccacctgatgattattcctggagaaagtatggtcaaaagcccatcaaaggctctcctcatcccaggggatactacaagtgcagtagcgtgagaggatgtccggcacgcaaacacgtggagagagctctagatgactcgatgatgcttgttgtgacctatgaaggggaacacaaccactctcatccaatcgatgaagcacccggtgctcttgtccttgaatcatcttaa

SEQ ID NO: 23 - DNA, 1707 bp, Populus trichocarpa
  source 1..1707: mol_type = genomic DNA
atggaaagagataagttgtttgtgagcgagggagcgaacacagcagcaaccatttggaattcttgcagttttggaatggaaatacaagccaatgagctgagttgtggtccagagaaacttgtcaattgctttctcaatcccaattgggacaactcattggatcagagcgatccctttgagtctgctttgagctccattgtatcctcacctgttgcatccggtgccaatgcaaacgccaacgccgttcctaatgctggcgttggtggtgacggttttatgattagagaactcattggaagactaggaaacatttgcaattctggagacatttcaccacaatcttttgttaacaataataacaatagcactaacacttcttgctatagtacccctttgaattcccctccaaagctgaatctttcgatgatggattcacaaatgagaggaaatctgccaattcctgggaacagcgtagtaaagcatccaggtttagcaccatttccagctgattttgtagagagggctgcacgattttcttgctttgggagcaacaatcttggaggcctcaataaacaattcggattgaatgaatccgaattgattaataggttgatgccacgagtagaacctggtaagctctcgagagtttcgagtaacaattcaatgaaggtcactgtatcgcaagcaaatgttcaagaaagcaacaagagctcaccccaggatgggagtttgaattctgataaaaaattcagtaggcagtcaaggccttcaacatcagagaatggagattccagggaggaatcttcagtgtctgagcaagtcccaggtgggaaattgagcatgaaatcccagaatgacgccaattccaggaaaagaaaatcaattcccagaggaaaagccaaagaaactccctcttcatctccatctgcttctgatgtcaaggttgcagcagagaatgatgactcgaaggcaaaaagaagcaaatcggatgaaactaatggcagtgacaaggatacagcaaaggaaaaggaagaagaaaatggaaatcagaaacagaacaaaaataattcaaagccgccagagccaccaaaggattatatccatgtcagagccagaaggggtcaggctacagatagccacagtcttgctgaaagagttagaagagagaaaatcagtgaaagaatgaagttcctccaggatcttgttcccggatgcaataaggttactggtaaagcagtgatgcttgacgagattataaactatgtacagtcattgcagcgccaggttgagtttctgtccatgaagctgtcatctgtgaatccgagaatggagttcaacatggaaactctgttgtccaaggatattttccaatcccgtggatccatgcctcatagtctttatccatcagatgcctccacgccggcattcccttatggataccaatcccagcaagggctggccctgcaaaatggcatgccaagcaatgcagaaacccagttctccatgaacccattaaacgctgcgttgcggcgaaacccgagcatgcatctgccaccccttgatggttttggtgatcctgctgttcttcaggcctcagccatgtgggaagacgaccttcaaagtgttgtgcagatgggatatggtcagaatcatcaggagagctttcaaggctcagtgccctcaactcacatgaaaattgagctgtaa

SEQ ID NO: 24 - DNA, 1044 bp, Populus trichocarpa
  source 1..1044: mol_type = genomic DNA
atggagggggttgaagaagctaaccgggcagctgtggagagctgccatagagttataactttgctatcccaaccccaagatcaggttcaatataggaatttaatggtggaaactggagaggctgtgtttaggttcaagaaagtagtttcccttttaaatactggtttaggtcatgcaagagttcgaaaacttaagaagttaccgacccctttatcccaaagcatccttttagacaacccactgagcagtacaggccacccatccaaaacatcccagtttctccagtccagtagttacctggaaagccaatcaattcaagaattgggctcaattgctaaaaattgtctatctctgggaaccccatccctggaattgagttcaaatgggaaaaaccctcttcagcttggacaacccacgccagcagcgccctatcagttccttcagcaacagcaactgcataggctacagcttcaacagcagcagcaaatgaagcagcaagctgagataatgtttagaaaaaacaatagtgggatgagcttgaatttcgatagttctagctgcactcctacaatgtcatccaccagatcttttatatcgtccttgagtattgacggtaatgtggctaatttggaaggaactgcattccatttaacgggggcggctcgctcctcagatcagagttcacagcaacacaagaggaaatgttccggaaggggagaagatgggagtatgaaatgtggaagcagcgttagatgtcattgctcaaagaagaggaaacatagggtgaagaggtcgatcaaggttcctgctattagcaacaagcttgctgatatccctcctgatgattactcatggagaaagtatgggcaaaagccaatcaagggttctcctcatcctaggggatattacaaatgtagcagtatgagaggttgtcctgcaaggaagcatgtggagaggtgcttggaagatccgtccatgcttattgttacctatgagggtgaacataaccacccaaggattccagcacaatccacaaacacgtaa

SEQ ID NO: 25 - DNA, 906 bp, Populus trichocarpa
  source 1..906: mol_type = genomic DNA
atggctgtggatctagttaggtattcaaagatggaagatcagatggctatacaagaagctgcatcagctgggctcgagagcatggagcacttgatctttgcactctctaaccaaactcgacaaagccaccaacttgactgcggagaaatcacaaacttcaccgttgccaagttcaagcaagtcatctccatgttgaaccggaccggtcatgcccgttttcgccgtggaccaacttcttctccttcttcctacccggttcccgtccgacctgtccctcaagaacctcaaaaactgaaccttgattttgttaacagtaagagcccccctaaagctgagtcgaaaaatgacctgtctttgggtagtcagtattcaaaggatagccttagctctggcaccactacctcatccttcgtgtcttctgttacagctgatgggagtgtctctaatgggaaacaaggtggctcttctcttttcggaactcaagctcgatctaccggaaagccacctctctcatcgacccaccgcaagaaatgccacgatcatgccctttccgccagaaagatctcctccggtggcagctgtcattgctccaaaagaaggaaatcaagggttaagaggacaataagggtaccagccgtgagttccaagcttgccgatataccagcagatgagtactcatggagaaaatatggtcaaaagccaatcaagggctcaccatacccaagagggtattacaagtgtagtagtgtgaggggatgtcccgcaaagaagcatgtggagcgtgccgtagatgactcggccatgcttattgtgacttacgagggggagcaccgtcactcacatactccgttgccggaagacgtcacggctagtgctgcaatgcgacacgtgtttcactcaacatga

SEQ ID NO: 26 - DNA, 1802 bp, Populus trichocarpa
  source 1..1802: mol_type = genomic DNA
gcgagcctgtcaaaacaaaccattgacaccaatcttggatagaataaatgtgggaagaggaaattgaaaaaaagagagaagaaattggcaccgtggactgctaataggtctgcatctgaaacaatggattgcctaccccaattagtttttgttcatcatgtactgtgttctcaaatcaagatcatacttgggaaagtacaattgacggtttggaattaacggtgcagtgttgatctcatgttcttaaaattcactcagtcatttcaaacatctaacatgatcacacctatgaactgtcaacaacgaaaagttgataaaagtaattttgcctttcaattctcttttgaaaatttgccagggaatgctattttgagccgttggcgtttgagaaggttgaaattgcctatcttcaaaagatgctggacccgttttctatttaaaaaaacggttcgaatttagagagaaacggctcttttggagtcacattaatgtatttttagaagttgggtaaaagttaatttttaaaaatattttttatttaaaaatatattaattttttaaaaaacataattaaatcacagtcataaacgcgtccgtagtgagtataatctgcagggcggcattttttataataaaggtaatttaaatcaggtcttctttccggtgacttaacactgattttactccgactccaacttaattccgtagataattatgtaaaataaataaatgacgcttaaagttaagctttgtcccctaaactccttaatttaatttaatttctccttcttcttattaactcgactgagttatcgatactacgtgcaatatattatctccactaatggatattatttatgtagaaccaaaaaataaaaaaataaacaattacttttgatattaattaaattccaatatcaggaattgagtaaatagatattatttacttttgagattatgatttttttttaaatcatcgatgaggtatttaatcaggaaagatttaaagcgaaatccgaagcaaacatctttcaactccctgaaacgattttgttgctgtatttattgttattattataactattcatgaattaaaaaaaaaaaaagtctcttgtgagtcccaaacaaaatatatgtgcccattaagttaaagaaattaaactaaaggacaaaacacacctttgaatttgagtaaatagatattattttttaaaatattttttatttaaaaatacaataaaataaattttttatattttaaaatttatttttgatattactaacaaaataataaaaaaattaatttaaaactaaaaatatttacaataacattattgaaacacaatgtgcatggtgtagaatatgtagatgtagtttaatattacgacagtatttgttttttaaagtgttttttattttaaaatatattaaaataatatatattttattattttaaaatttattttttaaaaaaaataaaaaaaatttaaaataaaaaataaaaaaatttaaattttttgaaagcaaaattaaatctcgtaggcactagaaaacgtcgtaaaatatatttttaactggaatctagcacagcagcagcaacctccctagttctcaaagtgtctttccatttgaaaaaccattaaataaatcgtttctcctcctcctcctcctccctaccatcccaacacacacacaaaaagaagaaaaaattcaaacttcaaactcaagctcagatcccatcctctctcaaacgccaccttttcctccagatccagcctcgatctaccaaatcaccgtcaccg

SEQ ID NO: 27 - DNA, 2000 bp, Populus trichocarpa
  source 1..2000: mol_type = genomic DNA
gtgttttcccatatgattttttatgatttttttcaaaattatctttgtcgatttttgtttaatattgagttggttaagaattataattataatgaagctaaatcatatgggaaaagcgttgcagattttctcacaaaacactatgaattgctacagtatttctctaaatggttttttattttattttatcggggaaagcattgtagttttcctcacaaaacattatcaattgctacagtgttttttttcataggtattttcattccaaaattatctttgttgatttttttttaatattaagttggtaaagaatttagctttataattctttttgctttttattaacagaaaatctaaatcatgtggtgaaaacactgtgtctttcctcccaaaacactgtgaattgctacagatcattttgttaagtctctaagttttttatcaccaatataactttatttccatcataaaatattaattctatcatacttttaatttttattatttatctaatactgatacataattataacattattaatacttgtgtttataaattcatggcaacatgcgcgcatgccatctcatgataataaaatacaggagttcttaaggtacattggttttgccacttgcatgacgttgggtttctgctcagaacgctggctgacctgttgccaagaatctttgtgagcatgccatggcccaaattgaatataacccgagttatatatatatatatatatatatatgggtttgaccaataaacaatttcttcgcaaatgtttaaaagaattataattatctccttttaggatctctgttatgctagtttgaaaatgtaattgtgattattttttaaaaaaattttatttaaaaatatattaaaataatatttttttttatttttttaaaattatttttgacatcagcacatcaaaatgatttgaaaacattaaaaaaatattaatttgaaataaaaaaataaaaaaatttattttttttttaaaatgcttttacaatacaaaaacaaatcaaatctaaacactaatactttcattaaattggtataagtcacggcaactttcactcaatagatctttcatgtttttatatataaaaaaatttagacactaatactaacttgattttataattaagttatttcttatattaatttcaacttaaaactgttttaattcgtcgtcaatgacttctgttaaaatcatccaacaagaattattattattattattattatttaaaataatgttgtttctgttttaaaaagtataattttggtaaaaaaaatataaaagttttaaaaaattcacataaaagttcttaattatactcttttaatttaaaagtgaaattaaaaaaaaatcatttgacaataaaatcgaaaggtgggtatttttgagatctacttttcaagtttctagcaacggaaaaccgtgaaacttgtgtggcagcaaattcaggcttgacctcgcaggctgctgatgtcagcagagagctcatgcaattagtccacgacaagagccttccgttgctgggcccaacaactttgagcccatcggacatactccgaatattcccacttccagaacattaagactcccacggtcattaaggaaagtgcgctataccttcctcttacgcctctccctcatagataaacaaccagtcaaccacccaagactgaatccaaatgccacaaaatcgcgtgaatttcctttttaaaatcagcggctaatacaacggcttttcaaaattccaatcagcggcttaaaaccaagctaattgtttctttcgttttgcccaagaaaaaaaaaaccgctcacgtgacctaaccccaccacgcacctactcttctttatatgaccctcaattagcagccactctcctcactaaaattatctctgtttaagcttatccctcaaatctcccctcttaattctaaatctctcctctgtgtcccgttactatcaacaaca

SEQ ID NO: 28 - DNA, 2000 bp, Populus trichocarpa
  source 1..2000: mol_type = genomic DNA
agttcgcgtctcattacaagcgtggggtgaagatgatcaattacatgtaactttggttcttgagagcaagattcttgctggatcaagttgtaaacttttcttcaaacttcaaattagtaatctaagagacaattccacatcaaatagtaattaacaggacatggatcaaatgttaaggaaaaaataattaaaagggttatcctaaacttttaaaggggttgatgcaaattttaaaggaaaaaaatggtcgttggtgccaaactagaggtgcatttagtgcacccttcgcggggggctccaaaatctttcaaacactatcatagaaggcgatgtttggtgattaaatgtccccacacttgtcgtccacttgttagggcatgcaaaaaaattttaaaattattttttaaaatacaaaaatacccctgagttatcttgatattaaaaaactatgatgaaaagacaaaaaaaaaacttgaaaaatagttttgttaatttcaaacccaagtataattaagtaatttaacttcaatagaaaattaaaaaagacataaaatcctctgggtttaagcccttttttttttgttccgggatatttaaataacttaactgttttttttttttaaaagactaatctaccataaaataatttgaaaatgacgttgaagaggatatgtatttttttgtttcgacagtaagaatattataccgagctagcttgtgcgtcaacagaaattcaaatttttttagaatgattttaaaaatatattcttcacattgacatgataaattaaaaataataataattactaatactaagatttagatttaaaaaaaataaaagaactttcttaattaccgaacaatttcataggaggtaatggtttacagtttacaatacggcgatgatttggtttcaaggacagtcaaaatcggaaattgccccctgacgaccccaggggaagaaaacaataattacatgaaaagatgagtggtcaccaccactaccaccaccttgtaaaaatgtatgacgacccaaaacaaaaacgccaactacaccattaacaccacaaaacccattttaggcaactaatccttgcaccaggaagagggagatgtattttcatgcctcttctaaatacaatcatggtaattacattacattaccttacccatccccctctctctgtgcattaccgtaaggttataaagacagctcaatgcaaaaacaaagagagagagggggagaaaacgacttgttattattcactagaatggcattggttacaatcagaaaatcatacaccacacccaataacccaattgagttatagaagagagagagaaagggactctgagagagttgcaggttttatagcagaggtctctgacagtaagagagtatatattttaactttatctatcaaataattctctcttactctctctctctatataggagctagctgcttgttctctttatgtttctctctttcacacttgtatctttttggtactgatgaatactcaatgagtctactgtttttcttagagagatagagagacttgtgtgttgccagaacgatgtgatggaacgatataaagccaagccaagccagactgagagctagtcatgttataactacattacataagccccaaaagcaaaaacttattcaaaaagaaaatcataaccccatttgcataaacccttctctttctctgaaaaccttgtattttcatttcattgtcggcgacttcttctcaatgagagcttaaaagggtttcttctttctccaagttttaagtgacgggaatggttattttggctgcctagagtggttaagtttggaggaggggggtttttgaacgatggttgttctcaagtgagcaaattttggattctcttttttggaagagcagtgaagtgatggcatttcgtttggaacttgtcctttttgctcttcttctctctttggttgttgattctgtgcattctgatg

SEQ ID NO: 29 - DNA, 2500 bp, Populus trichocarpa
  source 1..2500: mol_type = genomic DNA
aacatcttggatttgtttactcatcaattggccccttatgtgggaactgataaaaaaactatcaatttctaaaacttatttttgacactttcatgtcattgcatgacacacaaccaaaacactcatgggttaagatgcaagataaatctacttgttatcacgaacatttctacccagctagactttcaacaactcacaagttagatgcaacttctcacccaaaccgtattggtaacccaaggtcaaaataagaccttaccacccccaacaagtcttggtaactatgacaacacacttgatcccatcaccatttccactggccgctcccaacgcccatagcccattgcactacccataagagcctgggtagaatgacctccaagtgagtattctgcctccatactcgtcaagtccctccaggggtcgctcgcgctattgtgatacccgtgcacatagccaggactccatattgctcccctaactaccgtaggatggaccttggttaccaatctcgttgtttcatgccaaactttttaggccatactcgtcaagaccatgaatgatgaaattgatgctattgaaaaaaaacaacacttgaaaatcaatgaagcttctaaaaagacaaaaaaccattgatgtaaaatggatttttaaaacaaaattgaaggggaatgataggttgacaagtataaaacatgcttggtaactaaaggtaacaagcaagagtacgatgtcgattactaagaagtttttgttttagttgccgggtatgatacaataatattttttctttggtaactcaaaatccatggtttatgttgaatgtaaaaccaatatttttacatgaagaccttggttataagagagttcttcatcattaaactgattgttcaagaacatcgatataaaggagaaatagttagatattagttgaaatcctattccaagtagaaattcttgatagaaaaggatttgtgtctaaaatccttattgaattagttaattataaatcttattggattatgattgttagaggttgcatcctataaatataaaacttttaccagattttctgtccttctgtagtggttgaaaaatgttgcagggccatgacgcacgctatttttgaaagaaaaatatacaggagaaatagacatgttgaaaacaaagaaaattcgaggcacacgcacttggtttttatagttttgtactgggcacatgggttaaaatctttcaggcctaaccatacttggttttatttatttatttatttatttggctctaatttgtttttacattttacaataaaatcaactgtaaataaatttattcagacattctcttttctagaaaaaataacgtgtcttaatactcgaatatcccttcttaaaactcacatgtacatccaatttatttttattatatgagaaaaaaatagtattatgttccaagcttacacttgaaaaaaaacaacgaaaaggataccgtttgattacgaacgcagcatcggaaaataaaataagaatttcgactttatcctcccggaggggcctttgatcatcattcccggaatcttttaaatatatcacagacccacttccccaccttttgccgtggaagcaggtgctcactccacaccgaatgacccaaagtcgttaaaaaagcaaatggagtctctgttctttttcgcatttatattcaaatccgaaaagaaaaaggtttaattttacaccacgcgctctatatagcgtggcacgtgacaatcgcgttttgttttttttacatcatcttaagtgattttcttttccttttcaatgaaaatatttaccctttcttcatttgtaatttcaagagattatatattgcctgcactttaacatgacgtcaatgaaaccgttaatccctttcaaaataaattccgacatctgtttttttatccaaagcattctgtatgtatcttttaaatttttgaaaaaaatggttttttataccaacagctcccttttttttaatattttatttattctggtcggaactaatttctctgattgaacttcccttttaactttttttttgtaattataaattaactttattaagctaaaaaacaaatattttagaggtaattatgagtacgatagcgttgttatttttaaagtatttttacttataaattcatttaaataatactttttattttttaaaatttatttttgatattaacatatcaaaaataatttataaataaaaaaattaattttaaataaaaaaattaaaattttatcgaaactcacaccagcaccgaacgggccgaagttgctcacttcacctaatgttttatagataaacaaaaagtcacgctagcaattcacagggaacagtcaccaccaatatcatgctgccagatgaataaagcagaaagtcaaaaatggtcacatcgatgacccgcataagaagctttcaatccaaatgaacggtgggtgatggggctgtgagagca

SEQ ID NO: 30 - DNA, 2700 bp, Populus trichocarpa
  source 1..2700: mol_type = genomic DNA
aacatgcctgtaataataataatttaaaaaaattgatattacggaagttttgctattgtgcgctgaaacggtggtcttaatacaagctacaagcggactcctcgttgacttaggttttgatttatagtattaagtcaagcaatagattaattaggattattattattattagcatgggcttatgtttagagtaattgtgttaagagcaacagggaccacgtacgtgcctgatagtgtcacgcttcccaataacatgaaaccgccgtagccctcaatcactctgactgcaggtggcatatgcaacctttataaaaatcgagagaaaagttgacttgttaaagtcagagatatccatggctgtgattttgcccttcaatacccttaaataacaggcttgttgtatctgctatgttttttttttttttttaaaaaaatggattcacgtcatgtgtgtttaaacaggagagcgcatgattacaggaggaggagtgataatcaacccacaaaactagaaagcagcaaaacgacggaatgtggcgcatttgaagcgctgaaatcgagcgtacaccgttgcctctttttttttttttttgtctatatctcttcctctgtaaaccccattaatttaatgcgcctctcacttcccactctgtagacactgctacctactctctctactctcttagctctgtctttctctgcaaacactgtgctgtcagttgctgttgtggagatatataatggtggtggctgtgatggaggaggtgcaccggagtagctgaccctaggacattccttattatataaaaaaaaaaggaaaatcttgtttttgccctttgtcattcgcgtgccccctttttaagcctagtgtcagtcgtcagatctctctctttctacatgtttaagtttgttagtgtttttctcgaaaaattaattttctttgcagtctcaagtctaatctctcatttctgtctttgctctctcgtattctgttccgttcctgctcgttcctctctctctagctaggcaggcatctttctccctctcctccagggtttgttttagcccttttcaattcccattttatataaattaacagtattacattcgagatgcgtcgaaaaagaagttcggtttcaaccacttgaagaagaatgtacgtgaccatgttcctctttaatatagtttttcttttcctgctatcccttgcttcttccttccaaataagaggtttggttgctggattgtttttttctgtatgtatatatatatatatctctggatctaatgactgcagcttttgctttctcgtttattattagagtagtaaaaaaaccgaagtaacggtaaagattttcacacaaaaattgttttgtcagcccggtaataaagtatagttgcttgttgtgttttttttttttttttttgagattgtgttgtttcgatgaagagtgctattgttaatattttataacattgttgcagtagcttaaacgattatctttttttcagatgcctctgatttgtggctaatatataagttgttttcttagatttttcagaaatattgatatttgaaacacaaccattagatttggaggacgttgatagaaatttggtttttttttttcttaagctgcaggtctatgtatagttttgtattttccgttgtggcttgaaaaaataaataaataaaagttatgatatcaagaacaatggatcatacggttaacaagtttcgctttttgcaccttatttgttggtaattgattatgggatgctcacgaccattattagtattttgtgcagcaaacaaatccacaaagatgcatttgtcataatatgcccttttttttatgctgaagtttatgattgcttgaggaattctggtagatataagtgaagtttcactgaattcttttgggtttacaaatcattgctatacacgtttttgggagttacatatgatatggtaggggaaacaaggatttgatgagattgtgtatttggatgaatttctcaagtattagaccacaccacatgctgtttcgttgctgatgcctatggcccaataccaatggtgaattattggtgctgagcatttctaaaagtggagctacttggtgaattattgcttgacatcatttgctgtttgggctggaattctatcatcataacccagtaaattgctcaatctgttatcggttgtaattgcttacttgtgcctgacttatgacatgataagaaattcatcttgcttattccgtgattgtttgttgccaatgatagaagcaatgtgtttgttgtgtgttttaggattattttattttttattaattctatgtctttttatggatactgctactaaacttgttcaggtgtgtatatatatcatacacatgaaggcgagcttttcattactccgtttgtacattcttgatgcaggttatggcccttgttcgcgggtccaaggttgataattttgttgggatccttatgttaatttctgcagtgcggacctacttatttagatgttatatattcatcttgggctaaaccctgcttttcttttgatgtggaatagtagagtaattatttacatgttttgccaaggtgaaggagattgatgccatgttctgctatgtttcagatacattatgaagagttcaaggcaatggaaagtggtgtctacacctagaa

SEQ ID NO: 31 - DNA, 2500 bp, Populus trichocarpa
  source 1..2500: mol_type = genomic DNA
ctctataattgtagaaatcttagattattgaacctgaataccatcaacaaacacaataaaccaagaaaaattacaatactagtaaagaacttaaactttttgcgtttgacatataattgttctttccttaaagtgccaaaaatagcccttaaatcctcagaatataactataaatcaggatgtcgttaaaataaaccacaacaaatttgcctataaatggtctcaatttctaactcataaacctcatgaaagtgctgagagtattgcacatatcatatagcataaccatccattcatataaaccatattgtgttttaaaggctatcttttactcatcctcgacttgattttgatataatggtggtcattcttgagatcaatcttggaaaacacctttgaacctgccagttagtcaaacatgtcatctagttatgatattgagtactagtattttattgtgacctgttaatggctcaaccccatactcatatacgtcatgaaccattatttttagggaataagagagctaaaactttacatgagctcatactttcttaaatatactccttttttagcaacttcaccatttgtttcaacaaattttcttcttttttaaggctagtctataagttagtatattcaatagactaaaacttagtactagatcaatatggtgttgtatctttctcatgaaaggtaatttctagggcaactcttttgacatcaaatctagaaattttgttagcataccatgcatttaattgtaaatgtcattaacattgtcctctttcccaactgcatttcaacttgtagtatcaacttctctcaacctcgttcaaccaatttatgaattcctctatttgtaaaattcctactaactcaaggattttgatatgaaatccaaaatccttcaatatgttctctttttgatcaagaggttgtctcctctgattctgcctttggtctttagtgttggctagggacccatggttgttcttctagtcttggtctcccaggtccctcttctctaacctgccgttcaactctgcaatctgcctccgtatgtcctcaatgatgaccttctaaatgtttgtctctaatgaggtaacctcctaatcccctccttgatcatgccacacttcgtaacctcatcctttcatggttattaatagcaaacaacaaataaataagtatccacaagattgtaacacaacaaacaatgaacctgagatcattaggctctgataccaattaatactagcaaaatcacaatagacaacaacaagcataaaaaaaatagaatttttgatgagtgaaaaggctgcagatataagccttaaattcataatttattattctaaaataaagtctaataatatctacctcctaagaggttatataagatatttaaatatgtcagaaaaccctatctctattaggatacaaatcaaaatttgaaaaagaaaaattaacataaaatccaaaataaaataataaatccaaaaaaatcaagaaaaaattacaaaattggcccaaataacatcttctagatctaattttgggagatccattagtttgatgaacgacaaatcatttttttatatgaacatggccgttagaattatctaatcaaatttaaaatcgattcaacgatcagatcttctgttatgatttgttttagttattattttggtctgacataaccaagctttttttttgacctagacttttctcaattatccataaaaacatttattagatcatccatatagtttgtttgggacaaaacctcattcaaatgctcaaaatttcagcttttacaatctcgactacattgtctcattacatatatgtttagatgaagttttattatatatatattttttttttgcatgtacatcattttcaatttaagaaactcctttgtcattgtgattacaatttaattttatatacaaattatgatgatttatttcttttaatttcaaagttttttgttttatatttaaatttttaaatatagtcgaatcgccgatgacaatacataataagtgtactttctcctctcatcttgcaaaaactagccacatcggatataggccacccaatctgataatggggttgggtaagggttaggttttccattgattttaatatttattgaccttttaattatataaacatttgttaaaaaaaatgaagaaatttagttgtactaataataacgaaaatacaaaaataatattggtagcgaagtaatagaggaaaggtaataagaaattaataacgaaaaaaaaaaaaaggacaggcgtgaaagggttctcagataaaatccttcccttccctactgtatttgtctctctctattactccatctctcggtagggcctaaaaagagagaaagatatgcaatatatacaaagagaagcttgagagattttaccttccttttcattttcatggttttctataatgagaagaggattgaatctcttcttct

SEQ ID NO: 32 - DNA, 2101 bp, Populus trichocarpa
  source 1..2101: mol_type = genomic DNA
gcggtcgggagagagggagaaagggtcatgtcagccaggcagccaaatctttatacttgaattcttgtttaccggaaatattgtatgtaatgaattttatccacaaggaaattttttttatgggaattatagccttttagatgcatgaaagttaccctagagtcatgccctaaagaatttttgttacatcgatcacatgcactgagaatgttttccactgctatcatgtgggaaatgactttcagtccctcactctatggtctatgcctatgaatgtctcttgtctaactgcttccagaaaagaggtggttttttgaggaaaaaatacgtcctccgagctgcatattccaccttaatatcctcacaaaatcagtgcaaattcacaatagatcatgactcatcaccacacgactaggtcaagataagaagttgtggggatgtttttccaaaccaatacaaatgtgtttgaaagttttgaagaaatagcatcgaggataaaaggattttgaaaaatagcacaataaacaaaaaaactacaataaaaatgaggtatttaaacaaaacgtagttgagaattgtgatatttaaatgaagatacctttatactaaatgctatttaaaaaagaaaaaaaaaccagtagtttctgtgatttaaaaaaaaatcgcatgcaagaatcacagattttctagctgtgcttcttttcaaaaaaacgtttttatacgtactagctagtcaaatttttaaaaaagctctgctaatttcttataataaaattgaaggtttcaccacacaattattgaaaacaaatttagtgcataaacgacatatttgaagtcaattttttagcaattactaccagaatcttgtccattagaggaaaaacatgctcagctcagtcgaggtttcctttcaacagtactcaataaactctggtctgtccacactgcccccctaagaaaatcacagctacgaagggcaatcatataagaaaaatttaccatttggcaaatgtttgcgataatgattcatgacaatgataattctgattaatcaagcacatctgctgtttatgtctgatcacaatttctttgcccacccaatcacacgtgctatatggaatcacttattgacagttcaaagtgcatgatgcccgtgattgcagtcagtcgaaatccttctgcaatcctccagtttcactgagaaactaatcataatgcacatgaccagaactattattctctccaaatgttattatacgtgcttatcatcaactaatcagtggtctgtcagataatacataaatatttaggctatggcttgtttacgcagttcatatctccgagtgttatttatagtctcgcttcgcttgaaaaattattaaaaaaaaattatctttttcaattcatacggagttgaaaatgccgcagtaccaaacaaacaaacacggaacaggacatgagaaggggggataaatgggtggggtttggtggtttttatagtttatactatgtgggaaattggagctacagatcccagagaagaggaattggatttggatatggcatgcactactgaaataaatatatattagggttatttatgttttaatttttcaacgttctctaccagttaaaagacgacggcattcaccaatatttatccgtatcttgaaaagcaaagaagttccattttcaagtttagggattggaggaatctttttcatcctctccctccctctctccctctctttactttatactgtaatgaaaaagcttgagagagaaccagagaggccaccgaaaagcatggacaccttatcatgaaagcaagaaaaaaacgaaccgatttgacgggccaacccacacgcataaacacacagttactcactcactaacacacccacacgctcgatcgatcacacatttgtgtcactcactgtgtgttaatttttgggtgtctttccttttgttaaaaatatggaaatcataagtcaacctcccctcatgatttggtcttggtttcttctaagcaccacctcatatcccactttctttagcaccctctatataattaccactccctttcattcagtccttcacc

SEQ ID NO: 33 - DNA, 1801 bp, Eucalyptus grandis
  source 1..1801: mol_type = genomic DNA
tggtgcgccgggaccatctaatattttctcgggactgaaacggtagaattgtcaactttttctcaacctttatgcgactgtgattggttagccatttaaaaaggataatagaatttaaaagaaaaaagttcaaaagagaaaagagaaaaattggagatttttaaaagaaaaattgcagttgcgatttgttattctctggaaaaaaaatataatttaaacgaaaaaagagtaaaaaagaaaaaaaagaaaaattggggatttttactaatggacggtggattgggagctgattatggaatctacgcgaacgatttattatattaaatattcagaaattcgcatgcgtgggacatctaggccaagtgatttcaagcgcgagatccacacgtgtcagctctagagtaatactgcggtaaaaaagtcaacctttgaaccatcgagctttcgagaggagaaaaaaattattgagcttaatggcgtcgagcaccggacgtgaaccagaagttaaatttcgtcgtataatgatttataacgagggttaatatctttaaaaatcttaaattattattacatgataccttaattccaaactaattttcacctcataaatgatcctaaatcagtgcttccatggcacttttatctcaaattgattctcatatcacaaaaatttccaaaccggtatctaatgtcacattcacttgtattaataattttcatctcactgaaaatatcaaactaagaccctaaccaagtctgccttccagtagtattgtgcacaatatcccattagaaatgacaactataagatagatcgacgtataaaatgctaacgaatagcaaattcgggtcgaggagtgctagttggggaatttttgttgaatgagaagtagttcattaacaaaaaattggatgaaaattgaattgagttaaaggtttaatacctcaaaaaatctaaaactataccaattgtaacacaaatagcgaaaaacttttttcttgtcactaaaaaacctcaaacttacccattgtaacacaatttccaagctttttttgagttataaaaaacccaaaacccatatcaatatgataaatatatcttaaatagaactaaaatagagaatatatatgtcacacatatataggtttaaatttttttgtgataaaaaaatattttatttatgtcatggtaaacttaatttggagctttttgttatatttaccttaagataaatatgcttaattgatatcaacttgtaattttcagtagtaattaattgttataataacatgggaggttagaaagtcgatgacaagacgatagcgagagggtgaggtgaacaggctgaataatgatggtcattagggttgagataattattatagattatgatgattgggcaaaagaaaaacaaaaaagaaaaagacaaaaagacaaaggaggacaaactagccgttccagtcccgcagcgaatccaattaaaattaaagcaaaagcggacggtgggaccgccgcgagcacgacgggtgtgctggacctcttttttattattataaattgtcattattatatttttgtttttccaacggtcagatttttgctccacagtgccgaatgtgaccgtttgagcatgtcattcggccggatataagaaccatctctccgtctttccatcccaattcactcctcaaactcctcctctcttctcttcttcagcatcatcatctagccagcttcgaaagagctttccttgaattactcagatttttgccaagcgaaatcaagggaagaaaaagaaat

SEQ ID NO: 34 - DNA, 600 bp, Populus trichocarpa
  source 1..600: mol_type = genomic DNA
ttttaatgcacttgatgacatattttcaagctattaaaatttaaaaaataaccgctatcttcctttccggcaccgtccgagcccacaacactacaattcgttgcctgccacttcatccaaatgtgatgtgaacttaaatgagtagagcccatatgtatgagcttaactaagatcatctggtcaatagccgagcaccacaagggtggtcaggtgcatcttaaaggggcttgaaagagagagtagaagccgaagctcgactcggcgataacctcaaatgaactagtatcacacatccatgtcctcccgaggatgagataaggtcctggagcttcttccacgtggcaaatccattgtggtgcctaacgataaggctagcattccaagaatttccaccctcatgtggatagaatggtgtcatgtcatcacttatcatatccaacggtcacattttgctcattatcaaccacgatagcttgtgtccataggatatgagatagacaagttgaggcattatatatacggtgtggcaggagtacttcaatcttacaatactagcaaaaccacatatagagggtgtaatagctaagtagcctgtaagag

SEQ ID NO: 35 - DNA, 1800 bp, Populus trichocarpa
  source 1..1800: mol_type = genomic DNA
taaaacttgatctctaataaatttaatattaaaagataaattaaaaaaaaaactaaattaaaaaaatatatcaagttatcaaattaataacttgaattataagattacatgattctatccaaaacaaattaaaataaaatataatgcaaaacataactcttgctttatccattaccatgacatcatatgtgacaattctccattatctagtaaatctaataatgaattataaatttttaaaaaactaaattataaaaaataaaaaaaattaataaattcaatatttcaacaaaccttgttttatgaggatatatatatatatttgttaaatcaaacatgtacaaattcataaaacaaaggacataatcaataagagctagcagggaagaaacggagaggagttgtggtggtagcattgagtgagaaaaaaataataatggtctgttatggtatgagcccacatgctctttacagatggccatgagaagcttctattggccaattgaaagtttgaaacatctagaccatttcctttgttttcccctgaatatgtagcattgggtttcataaatacactggaccggaccatcaaagccttgtctggtggactaaattgcatcatttaaacatttaaagggcctgatgcataattttgactgtgacacttaacaggcctacacggtccaaactcgagggacctgaacatctatggggccttcagctagtttagggtttcattttcgataatggaagctccatcccttttctaataattcaagtttaaagtttctcctaaaagctccactgtgcaattgaagattgagtgtaaaagacgctccaagagtcattttcgatcacattgacatgccaatcgaaagtgttcttttttattattattattaatgtgaatgtctggactaatttgcgcgtattttgattaattctacgagccatgaagttaacgaccatgtaaacttccaatgaccctgaactttatgagattcgaattggtaacttctagaaaacaaacttaaaacctgatcagataagctacacctctcgagattgtgttcttcttctaagcagtgggtgccaaaaaaatcaacccatgcatgcaatctggtatcaatttcttagaaaaaattgattttgaaaaaagaaggttacacataatttaggggcatggttcactttcattctttttaatgcacttgatgacatattttcaagctattaaaatttaaaaaataaccgctatcttcctttccggcaccgtccgagcccacaacactacaattcgttgcctgccacttcatccaaatgtgatgtgaacttaaatgagtagagcccatatgtatgagcttaactaagatcatctggtcaatagccgagcaccacaagggtggtcaggtgcatcttaaaggggcttgaaagagagagtagaagccgaagctcgactcggcgataacctcaaatgaactagtatcacacatccatgtcctcccgaggatgagataaggtcctggagcttcttccacgtggcaaatccattgtggtgcctaacgataaggctagcattccaagaatttccaccctcatgtggatagaatggtgtcatgtcatcacttatcatatccaacggtcacattttgctcattatcaaccacgatagcttgtgtccataggatatgagatagacaagttgaggcattatatatacggtgtggcaggagtacttcaatcttacaatactagcaaaaccacatatagagggtgtaatagctaagtagcctgtaagag

SEQ ID NO: 36 - DNA, 2000 bp, Populus trichocarpa
  source 1..2000: mol_type = genomic DNA
gtcaaacaaactaaaagcaaggtgacccatgttattttaaaattgaccacaaaaatctcatataaagcgaattaaaaaaataatagaatagtaaagtataaaaaaaggctttaaaaaagcaaaaaaacacggaaacctgtgtccatgtcaacttcgttaatcaaagtcaatctcttaaatctataaatcattaaattctacacctggactaaactaagaaacctaacccgatcaccctgcgacccaagatatgagatgaggattacccgacaaaaaagaaagttgaacaaatcacaaagctcatagcctaataattcaatgtcaaatgataaaaataaaaaaaattctgaaaaggattttaaaaaataccaaactcaaactaagtaaatcgttgaaactagtgaactaggtcataaaaatgagacaaccctgtaaaagaaagaaataatcaaaaagcaaaatcctcaattataaaaaattaaaaaaataaacaataaggaccaaatatgatattaaaaataattgaaaaaaactaattagggacaaaactgaaaacaaaataaatgaagaaaatgatagaaaaggagtaatgaaaaaaataagtaccaaaattttaaaaaaatgaattaaaattttaaaggacaaaatcaaaaaaataaaataaatgcaaaggatgaaaaaacaacaattgaaagaataaggaccagattgtaaataaaaaaattaaattaaatgcagagagggattaaattgaaaaaaataaatttctaaaagcattaaaatcaaagcaaatagaaatcaaaagaatgaggttcgaatccaaaacaaattcaaaatgaaggacaactaaatttttcaaggcttggcatgaaaaccaagaccgagagagaaacaaagggcaagagaaaaaaccccatcgccaccaaaccgctgcccgtcgtggagcacgcgccactccagcggctgcgccacatccttgcatattgattgagatggcgatctgttttattggccatcgtccttggccacacgcaccttcagaatagcatgaaggattctgacacgttgatgcgtgtgtggcatacgttatcatattttactgatttattaatattttaataagaaaaattacaaaaaagagcccaatgacataccattattacaaagaaatcatgatgcaaagattgaaacaccctcggaagctatgctttaaatatggaaagtcaaaggtcaataacgtaatttttctgtacaaaaataatagtcaaaggtcaataacgtaatttttttgtataaaaataatacaaagacaaataaatcccttattgcaaggtcattttgtttgagcacccaagggtataatcattttactgtgtaaattaaaaaaaaatcttagtaggagaccatttttatcagatttcaagggtatataaaaatatattttcactgtgcattataatttagaatgaaatctttgcccctgatgaattctgaaataccatttgaatctcatggacaatattattttaccccagtatcatagataaaaaattataattacactgcttgaatttacggtgattctccttatatgctacagtgttttctttcgcaaggagcccttaacccgagtccctagtcgaactaaaagattatcgccatggccatttaatctacgcttttttttttttttcttatgaacttacaattaatttatttttcaatttttttttggtataaaagaactatttctcgtaaagaatctcgttaaattcgttgggaaccgaagaggttttattttctaaaatctcggagaatccagagacagaaggagtagatctttggtggacattgaactccgactcttcccgggacacataaaccgtatcctgctaggactattacgcttgattccccattctataaatatatcaaactcggtttagaaaaggagctctcagcaaaaagtttcagatcaggaaggcaggggaacctcaacc

SEQ ID NO: 37 - DNA, 2000 bp, Populus trichocarpa
  source 1..2000: mol_type = genomic DNA
aaaaaaattaattttttttaaaatatttttaaaacaaaaaaataaccagttttaaataaagatgagatggcaagcttttccactagtcacccaaatacggcttcaaagttcaaataggtatacatttttttcctttttttcggtgggactttaaataccgaccacaatttattttaatgttgtcctgaaaccttatgtagattaatatacgtttaatgaaacagatccacatcatgatcaataaataattgccgactcactcaacatagaaaactccacatctctcttcttgatactgttctcatcattgaaggattcgtgtcttgaaaaacagtccattatcatcattaatttgaatgtttttcgtaaattttatctgttagacgtctgctatatatatataatttatatatatatatatatatatatatatatatataattttcttttaaaattacaattaattgttagagacttagaggacgtaaatacctagtgtttaattatcaagtgcaaatgtaaatttgtaatatatcactttcgtgtttccattttcgattatagtaattaaatcagaaattgaagcagaaagaaatatggtttgatcaaattaattagcttctcaagactgagaattaatttcacaagaaccttatcgatactctaaaggaagattgtaactcattgacttggatgctattgccagctcttgaaatggacgtccccgtcacaatcatgacgggattatcgactatatatatatatatatatacctaattctaattctctaaacaaagctaattattatctatacatttgaagtggaaaacacgatttatataaaaacaaaaaccacctcaagtttattttatttttttcaactcctttttattatttgggacctttcaactcctttttattatttgggtccctttttcattcaattttatttattgtatcgttttgcttttcatttaacaattgtagtgcagtttcataaaattgatgctccataaaccatgaaataatgtttgaaattacaaatatgtaaaacaatgtaaggtgaaagaaatattaaaaaaataataatggaaaaacaatatagaaaaaagacaatccactaagagttgcaataattacccataatgtattatttttatttttttgtcgcttgattcttttatttttaatttaatcatttcatattaaatttatttgtgattgaatttgatgttttgttttgattgactctaacatagggctctcacaatattaaaaataaattttgatgttgtgttgaattaaaaattttgagatccaatttgaaattaaaacaaatattcaatttgttttttttaaataacatttttattttagcttcccaactcaatttttaattgtaattaatagatttattattatttattaacatatataattttttatttattctattaaatatatgcatgtataaacttttcagttcgcaataaaaatatttaacattgtggaaaatcaaactagcttgattcatgagaaatgccccccctttttttatcgtttgatacgaggaaaatgacataggagggcattaatcatttccaagttgtagcttactttggtgcaaatgaattattctatcaaaacatgcttatcctcacaagttataagatgttggctttgtgtctttgtttccgtaactaacatttagcagggtttattttagttgtcttatgctcataattaatgtataattattactaatcaaataaaaggcggttggtcggcttccaatcatatagtgctcctccattcatatgggtcccatggtcggtgccggctatgatgcttattgcaaaattagcatcgcagagcgccagtggtgctataatataaaccacgcccatgaaatcaatgcagcccatagaaacacatacaagaacttgagctagctagcagtaattaagcttaattggcccaggactactgatcaagcaaga

SEQ ID NO: 38 - DNA, 2500 bp, Populus trichocarpa
  source 1..2500: mol_type = genomic DNA
ttttttgactcgataaataataaatataaactcgatttaatttgatgagtactcactatcatctttagccacggtgaatccagcttgccacttgtttgatttaaataaaacagtaacaagattcgttgctccacgtgcaaatgctaaatcgaaccagctagatttcatcgaccttgtaaatccaatgcacgtggctcacccatggcgggaaaacacaactccggtggtgaataataatagcacgggaagaaaagcagtggaaaaaaatggtcactgaagcctatatttcttctttttttcttgctcttcgagcaactaggatattaatttttattaatctttgaaaaaataataataataaatatcttataaagcttaaagaatcgtgaatattgttaaaaaaaatcactataattgatgtaatgcttatctaacccttgaaaaaataacaatgatgttgatactcttattatcatagttttaaaactcggtctagcaggttaatcctgaatccgacagatccggggttaaaatcgagccgagttgagaaaaaaatagagaaagaaaaaacccggtgtgacccggtcaaaaatccggatacaacctgttgacttttgtttttttatttaactaaaaaattgacccggacgacctggtcaaaaccagaacccgaaccttgaaccgggccggattttaaaaaatatggttcttgtattagttttctcctcctttaatatttttaatttcttttagtgataaagattcagacaacataggatgacgattatccttttatatatatatatatatatatatctgacatttttacatgagaaaaataacttaaagtatagggacttaaaaatgccattaaactaacggaaaacagcaggagagtggatcgagagacaagttttattacagtgctcttttcaatcgaattcgtttagaatttcaagctgtgtatcataaaaatttaagattttattttatttaagattaattttttatattattaaatcgttttaatatattaatataaaaatacattattaaaaactaaaaatatattattttaatatatttttaaacaaaaaaacattttaaaaaccacctttacaaccttaccaaaaactcataaaagcaacctcggcgcgtgcacagtctaccgttacagattctgacagtctccgttcgaatacggtcaacgcttttgacgctttccagaaccacacttacatctgcgcgtgtcaagcacatacacggatctgtgagaacttgtaaagcacgcgttttcttgaaaggcgacgttagaaatcggttttggtatggtcatcatatgcttactcgcaggttatctcttttagtttttagatttttatttttatttttctcgggaaatatcggtgacgcccgcccttcgagtggcggttagtttttcctatccccttgatttataatttgtaacttttattatcattgttggatttatcttattataaataaaaaaaataatgatattgataatattataattcaagtttcagtatatttattatattttttatttctctacttttttgtatatttaatatttttaaagtatattattattctaaatatcaacaaattataaaaatcatgttttttaattatgatatatataaactgtgttattttatcaatacttttaaaatatatgttaaaaataaaaaataaccctctttccattaactatccatacgaaagggaaaaaaaaatctatcaaggatttaggatttaattctaaaaacaagttatgaataattatttttttcattttacaacctttgttgtttaattattctttcaaaatatattaacaattataaaaaaaaactaaaaaagaagtgatttttagtgtatttctccacacaaaatattatttattataataatgtttttatttttttatcatttgatatttttttatttagtattttttttaatttcatcatttaatattaaatttattttttattgagttattttattttcatgacatgaattatgagttttgcatattatattaactaactcagtttttttaattaatttttatttttttattttattttttaatattaatttgattaataaaaaatctaaataataataaatttttacttgtataatactatttgctaaaatattatgtttttttcaaataaataaatttaacgaaaatacaagaaaataatagcaaggtaataacggaaagcgcagtgagaaaaagaaaagaaaagaaaagggcaaaaaaagacaggcgtgaagagttctcagataaaaccccttcactcgcttactctatttgtctctctccattacagtagggcctcatagagagagaaagatatgcaatatatgcaaagagaagcttgagagatttttatctccctttttcatttttgatggtgttctataatgagaagaggattaaatctcttcttctctgaagaag

SEQ ID NO: 39 - DNA, 2101 bp, Populus trichocarpa
  source 1..2101: mol_type = genomic DNA
atgctcaacctttttaattttttaaaaaaaatctctttcactacttttcttgttttatataattcaagaattggttgctatacgaagatccaagttgttgatgataaaaagaaaggaaagaaaagaaatccaagttgtcggttctttaaattgtgcaaggaaagcaagagcagataagcttaaatgttaagaggaaagccaaaggtctgcatgtcgtctgtgtataaacccgtcaagagtttagaatgagttgttcagcaacccaaatccatatgtcaggccaaaaacattgttagctgaaggatcttattgtactacattgcaaatatgtggtcgagagagagaggttcacatcaactcggtcgccaaatctttatcttagaagcctttgtttaccggaaaaaacaaaatatagtgaattttgtccataggtaaaaatcatatggataatatagccagctagatgcatgaaagttacactagagtcatagcctgaagaatttttgttacattagttaatcaaatgtactcgtaatgtttttaacagcttattaatttaacatgttttttaaaatcctttttaaaatttgttttatataaaaagaatattaaattaatatttttcactgttatcacgtggaaaatggctttatccctcattttaaacatgttttttaatatgctttttaaattagttttttattaaaaaaatattaaattgattttttaagatgttttttgatttgttaatataaaaaatatattttaatacatttttatttcacattaatatcccccacaaaatctgtgaaaattaataatggacgactccccaaccaagtcaagacacgaggtctctccacacaattactggaaacaaatttaatgcgtttaggacatgtatttgaagtgcaatcgcttccaagaacgtaaccttgtccataagaggaaaaacatgctcagctcagtcgagattcctataaatagtcaatgaactctggtctgtccacattggcacccttaattactaaaccactagagaaagttacagccacacaagggcaatcatatatatataataattaagaacataaatttaccatttggcaaatgtttttttatagtgattgatgataatccagattaatcaagcacgtctgctgttaatgtctcccaattcatttaccaaatcacacgtgctacgcagaatcacttattgacagtccaaagtgcatgatggccgtgattgcagtcagtgagatccttctgcgatgctctagtttcactcaacaactaattataacgcacagaatcagaattattattcaccccaatgttataatacgtacttatcatcaactaatcagtggtttatgagataatacattatatagttagtgtaataagacacgataaggtactgccatagaattaagaaaaaggggaataagaggtggggttcggaggtttttataatttagagaggttattctacgtgggaaactggagctagctacagctctcgaagaagaggagttggatttggatatggtatgctgctactacattcaaatagatatattgtgattatatatgctttgttttttcaacattctctaccacttcgagacgacggcattcactcgctggcttctctcagtcaccaatatttttccgtatcctgaaaagcaaagaagttccattttcaagttttgggattggaggaatctttttcattctctccctctctactttatactataataacaaaaaaaaaagcttgagagagaaccgaagaggccaccgaaaaacatgggacaccttatcatgaaagcaagaaaaaaaacgacccgattagacgggccaacccacacgcatatacacacagttactcacacacccacacgctcgatcgatcacacatggtctctctccctctgtgttaaatttgtgtgtgcctcgttctttcttttaaaaatggaaatcataagtcaacctcccctaatgatttggtcttgggttcttctaagcaccacctcatatcccactttctttagcatcctctatataattaccactccctatcattcagtccttcgcc

SEQ ID NO: 40 - DNA, 600 bp, Populus trichocarpa
  source 1..600: mol_type = genomic DNA
aaatctagtgaatccagccaaacctaattggttaactctaaatttttttattttaaatatgaaacaatattatttttaaaaaatcttattgacccaaattgacattaactttttttttatatatatatatatatgaagtaaactaagctctaaaatcactgtgtcaatggcaagctgccaagaaccacagaagtggtcataaatatcttaaattaagggagctgaaagagagagtggaattaatcgaaaggctgcctcagcaagaaccataaagccataaattaactagcatcacacatccatgacgtcctccatgggatgagataaggtcatcgagcttcttccacgtggaaacctctttgtggtgctaacgatgaggctagcatcaaaagaattcccaccttcatgtggacagaatagtgtaatgtcatcacttatcgtatcgaacggtcaaattccgctcattatcaactccactgatttgtgaccgtaggatacgagggggacaagttgaagcactatatataggaagtgacagtaaagcgagttcagaatatcacatttatagggagcactgcatagcttacaagcttgtaagag

SEQ ID NO: 41 - DNA, 1800 bp, Populus trichocarpa
  source 1..1800: mol_type = genomic DNA
cattcaaataaataagagattttaggagggaatcatatcagcatccaacctatccatcaattttaatcggatcccagaactacaaattaattttcccaattgaattcctaaaggattggacatttctcaattgagtattccaacaactatatttattggcattttccgttatttcatgcaataaatagcatgaaactattttgaaaacaatttgacatttttctattaaaaagctacaatgatgtcaaatgaaacaagaatttcctaacacgtgagaattttttttataaatttaatcattagaagttcataacacattggagtcgaaaacaaaattcttacaaaatcttcaccatctaagttgtccatagcaaattacattttttacaagagcgatagcgtttgaaaacgcgggataactcgctttctaaaaagttttaatttttgttttttgctaaaaattaaatttttttttatgttttgaattgttttgatacgctgatcttaaaaataatttttttaaaataaaaaaaattattttaatgtatttcaacatgaaaaatactttaaaaagtaatcacaatattctctcaaacaggtccaaaataaaaaaaagaaaaaaaaaaagaagagggagttgctgggctgtttagtaatgtgtaacggaggtgagattataatgggcggaaatggttttagcccgacttctctttagaaatagccatgggcccatggcattcttctttgggctaatttaaattgctagaccattttctttttttttccctaagaaagtactggaccagaccattaagaaccctgtccactccttgtttacttggccattttcaatgcatactgagctggatttgatcgttagaggacctaaagcattgcttacttgtgaccacgtattgctttaaattttgaggttttgagggtgttgttttttaaattattattttttatttagagttgtattaaaataatatttttttatttattaagatttattcttgacatcaaaataataataaaaaatataaaaaaataattttaaataaaaaaaagtaaattttcaatgatctttatttggatcgcaattccaaaccaatactaaaaaccagattgttgagctttcatgaatgttttggttttactttccataatgaaattgctccaactaaaaaaaaaaaggtaaaatctagtgaatccagccaaacctaattggttaactctaaatttttttattttaaatatgaaacaatattatttttaaaaaatcttattgacccaaattgacattaactttttttttatatatatatatatatgaagtaaactaagctctaaaatcactgtgtcaatggcaagctgccaagaaccacagaagtggtcataaatatcttaaattaagggagctgaaagagagagtggaattaatcgaaaggctgcctcagcaagaaccataaagccataaattaactagcatcacacatccatgacgtcctccatgggatgagataaggtcatcgagcttcttccacgtggaaacctctttgtggtgctaacgatgaggctagcatcaaaagaattcccaccttcatgtggacagaatagtgtaatgtcatcacttatcgtatcgaacggtcaaattccgctcattatcaactccactgatttgtgaccgtaggatacgagggggacaagttgaagcactatatataggaagtgacagtaaagcgagttcagaatatcacatttatagggagcactgcatagcttacaagcttgtaagag

SEQ ID NO: 42 - DNA, 2000 bp, Populus trichocarpa
  source 1..2000: mol_type = genomic DNA
tgggaagggttgtcgggactctcgtgagggtacgacaaaaataataacaaaatgataaaagatggaaacaaaaagcttttaaaaaaacaaaagcaccatggaaacttgtgtccctatcagcttcattaatcggagtcaatctcttaaacttgtaaaccgttaaattctacactcggactaaacttagaaacttaacccgaccaacctacgacctgagatatgaaatggggataactcgacaaaaaaaaaagctaaacaaatcacaaagctcatagcccaataattcaaagtcaaatgaaaaaaattaaataaaaacaaattaataagtcaatgtcaaatgataaaaataaacaaaaaaaaccgaattctaaaaaggattttaaaaaatatcgaactcaaactaggtaaatcattgaaactggtgaactgagtcatgagaacgggacaaccatgtaaaagaaagaaataatcaaaaagaaaaatcctcaatttaaaaaaaaattaaaagataaccaatgaggaccaaatcttgcacaaaaaataattgaaagaaagtaattagggacaaaattgaaaataaaataaatgaagaaaaggaaaaaaaatgagtaatgaaaaaatgaggaccaaaatttgaaaaaaatgaattaaaattgtaaaggacaaaatcaaaaaaataaaataaatgaaaaggatgaaaaaacaacaattaaaagattaaggaccagattgtaaacaaaaaacaaaataaattaaatgcaaagaaggatgaaactgagaaaaataaatttcaagaagcattaaaatcaaagcaaatagaaatctaaagaatgaggaccgaacccaaaacaaattcaaaatggaggacaactaaattttttaagatctagcatgaaaaccaagatctaagagagagaaacgaagggcaggagaaaaaacccattgccgccaccaatcgtggagcacgcgccactccagcagctgcgccacatccttgtgtatcagttgagacggagatcggttttattggccatcgtacttggccacacgcgccttcggaatagcgttgaggaccctgacacactgatgcatgcatgatacacgtcagcatattttactgatttattaatattttaataagtaaaattacaaaaaaaatccaaggacataccattattacaaagaaaccatggtgcaaagactaatatgccctcagaagttaagctttaaatccggaaagtcaaaggtcaacaatgtaaattttagtataaaaataatgcaaagacaaataaacccctccttgcaaggctattttttgtgagcactcagagctataaatgttattttactgtgtaagttaaaaaaaaatacaaaaaaaaactaattattattagattttaagagcaaaatagtattttttaccgtgcattatagtttggaatgcaatctatgcccctgatcaattctaaaataacatttaaatctcattaaaaaatataaagataattattaagaataatgaagagcaaaaccataattacactgcttgaatacgtaattaatcacctcatgatacagcgaaaaactacactgaggccatttaatttttttttgtgagcccttcacccgagtccctagtcgaactaaaagattatcgccatggccatttaatctacgctttttatttattttttcttgagaacttacaattaatttatttttcattttttttttgtataaaagaactatttctcgtaaagaatctcgttaaattcgttgggaaccgaagaggttttattttctaaaatctcggagaatccagagacagaaggagtagatctttggtggacattgaactccgactcttcccgggacacataaaccgtatcctgctaggactattacgcttgattccccattctataaatatatcaaactctgtttagaaaaggagctctcagcaaaaagtttcagatcaggaaggcaggggaacctcaacc

SEQ ID NO: 43 - DNA, 2000 bp, Populus trichocarpa
  source 1..2000: mol_type = genomic DNA
aatttttttatataattttttatttgattatataataaaaatgatgtttgtaaaatcaaaaaccaactaaatatcagagtgttttattcaagattctgataaacctatgaatatcaaatataaataatttatgtttcccaataaaaaatcaataaaatattaaagaataagattgaaaagaattaagcaagaaaaatcaataaaaatccaattatgaatgatcaaattgaaaaaaaaacaattcaaaaaaataaataaaagaaggcaaaaataaaaagagccaattgtgaaggatcaaattgaaaaaaaaggcccatgagtgcaagcttgaggctgttaaattttttttcttttattgtccacctatggtttttttgaaaaaaaatagcaggcaacttgttgccttctctatccagattttgattgtcggccataggtgttaaacaccataaaaatttatttaaaccataattttggccccaaaacacttgataaacatcttgaagacttaaataaacttgtttatacctttaaataattcaaaaatgataaacaaacaaaaaatcaagtcgaaatcaaaatactttctgagatcagatttacttttttaagaaaactataggtgaaagaacaccttactagaactctttccggcatataaaactcgtacaaaccaatatcgactattttggtagcataattcgttgtcaaccaatattttttctccactagcagaatttaatttaatgactttctctcttccatcttgaactagaaaatggacaaaaaaaatttgttccacaattgagtttttgaaatatttagggaccgagaagttatcatttaaaaagtaagaaggtcaaagtttactttttatccctttttgataacaccacatgtttcctccttgttttacttatcaatctcatttgttttaatcttgtctctcattaacaatttttgataaatcatccacgaaaattgggcattgaaggacacaattgaatagaaaaaaattgaataaacaattatagaattgtgaattgcaacactaaaaaactcatgcattttagagtttttgttaataaataactaatataaaattaatatataaaattaatataatattttaatttttttaaaatgaatcttaaacttaattatcaagtttaatatgatataataattttaataatggtctataattttaatttcaatattactcattcccacttactaaaatagtaaacttaagcaaaagttgtaattagtcatcatgaccgtgaagactcataatatataaaatataataaaaaattactaaaaaaattcaatttaatatatttttaagttaaaaatatttttaaaaaatatctagaagcaaaaccagaaattataccgaacaactcagatgacattgctagctctagaaagagttacccatcacaatcatgatggaattatcaaccatgtaccatatttaaaaaaaaaaaaaaaaacacaccaactctttgaacaaagctaattatttcatatatgataaaacgccctaatttttatcatttgatacggggaaatgacacgtggacatcgatcattaccatattaattattggtgctgaattgatcatatcccatcaaaacatgctttgtttcctttcttaacacacacacataaataacaagtaaattagttgtcttgtgttcataattagtgatgacatattagttattactaatcaaataaaaggcggttggtcggcttccattcttttagggctcttccattcatatgggtcccacggtcggcgccggctatgatgcttactgcaaatttagcatagcaaatcaccagtaggagcgtgtatttaaaccacgcccatgagaccaacgcattgcagcccatcgagaaacacacacaagaactcgagcgagctatattgatagttctaataattaattaagtttaattagtctaataattagtgaaacaagaaaga

SEQ ID NO: 44 - DNA, 1757 bp, Populus trichocarpa
  source 1..1757: mol_type = genomic DNA
ttcgcaattcaaagcctttttttttttaatgattttgatacatgaaaattaatttaaaaaacattattttaaaaaaaaaaatatttcaccatattactgaaaatacacttattctagttaggctattaatcaaatagattaagtttttaatttgctaaaacttgttaagtttagttaattttttaataatctaaatgacatgataaagtcaaataaaaaggtttaagacctaaattataagtttaacgagttgagttaatttatttcaatccaaaacaatgttatatttttttaaaaattgaaacaatatgattttgaaatatatatatatatatatatatatatatatatatatattttataattttatagtcaaatcaaatttcatccagaaatttaacttctataaaattaattatatttaactaaattaattttcactttatttaatataaaacccgattcaaatcaggttttaaaacaccttaattgaaaatattcaaagattattaactattgtaggaaaaatcattacttttctttgtctcttttatttcacacttggctcgagatttacttgaaataatatgcaacaatttagaatttcatgtggtgggatagatgttttgcaagcttgaagcatgtctttgtgcatatagatttcatgttatagactttagaatttcattttaacagcaatctacctaacaattatttttatataagaagtgatgcccatgtctggattattttttactttttatgtataagaaaatatttttaaaaatattttaattattttctccatttaaactggccaacaattttcatagttggtgggatgctagcaagcacgaaggatggcttgtctgtgactaatgttagcttaatatcttgtaaagatgagattcaaccagcaagaatatatctcatgtttatagacttaattaaataattttctatataatagtaattgaacaaataaagataataatgttattttcatttttcttgtataaaaaagtagcatgtattgattttttatgctgataatcattatgttatttgtccattaacccatctagatattctagacgtgattgagaccctaaccctttttaaattaaatataatcaacaatatattaaagattaaaaaaaaaattgtacggataggtagttaaatatatatatatatatatatatatatatatatatatatatatatatatttcagaaaaatgaagaatattccaaggacattgaagggcatccggtcatggtttcagaagacatcgctggatttcttgctagttaattagctctgaaaaaacttaggaatatcccagaacattaaatttgaccctcagtagaatgattagcatggagaggtaagttacatattgatctctttccttgaggcaacagagagagggacatatttgttttctaatataatttaatttgtatcttcgttatgagcaagagtgtatgaaagcttaaccatacttgttttcagtcataattattttttaaaaaatttatttagttttctaaattagattaattataattccaaatgggtatatcctcaatattccctttctaatttcttgttattctcccatggcttagtgttcatcgagctcgacgtgcattattttttctttatcatattgcttaaaataccaattatataaccatgaaatcctaggcaagaaaattaaccactcccgccaaaagaacgaaaagaaagac

SEQ ID NO: 45-49 - skipped (5 entries)

SEQ ID NO: 50 - protein, 186 aa, Populus trichocarpa
MSGSSGGSSSSSSSRIIDQESGASASQRKFKGARRRKWGKWVSEIRIPGKQERLWLGSYSTPEAAAVAHDIASYCLRGPSSLESLNFPLMLPASVREDMSPKSIQKAASDAGMAIDAQMILNRSLQNEVKVGPENVAINHGPQTQLWEPAAGGGGDSSNRCENWHENNIGTRVGDDLNISIEDYLM

SEQ ID NO: 51 - DNA, 561 bp, Populus trichocarpa
  source 1..561: mol_type = genomic DNA
atgagtggtagcagcggcggcagcagcagcagcagcagcagcaggataatagatcaagaaagtggagcaagtgcatctcaaaggaaattcaagggagcaagaaggcgaaaatggggcaagtgggtgtctgaaataaggattcctgggaagcaagaacgactttggttaggttcttattctacacccgaggcagctgcggtggctcatgacatagcttcttattgtttacgtgggccttcttcgttagagagtctgaatttccccctaatgttgcctgcaagtgtaagggaagacatgtcacccaagtccatacagaaagctgcgtcggatgctgggatggcaatcgatgctcagatgatactgaacaggtcgctccaaaacgaggtcaaggttggacctgagaatgttgcgattaatcatgggccacagacacagttatgggaacctgcagcaggtggtggtggtgatagtagtaatcgttgcgagaactggcatgaaaacaatatcggaacgagagtaggggacgatttaaacatctccattgaagattatctcatgtag

SEQ ID NO: 52 - protein, 168 aa, Populus trichocarpa
MSGSSSSRTDQESGASAARKKFKGVRRRKWGKWVSEIRIPGKQDRLWLGSYSTPEAAAVAHDIASYCLRGPSSIESLNFPLMLPASVREDMSPKSIQKAASDAGMAIDAQMILNRVPENEVKFWTASGGVNHGLEIELCEPAGGDHGGNWHGNNTGMREGDISIEDYL

SEQ ID NO: 53 - DNA, 507 bp, Populus trichocarpa
  source 1..507: mol_type = genomic DNA
atgagcggtagtagcagcagcagaacagatcaagaaagtggagcaagtgcagctcggaagaaattcaagggagtgaggaggcggaaatggggcaagtgggtgtctgaaataaggattcctggcaagcaagatcgactttggttaggttcttattcaacacccgaggcagctgcggtggctcatgacatagcctcttattgtttacgcggaccttcttcgatagagagtctcaatttccctctaatgttgcctgcaagtgtaagggaagacatgtcacctaagtccatacagaaagctgcttcagatgctggcatggcaattgatgctcaaatgatactgaacagggtaccagaaaatgaggtcaagttctggactgcaagtggtggggttaatcatgggctggagatagagttatgcgaacctgcaggtggcgatcatggtggtaattggcatggaaacaatactggaatgagagaaggggacatctccattgaagattatctctag

SEQ ID NO: 54 - protein, 155 aa, Eucalyptus grandis
MSSSSSEEPRNAKYAGVRRRKWGKWVSEIRVPGTQERLWLGSYTSPEGAAVAHDVASYCLRGPSCLDKLNFPSLLPPSARPGVSPRSAQRAASDAGMAVDARYIARRSDEEADRTSPGHRPEVGWECAQGNEASSGTRGGRGGGDGLSISVDDYL

SEQ ID NO: 55 - DNA, 468 bp, Eucalyptus grandis
  source 1..468: mol_type = genomic DNA
atgagcagctcgtcgtccgaggagcccaggaacgccaagtacgccggggtccgccggaggaagtggggcaagtgggtgtcggagatccgggtccccggcacgcaggagcggctctggctcggctcctacacctcccccgagggcgccgccgtcgcccacgacgtcgcctcctactgcctccgcgggccctcctgcctcgacaagctcaacttcccctcgctgctgccgcccagcgcgcggcccggcgtgtcgccgaggtcggcgcagagggccgcctcggacgccggcatggcggtcgacgcgcggtacatcgccaggaggtcggacgaggaagcggacaggacgagccccggccaccgaccagaggtcggttgggagtgtgcgcaagggaatgaggcttcttccgggacgcggggagggagaggaggaggtgatggtttgagcatttcggtggatgactacctgtga

SEQ ID NO: 56 - protein, 388 aa, Populus trichocarpa
MPLAFERGKVNQSIERKRDSVLDGVKDMAVELMMGYSGDSFATKMQENDVREAATAGIQSVEEVIKLLKQNQLEQQQKQQYYQELSAASSSSNLGTDNIMAVTDMAVNNFKKVISLLGRTTRTGHARFRRAPVACPPQQQIQEPEPGPQQQKQQVQEPVPYVRAINSQPTEQGSAFRVYQPTPIHRLPPLPHNQQQKTLLVTKNGLSDRNEMATTINFANSPTISAPSFLSSLTGETDSFQCSKSSGFQFTQPSAGKPPLSSSSLKRKCNSMDDAALKCGSSSSRCHCSKKSRKSRIKRVVRVPAISSKMADIPPDDYSWRKYGQKPIKGSPHPRGYYKCSSVRGCPARKHVERALDDSMMLIVTYEGEHNHSHPIDEAPGALVLESS

SEQ ID NO: 57 - DNA, 1167 bp, Populus trichocarpa
  source 1..1167: mol_type = genomic DNA
atgcctttggcatttgagagaggaaaggtcaatcaatccatagagagaaagagagattcagtcttggatggtgtaaaagacatggctgtggagcttatgatggggtattctggtgatagttttgctacaaaaatgcaagagaatgatgtgagagaagccgcaactgctgggatacaaagcgttgaggaagtcataaaactgctcaaacaaaatcaactggaacagcaacaaaaacaacaatactaccaagagttgtctgcagcctcttcaagttccaatcttggcacggataatatcatggctgttactgatatggccgtgaacaatttcaaaaaggttatttctttactgggtcgcaccacaagaactggccatgctcgatttagaagagctcctgttgcctgccctcctcaacaacaaatacaagaaccagaaccaggaccgcaacagcaaaaacagcaagttcaagagccagtaccatatgttcgagcaattaattcgcagccaacagagcaaggctctgcttttagagtttatcaaccgaccccaattcatcgtctccctcctttgcctcacaatcagcaacaaaagacactgctggttacgaaaaatggattatcagatcggaatgaaatggctactacgatcaatttcgctaattcgccaacaatatctgctccttctttcttgtcttctttaacaggggaaactgatagcttccagtgttctaagtcttctgggtttcagtttacccaaccttctgctggtaaacctcctttgtcctcttcttctcttaagagaaagtgcaactccatggatgatgctgctctcaagtgtggctcttcttctagtcgctgccactgctccaagaaaagcaggaaatcaagaattaaaagggtggttagagttcctgcaattagtagtaagatggcagatatcccacctgatgattattcctggagaaagtatggccaaaagcccatcaaaggctctcctcatcccaggggatactacaagtgcagtagcgtgagaggatgtccggcacgcaaacacgtggagagagctctagatgactcgatgatgcttattgtgacctatgaaggggaacacaaccactctcatccaatcgatgaagcacctggtgctcttgtccttgaatcatcttaa

SEQ ID NO: 58 - protein, 358 aa, Populus trichocarpa
MAVELMMGYSGDCFATKMQENAVREAAASGIQSVEEVIKLLKQNQLEQQHYQELSAASSSSNLGTDNIMTVTDMAVNNFKKVISLLGRTTRTGHARFRRAPDTPPTQQQIREEPESQQEKRQVQEPGPSVRAINSQPTEQASAFRVYQPTPIHRLPPLPHNQQQKSPLLVTKKGLSDRNEIPTTINFSNSPSISSATSFMSSLTGETDGFQRSMPSRFHFTQPSAGKPPLSSSSLKRKCNSMDDAALKCGSSSGRCHCSKKSRKSRAKRVVRVPAISNKMADIPPDDYSWRKYGQKPIKGSPHPRGYYKCSSVRGCPARKHVERALDDSMMLIVTYEGEHNHSHPFDDAPAALVPESS

SEQ ID NO: 59 - DNA, 1077 bp, Populus trichocarpa
  source 1..1077: mol_type = genomic DNA
atggctgtggagcttatgatggggtattctggtgattgttttgctacaaaaatgcaagagaatgccgtgagggaagcggcagcttctgggatacaaagcgtcgaggaagtcataaaattgctcaaacaaaatcaacttgaacagcaacactaccaagagttatctgcagcctcttcaagttccaatcttggcacggataatatcatgactgttactgatatggcggtgaacaacttcaaaaaggtcatctctttactgggtcgcaccacaagaactggccacgctcgatttagaagagctcctgataccccccctactcaacaacaaatacgagaagaaccagaatcgcaacaagaaaaacggcaagttcaagagcctggaccatctgttcgagcaattaattcgcagccaacagagcaagcctctgcttttagagtttatcaaccgaccccaattcatcgtctccctcctttacctcacaatcagcagcaaaagagccctcttctggttacaaaaaaagggttgtcagatcggaatgaaattcctaccacaatcaatttctctaattcgccatcaatttcttctgccacttctttcatgtcttctcttacaggggaaactgatggcttccagcgttctatgccttctaggtttcattttacccaaccttctgctggtaaaccccctttgtcctcttcttctcttaagagaaagtgcaactccatggatgatgctgctctcaagtgtggctcttcttctggtcgctgccattgctccaagaaaagcagaaaatcaagggctaaaagagtggtcagagttcctgcaattagcaataagatggctgatattccacctgatgattattcctggagaaagtatggtcaaaagcccatcaaaggctctcctcatccaaggggatattacaagtgcagcagcgtgagaggatgtccagcacgcaaacatgtggagagagctctagatgactccatgatgcttattgtgacctatgaaggggaacacaatcactctcatccattcgatgatgcacctgctgctctcgtccctgaatcatcttaa

SEQ ID NO: 60 - protein, 437 aa, Eucalyptus grandis
MPRGGKARGQIYLYVLRPLSSRLLPLLLIDPPRARLLLHMCSFSSRSRERGMAVELMMGYGSGGDGGVGFAVKKEETALREAASAGIQSVENLIKMLSSSPSSPSGGGVGGGRRAGQDSPSPSSSSSGGPSPAVDIEAATDAAVNKFRKVISLLDRARTGHARFRRAPVADPLPENREAFTPVRPAENPVGSARLQSAEPVSAFKVYCPTPIQRLPPLPHNHHHHHNHNHHHHHHQVQQNPIPVVPKVERKESATPTTINFSTSPPISAANSSFLSSLTGETDSVQRSFSSAGFQMTHHLSQVSSADKPPLSYSLKRKCSSMDDAALKCGSSSGKCHCSKKRKSRLKRVVRVPAVSSKMADIPPDDFSWRKYGQKPIKGSPHPRGYYKCSSVRGCPARKHVERALDDPGMLIVTYEGEHNHSQSVADVKAPLVLESS

SEQ ID NO: 61 - DNA, 1314 bp, Eucalyptus grandis
  source 1..1314: mol_type = genomic DNA
atgccaagaggaggaaaggcgagggggcagatctatctctatgtcctgcgacccctttcctctcgtcttcttccccttcttcttatagacccccccagggctcggcttcttcttcatatgtgttccttctctagccgctccagggagagaggtatggccgtggagctgatgatgggttacgggagcggcggcgacggcggcgttggcttcgcggtgaagaaggaggagaccgccctccgggaggccgcctccgccggcatacagagcgtcgagaatctcatcaagatgctgtcttcctcgccttcctccccctccggcggcggcgtgggcggcgggcggcgggccgggcaggactcgccgtcgccgtcgtcgtcctcttccgggggcccgagccccgccgtggacatcgaggccgccacggacgccgccgtcaacaagttcaggaaggtcatctcgctgctcgacagggcgaggaccggccacgcccgcttcaggagagcccctgtcgccgaccccctccctgagaaccgggaagcgtttactccggtccgaccggcggagaacccggtcgggagcgcccggttgcagagcgccgagccggtctcggctttcaaggtctactgcccgacgccgatccagcggctgccgcccctgcctcacaaccaccaccaccaccacaaccacaaccaccaccaccaccaccaccaggtccaacagaaccccatcccggtggttccgaaggtggagagaaaggagtccgcgacgccgacgactataaatttctccacctcgccgccgatctcggctgcgaactcctcgttcctgtcctcactgaccggggagacggacagcgtgcagagatccttctcctctgcagggtttcagatgacgcaccatctatcgcaggtttcgtcggcggacaagcctcccttgtcgtactcactgaagaggaagtgcagttccatggacgacgcggctctgaagtgcgggtcgtcctccggcaagtgccattgctcgaagaagaggaaatcgagattgaagagagtggttagagtcccagcggtcagttcgaaaatggcggacatcccaccagatgatttctcatggaggaagtatggacagaagcccatcaaaggctcccctcatccaaggggctattacaagtgcagcagcgtaagaggatgcccggcgaggaagcacgtggagcgggccctggacgacccgggcatgctcatcgtgacctacgagggcgaacacaaccactcccagtccgtcgccgacgtgaaggcgccgctggtcctggaatcttcctag

SEQ ID NO: 62 - protein, 353 aa, Arabidopsis thaliana
MTVELMMSSYSGGGGGGDGFPAIAAAAKMEDTALREAASAGIHGVEEFLKLIGQSQQPTEKSQTEITAVTDVAVNSFKKVISLLGRSRTGHARFRRAPASTQTPFKQTPVVEEEVEVEEKKPETSSVLTKQKTEQYHGGGSAFRVYCPTPIHRRPPLSHNNNNNQNQTKNGSSSSSPPMLANGAPSTINFAPSPPVSATNSFMSSHRCDTDSTHMSSGFEFTNPSQLSGSRGKPPLSSASLKRRCNSSPSSRCHCSKKRKSRVKRVIRVPAVSSKMADIPSDEFSWRKYGQKPIKGSPHPRGYYKCSSVRGCPARKHVERALDDAMMLIVTYEGDHNHALVLETTTMNHDKTL

SEQ ID NO: 63 - DNA, 1062 bp, Arabidopsis thaliana
  source 1..1062: mol_type = genomic DNA
atgactgttgagctgatgatgagcagctacagcggcggcggaggaggaggtgatggttttcctgcaatcgccgcggcggcgaaaatggaagataccgctttgagagaagctgcttctgcagggattcacggtgtggaggagtttcttaaactgatcggtcaaagtcaacaaccaacggagaagagtcagacggagataaccgcggtgactgacgtcgccgttaacagcttcaagaaggtcatttctctactcggtagatctagaaccggacacgctagattcagacgagctcccgcgtcaacgcaaacgccgtttaagcaaacgccggtggttgaggaggaggtggaggtggaggagaagaagccagaaacaagctccgtgttaacaaaacagaaaacagagcaatatcacggtggtggatctgcgtttagagtttattgtccaacaccaattcatcgtcgtcctcctctatcacacaataacaacaacaatcagaatcaaacaaagaacggttcgtcttcttcatctcctccgatgctcgcaaacggagcaccgtcaacgataaactttgcgccgtcaccaccagtctcagcgacgaactcattcatgtcttctcatagatgtgacaccgatagtactcacatgtcatcaggattcgagttcactaacccatctcagctctctggttcgagaggtaaacctcctttatcatcagcttcgttgaagagaagatgtaattcatctccctcaagccgttgccattgctccaagaaaaggaaatcaagagtaaaaagagtgattagagttccagcagtaagtagcaaaatggctgatataccatcagatgagttttcatggagaaaatatggtcaaaaaccaatcaaaggctctcctcatcctcggggatattacaagtgcagcagtgtaagaggttgtccggcgcgtaagcatgtggagcgtgcactagatgatgcgatgatgctaatcgtgacgtacgaaggagaccacaaccatgctttggttctcgagacgacgacgatgaatcatgacaaaactctttag

SEQ ID NO: 64 - protein, 572 aa, Populus trichocarpa
MERDKLFVSEGANTAATIWNSCSFGMEMQANELSCGPEKLANCFLNPNWDNSLDQSDPFESALSSIVSSPVASGANANANAIPNAGVGGDSLMIRELIGRLGNICNSGDISLQSFVNNNNNSTNTSCYSTPMNSPPKLNLSMMDSQMRGNLPIPGNSVVKHPGLAPFPADFVERAARYSCFGSNNPGGINKQFGLNESELINRLMPRVEPGKLSRVSSNNSMKVTVSQANVQESNKSSPQDGSLNSEKKFSRQSRPTTSENGDSREESSLSEQVPGGKLSMKSQNDANSRKRKSIPRGKAKETPSSSPSASDVKVAAENDESKAKRSKSDETNGSDKDTAKEKEEENGNQKQNKNNSKPPEPPKDYIHVRARRGQATDSHSLAERVRREKISERMKFLQDLVPGCNKVTGKAVMLDEIINYVQSLQRQVEFLSMKLSSVNPRMEINMETLLSKDIFQSRGSMPHSLYPLDASTPVFPYGYQSQQGLALQNGMPSNAETQFSMNPLNAALRRNPSMHLPHLDGFGDPAALQLEFYSSGLSHVGRRPSKCCADGIWSESSGELSRLSALNSHEN

SEQ ID NO: 65 - DNA, 1719 bp, Populus trichocarpa
  source 1..1719: mol_type = genomic DNA
atggaaagagataagttgtttgtgagcgagggagccaacacagcagcaaccatttggaattcttgcagttttggaatggaaatgcaagccaatgagctgagttgcggtccagagaaacttgccaattgctttctcaatcccaattgggacaactcattggatcagagcgatccctttgagtctgctttgagctccattgtctcatcacctgttgcatccggtgccaacgcaaacgccaacgccattcctaatgctggcgttggtggtgacagtcttatgattagagaacttattggaagactaggaaacatttgcaattctggagacatttcactacaatcttttgttaacaataataacaatagcactaacacttcttgctatagtaccccaatgaattcccctccaaagctgaatctctcgatgatggattcacaaatgagaggaaatctgccaattcctggaaacagcgtagtaaagcatccaggtttagcaccatttccagctgattttgtagagagggctgcacgatattcttgctttggtagcaacaatcctggaggcatcaataaacaattcggattgaatgaatccgaattgattaataggttgatgccacgagtagaacctggtaagctctcgagagtttcgagtaacaattcaatgaaggtcactgtatcgcaagcaaatgttcaagaaagcaacaagagctcaccccaggatgggagtttgaattctgaaaagaaattcagtaggcagtcaaggcctacaacatcagagaatggagattccagggaagaatcttcattgtctgagcaggtcccaggtgggaaattgagcatgaaatcccagaatgacgccaattccaggaaaagaaaatcaattcccagaggaaaagccaaagaaactccctcttcatctccatctgcttctgatgtcaaggttgcagcagagaatgatgaatcgaaggcaaaaagaagcaaatcggatgaaactaatggcagtgacaaggatacagcaaaggaaaaggaagaagaaaatggaaatcagaaacagaacaaaaataattcaaagccgccagagccaccaaaggattatatccatgtcagagccagaaggggtcaggctacagatagccacagtcttgctgaaagagttagaagagagaaaatcagtgaaagaatgaagttcctccaggatcttgttcccggatgcaataaggttactgggaaagcagtgatgcttgacgagattataaactatgtacagtcattgcagcgccaggttgagtttctgtcaatgaagttgtcatctgtgaatccgaggatggagatcaacatggaaactttgttgtccaaggatattttccaatcccgtggatctatgccacatagtctttatccattagatgcctccacgccggtattcccttatggttaccaatcccagcaagggttagccctgcaaaatggcatgccaagcaatgccgaaacccagttctctatgaacccattaaatgctgcgttgcggcgaaacccgagcatgcatttgccacaccttgatggtttcggtgatcctgctgctcttcagctagaattttattcttcaggcctcagccatgtgggaagacgaccttcaaagtgttgtgcagatgggatatggtcagaatcatcaggagagctttcaaggctcagtgccctcaactcacatgaaaattga

SEQ ID NO: 66 - protein, 567 aa, Populus trichocarpa
MEKDKLFMSEGASTAAPIWNSCSFGMEMQTDELNCSSGQLANCFLNPNWDNLLDQSDPFESALSSIVSSPVASSVNANVISNAGVGGDSVLIRELIGRLGNICNSGEMSPQSYINNNNNSTNTSCYSTPLNSPPKLSISMMDSQMRGNLPILGNSLVNHPSLAPFPADPGFVERAARYSCFGSNNLGGLNGQFGLNESELINRMMPRVEPGKLSRVSSNNSMKVAGSQANVQESNKSSPQDGNLNSDKKFSRLSRPSTPENGDSREESSVSEQIPGGELSMKSQTDANSRKRKSIPRGKAKETPSPSPSASDVKVAAENDESSAKKSKSEDTNGSDKDSAKAMEEENGNHKQKKDNSNPPEPPKDYIHVRARRGQATDSHSLAERVRREKISERMKFLQDLVPGCNKVTGKAVMLDEIINYVQSLQRQVEFLSMKMATVNPKMEINMETFLSKDIFQSRGSMPHGLYPLDSSTPAFPYGYQSQQGLALQDGMSRNAESQFSMNPLNAALRRSSSMQLPALDGFGDASHQASAMWQDDLQSVVQMGYGQNQQQDFQGSVPPTQMKIEL

SEQ ID NO: 67 - DNA, 1704 bp, Populus trichocarpa
  source 1..1704: mol_type = genomic DNA
atggaaaaagacaagttgtttatgagcgagggagccagcacagcagctcccatttggaattcttgcagttttggaatggaaatgcaaaccgatgagctgaattgcagttcagggcaacttgccaattgctttctcaatcccaattgggacaacttattggatcagagcgatccctttgagtctgctttgagctccattgtctcatctccagtggcatccagtgtcaatgccaacgttatttctaatgctggcgttggtggtgacagtgttttgatcagagaacttattggaagactaggaaacatttgcaattccggcgagatgtctccacaatcctacattaacaacaataacaacagcaccaacacttcttgttatagtaccccattgaattcccctccaaagctgagtatttcaatgatggattcacagatgagaggaaatctgccaattcttggaaacagcttagtaaaccatccaagtttagcaccatttcctgctgaccctggatttgtagagagggctgctcgatattcttgctttggaagcaacaatcttggaggcctaaatggacaattcggattgaatgaatcagaattgatcaataggatgatgccccgagtagaacctggtaagctgtcgagagtttcgagtaacaattcaatgaaggtcgctggatcgcaagcaaatgttcaagaaagcaataagagctcaccccaggatgggaatttgaattctgataagaaattcagtcggttgtcaagaccttcaacaccagagaatggagattccagggaggaatcttcagtgtccgagcagatcccgggtggggaattgagcatgaaatcccagactgatgccaattccaggaaaagaaaatcaattcccagaggaaaagccaaagaaactccctctccctctccctctgcttctgatgtcaaggttgcagcagagaatgatgaatcgagtgcaaaaaaaagcaaatcagaggacactaatgggagtgacaaggattcagcaaaggccatggaagaagaaaatggaaatcacaaacagaaaaaagacaattcaaacccgccagagccaccaaaggactatatccatgtcagagccagaaggggtcaggctacagatagccacagtcttgctgaaagagttaggagagaaaaaatcagcgaaagaatgaagtttcttcaggatcttgttcctggatgcaataaggttactgggaaagcagtgatgcttgatgagattataaactatgtgcagtcattacagcgccaggttgagtttctgtcaatgaagatggcaactgtgaacccgaagatggaaattaacatggaaactttcttgtccaaggatattttccaatcccgtggatccatgccacatggtctttatccattagattcctccacgccggcattcccttatggttaccaatcccagcaagggttagccctacaagatggcatgtccaggaatgcagaatcccagttctctatgaacccattaaatgctgcattgcggcgaagctcgagtatgcagttgcctgcccttgatggctttggtgatgcttctcatcaggcctcagcaatgtggcaagatgatcttcaaagtgttgtgcagatgggatatggtcagaatcagcagcaggactttcaaggctcagtgccaccaactcagatgaaaattgagctatga

SEQ ID NO: 68 - protein, 531 aa, Eucalyptus grandis
MEGSSTPLTQNSSTFGIGIHPNDQLPNCFLVAGWEDSLDQGDPFESALSSIVSSPVAPHAGEGEAGMIRELIGRLGSICDSGQASPGSYNHSTTNTPCYSTPLNSPPELSLSMADQLAHGGFPAPGSHFPGHPSFVSVMADPGFAERAARLSCFGPRNVNAANLNGGLGLNASEFLGKQANKLDSGRFCRVPSSQSLRANVGSQICAQGNERSFLQPGVSISTPENGEMGESREGSSISEQVTHGEMRANGQIDVNSRKRKPILKAKGRNSAVSPQSAKDSEVPMENEEPITKKIKPAETDGNEKDAAKGDQKQSKENSKMAEPQKDYIHVRARRGQATDSHSLAERVRREKISLRMKALQDLVPNCNKVTGKAMMLDEIINYVQSLQRQVELLSMKLSTLNPTVDFNLEAFLSKDILHPQGPLPNSLYPVDSSGSMLYGYQPPQGLMPMPSVVSSNMDAQFPVNFLNSGIHRDASLNLPLCVDNYGEASAPAPALWEDDLQSVVQMGLGQIEPQRVEQGSLATSQMKVEL

SEQ ID NO: 69 - DNA, 1596 bp, Eucalyptus grandis
  source 1..1596: mol_type = genomic DNA
atggaaggaagctccactccattgactcagaattcatccacctttggcattggcattcaccccaacgaccagctccccaactgcttcctcgtcgccggctgggaggactccctggatcagggcgatccctttgagtccgccctgagctccattgtgtcgtccccggtcgcgccccacgctggcgagggcgaggcgggcatgatccgggagctaatcgggaggctcggcagcatctgcgattccggccaggcctccccggggtcctacaaccacagcaccaccaacactccctgttacagcacccctctgaattcgccgcccgagctcagcctctcgatggcagatcagctcgcccacgggggcttcccggcccccggcagccattttccgggccacccgagtttcgtgtcggtcatggcggatcccgggttcgccgagagggcggcgaggctctcttgcttcggccctaggaatgtgaatgccgccaatctaaacggtggcctcgggttgaatgcatcggaattcctggggaaacaggcgaataagctcgattcggggagattttgcagggttccaagcagtcagtcactgagggctaatgtcggatctcaaatctgtgcacaagggaatgagagaagcttcctccagccaggggtctctatttcgacccctgaaaatggagaaatgggggaatcccgcgaggggtcttcgatctccgagcaggtcacgcatggggagatgcgtgccaatggacaaatcgacgtgaattcaagaaagaggaaaccgatcctgaaagcaaaagggcgaaactcggctgtttctccacaatctgctaaagattctgaggttcccatggagaatgaagaaccgataaccaagaaaatcaaacccgccgagactgacggcaatgagaaagatgcagcaaagggggaccagaaacagagcaaggagaattcgaagatggcggagccgcagaaggactatatccatgtgagagcaagaaggggtcaggccactgacagccatagccttgctgaaagagtcaggagagagaagataagtctgaggatgaaggctctccaggatctcgtgcctaactgcaacaaggtcactgggaaagctatgatgctcgatgagatcattaactacgtccagtccttgcaacgtcaggttgagcttctctccatgaagctgtcgactctgaaccccacagtggatttcaatctggaagctttcctgtcaaaagatatcttgcaccctcaaggacccttaccgaattctctctatccagttgattcgtcggggtccatgttgtatggttatcagcccccgcaaggactaatgccgatgccaagcgtcgtttccagcaatatggatgcccaatttccagtgaactttttgaattccggaattcacagagatgccagcttgaacttgcctctttgtgtggacaattacggcgaagcttctgctccggccccagctctatgggaggatgacctccaaagtgtagtccaaatgggtctcggccagattgagccccagagagtagaacaaggctccctggcgacttctcagatgaaagttgagttgtag

SEQ ID NO: 70 - protein, 498 aa, Arabidopsis thaliana
MDNELFMNTEFPPPPEMATHFEHQQSSSSAMMLNWALMDPNPHQDSSFLWEKSTEQQQQQSIFDSALSSLVSSPTPSNSNFSGGGGDGFLIRELIGKLGNIGNNNNNSGEIYGTPMSRSASCYATPMSSPPPPTNSNSQMMMNRTTPLTEFSADPGFAERAARFSCFGSRSFNGRTNTNLPINNGNNMVNNSGKLTRVSSTPALKALVSPEVTPGGEFSRKRKSVPKGKSKENPISTASPSPSFSKTAEKNGGKGGSKSSEEKGGKRRREEEDDEEEEGEGEGNKSNNTKPPEPPKDYIHVRARRGQATDSHSLAERVRREKIGERMKLLQDLVPGCNKVTGKALMLDEIINYVQSLQRQVEFLSMKLSSVNDTRLDFNVDALVSKDVMIPSSNNRLHEEGLQSKSSSHHHQQQLNIYNNNSQLLPNISSNNMMLQSPMNSLETSTLARSFTHLPTLTQFTDSISQYQMFSEEDLQSIVGMGVAENPNNESQHMKIEL

SEQ ID NO: 71 - DNA, 1497 bp, Arabidopsis thaliana
  source 1..1497: mol_type = genomic DNA
atggacaacgagctgtttatgaacacagagtttccaccaccgccggagatggcgacgcatttcgaacaccaacagtcttcttcatcggccatgatgcttaattgggctttaatggatccaaatccgcatcaagattcttcctttttatgggaaaagtcaacggaacaacaacaacaacaaagcatctttgactctgctttaagctcattagtctcatcaccgacgccgtcaaattccaacttctccggcggtggcggtgacggttttctcatcagagaactcatcggaaagcttggaaacatcggtaataataataacaactccggtgagatctacggaactccgatgtctcgctccgcctcatgttacgcaactccgatgagctctccaccgccaccgacgaattcgaattctcagatgatgatgaacagaacgacgccgttgacggaattctcagcagatccgggttttgcggagagagcagctagattctcttgttttggtagtcggagctttaacggaagaaccaatacaaatcttccgattaacaacggtaataacatggtcaacaactccgggaagctgacacgtgtctccagcacaccagctcttaaggctcttgtttcaccggaagtcacacccggcggcgaattttcccggaagagaaaatctgtgcctaaaggaaaatccaaagaaaaccccatttctacagcttctccatctcctagtttctcaaagacggcggaaaagaatggtggaaaaggaggaagtaaaagttcagaagaaaaaggaggaaaaaggagaagagaagaagaagatgatgaagaagaagaaggagaaggtgaagggaacaaaagcaataacacaaaaccacctgagcctcctaaagattacattcatgttcgagctcgacgaggccaagcaaccgatagtcacagcctcgccgaacgagttcggagggagaaaattggtgaaaggatgaagcttcttcaagatcttgtgcctggatgcaataaggttactggaaaagcactgatgcttgatgaaattataaactacgtacaatcattgcaaagacaagttgagttcttgtcaatgaagttatcatcagtgaacgacaccaggctggattttaacgtggacgctcttgtgtcaaaggatgttatgattccatcaagtaacaaccgattgcatgaagaaggactccaatcaaagtcttcaagtcatcatcatcaacaacaacttaatatttataacaataattcacaattacttcccaatatttcttccaataacatgatgctccagtctcctatgaactctttggaaacctctaccttagccagaagcttcactcacttaccaacacttacccaatttactgactcaatttctcagtatcaaatgtttagcgaagaagatttacaaagcatagtaggaatgggagtggcagaaaaccccaacaatgaatctcaacacatgaaaattgagctttga

SEQ ID NO: 72 - protein, 456 aa, Arabidopsis thaliana
MENELFMNAGVSHPPVMTSPSSSSAMLKWVSMETQPVDPSLSRNLFWEKSTEQSIFDSALSSLVSSPTPSNSNFSVGGVGGENVIMRELIGKLGNIGDIYGITASNGNSCYATPMSSPPPGSMMETKTTTPMAELSGDPGFAERAARFSCFGSRSFNSRTNSPFPINNEPPITTNEKMPRVSSSPVFKPLASHVPAGESSGELSRKRKTKSKQNSPSAVSSSKEIEEKEDSDPKRCKKSEENGDKTKSIDPYKDYIHVRARRGQATDSHSLAERVRREKISERMKLLQDLVPGCNKVTGKALMLDEIINYVQSLQRQVEFLSMKLSSVNTRLDFNMDALLSKDIFPSSNNLMHHQQVLQLDSSAETLLGDHHNKNLQLNPDISSNNVINPLETSETRSFISHLPTLAHFTDSISQYSTFSEDDLHSIIHMGFAQNRLQELNQGSSNQVPSHMKAEL

SEQ ID NO: 73 - DNA, 1371 bp, Arabidopsis thaliana
  source 1..1371: mol_type = genomic DNA
atggagaacgagctgtttatgaatgcaggagtttcgcatccgccggtgatgacgtcaccgtcttcttcgtcggcgatgcttaagtgggtttcaatggagactcagccagtggatccaagtctcagtcgcaatctgttttgggaaaagtcaacggaacagagcatttttgattcggctctgagctcactcgtctcttctcctacgccgtcaaattcaaacttttcggttggcggagtaggtggagaaaatgtcattatgagagagctcatcgggaaactgggtaatatcggcgatatctatggaattacggcgagcaatggaaactcgtgttacgcaactccgatgagctctccaccgcctggaagtatgatggagacgaaaacgacgacgccgatggcagaactgtccggcgacccgggattcgctgagagagcggcgaggttctcgtgttttggtagccggagttttaacagcagaacaaactcgccgtttccgattaataacgaaccacccatcacaaccaatgagaaaatgccacgtgtatcaagcagcccagtttttaagcctcttgcgtctcatgttcccgccggcgaatcgtccggtgaactttcccggaaaagaaaaactaaatccaagcagaattctccttctgcagtttcatcatcaaaggagattgaagagaaggaagattctgatccaaagagatgcaaaaaatcagaagaaaatggagacaaaactaaatcaattgatccttacaaagattacattcatgttagagctcgacgaggccaagccaccgatagtcacagtctcgccgaacgagttcgaagagagaaaataagcgaaagaatgaagctgcttcaagatcttgtccctggatgcaacaaggttactggaaaagcactgatgttggatgaaattataaactatgtccaatcattgcaacgacaagttgagttcttgtcgatgaagttatcgtcagtgaacaccaggctggactttaacatggacgctctcttgtctaaggatatatttccttcaagcaacaatctaatgcatcatcaacaagtacttcaattagattcttcagcagaaacgttattgggtgatcatcacaacaaaaatttgcaattgaaccctgatatttcttctaataacgtaataaaccctttggaaacttctgaaactagaagcttcatttctcacttaccaacacttgctcatttcactgactctatttctcagtattcaacattttccgaagatgatttacatagcattattcatatgggttttgcgcaaaaccgtctccaagaattgaaccaaggttcatcaaaccaagtaccatcgcacatgaaagctgaactttga

SEQ ID NO: 74 - protein, 301 aa, Populus trichocarpa
MAVDLVRYSKMEDQMAIQEAASAGLESMEHLIFAFSNQTRQSHQLDCGEITNFTVAKFKQVISMLNRTGHARFRRGPTSSPSSYPVPVRPVPQEPQKLNLDFVNSNSPPKAESKNDLSLGSQYSKDSLSSGTTTSSFVSSVTADGSVSNGKQGGSSLFGTQARSTGKPPLSSTHRKKCHDHALSARKISSGGSCHCSKRRKSRVKRTIRVPAVSSKIADIPADEYSWRKYGQKPIKGSPYPRGYYKCSSVRGCPARKHVERAVDDSAMLIVTYEGEHRHSHTPLPGDVTASAAMRHVFHST

SEQ ID NO: 75 - DNA, 906 bp, Populus trichocarpa
  source 1..906: mol_type = genomic DNA
atggctgtggatctagttaggtattcaaagatggaagatcagatggctatacaagaagctgcatcagctgggctcgagagcatggagcacttgatctttgcattctctaaccaaactcgacaaagccaccaacttgactgcggagaaatcacaaacttcaccgttgctaagttcaagcaagtcatctccatgttgaaccggaccggtcatgcccgttttcgccgtggaccaacttcttctccttcttcctacccggttcccgtccgacctgtccctcaagagcctcaaaaactgaaccttgattttgttaacagtaatagcccccctaaagctgagtcgaaaaatgacctgtctttgggtagtcagtattcaaaggatagccttagctctggcaccactacctcatccttcgtgtcttctgttacagctgatgggagtgtctctaatgggaaacaaggtggctcttctcttttcggaactcaagcgcgatctaccggaaaaccacctctctcatcgacccaccgcaagaaatgccacgatcatgccctctccgccagaaagatctcctccggtggaagctgtcattgctccaaaagaaggaaatcaagggttaagaggacaataagggtaccagccgtgagttccaagattgccgatataccagcagatgagtactcatggagaaaatatggtcaaaagccaatcaagggctcaccatacccaagagggtattacaagtgtagcagtgttagaggatgtccagcaaggaagcatgtggagcgtgccgtagatgactcggccatgcttatcgtgacttacgagggggagcaccgtcactcacatactccgttgccgggagacgtcaccgcgagtgctgcaatgcgacacgtgtttcactcaacatga

SEQ ID NO: 76 - protein, 300 aa, Populus trichocarpa
MAVDLVGYSKMEDQMAIQEAASAGIKSMEHLIFALSNQTQQSHQLDCREITSFTVAKFKQVISILNRTGHARFRRGPTSSNPVSVRPVVQEPQKLNLDFFKSNNTFKSETKNDLSFGSQYSKDCFSSGTTTSSFLSSVTADGSVSDGKQGGSSSLFGTHPRPTGKPPLSSIHRKKCHDHTLSTSKISSSGGSCHCSKRRKSRVKRTIRVPAISSKVADIPADEFSWRKYGQKPIKGSPYPRGYYKCSSVRGCPARKHVERAVDDPAMLIVTYEGEHRHSHAPLPENVTANAAMRHVFQST

SEQ ID NO: 77 - DNA, 903 bp, Populus trichocarpa
  source 1..903: mol_type = genomic DNA
atggctgttgatctagttgggtactcaaagatggaagatcagatggctattcaagaagctgcatcagctgggattaagagcatggagcacttgatctttgcactctctaaccaaacccaacaaagtcaccaacttgactgcagagaaatcactagcttcaccgttgctaagttcaagcaagtcatctccatcctgaaccggaccggtcatgcccgtttccgccgtggacccacttcttccaacccggtttccgtccgacctgtcgtccaagaacctcaaaaactaaatcttgatttctttaagagtaacaatacctttaaatctgagaccaaaaacgacttgtcattcggcagtcagtattccaaggattgttttagctctggcacgactacttcgtccttcctgtcttctgttacagctgatgggagtgtctctgatgggaaacaaggtggctcgtcttctcttttcggaactcatcctcgacctaccggaaaaccacctctctcatctatccatcgcaagaaatgccatgatcataccctctccaccagcaagatctcctcctccggtggcagctgccattgctccaaaagaaggaaatcaagggtgaagaggacaataagagtcccagcaataagttccaaagttgctgatataccagcagatgagttctcatggagaaaatatggtcaaaagccaatcaagggttcaccttacccaagagggtactacaagtgtagtagtgtgaggggatgtccagcaaggaagcacgtggagcgtgccgtagatgatccggccatgcttattgtaacatacgaaggggagcaccgtcactcacatgctcccctaccagaaaatgtcacagccaacgctgccatgcgacacgtgttccagtcaacatga

SEQ ID NO: 78 - protein, 343 aa, Eucalyptus grandis
MAIELHLGFSKMEDHTAIQEAASQGLKTMEHLIGVLSRQNLHHPGAVDCTDLTDRTVSKFRKVISLLDRTGHARFRRAPLPSSSSSSSKSAPVASPVPPAVQSRSQPLAPTPIQAPTSSQPSPASFLHAQPKQSLTLDFTRPSILGPNSKGVSEIEFAKDSFSVSSNSSFMSSAITGDGSVSNGKLGTSMFIAPASGPASSAGKPPISSVPYKKRCHEHDPSDNISGKHSGSGSGKCHCSKRRKNRVKKVTRVPAISNKIADIPADEFSWRKYGQKPIKGSPFPRGYYKCSTMRGCPARKHVERAPDDPTMLIVTYEGEHRHSQSASQEIVPAGAMNLVFKST

SEQ ID NO: 79 - DNA, 1032 bp, Eucalyptus grandis
  source 1..1032: mol_type = genomic DNA
atggcgatcgagctgcatcttggcttctccaagatggaggatcacacggcgatacaggaggccgcgtctcaggggctgaagaccatggagcacctgatcggcgtcctctcccgccagaacctccaccaccccggcgccgtcgactgcaccgacctcaccgaccggaccgtctccaagttcaggaaggtcatctccctcctggaccgcaccggccacgcccgcttccggcgggccccgctcccctcctcctcctcttcctcctccaagtctgcccccgtcgcttcccctgtccccccggcggttcagtctcggagccagcctctagcgccgacgcccatccaggccccgacgagctcgcagccgtctccggccagcttcctccacgcgcagcccaagcagagcttgaccctcgacttcacccggccgagcatcctgggccccaactccaaaggcgtttccgagatcgagttcgcgaaggacagcttcagcgtgtcgtccaactcttccttcatgtcttcggcgatcaccggcgacggaagcgtttcgaacgggaagctggggacgtcgatgttcatagctccggcgtccggcccggcttcgtccgccggaaagccaccgatctcgagcgtgccgtacaagaagcggtgccacgagcacgacccctccgataacatctccggcaagcactccggctccggcagcggcaagtgccactgctccaaaagaaggaaaaaccgagtgaagaaggtgacgagagtgccggcgatcagcaacaagatcgcggacatcccggccgacgagttctcgtggaggaagtacggacagaagccgatcaaagggtctcccttcccacgaggttactacaagtgcagtacaatgagagggtgccccgcgaggaaacacgtggagagggcccccgacgatccaacgatgctgatcgtgacgtacgagggggagcatcgtcactcccagtccgcgtcgcaggagattgtgcccgccggtgcgatgaacctggtattcaagtcaacctga

SEQ ID NO: 80 - protein, 325 aa, Arabidopsis thaliana
MAVDLMRFPKIDDQTAIQEAASQGLQSMEHLIRVLSNRPEQQHNVDCSEITDFTVSKFKTVISLLNRTGHARFRRGPVHSTSSAASQKLQSQIVKNTQPEAPIVRTTTNHPQIVPPPSSVTLDFSKPSIFGTKAKSAELEFSKENFSVSLNSSFMSSAITGDGSVSNGKIFLASAPLQPVNSSGKPPLAGHPYRKRCLEHEHSESFSGKVSGSAYGKCHCKKSRKNRMKRTVRVPAISAKIADIPPDEYSWRKYGQKPIKGSPHPRGYYKCSTFRGCPARKHVERALDDPAMLIVTYEGEHRHNQSAMQENISSSGINDLVFASA

SEQ ID NO: 81 - DNA, 978 bp, Arabidopsis thaliana
  source 1..978: mol_type = genomic DNA
atggccgtcgatctaatgcgtttccctaagatagatgatcaaacggctattcaggaagctgcatcgcaaggtttacaaagtatggaacatctgatccgtgtcctctctaaccgtcccgaacaacaacacaacgttgactgctccgagatcactgacttcaccgtttctaaattcaaaaccgtcatttctctccttaaccgtactggtcacgctcggttcagacgcggaccggttcactccacttcctctgccgcatctcagaaactacagagtcagatcgttaaaaatactcaacctgaggctccgatagtgagaacaactacgaatcaccctcaaatcgttcctccaccgtctagtgtaacactcgatttctctaaaccaagcatcttcggcaccaaagctaagagcgccgagctggaattctccaaagaaaacttcagtgtttctttaaactcctcattcatgtcgtcggcgataaccggagacggcagcgtctccaatggaaaaatcttccttgcttctgctccgttgcagcctgttaactcttccggaaaaccaccgttggctggtcatccttacagaaagagatgtctcgagcatgagcactcagagagtttctccggaaaagtctccggctccgcctacggaaagtgccattgcaagaaaagcaggaaaaatcggatgaagagaaccgtgagagtaccggcgataagtgcaaagatcgccgatattccaccggacgaatattcgtggaggaagtacggacaaaaaccgatcaagggctcaccacacccacgtggttactacaagtgcagtacattcagaggatgtccagcgaggaaacacgtggaacgagcattagatgatccagcgatgcttattgtgacatacgaaggagagcaccgtcataaccaatccgcgatgcaggagaatatttcttcttcaggcattaatgatttagtgtttgcctcggcttga

SEQ ID NO: 82 - protein, 321 aa, Arabidopsis thaliana
MTVDIMRLPKMEDQTAIQEAASQGLKSMEHLIRVLSNRPEERNVDCSEITDFTVSKFKKVISLLNRSGHARFRRGPVHSPPSSSVPPPVKVTTPAPTQISAPAPVSFVQANQQSVTLDFTRPSVFGAKTKSSEVVEFAKESFSVSSNSSFMSSAITGDGSVSKGSSIFLAPAPAVPVTSSGKPPLSGLPYRKRCFEHDHSEGFSGKISGSGNGKCHCKKSRKNRMKRTVRVPAVSAKIADIPPDEYSWRKYGQKPIKGSPHPRGYYKCSTFRGCPARKHVERALDDSTMLIVTYEGEHRHHQSTMQEHVTPSVSGLVFGSA

SEQ ID NO: 83 - DNA, 966 bp, Arabidopsis thaliana
  source 1..966: mol_type = genomic DNA
atgaccgttgatattatgcgtttacctaagatggaagatcaaacggctatacaagaagctgcatcacaaggcttaaaaagcatggaacacttgattcgtgtcctctctaaccgtcccgaagaacgtaacgttgattgctctgagatcactgatttcacagtttctaagttcaagaaagttatctctcttcttaaccgttccggtcacgcccggtttagacgtggtccggttcattcccctccttcctcctccgttcctccaccggtgaaagtgacaactccggctcccactcagatctctgctccagcaccggttagcttcgttcaggcaaatcaacaaagcgtgacgttagatttcactagaccgagcgtttttggcgctaaaaccaagagctcggaggttgttgagtttgctaaagagagctttagcgtatcttctaactcttctttcatgtcttctgcgatcaccggtgatggaagtgtctctaaaggctcttcgatctttcttgctccggctccagcggtgccagtgacttcctccgggaaaccgccgctttctggtcttccttacaggaagagatgctttgaacatgaccactctgaaggcttttccggcaagatctctggctccggcaacggcaagtgccattgcaagaaaagccgaaaaaatcggatgaagagaaccgtgagagtaccggcggtaagtgcaaagatcgccgatataccaccagacgaatattcatggagaaagtatggacaaaaaccgatcaaaggctcaccacatccacggggttattacaagtgtagtacatttagaggatgtccagcgaggaaacacgtggaaagagctttggatgattcaacgatgttgattgtgacgtacgaaggagagcaccgtcatcaccagtccacgatgcaggagcatgtaactcctagcgtgagtggtttggtgtttggttcggcttga

SEQ ID NO: 84 - protein, 347 aa, Populus trichocarpa
MEGVEEANRAAVESCHRVISLLSQPQDQVQYRNLMVETGEAVFRFKKVVSLLNTGLGHARVRKLKKLPTPLSQSILLDNPLSSTDHPSKTPQFLQSSSYLESQPIQELGSIAKNCLSLGTPSLELSSNGKNPLQLGQPTPAAHYQFLQQQQLHRLQLQQQQQMKQQAEMMFRKSNSGISLNFDSSSCTPTMSSTRSFISSLSIDGNVANLEGSAFHLTGAARSSDQSSQQHKRKCSGRGEDGSMKCGSSVRCHCSKKRKHRVKRSIKVPAISNKLADIPPDDYSWRKYGQKPIKGSPHPRGYYKCSSMRGCPARKHVERCLEDPSMLIVTYEGEHNHPRIPAQSTNT

SEQ ID NO: 85 - DNA, 1044 bp, Populus trichocarpa
  source 1..1044: mol_type = genomic DNA
atggagggggttgaagaagctaaccgggcagctgtggagagctgccatagagttataagtttgctatcccaaccccaagatcaggttcaatataggaatttaatggtggaaactggagaggctgtgtttaggttcaagaaagtagtttcccttttaaatactggtttaggtcatgcaagagttcgaaaacttaagaagttaccgacccctttatcccaaagcatcctcttagacaacccactgagcagtacagaccacccatccaaaacaccccagtttctccagtccagtagttacctggaaagccaaccaattcaagaattgggctcaattgctaaaaattgtctatctttgggaaccccatccctggaattgagttcaaatgggaaaaaccctcttcagcttggccaacccacgccagcagcgcactatcagttccttcagcaacagcaactgcataggctacagcttcaacagcagcagcaaatgaagcagcaagctgagatgatgtttagaaaaagcaatagtgggattagcttgaatttcgatagttctagctgcactcctacaatgtcatctaccagatcttttatatcgtccttgagtattgatggtaatgtggctaatttggaaggaagtgcattccatttaacgggggcggctcgctcctcagatcagagttcacagcaacacaagaggaaatgttctggaaggggagaagatggaagtatgaaatgtggaagcagcgttagatgtcattgctcaaagaagaggaaacatagggtgaagaggtcgatcaaggttcctgctattagcaacaagcttgctgatatccctcctgatgattactcatggagaaagtatgggcaaaagccaatcaagggttctcctcatcctaggggatattacaaatgtagcagtatgagaggttgtcctgcaaggaagcatgtggagaggtgcttggaagatccgtccatgcttattgttacctatgaaggtgaacataaccacccaaggattccagcacaatccacaaacacgtaa

SEQ ID NO: 86 - protein, 351 aa, Populus trichocarpa
MEGVEEANRAAVESCHRVISLLSQPQDQVQYRNLMVETGEAVFRFKKVVSLLNTGLGHARVRKLKKLPTPLSQSILLDNSLNRTGHSSKTPQFLQSSSYLESHPIQELGSSAKNCLSLGTPSLELSSTGKNPLQLGQPTSAANYQFLQLQQQQLHRLQLQQQQQQQMKQQAEMMFRKSNSGISLNFDSSSCTPTLSSTRSFISSLSIDGSVANLEGSAFHLMGPARSSDQSSQQHKRKCSGRGEDGSVKCGSSGRCHCSKKRKHRVKRSIKVPAISNKLADIPPDDYSWRKYGQKPIKGSPHPRGYYKCSSMRGCPARKHVERCLEDPSMLIVTYEGEHNHPRIPAQSANT

SEQ ID NO: 87 - DNA, 1056 bp, Populus trichocarpa
  source 1..1056: mol_type = genomic DNA
atggagggggttgaagaagctaacagagcagctgtggagagctgccatagagttataagtttgttgtctcaacctcaagatcaggttcaatataggaatttaatggtggaaactggcgaggctgtgtttaggttcaagaaagttgtttctcttttaaatactggtttaggtcatgcaagagttcggaaacttaagaagttaccgacccctttatcccaaagcatccttttagataactcacttaatagaacaggccactcatccaaaacacctcagtttctccagtctagcagttacctggaaagccatccgattcaagaattgggctcaagtgctaagaactgtctttctctgggaaccccatctttggaattgagttcaaccgggaaaaaccctcttcagcttgggcaacccacttcagcagctaattatcagttccttcagcttcaacagcagcagctgcacaggctacagcttcagcagcagcagcagcagcaaatgaagcagcaagctgagatgatgtttagaaaaagcaatagtggaattagcttgaattttgatagttctagctgcacacctacactgtcatccaccagatcttttatatcttccttgagtatagatggtagtgtggctaatttggaaggaagtgcattccatttaatggggccggctcgctcctcggatcagagttcacagcaacacaagaggaaatgttctgggaggggagaagatggaagtgtgaaatgtggaagcagtggtagatgtcattgctcgaagaagaggaaacatagggtaaagaggtcgatcaaggtacctgctattagcaacaagcttgctgatatcccccctgatgattattcctggagaaagtatggacaaaagcccatcaagggctctcctcatcccaggggatattacaagtgtagcagtatgagaggttgtcctgcaaggaagcatgtggagaggtgcttggaagatccgtccatgcttattgttacctatgaaggtgaacataaccacccgaggattccagcacaatctgcaaacacataa

SEQ ID NO: 88 - protein, 343 aa, Eucalyptus grandis
MEEVEEANRAAVESSHRVLSLLSQPQDQVQCRNLMLETGDAVFRFKRVVSLLNTCLGHARVRKLKKLPTPLPQKALLDNPIVRTDQSSKSLQLLPPNYPENAIQELNTHHKASLSLGNPNLELSSNGKSPLHLAQQAPSAHYHFLQQQQQQQKLQFQQQMKHQAEMMYHRSNSGISLNFDSSSCTPTMSSTRSFISSLSIDGSVANLDANAFHLIGAARSSDQNSQHKRKCSGRGEDGSMKCGSSSRCHCSKKRKHRVKRSIKVPAISNKLADIPPDDYSWRKYGQKPIKGSPHPRGYYKCSSMRGCPARKHVERCLEDPSMLIVTYEGDHNHPRVPSQSANT

SEQ ID NO: 89 - DNA, 1032 bp, Eucalyptus grandis
  source 1..1032: mol_type = genomic DNA
atggaggaggttgaagaagcgaacagagcagctgtggagagctcccacagggttctgagcctcttgtcgcagccgcaagatcaggttcagtgtagaaatctgatgcttgagactggtgatgctgtattcaggttcaagagagtggtttctcttctcaatacttgcttgggtcatgctagagttaggaagttaaagaaactaccgacccctttgccccaaaaggccctcttggacaaccctattgttagaactgaccaatcttcaaagagtctccaactcctccctcctaattaccctgaaaatgcaattcaggaactcaatacacatcacaaagcctctctttctttaggtaacccgaacttggagttgagctccaatggcaaaagccctcttcatttagcccagcaagcgccatcagcacactatcactttctccagcagcagcagcagcagcagaagttacagttccagcaacaaatgaaacaccaagctgagatgatgtaccataggagcaatagtggcataagcttgaatttcgatagctctagctgcacgcccaccatgtcgtctacgaggtctttcatttcttcactgagtatagatgggagcgtggctaacttggatgcgaatgctttccatttgatcggggcggctcgctcatctgatcagaactcgcagcataagaggaagtgctctggcagaggagaggatgggagcatgaaatgcggtagcagtagtagatgtcattgctctaagaagaggaaacatagggtgaagaggtcgattaaggtacctgctataagcaacaagcttgcggatattcctcccgatgattattcgtggaggaagtacgggcagaagccaatcaagggttctcctcatcctaggggatactacaaatgtagcagcatgagaggttgccctgcaaggaagcatgtggaaaggtgcttggaggacccgtctatgctcattgtcacctacgaaggtgaccataaccaccccagggtgccatcacaatctgccaatacatga

SEQ ID NO: 90 - protein, 380 aa, Arabidopsis thaliana
MEEIEGTNRAAVESCHRVLNLLHRSQQQDHVGFEKNLVSETREAVIRFKRVGSLLSSSVGHARFRRAKKLQSHVSQSLLLDPCQQRTTEVPSSSSQKTPVLRSGFQELSLRQPSDSLTLGTRSFSLNSNAKAPLLQLNQQTMPPSNYPTLFPVQQQQQQQQQQQQQEQQQQQQQQQQQFHERLQAHHLHQQQQLQKHQAELMLRKCNGGISLSFDNSSCTPTMSSTRSFVSSLSIDGSVANIEGKNSFHFGVPSSTDQNSLHSKRKCPLKGDEHGSLKCGSSSRCHCAKKRKHRVRRSIRVPAISNKVADIPPDDYSWRKYGQKPIKGSPYPRGYYKCSSMRGCPARKHVERCLEDPAMLIVTYEAEHNHPKLPSQAITT

SEQ ID NO: 91 - DNA, 1143 bp, Arabidopsis thaliana
  source 1..1143: mol_type = genomic DNA
atggaggagatagaaggaacaaacagagcagctgttgagagttgtcatagagttcttaatcttttacatagatcacagcaacaagatcatgttggttttgaaaagaatttagtatctgaaactagagaagctgtgattaggttcaagagagttgggagtttgttaagcagtagtgttggtcatgctaggtttagaagagctaagaaacttcagagtcatgtctctcaaagtctcttacttgatccatgtcaacaaaggacaacagaagttccatcatcatcttctcagaaaacaccggtactccggtctggtttccaggaattgagcttgagacaaccttcagattcactcactttagggactcgctcttttagtttaaactcaaatgctaaagctcctctccttcagcttaatcagcagacaatgcctccttcgaattatcctactttgtttccagtacaacaacaacaacaacaacaacaacaacaacaacagcaggagcagcagcagcagcagcagcagcaacagcaacagtttcatgaacggttacaagctcaccatcttcatcagcaacagcagctacagaaacatcaagctgagcttatgcttaggaaatgcaatggtgggataagtttgagtttcgataactctagttgtactccaactatgtcatccactaggtcctttgtttcttcacttagcatagatggtagtgttgctaatatagagggaaagaactccttccattttggggttcctagttcaactgatcagaattcactacattctaagaggaaatgccccttgaaaggagatgaacatggaagcttaaaatgcgggagctctagccgatgccactgcgctaagaagaggaaacatcgggttaggagatcgattagagtaccggctataagtaacaaggttgcagatatccctcctgatgattattcatggcgaaaatatggtcagaagcccatcaagggctctccttatcccagaggatattacaaatgtagtagcatgagaggttgtccagcgaggaagcatgttgagagatgtttggaagatccggcaatgcttattgttacttatgaagcagagcataaccacccgaaattgccatctcaagctataacaacttaa

SEQ ID NO: 92-99 - skipped (8 entries)

SEQ ID NO: 100 - protein, 178 aa, Zea mays
MRRAEPVGEQADAERRMSGYKGVRRRRWGKWVSEIRVPGSRERLWLGSYATPEAAAVAHDTAVYFLRGGGGAGDVAALNFPERAAAAYGAGGRLSPRSVQRVASDAGMAADAQLVAAREDTRALRAGIGGGASARPRDRDAGDACAGRAHNASLHSTGAGREQPVSGEISVDDMDILL

SEQ ID NO: 101 - DNA, 537 bp, Zea mays
  source 1..537: mol_type = genomic DNA
atgaggcgcgcggagccggtcggcgagcaggcggacgcggagcggcgcatgagcgggtacaagggcgtgcggcggcgccggtgggggaagtgggtgtcggagatccgggtgcccggctcgcgggagcgcctgtggctcggctcctacgccacgcccgaggccgccgccgtcgcgcacgacacggccgtctacttcctccgcggcggcggcggcgcgggcgacgtcgcggcgctcaacttccccgagcgcgcggcggccgcgtacggcgcagggggccgcctgtcgccccggtccgtgcagcgcgtggcgtccgacgccggcatggccgccgacgcgcagctcgtcgcggcgcgggaggacacccgcgcgcttcgcgccggcattggcggtggcgccagcgcgcgcccgcgcgatcgggatgctggtgacgcctgcgcgggccgtgcgcacaacgctagccttcacagtacgggcgccggaagggagcagcctgtctccggggagattagcgtggatgacatggatattttgctgtag

SEQ ID NO: 102 - protein, 186 aa, Zea mays
MSRAATNSGAERRCRYRGVRRRAWGKWVSEIRVPGTRERLWLGSYAAPEAAAVAHDAAACLLRGCAGRRLNFPGRAACYYACGGQQPLSPRSVQRVASDAGMAADAQIVDARAALASPPPVVQPAALAGIIGGAAREGGGGVRGPACAPAPPSNGAGSSSTYWSTPSSEPPLVYGDISVDDIEILI

SEQ ID NO: 103 - DNA, 561 bp, Zea mays
  source 1..561: mol_type = genomic DNA
atgagccgcgcagcgaccaacagcggcgcggagcggcggtgccggtacaggggcgtgcggcggcgggcctgggggaagtgggtgtcggagatccgggtgccgggcacgcgggagcggctgtggctgggatcctacgcggcgcccgaggccgccgccgtcgcgcacgacgccgccgcgtgcctcctccgcggctgcgcgggccgccgcctcaacttcccgggccgcgccgcctgctactacgcctgcggcgggcagcagccgctgtcgccgcgctccgtgcagcgcgtcgcgtccgacgccggcatggccgccgacgcgcagatcgtcgacgcgcgggcggccctcgcctcgccgccgcccgttgtccagcccgccgctctcgctggcattattggcggcgccgcgcgagaaggcggcggaggcgtgcgaggccccgcgtgcgcgccggcgccgccaagcaacggcgctggcagcagcagtacgtattggtccacgccgagcagtgagccgccgcttgtttacggggacattagcgtagacgacatagagatcttgatttga

SEQ ID NO: 104 - protein, 316 aa, Zea mays
MAVDLMGCYAPRRANDQLAIQEAAAAGLRSLELLVSSLSTQAAAPHRAAAHQLQKPPSQPPIGEIADQAVSRFRKVISILDRTGHARFRRGPVVEAPPPVPPPAVSAPALPVAHVVAPVGAAQPQSLTLDFTKPNLAVSGGATSVTSTSFFSSVTAGEGSVSKGRSLVSSGKPPLSGHKRKPCAGAHSEATTNGSRCHCSKRRKNRVKRTIRVPAISAKIADIPPDEYSWRKYGQKPIKGSPYPRGYYKCSTVRGCPARKHVERATDDPAMLVVTYEGEHRHTPGAPAPAPSPLAAASPVPASAAAAVSAGNNGLV

SEQ ID NO: 105 - DNA, 951 bp, Zea mays
  source 1..951: mol_type = genomic DNA
atggccgtggacctgatggggtgctacgccccgcgccgcgccaacgaccagctcgccatccaggaggcggcggcggcggggctccgcagcctggagctcctcgtgtcgtcgctgtccacgcaggccgccgcgccgcacagggccgcggctcaccagctgcagaagccgccttcgcagccgccgatcggcgagatcgccgaccaggccgtctccaggttccgcaaggtcatctccatcctggaccgcaccggccacgcccgcttccggcgcgggcccgtggtcgaggcgccgccaccggtgcctcctccggccgtctccgctcccgctctccccgtggcgcacgtggtggctcccgtcggcgcggcgcagccccagagcctgaccctggacttcacgaagccgaacctggccgtgtcgggcggcgccacgtccgtcacctccacgtccttcttctcctcggtcacggccggcgagggcagcgtgtccaagggccgcagcctggtgtcctccggcaagccgccgctgtccggccacaagcggaagccctgcgccggcgcgcactccgaggccaccaccaacggcagccgctgccactgctccaagagaaggaaaaaccgcgtgaagaggaccatcagagtgccggcgatcagcgccaagatcgcggacatcccgccggacgagtactcgtggaggaagtacggccagaagcccatcaagggctccccctacccacggggctactacaagtgcagcaccgtgcgcgggtgcccggcgaggaagcacgtggagcgcgccaccgacgacccggccatgctggtggtgacgtacgagggcgagcaccgccacacgccgggcgcgcccgcgcccgcgcccagccccctggcggccgcgtcgccggtgcccgcctccgccgccgccgccgtctccgccggcaacaacgggcttgtctag

SEQ ID NO: 106 - protein, 331 aa, Zea mays
MTTLDLMGGYGRVDEQVAIQEAATAGLRGMERLILQLSQAGTGERSLSPPAVQAQRQQQKQLEQIQQQVDCRELTDMTVSKFKKVISILNRTGHARFRRGPVAARSQSQSQGPASPEPAQSAPAPAARPLTLDFTKSVSGYSRDSGFSVSGASSSFLSSVTTGDGSVSNGRAGGSSFLMFPPAPGAASCAKPPPAGAAQKRKCHDHAHSENVAGGKYGANGGRCHCSKRRKHRVKRTIRVPAISPKVADIPADEYSWRKYGQKPIKGSPYPRGYYKCSTVRGCPARKHVERDPADPSMLIVTYEGEHRHSPASGQDPPPPSLAPLPELPSH

SEQ ID NO: 107 - DNA, 996 bp, Zea mays
  source 1..996: mol_type = genomic DNA
atgacgaccctcgatctgatgggagggtacgggcgggtggacgagcaggtggccatccaggaggccgccacggcggggctgcgcgggatggagcgtctcatcttgcagctctcccaggctggcaccggggagcggtcgttgtccccaccggcggtacaggcgcagcgccagcagcagaagcagctggagcagatccagcagcaggttgactgccgggagctcacggacatgacggtgtccaagttcaagaaggtgatctccatcctgaaccgcacggggcacgcgcggttccggcgtggccccgtggcggcgcggtcgcagtcgcagtcgcagggacctgcctcccccgagcccgcgcaatcggcgccggctcccgccgcgaggcccctgacgctggacttcaccaagtcggtgtccggttacagcagggactccgggttcagcgtgtccggcgcgagctcgtcgttcctgtcgtcggtgacgaccggggacgggagcgtgtcgaacgggcgcgcgggaggctcgtcgttcctcatgttcccaccggcgcccggcgcggccagctgcgcgaagccgccgcccgccggtgcggcgcagaagcgcaagtgccacgaccacgcgcactcggagaacgtcgccggcggcaagtacggggctaacggcgggcgctgccactgctcgaagcgcaggaagcaccgtgtgaagcgcacgatccgcgtgccggcgatcagccccaaagtggcggacatccccgccgacgagtactcgtggcgcaagtacggccagaaacccatcaaggggtcgccctacccacgcggctactacaagtgcagcacggtgcgcggctgccccgcccggaagcatgtggagcgcgaccccgccgacccgtcgatgctgatcgtcacctacgagggcgagcaccgccacagccccgcctccggccaggacccgccgccgccgtcgctcgcgccgctgccggagctgcccagccattga

SEQ ID NO: 108 - protein, 350 aa, Zea mays
MDSDYIASLLMGSSAPALSFAALDAGFLDTLRGGGGGGLFGVPAEAGCGGGSPEGSSVSDPAWARARDYGNARKRKAPPTGSAGGKEACLSKVAEAKGPDGKRCRVVGASDSPVKPKEEEEEAAASDALVEVKAQKKGKGKSSKPAVEPPKDYVHVRARRGQATDSHSLAERVRREKISQRMKFLQDLVPGCNKVVGKALMLDEIINYVQSLQQQVEFLSMKLATVNPELDFSNLSTLLHKDMYQQPCGGPSASSVFPLESAGAAFPFCEQADLFHSFGSGGSGSGSGMEDQCSLSLLLDTALPHAASPQFAFQKQQRDLWEDGLQHALPTPTTGSEQRQEEDGLLVPDL

SEQ ID NO: 109 - DNA, 1053 bp, Zea mays
  source 1..1053: mol_type = genomic DNA
atggacagcgactacattgccagcctgctcatgggctcatccgcccctgcactcagcttcgccgcgctggacgccgggttcctcgacacgctacgcggcggcggcggcgggggcctcttcggggtgcccgcggaggcagggtgtggcggcgggtctccggaggggtcgtcggtgtccgacccagcgtgggcgcgcgctagggactacggcaatgccaggaaacgcaaggcgccgcccacggggtccgccggtggcaaggaggcctgcttgagcaaggttgcggaggccaagggtccggacgggaagaggtgcagagtagtgggtgccagcgacagtccggtgaagccgaaggaagaggaggaggaggcggcggcgagcgacgccttggttgaagtcaaagcgcagaagaagggcaaggggaagagctcgaagccggcagtcgaaccgcccaaagactacgtccatgtccgggcgcggcgggggcaggcgactgacagccacagccttgcagagagggttagaagagagaagattagccagaggatgaaatttctacaggacctagtgccaggatgcaacaaggtggtcggcaaggcactcatgctcgatgagatcataaactacgtccagtcgctgcagcagcaagttgagttcctgtccatgaagctcgccactgtgaacccagagcttgatttcagcaacctatcgacactcctgcacaaagacatgtaccaacagccatgtggtggcccttcggcaagttcggtctttccactggaaagcgccggcgcagcttttccattctgcgagcaggcggatcttttccacagctttggctcgggtggtagcggtagcggtagtggtatggaggatcagtgctctctaagcctgctgctagacacggctctgccccacgcggccagcccacagtttgcttttcaaaagcagcaaagggacttgtgggaggatggcctacagcatgctttgcctacccctactaccggtagcgagcaaaggcaggaggaggatgggcttttagtacccgacctttga

SEQ ID NO: 110 - protein, 460 aa, Zea mays
MDCGPPDQLPPSSAPACFLNLNWDQSMAAATAGDHLDLVSSPASNSTAADGLALHGISPQPQYGGTPLSSPRKLNLSMMGQFHHYPPMGHLDQFLADPGFAARAARLSGFDGRPGGSGYGGAVPGQFGLPDAGPIGGALRELELGNGRDESSVSDPASASAEMALKAPSDGNAKKRKASGKGKGKDGPGSTAATKEESSGKRCKSAEESNGAEENSGKGKAAAQSNSDNGGKKQGKDGASKPPEPPKDYIHVRARRGEATDSHSLAERVRREKISQRMKLLQDLVPGCNKVVGKAVMLDEIINYVQSLQRQVEFLSMKLATVNPQLDFNNLPNLLPKDMHQSCGPLQNSHFPLETSGAPLPYLNQGNPLIGCGLPNGMDNSQSSMHPLDPAFCRPMSSQQHPFLNGVSDAASKVGTFWQDDLQSVVHMDMGQQSQQEMAPTSSNSYNDGSLQTVHMKMEL

SEQ ID NO: 111 - DNA, 1383 bp, Zea mays
  source 1..1383: mol_type = genomic DNA
atggactgcgggccgcccgaccagctgccgccgtcgtcggcgccggcgtgcttcctcaacctcaactgggaccagtccatggccgccgccacggctggcgaccacctcgacctggtctcctccccggcgtccaactcgacggcggctgacggcctcgctctccacgggatctcgccgcagccgcagtacggaggcactccgctcagctcgccccgcaagctgaacctctccatgatgggccagttccaccactacccgccgatgggccatctagaccagttcctcgccgacccaggcttcgccgcgcgcgcggcgaggctctccggcttcgacggccgccccggtgggagtggctacggcggcgccgtcccgggacagtttggcctccccgacgccggccccatcggcggcgcattgagggagctggagctcgggaacggccgggacgagtcatcggtgtccgatccggcgtccgccagcgccgagatggcgctcaaggcgccttccgatggcaatgcgaagaaacggaaggctagcgggaaggggaaaggcaaggacggccccgggtccaccgccgccaccaaggaggagtccagtgggaaacggtgcaaatcggcggaggagagcaatggcgcggaggagaactccggcaagggtaaggccgccgcgcagagcaacagcgacaacggtgggaagaagcaggggaaggacggcgcgtccaagcctccggagccgcccaaggactacatccacgtccgggcgaggcgcggcgaggcgacagacagccacagcctcgctgagagggtgagaagggagaagatcagccagcggatgaagcttctgcaggatctcgtcccgggctgcaacaaggtggtcggcaaggcggtgatgctggatgaaatcataaactacgtgcagtccttgcaacggcaagtcgagttcctgtccatgaaactggccaccgtgaatccccagctggacttcaacaacctgcctaacctccttcctaaagatatgcaccagtcctgcggcccgctgcagaactcgcatttcccgctggagacctcaggcgcgccgctgccatacctcaaccaggggaaccctctaataggctgcggcctacccaacggcatggacaacagccagagctccatgcacccgctcgacccggcgttttgccggccgatgagctcgcagcagcaccctttcctcaacggtgtcagcgacgcagcgtccaaggtcgggactttctggcaagatgaccttcagagcgtagtccacatggatatggggcagcagagccagcaggagatggctcccacctcctccaacagctacaacgacggttcgttgcaaacagtgcacatgaaaatggagctttga

SEQ ID NO: 112 - protein, 404 aa, Zea mays
MKEGRPGAGDYIASLLSSSPRLDFGGEEEEDCLDRFCGDPGFAARAARLSSFSGQRFAVTAGLFGLPPPLPAASGGGEFAGSREASSVSDPASAMKDANAKKRKAPAAAAAKGKGREPSAQAQAGEPKGPDAKRCCKAEGGEGEEGSPVKLPKPEQAGSDSSVEDGGAQNQKPPPPVKGKNAKPVEPPRDYVHVRARRGQATDSHSLAERVRRERISQRMKVLQDLVPGCNKVIGKALMLDEIINYVQSLQRQVEFLSMKLATVNPLDLSNLPTLLQKDMFQACGASASSVFSLESCSPGFPFGGQGDVFQSFVPNGLENPCGGLNPLDLALSQATGGQFGFQDGTAGTNLQQRNYWEEEEEDLQSVFHIDDNGQSQEHGASASAQSFHGQLQPQEGHMKMEFF

SEQ ID NO: 113 - DNA, 1215 bp, Zea mays
  source 1..1215: mol_type = genomic DNA
atgaaagagggcagacccggcgccggcgactacattgccagcctgctaagctcgtcgccgaggctcgacttcggcggggaggaggaggaggactgcctggacaggttctgcggcgacccggggttcgcggctcgcgcggcgcggctgtccagcttcagcgggcagcgcttcgccgtcaccgcgggcctcttcgggctgcccccgccgctgcccgccgcgagcggcggcggcgagttcgccgggtcccgggaggcgtcgtcggtgtcggacccggcgtcggcgatgaaggacgccaatgccaagaagcggaaggcgcccgcggcggcggcggccaagggcaaaggcagggagccatctgcgcaggcacaggcaggggagccgaagggtccggacgccaagaggtgctgcaaggcggagggaggcgagggggaggaggggagccccgtgaagctgcccaagccggagcaggccggcagcgacagctccgtcgaggacggcggcgcgcagaaccagaagccgccgccgccggtgaagggaaagaatgccaagccggtggagcctcccagggactacgtccatgtccgggccaggagagggcaggctaccgacagccacagcctcgcagagagggtgagaagggagaggatcagccagaggatgaaggtccttcaggacctggtgccaggatgcaacaaggtgatcggcaaggcgctgatgctcgacgagatcataaactacgtgcagtcgctgcaacggcaggtcgagttcctctccatgaagctcgcaaccgtcaacccactggacctgagcaacctgcccacgctcctacaaaaagatatgttccaggcctgcggcgcttcggcgagctcggtcttctcgctggagagctgcagcccggggttcccgttcggcgggcaaggggacgtgttccagtccttcgtcccgaacggcctggagaacccgtgcggcggcctgaacccgctggacctggctctgtcccaggctaccggcgggcagttcggtttccaggacggaacggccggcacaaacctgcagcaaaggaactactgggaggaggaggaggaggacctgcagagcgtgttccacatcgacgacaacgggcagagccaggagcacggggcctcagcttcagcacaaagcttccatggtcagcttcaaccacaagagggccacatgaagatggagttcttctga

SEQ ID NO: 114 - protein, 397 aa, Zea mays
MEGMEEANRTAVESCHRVLALLSNPHGQLVPSKELVAATGEAVAKFGSLTAKLSNSNGDGLLQGHARVRKVKKPLHIFDSNLFLESSAVAAAAAPAKTPSPSPILGLQLFPRYHQFEGSSSKDPVRIPTQFPKRLLLEKPTAGMEGSTSQSPPIVQMVQPVSVAPPAGTPTPALPPAHLHFIQQQQSYQRFQLMQQMKIQSEMMKRSNLGDQGGSLSGGGGGGRKGVNLKFDSSNCTASSSRSFLSSLSMEGSLASLDGSRTSRPFQLLSGSQTASTPELGLVQRRRCAGREDGTGRCATGSRCHCSKKRKLRIRRSIKVPAISNKVADIPADEFSWRKYGQKPIKGSPHPRGYYKCSSVRGCPARKHVERCVDDPSMLIVTYEGDHNHNRVLAQPA

SEQ ID NO: 115 - DNA, 1194 bp, Zea mays
  source 1..1194: mol_type = genomic DNA
atggaggggatggaggaggccaacaggacggcagtggagagctgccaccgggtgctggcgctcctctccaacccgcacggccagctcgtccccagcaaggagctcgtggccgccaccggggaggccgtcgccaagttcggctcactgacggccaagctctccaactccaacggcgatggcctactgcagggccacgctagggtcaggaaggtcaagaagcccctgcacatcttcgacagcaacctcttcctcgagagctctgcggtggccgccgccgctgctccggccaagacgcccagcccgagcccgatccttggcctccagctgttcccgaggtaccaccagtttgagggctcgtcgtctaaggatcctgtcaggatccctacccagttccccaagaggttgctgctagagaagccgactgccggtatggaggggtcgacgtcgcagtcccctccgatcgtccagatggtccagccggtgtccgttgcgccccccgcagggacgcccaccccggcattgccccccgctcaccttcatttcatccagcagcagcaaagctaccagaggtttcagctcatgcagcagatgaagatccagagcgagatgatgaagaggagcaatcttggtgatcagggtggtagcttaagtggcggcggcggcggcggtcgtaagggtgtgaatctcaagtttgacagctctaattgcacggcgtcatcgtctcgctcgttcctttcgtctctgagcatggaagggagtcttgcgagtttggatggaagccggaccagcaggccgttccagctacttagtggctctcagacagctagcacgccggagctgggcctggtgcaaaggagaaggtgcgctggcagggaggatgggactggtcggtgtgcaaccgggagccggtgccattgttcaaagaaaaggaagctaaggataaggaggtccatcaaagtccctgcaataagcaacaaggttgcagacatcccagctgatgaattctcatggaggaagtatgggcagaagccaattaagggatccccacatcctaggggttattacaagtgtagcagtgtgagaggctgccccgcgaggaagcatgtcgagaggtgcgtggacgacccctcgatgctgattgttacctatgaaggtgaccacaaccacaaccgagttctagcccagccagcctga

SEQ ID NO: 116 - protein, 374 aa, Zea mays
MEEVEVANRAAVESCHRVLALLSQQRDPALLKSVASETAEACAKFRKVAALLGSGVGHARGRFSRRVRPLGLVGHKSPVGSGGNNPVEMLPSAAAVTSPSPSTSYPPQMRARLNGVPDPRGLDMACSSSSKSGGVGGGGAHPFGGAPKLVQPLSVQFQIGNVAHRYPFHQQPPSRQKLQAAEMFRRSSSGTISLKFDSPIPSGGGGAAGTVSFVSSLSMDGSVGVASLDGKRPFHLVGTPVAASDTAADAHRAPKRRCTCRGGEEDGRGNKCGTSGRCHCSKRRKLRIKRSIKVPAISNKVADIPPDEYSWRKYGQKPIKGSPHPRGYYKCSSVRGCPARKHVERCVDDPAMLIVTYEGEHGHTQLPAQPSTQT

SEQ ID NO: 117 - DNA, 1125 bp, Zea mays
  source 1..1125: mol_type = genomic DNA
atggaggaggtggaggtggccaaccgggccgcggtggagagctgccaccgtgtgctggccctgctctcgcagcagcgggacccggccctgctcaagagtgtagcgtcagagacggccgaagcctgcgccaagttcaggaaggtagccgccctcctcggcagcggtgtcggccacgctagaggcaggttctccagacgagtccggcctctggggctcgttggccacaagagccccgtggggagcggcggcaacaacccggtggagatgctgcccagcgccgccgccgttacgtccccgtctccgtcaaccagctacccgccgcaaatgcgcgctcggctgaacggcgtgccagacccacgagggctggacatggcctgctccagcagcagcaagagcggcggcgttggcggaggcggcgctcatccattcggcggagcgccgaagctggtgcagccgctgtccgtgcagttccagatcgggaacgtcgcgcacaggtacccgttccaccagcagccgccgtcgcggcagaagctgcaggccgccgagatgttcaggaggagcagcagcgggacgatcagcctcaagttcgatagcccaatccctagcggtggcggtggcgccgctggcaccgtgtcgttcgtgtcgtccttgagcatggacgggagcgtgggcgtggcaagcttggacgggaagcggccgttccatctggtcggcaccccggtggcggcgagcgacacggcggcagacgcccaccgcgcgcccaaacgccggtgcacgtgtagaggaggcgaggaggatgggagaggcaacaagtgcggcacctccggcaggtgccattgctcaaagagaaggaagctgcggatcaagaggtcgatcaaagtgccagccatcagcaacaaggtcgccgacatacctcccgacgagtactcgtggcgcaagtacgggcagaagccgatcaagggctccccgcacccgaggggctactacaagtgcagcagcgtcaggggctgcccggcgaggaagcacgtcgagcggtgcgtggacgacccagcgatgctgatcgtgacgtacgaaggcgagcacggccacacccagctgccggcgcagccttccacccagacgtag

SEQ ID NO: 118 - protein, 367 aa, Zea mays
MEEVEVANMAAVESCHRVLALLSQQQDPALLKSVASETAEACAKFRKVAALLGSASGGGGCGHARGRFSRRVRPLGLVNQKSPLGIATGSGNPLEMMPSTAAAAGSPSPPQSTSYAQMRARLSSAPESSRGLDLACSSSRCSPHPFGAPKLVQPLSVQFQIGSVAHRYPFHQQQSRQKLQAEMFRRSNSGISLKFDSPSPSGGAAGTMSSARSFMSSLSIDRSMASLDGKRPFHLVGTPVASDPADAHRAPKRRCTGRGEDGRGKCATTGRCHCSKRRKLRIKRSIRVPAISNKIADIPPDEYSWRKYGQKPIKGSPHPRGYYKCSSVRGCPARKHVERCVDDPAMLIVTYEGEHSHTQLPAQPAQT

SEQ ID NO: 119 - DNA, 1104 bp, Zea mays
  source 1..1104: mol_type = genomic DNA
atggaggaggtggaggtggccaacatggccgcggtggagagctgccaccgggtgctggctctgctctcgcagcagcaggacccagccctgctcaagagcgtagcttcagagacggctgaagcctgcgccaagttcaggaaggtagcggccctcctcggcagtgccagtggcggaggtggctgtggccatgctagaggcaggttctccagacgagtccggcctctggggctcgtcaaccagaagagccccttggggatcgccaccggcagcggcaacccgctggagatgatgcccagcacagctgctgctgctgggtctccatctccacctcagtcgactagctatgcgcaaatgcgcgctcggcttagcagtgcgccagagtcctcacgagggctggatttggcctgctccagcagcaggtgtagccctcatccatttggagcccccaagctggtccagccactgtctgtgcagttccagattgggagtgttgcgcataggtacccgttccaccagcagcagtcgaggcagaagctgcaggccgagatgttcaggaggagcaacagtgggatcagcctcaagttcgatagccctagccccagcggtggtgctgctggcaccatgtcgtctgcgagatcattcatgtcgtcgttgagtatagacaggagcatggcgagcttggatgggaagcggccgttccatttggtaggcaccccggtggcgagcgacccagcagatgcgcaccgtgcgcccaaacggcgatgcacgggtagaggggaggatggaagaggcaagtgtgccaccactggcagatgccattgctcaaagagaaggaaactgcggattaagaggtcgattagagtgccagccattagcaacaaaatcgctgatatccctcctgatgagtactcgtggcgcaagtacgggcagaagccaattaagggttccccacacccgaggggttactacaaatgcagcagcgtcaggggctgcccagcaaggaagcacgtcgagcgatgtgtagacgacccggcgatgctaatcgtgacatacgaaggcgagcacagccacacccagctgccagcacagcctgcccagacctag

SEQ ID NO: 120 - protein, 369 aa, Zea mays
MEEVEEANREAVESCRRVLALLSHPHDPAQVRSIALGTDEACTKFRKVVSLLSNGEVGTGEAGPSGTSASASHPRAKLVSRRQNPGFLTQKGFLDSNTPVVVLNSAAHPSPTSAQVHPRAGALDTEGVHPLGVGGPPKLVQPLSAHFQFGNVSSRYQQLPSHHHRHQQQEKLQAAEMFKRSNSGINLKFESASGTGTMSSARSFLSSLSMDGSVVASLDGKLPSSSSSFRLIGAPAMSDPANAAQQAPRRRCTGRGKDGTGKCALAGRCHCSKRSKKLRVKRSIKVPAVSNKIADIPPDEYSWRKYGQKPIKGSPHPRGYYKCSSVRGCPARKHVERCVDDSAMLIVTYEGEHNHTGMPAQSAAAAAQV

SEQ ID NO: 121 - DNA, 1110 bp, Zea mays
  source 1..1110: mol_type = genomic DNA
atggaggaagtggaggaggcgaacagggaagccgtggagagctgccgcagggtgctagccttgctctcccatccgcatgaccccgcgcaggtcaggagcatagctctggggacggacgaagcatgcaccaagtttaggaaggtggtctccctgctcagcaatggagaagtaggaacaggagaagccggaccatcaggcacaagcgcaagcgcaagccatcctagagctaagcttgttagcagaagacagaatccagggttcttgactcagaaaggcttcctggacagtaacaccccggtcgtggtgctgaacagcgccgcccatccttctccgacctccgcgcaggtgcatccaagagccggagcattggatacggagggcgtgcaccccctcggagttggaggacctcccaagctggtccagcctttgtccgcgcattttcagttcggcaatgtgtcgtcgaggtatcagcagttaccgagtcatcatcatcgtcatcagcagcaggagaagctgcaggccgccgagatgttcaagagaagcaacagcgggataaacctgaagttcgagagcgccagcggcacgggtacaatgtcgtcggcgaggtccttcctgtcgtctctgagcatggatggcagcgtggtggctagcctggatggcaagctgccgtcgtcgtcgtcatcgttccgtttgatcggtgcgcctgcaatgagcgatccggcgaacgccgcgcagcaggccccgaggcggcggtgcacgggccgcgggaaggacgggactggcaagtgcgctttggcgggcaggtgccattgttcaaagagaagtaagaagttgcgggtgaagaggtcgattaaggttcctgccgttagtaacaagatcgctgatatacctccggatgagtactcgtggaggaagtatgggcagaagccgatcaagggttcccctcatcctaggggctactacaaatgcagcagtgtgaggggctgtccagctaggaagcacgtggaacggtgtgtggatgattcggcgatgctcatcgtgacatacgagggcgagcacaaccacaccggaatgccagctcagtcagcagcagcagcagcacaggtgtag

SEQ ID NO: 122 - protein, 354 aa, Zea mays
MEEVEEANREAVESCHRVLALLSQPHDPAQARSIALGTDEACARFRKVVSLLSNGGAGLGEAGPSCGSASASRPRAKLVSRRQNPGFLTQKGFLDSNTPVVVLNSAHPSTTSAQVYPRTGALVDAQSVHPLGVGGPPKLVQPLSAHFQFGSVPARYQFPNQQQQKLHAEMFKRSNSGVNLKFESASGTAGTMSSARSFLSSLSMDGSVASLDAKSSSFHLIGGPAMSDPLNAQQPPRRRCTGRGEDGTGKCAVTGRCHCSKRSRKLRVKRSIKVPAISNKIADIPPDEYSWRKYGQKPIKGSPHPRGYYKCSSVRGCPARKHVERCVDDSAMLIVTYEGEHNHTRMPTTQSAQV

SEQ ID NO: 123 - DNA, 1065 bp, Zea mays
  source 1..1065: mol_type = genomic DNA
atggaggaagtggaggaagcgaacagggaagccgtggagagctgccacagggtgctcgccttgctctcccagccgcatgaccccgcgcaggccaggagcatagctctgggcacggacgaagcatgtgccaggttcaggaaggtggtctctctactgagcaatggaggagcggggctaggagaagccgggccgtcatgcggaagcgcaagcgcaagccgtcctagagctaagcttgttagcagaagacagaatccagggttcttgacccagaaaggcttcctggacagcaacaccccggtcgtggtgttgaacagcgcccatccttctactacctccgcgcaggtgtaccctagaactggagctctcgtcgatgcacagagcgtgcaccccctcggagtcggaggacctcccaagctggtccagcccttgtccgcgcactttcagttcggcagtgtgccggctcggtatcagttcccgaatcagcagcagcagaagttgcacgccgagatgttcaagagaagcaacagcggggttaacctgaagttcgagagcgccagtggcactgctgggacgatgtcgtcggcgaggtccttcttgtcgtctctgagcatggacggtagcgtggctagcctggacgccaagtcatcgtcgttccatttgatcggtggacctgcgatgagcgatccgctgaacgcgcagcagcccccgaggaggcggtgcacgggccgtggggaggatgggaccggcaagtgcgctgttacagggaggtgccactgttcgaagagaagtaggaagttgcgggtgaagaggtcgattaaggttcctgccattagtaacaagattgccgatatacctccggatgaatactcgtggaggaagtatgggcagaagccaattaagggttcccctcatcctaggggttactacaaatgcagtagcgtgaggggctgcccagccaggaagcatgtggaacggtgcgtagatgattcggcgatgctcatcgtgacatatgagggcgagcacaaccacaccagaatgccaacaactcagtcagcacaggtgtaa

SEQ ID NO: 124 - protein, 395 aa, Zea mays
MEAMEEANRTAVESCHRVLALLSNPHGQLVPSRELVATTGEAVARFGSLAAKLSSSNGNGLQGHARVRKTKKPLPIFYSNLFLESSSAVAAAATVAKTPSPSPTTGLQLFPRYHQMEGSSCKDPVRIPAQFPKRLLLENLAAGLEGSSPPAPPVRMVQPVSVAPPAGMPTPALPAAHLHFIQQQQSYQRFQLMQQMKIQSEMVKRSNLGEQGGSLSGGGGGGKGVNLKFDSSNCTASSSRSFLSSLSMEGSLASLDGSRASRPFQLVSGSQTSSTPELGLVQRRRCAGKEDGSGQCATGSRCHCSKKRKLRIRRSIKVPAVSNKVADIPADEFSWRKYGQKPIKGSPHPRGYYKCSSVRGCPARKHVERCVDDPSMLIVTYEGDHNHSRVLAQPA

SEQ ID NO: 125 - DNA, 1188 bp, Zea mays
  source 1..1188: mol_type = genomic DNA
atggaggcgatggaggaggccaacaggacggcagtggagagctgccaccgggtgctggcgctcctctccaacccgcacggccagctcgtccccagcagggagctcgtggccaccaccggggaggccgtagccaggttcggctccctggcggccaagctctccagctccaacggcaatggcctgcagggccacgctagggtcaggaagaccaagaagcccctgcccatcttctacagcaacctcttcctcgagagcagctctgcggtggccgccgccgcgactgtggccaagacgcccagtccgagcccgaccactggcctccagctgttcccgaggtaccaccagatggagggctcgtcgtgtaaggatcctgtcaggatccctgcgcagttccccaagaggttgctgctggagaacctggctgccggtctggaggggtcgtcgccgcctgcccctccggtccggatggtgcagccggtgtccgttgcgcctcccgcagggatgcccaccccggcattgcccgccgcacacctccatttcatccagcagcagcagagctaccagaggtttcagctcatgcagcagatgaagattcagagcgagatggttaagaggagcaatcttggtgagcagggtggtagcttaagtggcggcgggggcggtggtaagggtgtgaacctcaagtttgacagctccaattgcacggcgtcatcgtctcgctcgttcctttcgtctctgagcatggaaggaagtcttgcgagcttggatggaagccgggccagcaggccgttccagctagttagtggctctcagacatctagcacaccggagctgggcctggtgcaaaggagaaggtgtgctgggaaggaggatgggagtggtcagtgcgcaaccgggagccggtgccattgttcaaagaaaaggaagctaaggataaggaggtccatcaaagtccctgcagtaagcaacaaggttgcagacatcccagctgatgagttctcgtggaggaagtatgggcagaagccaatcaagggatccccacatcctaggggttactacaagtgtagcagcgtgagaggctgccccgcgaggaagcacgtcgagaggtgcgtggacgacccttcgatgctgatcgttacctacgagggtgaccacaaccacagccgagttctagcccagccagcctga

SEQ ID NO: 126 - protein, 331 aa, Zea mays; identical to SEQ ID NO: 106

SEQ ID NO: 127 - DNA, 996 bp, Zea mays; identical to SEQ ID NO: 107
  source 1..996: mol_type = genomic DNA

SEQ ID NO: 128 - protein, 316 aa, Zea mays; identical to SEQ ID NO: 104

SEQ ID NO: 129 - DNA, 951 bp, Zea mays; identical to SEQ ID NO: 105
  source 1..951: mol_type = genomic DNA

SEQ ID NO: 130 - protein, 298 aa, Zea mays
MAVDLMGCYAPRRANDQLAIQEAAAAGLRNLELLVTSLSTQAAAPHRAADQPFGEIAGQAVSKFRKVISILDRTGHARFRRGPVEPPPPTPPPPPVVPGPAPLAAVSVAQPPQSLTLDFTKPNLAVSAATSVTSTSFFSSVTAGEGSVSKGRSLMSSGKPPLSGHKRKPCAGAHSEATTNGSRCHCSKRRKNRVKRTIRVPAISSKVADIPSDEYSWRKYGQKPIKGSPYPRGYYKCSTVRGCPARKHVERATDDPAMLVVTYEGEHRHTPGAVQGPSPLATASPVPVAVSAGNGLVV

SEQ ID NO: 131 - DNA, 897 bp, Zea mays
  source 1..897: mol_type = genomic DNA
atggccgtggacctgatgggctgctacgccccgcgccgcgccaacgaccagctcgccatccaggaggcggcggcggccgggctccgcaacctggagctgctggtgacgtccctgtccacgcaggccgccgcgccgcacagagccgctgatcagccgttcggcgagatcgccggccaggccgtctccaagttccgcaaggtcatctccatcctcgaccgcacggggcacgcccgcttccgccgcgggcccgtcgagccgccgccgccgacgccgccgccgcctcctgtcgtccccggtcctgcccccctggcggccgtcagcgtggcgcagccgccgcagagcctgacgctggacttcacgaagccgaacctggccgtgtcggccgccacgtccgtcacctccacgtccttcttctcgtcggtcacggccggcgagggcagcgtctccaagggccggagcctcatgtcctccgggaagccgccgctgtccggccacaagcggaagccctgcgccggcgcccactccgaggccaccaccaacggcagccgttgccactgctccaagagaaggaagaaccgcgtgaagaggaccatcagagtgccggcgatcagctcgaaggtcgccgacatcccttcggacgagtactcgtggaggaagtacggccagaagcccatcaagggctccccttacccacggggctactacaagtgcagcactgtgcggggatgcccggcgaggaagcacgtggagcgggccaccgacgacccggccatgctggtggtgacgtacgagggcgagcaccgccacacgccgggcgcggtccaggggccgagccccctggcgaccgcgtcgccggtgcccgtcgccgtctccgccggcaacgggctcgttgtctag

(19) United States
(12) Patent Application Publication
(10) Pub. No.: US 2025/0092409 A1
(43) Pub. Date: Mar. 20, 2025

(54) Plants with Improved Growth

(71) Applicant: SweTREE Technologies AB, Umeå (SE)

(72) Inventors: Anna Karlberg, Umeå (SE); David Jonsén, Umeå (SE); Linus Möller, Umeå (SE); Magnus Hertzberg, Umeå (SE); Pär Jonsson, Umeå (SE)

(21) Appl. No.: 18/896,139

(22) Filed: Sep. 25, 2024

Related U.S. Application Data
(63) Continuation of application No. 16/769,393, filed on Jun. 3, 2020, now Pat. No. 12,129,472.

(30) Foreign Application Priority Data
Dec. 4, 2017 (SE) 1751493-6

(51) Int. Cl. C12N 15/82

(52) U.S. Cl. CPC C12N 15/8226; C12N 15/8227; C12N 15/8229; C12N 15/8261

(57) ABSTRACT

The present invention relates to genetically modified plants comprising a heterologous nucleic acid construct comprising a promoter sequence operably linked to a coding sequence encoding a gene product. The invention further relates to methods for producing such plants.

### CROSS-REFERENCE TO RELATED APPLICATIONS

This application is a continuation application of U.S. patent application Ser. No. 16/769,393, which is a U.S. National Phase of International Application No. PCT/SE2018/051245, filed Dec. 4, 2018, which claims priority to Swedish Application No. 1751493-6, filed Dec. 4, 2017.

### INCORPORATION BY REFERENCE OF THE SEQUENCE LISTING

This application includes a sequence listing submitted electronically as 55579A_SeqListing.xml; Size: 228,515 bytes; Created: Sep. 17, 2024 and is incorporated herein by reference.

### FIELD OF THE INVENTION

The invention relates to the field of plants with improved growth and yield properties, and in particular to plants comprising heterologous nucleic acid constructs comprising improved combinations of growth improving genes and promoters influencing their expression in the plants.

### BACKGROUND TO THE INVENTION

Plant growth is influenced by a large number of different growth influencing genes, where some are genes encodes for hormones, transcription regulators and other growth and development enhancing products. Some gene products are active throughout the plant life cycle, e.g. shoot elongation, the expansion and shape of leaves, flowering and seed germination other only for short periods. Several examples illustrating the importance of growth influencing genes can be found in the literature.

### Plant Growth

It is known to a person skilled in the art that the phenotypical effect of any gene in the plant is highly dependent on gene regulation. For example, spatial and temporal expression patterns as well as stress induction of genes significantly influence the plant phenotype. Conversely, controlling gene regulation can be used in attempts to improve the plant phenotype, for example, increasing plant growth. Gene expression can be modified using promoters which spatially and temporally direct gene expression in specific tissues and to specific levels. Positive phenotypical traits conferred by a gene can be modified to improve growth by controlling gene expression. Similarly, controlling gene regulation can also be used to attempt to prevent negative phenotypical effects of a gene.

Growth of plants appear at apical meristems and results in the development of sets of primary tissues and in the lengthening of the stem and roots.

In addition to this primary growth, trees undergo secondary growth and produce secondary tissue “wood” from the cambium. This secondary growth increases the girth of stems and roots. There are several factors such as different gene products that might need to be altered in order to enhance biomass production in trees. Growth in height, diameter, stem volume and wood density are important traits to observe for increased growth and biomass production. However, it is also known to a person skilled in the art that a specific spatial and temporal expression pattern of a gene may elicit different phenotypical effects under two distinctly different growth conditions, for example, the growth conditions to which the plants are exposed in the greenhouse compared to in a field trial environment.

### Promoters

Promoters are regions of DNA involved in binding of RNA polymerase to initiate transcription of coding sequences. Promoters can comprise several regulatory elements, usually called cis elements, generally located within a few hundred nucleotides from the transcription initiation site but that may also be positioned as far upstream as several thousand nucleotides as well as in introns. Trans-acting proteins then usually bind to these cis elements and then regulate transcription. The cis regulatory elements are separated along the nucleotide sequence by nucleic acid stretches that have no known regulatory effect on their own, the spacing of the cis-elements could however be important for their function.

Promoters may be constitutive, tissue-specific, rhythmic, or inducible by certain stimuli.

Constitutive promoters induce expression of the coding sequence in most tissues of the plant, irrespective of developmental stage or environmental factors.

Tissue-specific promoters induce expression of the coding sequence in a specific tissue or region of the plant.

Rhythmic promoters is subjected to internal rhythms by an internal timer, these internal timers are for example influenced by light and temperature and their status influence long term expression patterns, for example yearly variations in gene expression.

Promoters can also have temporal variations in activity, for example could the activity of a promoter be reduced or increased during flower induction or dormancy related processes.

Inducible promoters are activated by chemical or physical factors, such as isopropyl β-D-1-thiogalactopyranoside (IPTG), light, or temperature.

The Cauliflower Mosaic Virus, CaMV, 35S promoter is the most frequently used promoter when studying effects of modified gene expression during development, since the studied genes are constitutively expressed when the promoter is operably linked to them. The use of the CaMV 35S promoter has generated a lot of data regarding gene function and effects of over-expression in laboratory tests. In some situation it can be useful to have access to a promoter that in combination with a gene is more specifically expressed in a certain plant tissue or plant part. Results from field tests have shown that plants genetically modified with a construct with the CaMV 35S promoter operably linked to a trait gene may be acceptable, but have also been shown to result in unimproved or adverse effects in the field.

### Wood Production

Wood is used for paper production and for constructions. In many situations there is a need for improved properties and improved quality of the wood used. The main need is the quantity of wood. This can be achieved by cutting down more trees, or by using more land for tree production or by using trees which grow faster and have better growth properties. The later can be done by traditional breeding programs or by use of gene modification. Both strategies lead to a shorter rotation time, i.e. the time from planting to harvest. A major disadvantage with traditional tree breeding, especially for forest tree species, is the slow progress due to their long generation periods. Breeding programs are also dependent on the genetic variation present in a tree population. However, by taking advantage of recent developments in gene technology the time required to produce a new variety could be reduced significantly and the effect could be additive to effects produced by breeding.

### Growth Improving Genes

### The Gene G47

The gene called AtG47, Seq ID No.: 1, is expected to be an ERF/AP2 transcription factor, belonging to the CBF/DREB subfamily, very little is known about its function, some phylogenetic information can be found in the scientific literature.

Mendel Biotechnology Inc. have performed research on effects of over-expression of the G47 using the CaMV 35S promoter and that information can be found in a number of patent documents from Mendel Biotechnology, WO2004031349, WO2004076638, WO2005047516, WO2006069201, WO2006130156, and WO2014100289.

In WO2006130156 it is shown that seedlings of Arabidopsis over-expressing G47 under the CaMV 35S promoter have brighter green color leaves, more narrow bundles of xylem vessels, than wild type Arabidopsis plants. Further, the inflorescences from Arabidopsis over-expressing G47 plants appeared thick and fleshy, had reduced apical dominance, and exhibited reduced internode elongation leading to a short compact stature. Over-expressing G47 had an increase in the number of xylem vessels in stems, as well as increased lignin content.

In WO2004031349 and WO2006069201 Arabidopsis plants over-expressing overexpressing AP2 polypeptides under the control of the 35S promoter had increased stress tolerance but often grew at a reduced rate, were smaller, and may have experienced delayed flowering with respect to wild type plants.

In WO2005047516 it is shown that over-expression of G47 resulted in a variety of morphological and physiological phenotypic alterations. 35S::G47 plants showed enhanced tolerance to osmotic stress, enhanced survival and drought tolerance in a soil-based drought assay, delay in flowering time and caused a marked change in shoot architecture.

As a summary, over-expressing the gene G47 resulted in some positive and some negative growth effects when using the CaMV 35S-promoter linked to the gene G47.

In JP2016127811 the inventors have coupled an Arabidopsis thaliana promoter NST3 to a construct comprising the sequence of the gene SGF1 (100% sequence identity to G47) and the very strong VP16 transcription activator domain. This construct was then introduced into double NST1/NST3 mutant of Arabidopsis thaliana, which thus lack NST1 and NST3 proteins. When the construct proNST3:SGF1-VP16 was expressed in this double mutant Arabidopsis thaliana it showed enlarged flower stalks (flowering stems). JP2016127811 does not show how a wild-type perennial woody plants will grow in a greenhouse or in the field, when a promoter linked to the unmodified SGF1 gene, without VP16, is expressed in said woody plant.

### The Gene TF0002

In WO2009084999 it is shown that a DNA sequence coding for a TF0002 polypeptide under the control of the CaMV 35S promoter inserted in a poplar genome results in increased growth compared to unmodified wild type trees when potted in soil and grown in the greenhouse. Similar results are presented in WO2011109661. However, the biological function of the TF0002 gene is not clearly defined.

In WO2002015675 it is shown that the overexpression of the Arabidopsis ortholog of TF0002 using the 35S promoter in Arabidopsis plants resulted in increased size of the plants.

The Populus tremula x tremuloides TF0002 gene, PttTF0002, Seq ID No.: 2 is a WRKY transcription factor, belonging to WRKY subfamily IId according to the WRKY gene classification presented in Eulgem et al. 2000. The WRKY domain, a 60 amino acid region defined by a conserved amino acid sequence (WRKYGQK) at its N-terminal end and a novel zinc-finger-like motif, has a sequence specific DNA-binding activity. Reviews of the current state of understanding on WRKY transcription factors can be found in, for example, Bakshi and Oelmüller (2014) and Ülker and Somssich (2004). The WRKY subfamily IId transcription factors interact with calmodulin (CaM), which is a ubiquitous Ca²⁺ binding protein known to regulate diverse cellular functions by modulating the activity of various target proteins. The CaM-binding domain (CaMBD) is a conserved structural motif (C-motif) found in subfamily lid of the WRKY protein family (Park et al. 2005). The WRKY transcription factor family subfamily lid genes in Arabidopsis thaliana are AtWRKY7, AtWRKY15, AtWRKY11, AtWRKY17, AtWRKY21 and AtWRKY39 (Eulgem et al. 2000). The putative ortholog to the PttTF0002 gene in Arabidopsis thaliana is AtWRKY7, accession number AT4G24240. The amino acid sequence identity between PttTF0002 and AtWRKY7 is 54%, determined using the ‘Align Sequences Protein BLAST’ tool at NCBI (https://www.ncbi.nlm.nih.gov/).

The biological function of the PttTF0002 gene has not been described in prior art.

The Arabidopsis thaliana WRKY7 gene (AtWRKY7) is induced by pathogen infection and salicylic acid (SA) treatment and may therefore play a role in plant defence responses. AtWRKY7 is localized in the nucleus, recognizes DNA molecules with the W-box (TTGAC) elements, and functions as a transcriptional repressor in plant cells. T-DNA insertion and RNAi mutant plants display enhanced resistance to a virulent strain of the bacterial pathogen Pseudomonas syringae as measured by significant decrease in both bacterial growth and symptom development as compared to those in wild type plants. The enhanced resistance in the loss-of-function mutants was associated with increased induction of SA-regulated Pathogenesis-Related 1 (PR1) gene expression by the bacterial pathogen. Transgenic plants that constitutively overexpress AtWRKY7 have altered leaf growth. AtWRKY7-overexpressing plants supported more growth of P. syringae and developed more severe disease symptoms than wild type plants. The enhanced susceptibility of the AtWRKY7-overexpressing plants correlates with reduced expression of defence-related genes, including PR1, but significantly increased accumulation of SA after pathogen infection, probably due to reduced negative feedback of SA synthesis. Thus, pathogen-induced AtWRKY7 transcription factor play a negative role in defence responses to P. syringae (Kim et al. 2006).

In summary, strong, constitutive over-expression of the TF0002 gene, using the CaMV 35S promoter may result in increased growth. However, no reports on the effect on plant growth of other promoters tested in combination with the TF0002 gene are available in prior art.

In conclusion, to anticipate the effect that a specific promoter-gene combination has on the plant is ingenious and nontrivial. Prior art does not provide information enough to foresee the effect that a specific combination of promoter and a TF0002 gene will have on the plant. Nor does prior art provide information enough to indicate which promoter should be used in combination with the TF0002 gene to improve plant growth or biomass production without the negative pleiotropic effects that strong constitutive 35S promoter expression may induce.

### The Gene TF0097

In WO2009084999 it is shown that a DNA sequence coding for a TF0097 polypeptide under the control of the CaMV 35S promoter inserted in a poplar genome results in increased growth compared to unmodified wild type trees when potted in soil and grown in the greenhouse. However, the biological function of the TF0097 gene is not clearly defined.

The Populus tremula x tremuloides TF0097 gene, PttTF0097, Seq ID No.: 3 is a transcription factor belonging to the basic helix-loop-helix (bHLH) protein superfamily of transcription factors. There are three possible orthologs to the PttTF0097 gene in Arabidopsis thaliana, CIL2 (ACE3, AtbHLH077), CIB2 (AtbHLH078) and CIB3 (AtbHLH062), accession number AT3G23690, AT5G48560 and AT3G07340 respectively. In Arabidopsis the bHLH superfamily has approximately 160 predicted transcription factors with several subgroups, where the possible Arabidopsis orthologs CIL2, CIB2 and CIB3 all fall into subgroup XII, Pires and Dolan 2010. The amino acid sequence identity between PttTF0097 and CIL2 is approximately 50% over 76% of the polypeptide, determined using the ‘Align Sequences Protein BLAST’ tool at NCBI (The amino acid sequence identity between PttTF0097 and CIB2 is approximately 43% over 91% of the polypeptide and the amino acid sequence identity between PttTF0097 and CIB3 is approximately 46% over 100% of the polypeptide.

The biological function of the PttTF0097 gene has not been described in prior art. Some possible orthologues have been proposed and their function is discussed below.

Liu et al. (PLoS Genet 2013e1003861) studied the function of the possible Arabidopsis orthologs CIL2 (ACE3, AtbHLH077, AT3G23690), CIB2 (CRY2-interacting BHLH 2) (AtbHLH078, AT5G48560) and CIB3 (AtbHLH062, AT3G07340). Arabidopsis thaliana cryptochrome 2 (CRY2) mediates light control of flowering time. CIB1 (CRY2-interacting bHLH 1) specifically interacts with CRY2 in response to blue light to activate the transcription of FT (Flowering Locus T). CIB1 positively regulates floral initiation in a CRY2-dependent manner. Over-expression studies, using the 35S promoter, showed that when the genes CIB1 and CIB2 are over-expressed plants flowered significantly earlier than the wild type parents in long day condition, while transgenic plants over-expressing CIB3 or CIL2 showed no obvious flowering phenotype. The genetic analysis performed by Liu et al. show that CIB1, CIB2, CIB4, and CIB5 act redundantly to activate the transcription of FT and suggests that they are positive regulators of CRY2 mediated flowering. CIB proteins are involved in blue light signalling, they are specifically stabilized by blue light and are also blue light regulated. Similar to CIB1, the expression of CIB2, CIB4, and CIB5 proteins are regulated by blue light in a wavelength-specific manner.

In summary, constitutive over-expression of the TF0097 gene, using the CaMV 35S promoter may result in early flowering or increased growth. However, no reports are available in the prior art showing that promoters in combination with the TF0097 gene may have an effect on early flowering in plant.

In conclusion, to anticipate the effect that a specific promoter-gene combination has on a plant is ingenious and nontrivial. Prior art does not provide information enough to foresee the effect that a specific combination of promoter and a TF0097 gene will have on the plant. Nor does prior art provide information enough to indicate which promoter should be used in combination with the TF0097 gene to improve plant growth or biomass production without the negative pleiotropic effects that strong constitutive 35S promoter expression may induce.

### The Gene TF0132

In WO2009084999 it is shown that a DNA sequence coding for a TF0132 polypeptide under the control of the CaMV 35S promoter inserted in a tree genome results in increased growth compared to unmodified wild type trees when potted in soil and grown in the greenhouse. However, the biological function of the TF0132 gene is not clearly defined.

The Populus tremula x tremuloides TF0132 gene, PttTF0132, Seq ID No.: 4, is a WRKY transcription factor, belonging to WRKY subfamily IId according to the WRKY gene classification presented in Eulgem et al. 2000. The WRKY domain, a 60 amino acid region defined by a conserved amino acid sequence, WRKYGQK, at its N-terminal end and a novel zinc-finger-like motif, has a sequence specific DNA-binding activity. Reviews of the current state of understanding on WRKY transcription factors can be found in, for example, Bakshi and Oelmüller (2014) and Ülker and Somssich (2004). The WRKY subfamily lid transcription factors interact with calmodulin, CaM, which is a ubiquitous Ca²⁺-binding protein known to regulate diverse cellular functions by modulating the activity of various target proteins. The CaM-binding domain, CaMBD, is a conserved structural motif, C-motif, found in subfamily lid of the WRKY protein family (Park et al. 2005). The WRKY transcription factor family subfamily lid genes in Arabidopsis thaliana are AtWRKY7, AtWRKY15, AtWRKY11, AtWRKY17, AtWRKY21 and AtWRKY39 Eulgem et al. (2000). The putative ortholog to the PttTF0132 gene in Arabidopsis thaliana is AtWRKY21, accession number AT2G30590. The amino acid sequence identity between PttTF0132 and AtWRKY21 is 58%, as determined using the ‘Align Sequences Protein BLAST’ tool at NCBI (https://www.ncbi.nlm.nih.gov/). Two AtWRKY21 homologs, AtWRKY39 and AtWRKY74, accession number AT3G04670 and AT5G28650 respectively, are also possible orthologs to PttTF0132. The amino acid sequence identity between PttTF0132 and AtWRKY39 is 52% and the amino acid sequence identity between PttTF0132 and AtWRKY74 is 50%, determined using the ‘Align Sequences Protein BLAST’ tool at NCBI (https://www.ncbi.nlm.nih.gov/).

The biological function of the PttTF0132 gene has not been described in prior art.

AtWRKY39 is induced by heat stress, salicylic acid (SA) and jasmonate. AtWRKY39 knock-down mutants have increased susceptibility to heat stress while over-expressors show enhanced thermotolerance and increased expression of the gene Pathogenesis-Related 1, PR1 (Li et al. 2010).

The ortholog to AtWRKY39 in cotton, GhWRKY39, is induced by infection or NaCl treatment. Constitutive over-expression of GhWRKY39 in Nicotiana benthamiana increased resistance to bacterial and fungal pathogen infections, as well as the expression of several pathogenesis-related genes. The transgenic plants also exhibited less hydrogen peroxide accumulation than wild type plants following pathogen infection. Moreover, GhWRKY39-overexpressing plants displayed enhanced tolerance to salt and oxidative stress, increased transcription of antioxidant enzyme genes, and improved activities of the antioxidant enzymes SOD, POD and CAT after pathogen infection and salt stress treatment. This suggests that GhWRKY39 may positively regulate the plant response against pathogen infection and salt stress (Shi et al. 2014).

In summary, strong, constitutive over-expression of the TF0132 gene, using the CaMV 35S promoter may result in increased growth. However, no reports on the effect on plant growth of other promoters tested in combination with the TF0132 gene are available in prior art.

In conclusion, to anticipate the effect that a specific promoter-gene combination has on the plant is ingenious and nontrivial. Prior art does not provide information enough to foresee the effect that a specific combination of promoter and a TF0132 gene will have on the plant. Nor does prior art provide information enough to indicate which promoter should be used in combination with the TF0132 gene to improve plant growth or biomass production without the negative pleiotropic effects that strong constitutive 35S promoter expression may induce.

### The Gene TF0109

In WO2009084999 it is shown that a DNA sequence coding for a TF0109 polypeptide under the control of the 35S promoter inserted in a tree genome results in increased growth compared to unmodified wild type trees when potted in soil and grown in the greenhouse. However, the biological function of the TF0109 gene is not clearly defined.

The Populus tremula x tremuloides TF0109 gene, PttTF0109, Seq ID No.: 5, is a WRKY transcription factor, belonging to WRKY subfamily IId according to the WRKY gene classification presented in Eulgem et al. 2000. Reviews of the current state of understanding on WRKY transcription factors can be found in, for example, Bakshi and Oelmüller (2014) and Ülker and Somssich (2004). The WRKY subfamily lid transcription factors interact with calmodulin (CaM), which is a ubiquitous Ca²⁺-binding protein known to regulate diverse cellular functions by modulating the activity of various target proteins. The CaM-binding domain is a conserved structural motif found in subfamily lid of the WRKY protein family (Park et al. 2005). The WRKY transcription factor family subfamily lid genes in Arabidopsis thaliana are AtWRKY7, AtWRKY15, AtWRKY11, AtWRKY17, AtWRKY21 and AtWRKY39 (Eulgem et al. 2000). The putative ortholog to the PttTF0109 gene in Arabidopsis thaliana is either AtWRKY11 or AtWRKY17, accession numbers AT4G31550 and AT2G24570 respectively. The amino acid sequence identity between PttTF0109 and AtWRKY11 is 53% and the amino acid sequence identity between PttTF0109 and AtWRKY17 is 54%, determined using the ‘Align Sequences Protein BLAST’ tool at NCBI (https://www.ncbi.nlm.nih.gov/).

The biological function of the PttTF0109 gene has not been described in prior art.

Journot et al. (2006 The Plant Cell, 18, 3289-3302) analyzed the role of the WRKY subfamily lid transcription factors in the regulation of basal resistance to Pseudomonas syringae pv tomato (Pst). The levels of expression of AtWRKY7, AtWRKY11, AtWRKY15 and AtWRKY17 were induced after inoculation with avirulent and virulent strains of Pst, but not the other members of subfamily lid. Mutant analyses revealed that loss of WRKY11 function increased resistance toward avirulent and virulent Pst strains and that resistance was further enhanced in wrky11 wrky17 double mutant plants. This suggests that WRKY11 and WRKY17 act as negative regulators of basal resistance to Pst.

In summary, strong, constitutive over-expression of the TF0109 gene, using the CaMV 35S promoter may result in increased growth. However, no reports on the effect on plant growth of other promoters tested in combination with the TF0109 gene are available in prior art.

In conclusion, to anticipate the effect that a specific promoter-gene combination has on the plant is ingenious and nontrivial. Prior art does not provide information enough to foresee the effect that a specific combination of promoter and a TF0109 gene will have on the plant. Nor does prior art provide information enough to indicate which promoter should be used in combination with the TF0109 gene to improve plant growth or biomass production without the negative pleiotropic effects that strong constitutive 35S promoter expression may induce.

### SUMMARY OF THE INVENTION

In some situations, it can be useful to have access to a promoter that in combination with a gene, is specifically expressed in a specific plant tissue or plant part. Thus there is a need for new combinations of new functional promoters in combination with genes that are well functional in field use, i.e. when the plant is grown under realistic outdoor conditions, such as in the real environment of the plant of interest. The present invention builds on the idea that an in average weak but specific promoter showing desired results on the wanted phenotype, when operably linked to a gene selected from G47, TF0002, TF0097, TF0132 and TF0109, ID No.: 1 to 5, will give either an increased desired effect or a less pleiotropic and possibly less negative effects in the field and in the mass production of a selected transgenic plant.

Thus there is a need for new combinations of functional promoters in combinations with genes that are well functional in field use, i.e. when the plant is grown under realistic outdoor conditions, such as in the real environment when growing the plant of interest.

In view of the need to provide plants capable of enhanced growth, yield and biomass in a range of different environmental conditions, as well as changing environmental conditions, there is a continual need to provide plants with different genetic traits, comprising different sets of promoters and active genes.

Furthermore, in view of the need to provide trees capable of enhanced growth and biomass production in a range of different environmental conditions, including areas where water is a limiting factor, as well as changing environmental conditions, there is a continual need to provide trees with different genetic traits, comprising different sets of promoters and active genes.

Thus, in a first aspect the invention relates to genetically modified woody plants comprising a heterologous nucleic acid construct comprising a promoter sequence operably linked to a coding sequence encoding a gene selected from G47 (SEQ ID No: 1), or TF0002 (SEQ ID No: 2), and to genetically modified plants comprising a heterologous nucleic acid construct comprising a promoter sequence operably linked to a coding sequence encoding a gene selected from TF0097, TF0132 and TF0109, ID No.: 3 to 5, respectively.

Thus, in one aspect the present invention relates to a genetically woody modified plant comprising a heterologous nucleic acid construct comprising a promoter sequence operably linked to a coding sequence encoding a gene product, wherein the gene product is selected from the group consisting of: G47 and TF0002, and the promoter sequence is selected from the group consisting of promoters preferentially or specifically expressed in phloem tissue of said plant; promoters preferentially or specifically expressed in meristematic tissue of said plant; promoters preferentially or specifically expressed in xylem tissue of said plant; promoters preferentially or specifically expressed in root tissue of said plant; and constitutively expressed promoters pECO1 and pECO2.

In a further aspect, the present invention relates to a genetically modified plant comprising a heterologous nucleic acid construct comprising a promoter sequence operably linked to a coding sequence encoding a gene product, wherein the gene product is selected from the group consisting of: TF0097; TF0132; and TF0109, and the promoter sequence is selected from the group consisting of promoters preferentially or specifically expressed in phloem tissue of said plant; promoters preferentially or specifically expressed in meristematic tissue of said plant; promoters preferentially or specifically expressed in xylem tissue of said plant; promoters preferentially or specifically expressed in root tissue of said plant; and constitutively expressed promoters pECO1 and pECO2.

In one embodiment, the promoter is selected from the group consisting of pLMP1 (SEQ ID No.: 13 or 33), pEC1 (SEQ ID No.: 12, 32 or 39), pEL1.1 (SEQ ID No.: 16, 34 or 40), pEA1 (SEQ ID No.: 8 or 28), pEA2 (SEQ ID No.: 9 or 29), pECO2 (SEQ ID No.: 7 or 27), pEA3 (SEQ ID No.: 10 or 30), pEA4 (SEQ ID No.: 11, 31 or 38), pLMX5 (SEQ ID No.: 14), pEX5 (SEQ ID No.: 15), pEL1.2 (SEQ ID No.: 17, 35 or 41), pER1 (SEQ ID No.: 18, 36 or 42), pER2 (SEQ ID No.: 19, 37 or 43), pECO1 (SEQ ID No.: 6 or 26), and promoters that have the same, or essentially the same, capability of initiating transcription of a coding sequence when operably linked to said coding sequence.

In one embodiment, the promoter preferentially or specifically expressed in meristematic tissue of said plant is preferentially or specifically expressed in at least one of cambium, vascular meristematic tissue, and shoot meristem tissue of said plant.

In one embodiment, the promoter is not significantly expressed in at least one of mature xylem, stem phloem, whole leaves, whole roots and bark of said plant.

In a further aspect, the invention relates to a genetically modified woody plant comprising a heterologous nucleic acid construct comprising a promoter sequence operably linked to a coding sequence encoding a gene product selected from the group consisting of G47; and TF0002, wherein the promoter is selected from the group consisting pLMP1 (SEQ ID No.: 13 or 33), pEC1 (SEQ ID No.: 12, 32 or 39), pEL1.1 (SEQ ID No.: 16, 34 or 40), pEA1 (SEQ ID No.: 8 or 28), pEA2 (SEQ ID No.: 9 or 29), pECO2 (SEQ ID No.: 7 or 27), pEA3 (SEQ ID No.: 10 or 30), pEA4 (SEQ ID No.: 11, 31 or 38), pLMX5 (SEQ ID No.: 14), pEX5 (SEQ ID No.: 15), pEL1.2 (SEQ ID No.: 17, 35 or 41), pER1 (SEQ ID No.: 18, 36 or 42), pER2 (SEQ ID No.: 19, 37 or 43), pECO1 (SEQ ID No.: 6 or 26), and promoters that have the same, or essentially the same, capability of initiating transcription of a coding sequence when operably linked to said coding sequence.

In a further aspect, the invention relates to a genetically modified plant comprising a heterologous nucleic acid construct comprising a promoter sequence operably linked to a coding sequence encoding a gene product selected from the group consisting of TF0097; TF0132; and TF0109, wherein the promoter is selected from the group consisting pLMP1 (SEQ ID No.: 13 or 33), pEC1 (SEQ ID No.: 12, 32 or 39), pEL1.1 (SEQ ID No.: 16, 34 or 40), pEA1 (SEQ ID No.: 8 or 28), pEA2 (SEQ ID No.: 9 or 29), pECO2 (SEQ ID No.: 7 or 27), pEA3 (SEQ ID No.: 10 or 30), pEA4 (SEQ ID No.: 11, 31 or 38), pLMX5 (SEQ ID No.: 14), pEX5 (SEQ ID No.: 15), pEL1.2 (SEQ ID No.: 17, 35 or 41), pER1 (SEQ ID No.: 18, 36 or 42), pER2 (SEQ ID No.: 19, 37 or 43), pECO1 (SEQ ID No.: 6 or 26), and promoters that have the same, or essentially the same, capability of initiating transcription of a coding sequence when operably linked to said coding sequence.

In one embodiment, the invention relates to a genetically modified plant according to the invention, wherein the gene product is a protein from Eucalyptus grandis, Zea Mays, Populus trichocarpa, or Arabidopsis thaliana.

It should be further noticed that the annual plant Arabidopsis thaliana, differ in many aspects from woody plants such as poplar and eucalyptus. In short, the differences can be summarized as in the table below,

| Arabidopsis thaliana | Woody plants |
|---|---|
| Determinate growth | Unterminated growth |
| Leaves are produced in a | Leaves are produced on the branches |
| leaf rosette at the ground | of trees and the growth pattern of the |
| | branches is important for yield in the |
| | field |
| No branches | Have branches |
| No stem, no wood formed | Stem with wood |
| Minute xylem tissue, only | Secondary xylem main tissue |
| found flowering parts, such | in woody plants |
| as flower stalks | |

There is little doubt, that Arabidopsis is an important model for trees, but it is also apparent that true woody plant systems are necessary to investigate some ‘unique’ tree processes. One example is flowering time genes which will overlap between trees and annuals, but the effect flowering has on plant growth is completely different between Arabidopsis and trees. In Arabidopsis flowering ends the life cycle of the plant, whereas a woody plant continues to grow for many years after flowering.

Gene function is in many instances, partly similar between Arabidopsis and woody plants, but not all, such as Eucalyptus and Populus, however the effect the genes will have on growth and development will differ between species with such different life cycles and growth patterns as annuals such as Arabidopsis and perennial plants such as trees. The difference is extra clear when it comes to predict increased yield of tissues such as wood which is the main harvested and used tissue in woody plants trees. Arabidopsis have a growth cycle that is short and ends with producing seeds within a year, then the plant dies. The bulk of the biomass in Arabidopsis will be in the leaves and in the flower structures, very little will be in secondary xylem. In contrast a woody plant or a tree growth is indeterminately, flowering is usually after at least several years and the tree continue to live and grow after the first flowering have occurred. The main biomass of trees is also in the secondary xylem that make up the bulk of the root system and of the tree stem as well as the branches. The main goal in forestry is to produce trees with increased wood formation. Wood is produced by secondary growth, a growth phase clearly distinguished from elongation growth and production of leaves or flower structures.

In one embodiment, the invention relates to a genetically modified plant according to the invention having a modified trait as compared to a wild-type plant of the same species, wherein the modified trait is selected from plant yield, height, plant width, stem volume, stem dry weight, bark dry weight, wood density, leaf dry weight, average internode length, number of internodes, water use efficiency and drought tolerance.

In one embodiment, the invention relates to a genetically modified plant according to the invention, wherein the modified trait is increased as compared to a wild-type plant of the same species when said plants are grown under identical field conditions for a period of at least one year.

In one embodiment the plant is a woody plant, such as a hardwood plant.

In one embodiment the genetically modified plant is of the genus Eucalyptus or Populus.

In one embodiment the plant is a crop plant, preferably corn, soybeans or oil seed rape.

In one embodiment, the invention relates to a genetically modified woody plant according to the invention, wherein the coding sequence encodes the gene product G47 and the promoter sequence is selected from the group consisting of pLMP1 and pLMX5, and the modified trait is at least one of water use efficiency, plant yield, plant height, plant width, stem volume, stem dry weight, bark dry weight, water use efficiency and drought tolerance.

In one embodiment, the invention relates to a genetically modified woody plant according to the invention, wherein the coding sequence encodes the gene product TF0002 and the promoter sequence is selected from the group consisting of pLMP1 and pLMX5, and the modified trait is at least one of water use efficiency, plant yield, height, plant width, stem volume, stem dry weight, bark dry weight, and wood density.

In one embodiment, the invention relates to a genetically modified plant according to the invention, wherein the coding sequence encodes the gene product TF0097 and the promoter sequence is selected from the group consisting of pEC1, pEL1.1, pEA1, pEA3, pEX5, pER1, and the modified trait is at least one of water use efficiency, plant yield, height, plant width, stem volume, stem dry weight, bark dry weight, wood density, leaf dry weight.

In one embodiment, the invention relates to a genetically modified plant according to the invention, wherein the coding sequence encodes the gene product TF0132 and the promoter sequence is selected from the group consisting pEC1, pEA1, pEA4, pECO1, pECO2, and the modified trait is at least one of water use efficiency, plant yield, height, plant width, stem volume, stem dry weight, bark dry weight, wood density, leaf dry weight.

In one embodiment, the invention relates to a genetically modified plant according to the invention, wherein the coding sequence encodes the gene product TF0109 and the promoter sequence is selected from the group consisting pEA2, pEA1, pECO2, pEC1, pEX5, and the modified trait is at least one of water use efficiency, plant yield, height, plant width, stem volume, stem dry weight, bark dry weight, wood density, leaf dry weight.

In a further aspect, the invention relates to a method to make a genetically modified plant according to the invention, said method comprising the following steps;
a) providing suitable part of a plant;
b) providing a heterologous nucleic acid construct comprising a promoter sequence operably linked to a coding sequence encoding a gene product, wherein said coding sequence is selected from the group consisting of G47; TF0002, TF0097; TF0132; and TF0109, and wherein said promoter sequence is selected from the group consisting of promoters preferentially or specifically expressed in phloem tissue of said plant; promoters preferentially or specifically expressed in meristematic tissue of said plant; promoters preferentially or specifically expressed in xylem tissue of said plant; promoters preferentially or specifically expressed in root tissue of said plant; and constitutively expressed promoters pECO1 and pECO2.
c) introducing the heterologous nucleic acid construct into said suitable part of the woody plant; and
d) regenerating a genetically modified plant from said suitable part of the plant.

In a further aspect, the invention relates to a method to make a genetically modified plant according to the invention, said method comprising the following steps;
a) providing suitable part of a plant;
b) providing a heterologous nucleic acid construct comprising a promoter sequence operably linked to a coding sequence encoding a gene product, wherein said coding sequence is selected from the group consisting of G47; TF0002, TF0097; TF0132; and TF0109, and wherein said promoter is selected from the group consisting of pLMP1, pEC1, pEL1.1, pEA1, pEA2, pECO2, pEA3, pEA4, pLMX1, pEX5, pEL1.2, pER1, pER2, pECO1, and promoters that have the same, or essentially the same, capability of initiating transcription of a coding sequence when operably linked to said coding sequence;
c) introducing the heterologous nucleic acid construct into said suitable part of the plant; and
d) regenerating a genetically modified tree from said suitable part of the plant.

### BRIEF DESCRIPTION OF THE DRAWINGS

FIG. 1, shows greenhouse (G H) and field trial (F T) data for a prior art hybrid aspen, wherein a trait gene AtG47 is expressed under the constitutive 35S promoter. Percent numbers are compared to wild type reference plants.

FIG. 2, shows greenhouse (G H) and field trial (F T) data from a hybrid aspen, wherein a trait gene AtG47 is expressed using the phloem-specific promoter, pLMP1. Percent numbers are compared to wild type reference plants.

FIG. 3, shows improved drought stress tolerance analysis in green house experiments of the tree different constructs pLMX5:AtGA47, pLMP1:AtGA47 and p35S:AtGA47 compared to wild type plants. Y-axis, shows the percent of plants suffering of turgor loss during water shortage.

FIG. 4, shows greenhouse (G H) and field trial (F T) data from transgenic hybrid aspen plants with the promoter-gene construct p35S-PttTF0002. Percent numbers are compared to wild type reference plants.

FIG. 5, shows greenhouse (G H) and field trial (F T) data from transgenic hybrid aspen plants with the vascular/xylem specific promoter-gene construct pLMX5-PttTF0002. Percent numbers are compared to wild type reference plants.

FIG. 6, shows greenhouse (G H) data from transgenic hybrid aspen plants with the phloem-specific promoter-gene construct pLMP1-PttTF0002. Percent numbers are compared to wild type reference plants.

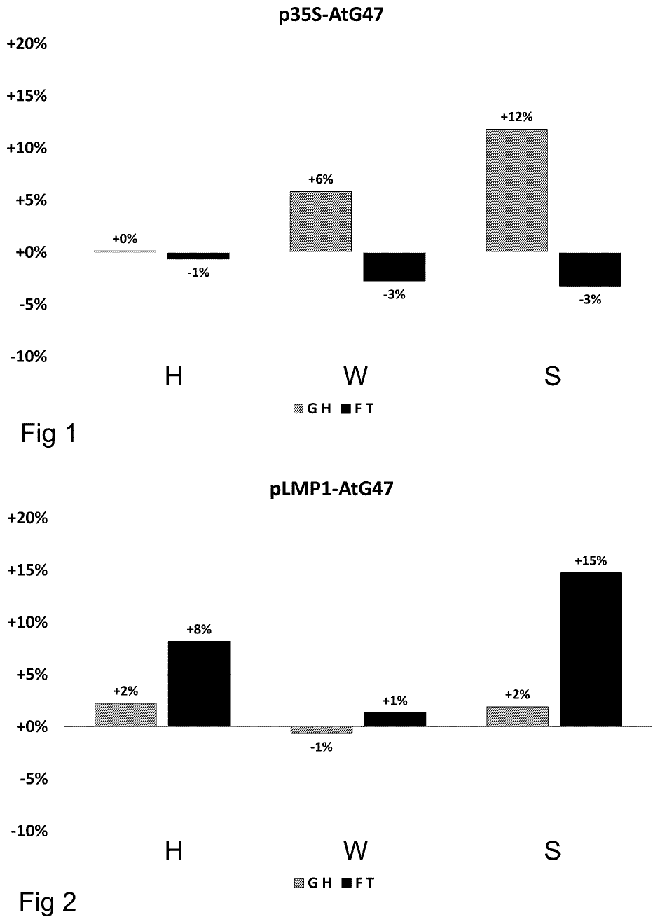

In FIGS. 1, 2, 4, 5 and 6, H represent height, W represent width of the stem and S represent stem volume.

### Definitions

All terms and words used in the present specification are intended to have the meaning generally given to them by the person skilled in the art of plant biotechnology. However, a few terms are explained in more detail below in order to avoid ambiguities.

The naming of genes presented in this disclosure originate from the inventors or others work. In brief, the first two or three letters denotes the plant name in Latin directly followed by the gene name, exemplified by the gene TF0097, from Arabidopsis thaliana it is denoted, AtTF0097. The same gene from Eucalyptus grandis is denoted EgTF0097. When an ortholog gene is know it will follow the name presented at the Phytozome Comparative Plant Genomics Portal (phytozome.jgi.doe.gov) using the latest version of Phytozome. At present the version 11.0 is used. Most ortholog gene names in the present disclosure are found in Phytozome.

A “p” in front of a gene denotes that this is the promoter of said gene, for example pRBCS is the promoter of the gene ribulose-1,5-bisphosphate carboxylase small subunit (RBCS).

If a promoter, when operably linked to a coding sequence, entails expression of the coding sequence in a certain tissue or region of the plant to a significantly larger extent than in another tissue or region, then that promoter is said to be “preferentially expressed” in that tissue or region. A promoter may be preferentially expressed in more than one tissue or region. Expression levels can be analysed as described herein.

If a promoter, when operably linked to a coding sequence, entails expression of the coding sequence in a single tissue or region of the plant to a significantly larger extent than in any other tissue or region, then that promoter is said to be “specifically expressed” in that tissue or region. Expression levels can be analysed as described herein.

By “ortholog” or “orthologous polypeptide” is meant a polypeptide expressed by evolutionarily related genes that have a similar nucleic acid sequence, where the polypeptide has similar functional properties. Orthologous genes are structurally related genes, from different species, derived by a speciation event from an ancestral gene. Related to orthologs are paralogs. Paralogous genes are structurally related genes within a single plant species most probably derived by a duplication of a gene. Several different methods are known by those of skill in the art for identifying and defining these functionally homologous sequences.

Orthologous genes from different organisms have highly conserved functions and can be used for identification of genes that could perform the invention in the same way as the genes presented here. Paralogous genes, which have diverged through gene duplication, may encode protein retaining similar functions. Orthologous genes are the product of speciation, the production of new species from a parental species, giving rise to two or more genes with common ancestry and with similar sequence and similar function. These genes, termed orthologous genes, often have an identical function within their host plants and are often interchangeable between species without losing function. Identification of an “ortholog” gene may be done by identifying polypeptides in public databases using the software tool BLAST with one of the polypeptides encoded by a gene. Subsequently additional software programs are used to align and analyze ancestry. The sequence identity between two orthologous genes may be low.

A promoter is said to be an “orthologous promoter” to a promoter in a different species when the respective promoters initiate transcription of orthologous genes in wild type plants of the respective species.

The term “plant” including “crop plants” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

A “woody plant” is a plant that produces wood as a structural tissue.

The terms “substantially identical” or “sequence identity” may indicate a quantitative measure of the degree of identity between two amino acid sequences or two nucleic acids (DNA or RNA) of equal length. When the two sequences to be compared are not of equal length, they are aligned to give the best possible fit, by allowing the insertion of gaps or, alternatively, truncation at the ends of the polypeptide sequences or nucleotide sequences. The “sequence identity” may be presented as percent number, such as at least 40, 50%, 55,%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% amino acid sequence identity of the entire length, when compared and aligned for maximum correspondence, as measured using a sequence comparison algorithm or by visual inspection.

The sequence identity of the polypeptides of the invention can be calculated as (N_{ref}−N_{dif})100/N_{ref}, wherein N_{dif} is the total number of non-identical residues in the two sequences when aligned and wherein N_{ref} is the number of residues in one of the sequences. The sequence identity between one or more sequence may also be based on alignments using the Clustal W or Clustal X software. In one embodiment of the invention, alignment is performed with the sequence alignment method Clustal X version 2 with default parameters. The parameter set preferably used are for pairwise alignment: Gap open penalty: 10; Gap Extension Penalty: 0.1, for multiple alignment, Gap open penalty is 10 and Gap Extension Penalty is 0.2. Protein Weight matrix is set on Identity. Both Residue-specific and Hydrophobic Penalties are “ON”, Gap separation distance is 4 and End Gap separation is “OFF”, No Use negative matrix and finally the Delay Divergent Cut-off is set to 30%. The Version 2 of Clustal W and Clustal X is described in: Larkin et al. 2007, Clustal W and Clustal X version 2.0. Bioinformatics, 23:2947-2948. The identity between two sequence (protein or nucleic acids) can practically be determined by using different BLAST tools at NCBI (https://www.ncbi.nlm.nih.gov/).

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group Glycine, Alanine, Valine, Leucine, Isoleucine; group Serine, Cysteine, Selenocysteine, Threonine, Methionine; group Proline; group Phenylalanine, Tyrosine, Tryptophan; Group Aspartate, Glutamate, Asparagine, and Glutamine.

In some aspects, the amino acid substantial identity exists over a polypeptide sequences length of 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, 450, 500, 600, 700 amino acids in the polypeptide with a “sequence identity” as defined above.

In certain aspects, substantial identity exists over a region of nucleic acid sequences of at least about 50 nucleic acid residues, such as at least about 100, 150, 200, 250, 300, 330, 360, 375, 400, 425, 450, 460, 480, 500, 600, 700, 800 such as at least about 900 nucleotides or such as at least about 1 kb, 2 kb, or such as at least about 3 kb.

A gene (nucleic acid molecule comprising a coding sequence) is “operably linked” to a promoter when its transcription is under the control of the promoter and where transcription results in a transcript whose subsequent translation yields the product encoded by the gene.

The term “increasing expression” is intended to encompass well known methods to increase the expression by regulatory sequences, such as promoters, or proteins, such as transcription factors. The terms “increasing expression”, “enhanced expression” and “over-expression” can be used interchangeably in this text. Increased expression may lead to an increased amount of the over-expressed protein/enzyme, which may lead to an increased activity of the protein of interest that contributes to its high efficiency.

The term “yield” as used herein generally refers to a measurable product from a plant, particularly a crop. Yield and yield increase (in comparison to a non-transformed starting or wild-type plant) can be measured in a number of ways, and it is understood that a skilled person will be able to apply the correct meaning in view of the particular embodiments, the particular crop concerned and the specific purpose or application concerned. The terms “improved yield” or “increased yield” can be used interchangeable. As used herein, the term “improved yield” or the term “increased yield” means any improvement in the yield of any measured plant product, such as grain, fruit, leaf, root, cob or fibre. In accordance with the invention, changes in different phenotypic traits may improve yield. For example, and without limitation, parameters such as floral organ development, root initiation, root biomass, seed number, seed weight, harvest index, leaf formation, phototropism, apical dominance, and fruit development, are suitable measurements of improved yield. Increased yield includes higher fruit yields, higher seed yields, higher fresh matter production, and/or higher dry matter production. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans or corn derived from a crop comprising plants which are transgenic for the chimeric genes of the invention, as compared with the bu/acre yield from untransformed soybeans or corn cultivated under the same conditions, is an improved yield in accordance with the invention. The increased or improved yield can be achieved in the absence or presence of stress conditions. For example, enhanced or increased “yield” refers to one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield; enhanced yield of harvestable parts, either dry or fresh-weight or both, either aerial or underground or both; enhanced yield of crop fruit, either dry or fresh-weight or both, either aerial or underground or both; and enhanced yield of seeds, either dry or fresh-weight or both, either aerial or underground or both. “Crop yield” is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. The yield of a plant can depend on the specific plant/crop of interest as well as its intended application (such as food production, feed production, processed food production, biofuel, biogas or alcohol production, or the like) of interest in each particular case. Thus, in one embodiment, yield can be calculated as harvest index (expressed as a ratio of the weight of the respective harvestable parts divided by the total biomass), harvestable parts weight per area (acre, square meter, or the like); and the like. The harvest index is the ratio of yield biomass to the total cumulative biomass at harvest. Harvest index is relatively stable under many environmental conditions, and so a robust correlation between plant size and grain yield is possible. Measurements of plant size in early development, under standardized conditions in a growth chamber or greenhouse, are standard practices to measure potential yield advantages conferred by the presence of a transgene. Accordingly, the yield of a plant can be increased by improving one or more of the yield-related phenotypes or traits. Such yield-related phenotypes or traits of a plant the improvement of which results in increased yield comprise, without limitation, the increase of the intrinsic yield capacity of a plant, improved nutrient use efficiency, and/or increased stress tolerance. For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/or the like). “Yield” can also refer to seed yield which can be measured by one or more of the following parameters: number of seeds or number of filled seeds (per plant or per area (acre/square meter/or the like)); seed filling rate (ratio between number of filled seeds and total number of seeds); number of flowers per plant; seed biomass or total seeds weight (per plant or per area (acre/square meter/or the like); thousand kernel weight (TKW; extrapolated from the number of filled seeds counted and their total weight; an increase in TKW may be caused by an increased seed size, an increased seed weight, an increased embryo size, and/or an increased endosperm). Other parameters allowing to measure seed yield are also known in the art. Seed yield may be determined on a dry weight or on a fresh weight basis, or typically on a moisture adjusted basis, e.g. percent moisture. For example, the term “increased yield” means that a plant, exhibits an increased growth rate, e.g. in the absence or presence of abiotic environmental stress, compared to the corresponding wild-type plant. An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or 3 seeds. A prolonged growth comprises survival and/or continued growth of the plant, at the moment when the non-transformed wild type organism shows visual symptoms of deficiency and/or death. When the plant of the invention is a corn plant, increased yield for corn plants means, for example, increased seed yield, in particular for corn varieties used for feed or food. Increased seed yield of corn refers to an increased kernel size or weight, an increased kernel per ear, or increased ears per plant. Alternatively or in addition the cob yield may be increased, or the length or size of the cob is increased, or the kernel per cob ratio is improved. When the plant of the invention is a soy plant, increased yield for soy plants means increased seed yield, in particular for soy varieties used for feed or food. Increased seed yield of soy refers for example to an increased kernel size or weight, an increased kernel per pod, or increased pods per plant. When the plant of the invention is an oil seed rape (OSR) plant, increased yield for OSR plants means increased seed yield, in particular for OSR varieties used for feed or food. Increased seed yield of OSR refers to an increased seed size or weight, an increased seed number per silique, or increased siliques per plant. When the plant of the invention is a cotton plant, increased yield for cotton plants means increased lint yield. Increased lint yield of cotton refers in one embodiment to an increased length of lint. When the plant is a plant belonging to grasses an increased leaf can mean an increased leaf biomass. Said increased yield can typically be achieved by enhancing or improving, one or more yield related traits of the plant. Such yield-related traits of a plant comprise, without limitation, the increase of the intrinsic yield capacity of a plant, improved nutrient use efficiency, and/or increased stress tolerance, in particular increased abiotic stress tolerance. Intrinsic yield capacity of a plant can be, for example, manifested by improving the specific (intrinsic) seed yield (e.g. in terms of increased seed/grain size, increased ear number, increased seed number per ear, improvement of seed filling, improvement of seed composition, embryo and/or endosperm improvements, or the like); modification and improvement of inherent growth and development mechanisms of a plant (such as plant height, plant growth rate, pod number, pod position on the plant, number of internodes, incidence of pod shatter, efficiency of nodulation and nitrogen fixation, efficiency of carbon assimilation, improvement of seedling vigour/early vigour, enhanced efficiency of germination (under stressed or non-stressed conditions), improvement in plant architecture, cell cycle modifications, photosynthesis modifications, various signalling pathway modifications, modification of transcriptional regulation, modification of translational regulation, modification of enzyme activities, and the like); and/or the like.

The term “water use efficiency” (WUE) has been defined in various ways in the literature, but is commonly known as a simple measure for the water productivity of a plant. An increase in water use efficiency is commonly cited as a response mechanism of plants to moderate to severe soil water deficits, and has been the focus of many programs that seek to increase crop tolerance of drought. Different plant species has different inherent water use efficiency.

Water use efficiency is preferably measured by the carbon isotope discrimination analysis for improved drought tolerance. It is known that carbon isotope discrimination is highly correlated with water use efficiency in C3 plants. The isotopic ratio of ¹³C to ¹²C (δ¹³C) in plant tissue is less than the isotopic ratio of ¹³C to ¹²C in the atmosphere, indicating that plants discriminate against ¹³C during photosynthesis. The isotopic ratio δ¹³C varies mainly due to discrimination during diffusion of CO₂ across the stomatal pore, where diffusion of ¹³CO₂ is lower than that of ¹²CO₂, and an additional effect caused by the preference of ribulose bisphosphate carboxylase for ¹²CO₂ over ¹³CO₂. Both processes discriminate against the heavier isotope, ¹³C, Farquhar, G. D., J. R. Ehleringer, and K. T. Hubick. 1989. Carbon isotope discrimination and photosynthesis. Ann. Rev. Plant Physiol. 40:503-537.

Specific plants have been genetically modified to better withstand drought and/or to improve their water use efficiency. For example, US 2016/0,272,990 describes a plant cell having a recombinant DNA construct which provides for an enhanced trait as compared to control plants. The said enhanced trait may e.g. be enhanced water use efficiency, enhanced cold tolerance, increased yield, enhanced nitrogen use efficiency, enhanced seed protein, or enhanced seed oil.

The terms “increase”, “improve” or “enhance” are interchangeable and shall mean in the sense of the application at least a 3%, 4%, 5%, 6%, 7%, 8%, 9% or 10%, preferably at least 15% or 20%, more preferably 25%, 30%, 35%, 40%, 50%, 60%, 70%, 80%, 90%, 95% or more yield and/or growth in comparison to control plants as defined herein.

### DETAILED DESCRIPTION OF THE INVENTION

On a general level, the present invention relates to controlling gene regulation in order to retain or further improve positive phenotypical traits provided by a trait gene when growth conditions change. Controlled gene regulation is used to tailor the expression pattern of the trait gene to the growth condition under which the plant is to be grown.

The present inventors have found that constitutive over-expression of a trait gene that provide improved growth under greenhouse conditions may not provide similar improved growth under field conditions, and may in fact lead to impaired growth, see Example 1.

These unexpected results led the inventors to test other combinations of promoters and genes. It is evident from the results disclosed in Example 1 that having a strong constitutive expression of a trait gene can, as with the CaMV 35S promoter construct, have disadvantageous effects under some field trial conditions. Furthermore, these results demonstrate the need for new promoters and new promoter-gene combinations to tailor the expression pattern of the trait gene to the specific growth condition and to retain or further improve the positive phenotypical traits provided by the gene when growth conditions change.

Consequently, the invention consists of combinations of promoters, in particular pECO1, pECO2, pEA1, pEA2, pEA3, pEA4, pEC1, pLMP1, pLMX5, pEX5, pEL1.1, pEL1.2, pER1 and pER2 promoters, and one of the following genes, G47, TF0002, TF0097, TF0132 and TF0109 that confer improved plant traits in field use.

### Novel Promoter-Gene Combinations

This invention discloses novel combinations of promoters and trait genes, selected from G47, TF0002, TF0097, TF0132 and TF0109. When any of these combinations are expressed in a tree a number of improved phenotypical effects might be noted, such as plant height, plant biomass, stem diameter, stem volume, wood density, stem dry weight, bark dry weight, average internode length, number of internodes.

The combinations of promoters and genes were designed based on scientific information about the function and expression pattern of the trait gene and the promoter established by the inventors and supported by information available in the prior art. Such information provides concepts where to direct expression as well as where to avert gene expression. However, it is known to a person skilled in the art that anticipating the effect that a specific promoter-gene combination has on the plant is ingenious and nontrivial.

The novel combinations of a promoter and a biological functional polypeptide selected from the genes G47, TF0002, TF0097, TF0132 and TF0109, as defined by their polypeptide sequence ID No.: 1 to 5, respectively, when it is introduced into the plant by use of a recombinant DNA construct, as explained herein.

### Crop Plants.

Crop plants that are useful in the methods of the invention include in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca 30 arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g.

Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amongst others.

### Woody Plants

The present invention further relates to genetically modified woody plants, such as genetically modified angiosperms, dicotyledonous woody plants, preferably trees.

The invention further relates to genetically modified woody plants from gymnosperms, such as conifer trees.

The woody plant may be a hardwood plant e.g. selected from the group consisting of acacia, eucalyptus, hornbeam, beech, mahogany, walnut, oak, ash, willow, hickory, birch, chestnut, poplar, alder, maple, sycamore, ginkgo, a palm tree and sweet gum. Hardwood plants, such as eucalyptus and plants from the Salicaceae family, such as willow, poplar and aspen including variants thereof, are of particular interest, as these groups include fast-growing species of tree or woody shrub which are grown specifically to provide timber for building material, raw material for pulping, bio-fuels and/or bio chemicals.

In further embodiments, the genetically modified tree is a conifer tree, such as a member of the order Pinales, with members of the family Cupressaceae, such as Cupressus spp., Juniperus spp., Sequoia spp., Sequoiadendron spp.; with members of the family Taxaceae (Taxus spp.) and with members of the family Pinaceae, such as the genera Abies spp., Cedrus spp., Larix spp., Picea spp., Pinus spp., Pseudotsuga spp., Tsuga spp.

Alternatively, the woody plants which may be selected from the group consisting of cotton, bamboo and rubber plants.

In another embodiment, the genetically modified tree is a deciduous tree including hybrids, and cultivars such as acacia (Acacia spp.), alder (Alnus spp.), birch (Betula spp.), hornbeam (Carpinus spp.), hickory (Carya spp.), chestnut (Castanea spp.), beech (Fagus spp.), walnut (Juglans spp.), oak (Quercus spp.), ash (Fraxinus spp.), poplar (Populus spp.), aspen (Populus spp.), willow (Salix spp.), eucalyptus (Eucalyptus spp.), sycamore (Platanus spp.), maple (Acer spp.), mahogany (Swietenia spp.), sweet gum (Liquidambar spp.). Genetically modified trees of the families Salicaceae and Myrtaceae are preferred, most preferred are genetically modified tree from the genus Eucalyptus and Populus.

In yet another embodiment, the genetically modified tree is a fruit bearing plant, including hybrids, and cultivars such as, apple (Malus spp.), plum (Prunus spp.), pear (Pyrus spp.), orange (Citrus spp.), lemon (Citrus spp.), kiwi fruit (Actinidia spp.), cherry (Prunus spp.), grapevine (Vitis spp.), and fig (Ficus spp.).

In a specific embodiment, the genetically modified tree is a woody plant whose leaves can be eaten as leaf vegetables include Adansonia, Aralia, Moringa, Morus, and Toona species.

### Promoters

A general method for identification of ortholog plants promoters, preferably tissue-specific promoter.

The following set of criteria may be used to identify plant promoters. Some of these criteria are:
Establishment of expression pattern:
   Select the plant of interest and identify promoters that corresponds to tested and verified promoters of plants similar to the selected plant.
   Identify promoters with a well-established expression pattern, preferably confirmed by extensive analysis from a plant similar to the selected plant.
   Selection of should preferably be based on expression pattern analysis performed in plant similar to the selected plant, for example, microarray or expressed RNA sequence analysis.
Identification of an ortholog promoter.
   Once a desired expression pattern is identified a phylogenetic analysis of the corresponding gene and closely related genes from another plant species similar to the selected plant can be done.
   Identification of an ortholog gene might be performed using publicly available genome database resources, such as the Phytozome database.
   The region upstream the coding sequence of the identified orthologues gene is selected and a putative promoter is chosen. The length of the putative promoter may be determined by using available scientific information together with homology analyses of promoter regions of orthologous genes from multiple plant species.
The identified ortholog promoter might be verified by different expression methods, such as GUS-expression.

### Identification of Eucalyptus Promoters

A set of criteria has been used to identify Eucalyptus promoters. Some of these criteria are:
Eucalyptus promoters that corresponds to tested and verified Populus promoters.
Eucalyptus promoters that corresponds to promoters with a well-established expression pattern confirmed by extensive analysis.
Selection of Eucalyptus promoters based on expression pattern analysis performed in Eucalyptus, for example, microarray or RNAseq analysis.
Selection of Eucalyptus promoters based on expression pattern analysis performed in Populus and/or Arabidopsis, for example, microarray or RNAseq analysis.

Once a desired expression pattern was identified a phylogenetic analysis of the corresponding gene and closely related genes from Eucalyptus grandis, Populus trichocarpa and Arabidopsis thaliana was performed using publicly available genome database resources. Mostly the Phytozome database was used for searches. Thus, orthology and homology within and between species was determined and a Eucalyptus gene with a putative expression pattern similar to the desired expression pattern was identified.

The region upstream the coding sequence of the identified Eucalyptus gene was examined and a putative promoter region length was determined using available scientific information together with homology analyses of promoter regions of orthologous genes from multiple plant species, such as Eucalyptus, Populus and Arabidopsis.

### Identification of Novel Eucalyptus Tissue-Specific Promoters

This invention has established a number of novel Eucalyptus tissue-specific promoters such as, such as apex active promoters, stem/cambium active promoters, root active promoters and promoters active in leaves. These promoters offer invaluable instruments to specifically control the expression of trait genes in a plant, more specifically in a tree and even more specifically in Eucalyptus.

The novel Eucalyptus promoters were identified by using scientific information available from multiple plant species, such as Eucalyptus, Populus and Arabidopsis, from gene expression analyses, expression of known promoters and the expression and function of the corresponding genes and of identified orthologous/homologous genes.

In order to identify the Eucalyptus promoters a strategy was formulated involving two steps, first identification of a set of promoters and secondly verifying that the identified promoter is functional.

Fourteen Eucalyptus promoters were selected for combination with the five trait genes. Two promoters from hybrid aspen were also included, see below for details. The constitutive Cauliflower Mosaic Virus 35S promoter, p35S was used in combination with all genes for comparison. For details about cloning of the genes, see the examples.

TABLE 1
Eucalyptus and hybrid aspen promoters and the CaMV 35S promoter.

| Promoters | | | Examples of orthologous promoters | | | | | |
|---|---|---|---|---|---|---|---|---|
| | | Promoter | | | | | | Promoter |
| Promoter | Sequence | region | Promoter | Sequence | Promoter | Promoter | Sequence | region |
| name | ID No. | length (nt) | name | ID No. | region | name | ID No. | length (nt) |
| pECO1 | 6 | 1084 | pECO1-ort | 26 | 1802 | | | |
| | | | poplar | | | | | |
| pECO2 | 7 | 2000 | pECO2-ort | 27 | 2000 | | | |
| | | | poplar | | | | | |
| pEA1 | 8 | 2000 | pEA1-ort poplar | 28 | 2000 | | | |
| pEA2 | 9 | 2500 | pEA2-ort poplar | 29 | 2500 | | | |
| pEA3 | 10 | 2700 | pEA3-ort poplar | 30 | 2700 | | | |
| pEA4 | 11 | 2500 | pEA4-ort poplar | 31 | 2500 | pEA4-para | 38 | 2500 |
| | | | | | | poplar | | |
| pEC1 | 12 | 2101 | pEC1-ort poplar | 32 | 2101 | pEC1-para | 39 | 2101 |
| | | | | | | poplar | | |
| pLMP1 | 13 | 1487 | pEP1 | 33 | 1801 | | | |
| pLMX5 | 14 | 1780 | pEX5 | 15 | 2001 | | | |
| pEL1.1 | 16 | 600 | pEL1.1-ort | 34 | 600 | pEL1.1-para | 40 | 600 |
| | | | poplar | | | poplar | | |
| pEL1.2 | 17 | 1800 | pEL1.2-ort | 35 | 1800 | pEL1.2-para | 41 | 1800 |
| | | | poplar | | | poplar | | |
| pER1 | 18 | 2000 | pER1-ort poplar | 36 | 2000 | pER1-para | 42 | 2000 |
| | | | | | | poplar | | |
| pER2 | 19 | 2000 | pER2-ort poplar | 37 | 2000 | pER2-para | 43 | 2000 |
| | | | | | | poplar | | |
| p35S | 20 | 942 | | | | | | |

The above identified nucleic acid sequences constitute promoter regions. As known in the art, promoter regions comprise a number of cis-regulatory elements, to which proteins involved in transcription bind. These regulatory elements are primarily located within a few hundred nucleotides upstream the start codon.

Thus, in one aspect the methods and products of the invention make use of the promoter regions in the plants and methods according to the invention.

In further aspects, the methods and products of the invention make use of the regulatory elements comprised in the promoter regions, i.e. polynucleotides that have the same, or essentially the same, capability of initiating transcription of a coding sequence when operably linked to said coding sequence, as compared to the promoter regions disclosed in Table 1.

In one aspect, the methods and products of the invention make use of the part of the promoter region that is located between start codon and 3000, 2500, 2000, 1800, 1600, 1400, 1200, 1000, 900, 800, 700, 600, 500, 400, 300, 250, 200, 175, 150, or 125 nucleotides upstream, or nucleic acid stretches that are at least 40%, 50%, 55,%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% identical to said part of the promoter region and that have the same, or essentially the same, capability of initiating transcription of a coding sequence when operably linked to said coding sequence, as compared to the promoter regions disclosed in Table 1.

In one aspect, the methods and products of the invention make use of promoters that are orthologous to the promoters disclosed in Table 1, i.e. promoters from different species that initiate transcription of orthologous genes in wild type woody plants of the respective species. Also such orthologous promoters should have the same, or essentially the same, capability of initiating transcription of a coding sequence when operably linked to said coding sequence, as compared to the promoter regions disclosed in Table 1.

Assessment of whether a nucleic acid has the same, or essentially the same, capability of initiating transcription of a coding sequence when operably linked to said coding sequence, can be done in a number of ways known to the skilled person. One way is to study expression patterns by histological studies of plants harbouring a promoter-β-glucuronidase (GUS) construct, as detailed in Example 1 and 2. The nucleic acid's activity as a promoter is then assayed using the established histochemical GUS staining technique, and compared to one or more constructs harbouring one or more of the promoter regions of the present disclosure.

Promoters from Eucalyptus and Hybrid Aspen
The Promoter pECO1

The dynamin protein, a GTPase that is responsible for endocytosis in the eukaryotic cell, was identified as a highly and constitutively expressed gene by studying expression data from hybrid aspen microarray experiments.

To clone the Eucalyptus pECO1 promoter, the amino acid sequence from Populus trichocarpa dynamin protein gene, accession number Potri.001 G090600, was used in a blast search followed by a phylogenetic analysis of the identified putative homologous and orthologous genes. The identified E. grandis ortholog, accession number Eucgr.E00053, has an 86.7% polypeptide sequence identity to the Populus gene product. The sequence immediately upstream of, but not including, the start codon of the gene Eucgr.E00053 was used for synthesis of the pECO1 promoter, Seq ID No.: 6. A putative orthologous promoter to the pECO1 promoter is the Populus tremula x tremuloides promoter pECO1-ort poplar, Seq ID No.: 26.

The promoter pECO1 has been established and used as a constitutive promoter in the patent application SE1651431-7.

The Promoter pECO2

A constitutively expressed gene encoding a housekeeping protein, glyceraldehyde 3-phosphate dehydrogenase, GAPDH, was identified as a constitutively expressed gene suitable as a stable reference for RT-qPCR analysis by Czechowski et al. Plant Physiology 2005, Vol. 139, 5-17. GAPDH catalyses a step in glycolysis and serves to break down glucose for energy and carbon molecules.

The GAPDH gene from A. thaliana, accession number AT1 G13440, was used in a blast search followed by a phylogenetic analysis of the identified putative homologous and orthologous genes. The identified Eucalyptus grandis ortholog, accession number Eucgr.H04673, has a 93.1% polypeptide sequence identity to AT1G13440. Avoiding to include the coding region of an adjacent gene, a 1084 base pair long promoter fragment immediately upstream of, but not including, the start codon of gene Eucgr.H04673 was used for synthesis of the pECO2 promoter, Seq ID No.: 7. A putative orthologous promoter to the pECO2 promoter is the promoter region, pECO2-ort poplar, Seq ID No.: 27, of the Populus trichocarpa gene with accession number Potri.010G055400.

The promoter pECO2 has been established and used as a constitutive promoter in the patent application SE1651431-7.

The Promoter pEA1

The gene ERECTA (ER) from A. thaliana (accession number AT2G26330) was selected based on publications regarding its known function and expression in shoot apex. The ER gene is homologous to receptor protein kinases and involved in specification of organs originating from the shoot apical meristem. The ER polypeptide contains a cytoplasmic protein kinase catalytic domain, a transmembrane region, and an extracellular leucine-rich repeat. ER has further been identified as a quantitative trait locus for transpiration efficiency by influencing epidermal and mesophyll development, stomatal density and porosity of leaves. ER has also been implicated in resistance to bacteria and to necrotrophic fungus. ER governs, together with ERL1 and ERL2, the initial decision of protodermal cells to either divide proliferatively to produce pavement cells or divide asymmetrically to generate stomatal complexes, Yokoyama et al. 1998, The Plant Journal, 15(3), 301-310.

The AT2G26330 polypeptide was used in a blast search followed by a phylogenetic analysis of the identified putative homologous and orthologous genes. This identified the E. grandis ortholog, accession number Eucgr.C00732. The orthologous gene of Populus trichocarpa is Potri.006G220100. Since the length of the promoter is unknown, a 2000 base pair long promoter fragment immediately upstream of, but not including, the start codon of gene Eucgr.C00732 was selected for synthesis of the pEA1 promoter, Seq ID No.: 8. A putative orthologous promoter to the pEA1 promoter is the promoter region, pEA1-ort poplar, Seq ID No.: 28, of the Populus trichocarpa gene with accession number Potri.006G220100.

The promoter pEA1 has been established and used as a tissue specific promoter in the patent application SE1651431-7.

The Promoter pEA2

The gene AINTEGUMENTA (ANT) from A. thaliana (accession number AT4G37750) was selected for its known function in cell proliferation and as a positive regulator of cell division and for its known expression in actively dividing cells. Loss-of-function Arabidopsis mutants lacking ANT have reduced cell division and cell number leading to reduced size of all lateral organs while over-expression increases cell number and thus organ size, Mizukami and Fischer (2000) PNAS, 97(2): 942-947.

The AT4G37750 polypeptide was used in a blast search followed by a phylogenetic analysis of the identified putative homologous and orthologous genes. This identified the E. grandis ortholog, accession number Eucgr.F02223. The putative orthologous gene in Populus trichocarpa is Potri.002g114800. Since the length of the promoter is unknown, a 2500 base pair long promoter fragment immediately upstream of, but not including, the start codon of gene Eucgr.F02223 was selected for synthesis of the pEA2 promoter, Seq ID No.: 9. A putative orthologous promoter to the pEA2 promoter is the promoter region, pEA2-ort poplar, Seq ID No.: 29, of the Populus trichocarpa gene with accession number Potri.002g114800.

The promoter pEA2 has been established and used as a tissue specific promoter in the patent application SE1651431-7.

The Promoter pEA3

The promoter of the Asymmetric leaves1 (AS1) gene, accession number AT2G37630, drives gene expression in the apical region of the plant, specifically in the leaf forming tissues of the leaf primordia. The AS1 promoter was selected based on its known specific expression pattern and the function of AS1 in leaf primordia, Byrne et al. 2000, Nature, 408(6815) 967-971.

The AT2G37630 polypeptide was used in a blast search followed by a phylogenetic analysis of the identified putative homologous and orthologous genes. The putative orthologous gene in Populus trichocarpa is Potri.006G085900. The identified Eucalyptus grandis ortholog, accession number Eucgr.K03130, has a polypeptide sequence identity of 67% to AT2G37630 over 98% of the E. grandis sequence. Promoter analysis in Arabidopsis has shown that the promoter is approximately 2.7 kb. The promoter, in both Arabidopsis and Eucalyptus, contains a large intron in the predicted 5′ UTR. A 2700 base pair long promoter fragment immediately upstream of, but not including, the start codon of gene Eucgr.K03130 was selected for synthesis of the pEA3 promoter, Seq ID No.: 10. Orthologous to the pEA3 promoter is the promoter region, pEA3-ort poplar, Seq ID No.: 30, of the Populus trichocarpa gene with accession number Potri.006G085900.

The promoter pEA3 has been established and used as a tissue specific promoter in the patent application SE1651431-7.

The Promoter pEA4

The A. thaliana gene AT5G67260 (AtCYCD3:2) encode CYCD3;2, a CYCD3 D-type cyclin, which is important for determining cell number in developing lateral organs and mediating cytokinin effects in apical growth and development. CYCD3 function contributes to the control of cell number in developing leaves by regulating the duration of the mitotic phase and timing of the transition to endocycles. CYCD3;1 expression is restricted to the shoot apical meristem (SAM), very young primordia, and young hydathodes, whereas CYCD3;2 and CYCD3;3 reporters are also active in older leaf primordia, with CYCD3;2 expression persisting longest in young leaves. The phytohormone cytokinin regulates cell division in the shoot meristem and developing leaves and induces CYCD3 expression. Loss of CYCD3 impairs shoot meristem function and leads to reduced cytokinin responses, Dewitte et al., 2007 PNAS, 104(36) 14537-14542.

The AT5G67260 polypeptide was used in a blast search followed by a phylogenetic analysis of the identified putative homologous and orthologous genes. The identified Eucalyptus grandis ortholog, accession number Eucgr.100802, has a polypeptide sequence identity of 51% to AT5G67260 over 94% of the E. grandis sequence. In Populus trichocarpa two putative orthologous genes are identified, Potri.007G048300 and Potri.005G141900; these two genes are considered paralogous genes. Promoter analysis in Arabidopsis has shown that the promoter fragment is approximately 2.5 kb. Therefore, a 2500 base pair long promoter fragment immediately upstream of, but not including, the start codon of gene Eucgr.100802 was selected for synthesis of the pEA4 promoter, Seq ID No.: 11. A putative orthologous promoter to the pEA4 promoter is the Populus trichocarpa promoter regions, pEA4-ort poplar, Seq ID No.: 31, and pEA4-para poplar, Seq ID No.: 38.

The promoter pEA4 has been established and used as a tissue specific promoter in the patent application SE1651431-7.

The Promoter pEC1

The WOX4 gene in A. thaliana is preferentially expressed in the procambial/cambial stem cells and is a regulator of vascular stem cell proliferation, Mizukami and Fischer (2000) PNAS, 97(2): 942-947. The expression pattern of the hybrid aspen ortholog (HB3/WOX4) was first identified in a high resolution expression profile over the vascular cambium, Schrader et al. 2004, The Plant Cell 16(9) 2278-2292, subsequently using more precise methods such as promoter:GUS analysis, real-time PCR and in-situ hybridization Nilsson, Doctoral thesis 2010:29, Faculty of Forest Sciences, Umen. These studies combined show that WOX4/HB3 is a cambium specific promoter well suited for tissue specific expression of chosen trait genes.

The Eucalyptus gene Eucgr.F02320 forms a phylogenetic group with the Arabidopsis WOX4 (AT1 G46480) and two P. trichocarpa homologs Potri.014G025300 and Potri.002G124100. Alignment of 4 kb fragments upstream of the coding sequence of the hybrid aspen transcripts with 4 kb upstream of the Eucgr.F02320 gene reveals major similarities of approximately 2.1 kb. This region was selected for synthesis of the stem/cambium specific promoter pEC1, Seq ID No.: 12. Putative orthologous promoters to the pEC1 promoter are the Populus trichocarpa promoter regions, pEC1-ort poplar, Seq ID No.: 32, and pEC1-para poplar, Seq ID No.: 39.

The promoter pEC1 has been established and used as a tissue specific promoter in the patent application SE1651431-7

The Hybrid Aspen Promoter pLMP1

The pLMP1 promoter, Seq ID No.: 13, from hybrid aspen has been established and used as a phloem-specific promoter, WO2004097024. The gene immediately downstream of the pLMP1 promoter was found to be preferentially expressed in the cambium and developing phloem by studying expression data from hybrid aspen microarray experiments. The phloem-specific expression pattern of the pLMP1 promoter was further verified in GUS expression assays, studying stem sections of transgenic hybrid aspen trees harbouring a recombinant DNA construct with the pLMP1 promoter and the beta-glucuronidase (GUS) reporter gene. The gene immediately downstream of the pLMP1 promoter in hybrid aspen is orthologous to the Populus trichocarpa gene with accession number Potri.013G127900.

Identification of an ortholog promoter to pLMP1. The amino acid sequence from the Populus trichocarpa gene Potri.013G127900 was used in a blast search followed by a phylogenetic analysis of the identified putative homologous and orthologous genes. The identified E. grandis ortholog, accession number Eucgr.B01456, has a 64% polypeptide sequence identity to the Populus gene product. The length of the hybrid aspen pLMP1 promoter is 1487 base pair. To allow for some interspecies variation, an 1.8 kb long promoter fragment immediately upstream of, but not including, the start codon of the gene Eucgr.B01456 was used for synthesis of the pEP1 promoter, Seq ID No.: 33. The pEP1 and pLMP1 are orthologous promoters.

The Promoter pLMX5

The pLMX5 promoter, Seq ID No.: 14, from hybrid aspen has been established and used as a xylem-specific promoter earlier and is presented in WO2004097024. The gene immediately downstream of the pLMX5 promoter was found to be preferentially expressed in the developing xylem by studying expression data from hybrid aspen microarray experiments. The xylem-specific expression pattern of the pLMX5 promoter was further verified in GUS expression assays, studying stem sections of transgenic hybrid aspen trees harbouring a recombinant DNA construct with the pLMX5 promoter and the beta-glucuronidase (GUS) reporter gene. The gene immediately downstream of the pLMX5 promoter in hybrid aspen is orthologous to the Populus trichocarpa gene with accession number Potri.002G101200.

The Promoter pEX5

To clone the Eucalyptus pEX5 promoter, the amino acid sequence from the Populus trichocarpa gene Potri.002G101200 was used in a blast search followed by a phylogenetic analysis of the identified putative homologous and orthologous genes. The identified E. grandis ortholog, accession number Eucgr.B00045, has a 55% polypeptide sequence identity to the Populus gene product. The length of the hybrid aspen pLMX5 promoter is 1.8 kb. To allow for some interspecies variation, a 2001 base pair long promoter fragment immediately upstream of, but not including, the start codon of the gene Eucgr.B00045 was used for synthesis of the pEX5 promoter, Seq ID No.: 15. The pEX5 and pLMX5 are orthologous promoters.

The pEL1.1 and pEL1.2 Promoters

The pEL1.1 and pEL1.2 promoters originate from the one of the best characterized light-inducible genes in leaves, the small subunit of ribulose-1,5-bisphosphate carboxylase (RuBisCo or RBCS) gene promoter. The Rubisco small subunit, RBCS, is a multigene family in Arabidopsis thaliana and consists of four genes; RBCS1A (At1 g67090), RBCS1B (At5g38430), RBCS2B (At5g38420), and RBCS3B (At5g38410).

It has been found that the promoter from RBCS genes contain an intricate assortment of positive and negative regulatory elements that are able to confer light-inducible and tissue-specific expression in transgenic plants (Gilmartin and Chua 1990, Mol Cell Biol, 10(10) 5565-5568). Anisimov et al. 2007, Mol Breeding, 19, 241-253, describes that the level of expression conferred by the pRBCS promoter differ depending on the length of the used promoter fragment. A longer promoter of 1.6 kb has an expression level that is four times higher than a short promoter fragment of 300-600 bp.

The four polypeptides of the RBCS multigene family from Arabidopsis thaliana were used in a blast search followed by a phylogenetic analysis of the identified putative homologous and orthologous genes.

The three identified loci, Eucgr.B03013, Eucgr.J01502, and Eucgr.K02223, were found to have 70-80% amino acid identity to query sequence. The highest scoring, Eucgr.B03013, has 79.7%, 80.2%, 80.2% and 79.1% identity, respectively, to the above-mentioned Arabidopsis thaliana genes. In the phylogenetic analysis the Eucgr.K02223 gene was identified as the closest homologue to Arabidopsis thaliana RBCS, from which the Eucalyptus promotor were cloned. In Populus trichocarpa two putative orthologous genes are identified, Potri.017G114600 and Potri.004G100000; these two genes are considered paralogous genes.

Based on the findings of Anisimov, et al. 2007, Mol Breeding, 19, 241-253, two promoter fragments of different lengths from Eucalyptus grandis were selected for synthesis; pEL1.1, Seq ID No.: 16, has a short promoter sequence of 600 bp, while pEL1.2, Seq ID No.: 17, has a longer promoter sequence of 1800 bp.

The putative orthologous and paralogous promoters to the pEL1.1 promoter are the Populus trichocarpa promoter regions, pEL1.1-ort poplar, Seq ID No.: 34, and pEL1.1-para poplar, Seq ID No.: 40, respectively.

The putative orthologous and paralogous promoters to the pEL1.2 promoter are the Populus trichocarpa promoter regions, pEL1.2-ort poplar, Seq ID No.: 35, and pEL1.2-para poplar, Seq ID No.: 41, respectively.

The pEL1.1 and pEL1.2 promoters have been established and used as a tissue specific promoter in the patent application SE1651431-7.

The Promoter pER1

The Eucalyptus camaldulensis EcPT2 gene, Genbank accession number AB242817, was found to be specifically expressed in the root by Koyama et al. 2006, Plant Biotechnology, 23, 215-218. The EcPT2 gene encodes an inorganic phosphate (Pi) transporter protein. Phosphate is one of the most important nutrients for plant growth and it is likely that Pi transporters in roots play a major role in inorganic phosphate uptake from the soil.

The EcPT2 nucleotide sequence was used in a blast search followed by a phylogenetic analysis of the identified putative homologous and orthologous genes. The E. grandis gene with accession number Eucgr.H00165 was identified as the ortholog to the EcPT2 gene. Putative orthologous genes in Populus trichocarpa are Potri.005G175700, Potri.005G175500, Potri.015G022800 and Potri.019G061900.

Since the length of the promoter is unknown, a 2000 base pair long promoter fragment immediately upstream of, but not including, the start codon of gene Eucgr.H00165 was selected for synthesis of the pER1 promoter, Seq ID No.: 18.

Putative orthologous and paralogous promoters to the pER1 promoter are the Populus trichocarpa promoter regions, pER1-ort poplar, Seq ID No.: 36, pER1-para poplar1, Seq ID No.: 42 and pE R1-para poplar2, Seq ID No.: 44, respectively.

The Promoter pER2

The Eucalyptus grandis TIP2 gene, EgTIP2, accession number Eucgr.D02090, has a root specific expression pattern as presented in Rodrigues et al. 2013, Plant Science, 213, 106-113 and Vicentini et al. 2005, Genetics and Molecular Biology, 28, 487-495. Tonoplast intrinsic proteins (TIPs) are plant aquaporins. Aquaporins are integral membrane proteins, facilitating water transport and have been implicated in multiple physiological processes, including adaptation to certain abiotic stresses. TIP proteins are involved in the regulation of cell elongation and turgor homeostasis by mediating the transport of water and small molecules across the vacuolar membrane.

The EgTIP2 nucleotide sequence was used in a blast search followed by a phylogenetic analysis of the identified putative homologous and orthologous genes. Putative orthologous genes in Populus trichocarpa are Potri.001G157000 and Potri.003G077800.

A short, 900 nucleotides long, EgTIP2 promoter drives expression in all vascular tissue in transformed tobacco seedlings, including but not limited to root and root tip (Rodrigues et al, 2013). Since a 900 nucleotide fragment is insufficient for root specific expression, at least in tobacco, and the promoter length is unknown, a 2000 base pair long promoter fragment immediately upstream of, but not including, the start codon of gene Eucgr.D02090 was selected for synthesis of the pER2 promoter, Seq ID No.: 19.

The putative orthologous and paralogous promoters to the pER2 promoter are the Populus trichocarpa promoter regions, pER2-ort poplar, Seq ID No.: 37, and pER2-para poplar, Seq ID No.: 43, respectively.

### Functional Tests of the Identified Promoters.

In order to verify that all newly identified Eucalyptus promoters including the two variants of the leaf specific promoter were functional in trees, transgenic hybrid aspen with the different recombinant promoter-GUS constructs were created and studied. The DNA sequence of the identified promoter regions of the genomic sequence were manufactured by DNA synthesis, creating identical copies of the identified promoter regions of the genomic sequence of Eucalyptus grandis.

The synthetic promoters were cloned into an expression vector, positioned in front of the beta-glucuronidase (GUS) reporter gene. The recombinant promoter-GUS constructs were used in Agrobacterium-mediated transformation of hybrid aspen.

The promoter expression pattern was determined by histological studies of transgenic hybrid aspen plants harbouring the promoter-GUS construct, where the expression of the GUS gene was monitored using the established histochemical GUS staining technique. Details for these experiments are found in Example 1 and 2.

Eucalyptus promoters having a desired expression pattern could subsequently be used for controlling gene expression, to specifically direct the expression of a trait gene in planta.

### Trait Genes

All trail genes below might by expressed in any cell type or specific tissue. The specificity of the above-mentioned promoters may make them ideal for affecting actively growing cells while minimizing side effects on cells not actively involved in growth in the plant.

As known there is a variation in the observed level of phenotypical effect of the genetic modification between the different independent transgenic lines. This variation is anticipated for a person skilled in the art, since plants are living, multicellular organisms impossible to grow completely uniformly and since the point of integration of the recombinant DNA construct in the plant genome to a large extent is random and are known to affect the expression of an inserted trait gene.

### Trait Gene G47

The gene called G47, Seq ID No.: 1, is as earlier mentioned expected to be an ERF/AP2 transcription factor, belonging to the CBF/DREB subfamily. The gene G47 can also be named SGF1 as in JP2016127811. Ortholog genes to the G47 are probably, in poplar, Potri.019G073300.1 or Potri.013G100300.1, in Eucalyptus, Eucgr.F02967.1 and in corn GRMZM2G307152 or GRMZM2G481668.

The G47 gene used in the present invention is a nucleic acid encoding a G47 gene product that preferably has an amino acid sequence of at least 50%, such as 60%, 70%, 75%, 80%, 85%, 90%, 95%, 99% or 100% identical to an amino acid sequence having the amino acid sequence according to Seq ID No.: 1 (AtG47, AT1 G22810). A summary of orthologous G47 genes and their peptide percent identities are found the Table 2, below.

TABLE 2a

| Gene name | | Amino acid | Number | Nucleotide | |
|---|---|---|---|---|---|
| or | Ortholog accession | sequence | of amino | sequence | Number of |
| Organism | number: | Seq ID No: | acids | Seq ID No: | nucleotides |
| AtG47 | AT1G22810 | 1 | 144 | 21 | 435 |
| | Potri.019G073300 | 50 | 186 | 51 | 561 |
| | Potri.013G100300 | 52 | 168 | 53 | 507 |
| | Eucgr.F02967 | 54 | 155 | 55 | 468 |
| Zea mays | GRMZM2G307152 | 100 | 178 | 101 | 537 |
| | GRMZM2G481668 | 102 | 186 | 103 | 561 |

TABLE 2b
Peptide percent Identity Matrix—created by Clustal 2.1.

| | Seq_ID_No_ | Seq_ID_No_50_ | Seq_ID_No_52_ | Seq_ID_No_54_ | Seq_ID_No_100_ | Seq_ID_No_102_ |
|---|---|---|---|---|---|---|
| | 1_AtG47 | Potri.019G073300 | Potri.013G100300 | Eucgr.F02967 | GRMZM2G307152 | GRMZM2G481668 |
| Seq_ID_No_1_AtG47 | 100 | 50 | 47 | 49 | 41 | 40 |
| Seq_ID_No_50_ | 50 | 100 | 82 | 54 | 41 | 39 |
| Potri.019G073300 | | | | | | |
| Seq_ID_No_52_ | 47 | 82 | 100 | 52 | 43 | 40 |
| Potri.013G100300 | | | | | | |
| Seq_ID_No_54_ | 49 | 54 | 52 | 100 | 50 | 47 |
| Eucgr.F02967 | | | | | | |
| Seq_ID_No_100_ | 41 | 41 | 43 | 50 | 100 | 62 |
| GRMZM2G307152 | | | | | | |
| Seq_ID_No_102_ | 40 | 39 | 40 | 47 | 62 | 100 |
| GRMZM2G481668 | | | | | | |

### Trait Gene TF0002

A TF0002 gene useful in the present invention is a nucleic acid encoding a TF0002 gene product that and preferably has an amino acid sequence at least 50%, such as 60%, 70%, 75%, 80%, 85%, 90%, 95%, 99% or 100% identical to an amino acid sequence having the amino acid sequence according to Seq ID No.: 1 (PttTF0002). A summary of orthologous TF0002 genes and their peptide percent identities are found the Table 3, below.

TABLE 3a

| Gene name | | Amino acid | Number | Nucleotide | |
|---|---|---|---|---|---|
| or | Ortholog accession | sequence | of amino | sequence | Number of |
| Organism | number: | Seq ID No: | acids | Seq ID No: | nucleotides |
| PttTF0002 | | 2 | 361 | 22 | 1086 |
| | Potri.014G024200 | 56 | 388 | 57 | 1167 |
| | Potri.002G123300 | 58 | 358 | 59 | 1077 |
| | Eucgr.F00187 | 60 | 437 | 61 | 1314 |
| AtWRKY7 | AT4G24240 | 62 | 353 | 63 | 1062 |
| Zea mays | GRMZM2G091331 | 104 | 316 | 105 | 951 |
| | GRMZM2G102583 | 106 | 331 | 107 | 996 |

TABLE 3b
Peptide percent Identity Matrix—created by Clustal 2.1

| | Seq_ID_No_2_ | Seq_ID_No_56_ | Seq_ID_No_58_ | Seq_ID_No_60_ |
|---|---|---|---|---|
| | PttTF0002 | Potri.014G024200 | Potri.002G123300 | Eucgr.F00187 |
| Seq_ID_No_2_ | 100 | 97 | 88 | 66 |
| PttTF0002 | | | | |
| Seq_ID_No_56_ | 97 | 100 | 89 | 62 |
| Potri.014G024200 | | | | |
| Seq_ID_No_58_ | 88 | 89 | 100 | 67 |
| Potri.002G123300 | | | | |
| Seq_ID_No_60_ | 66 | 62 | 67 | 100 |
| Eucgr.F00187 | | | | |
| Seq_ID_No_62_ | 59 | 59 | 58 | 57 |
| AT4G24240 | | | | |
| Seq_ID_No_104_ | 48 | 47 | 46 | 46 |
| GRMZM2G091331 | | | | |
| Seq_ID_No_106_ | 46 | 46 | 45 | 44 |
| GRMZM2G102583 | | | | |
| | | Seq_ID_No_62_ | Seq_ID_No_104_ | Seq_ID_No_106_ |
| | | AT4G24240 | GRMZM2G091331 | GRMZM2G102583 |
| | Seq_ID_No_2_ | 59 | 8 | 46 |
| | PttTF0002 | | | |
| | Seq_ID_No_56_ | 59 | 47 | 46 |
| | Potri.014G024200 | | | |
| | Seq_ID_No_58_ | 58 | 46 | 45 |
| | Potri.002G123300 | | | |
| | Seq_ID_No_60_ | 57 | 46 | 44 |
| | Eucgr.F00187 | | | |
| | Seq_ID_No_62_ | 100 | 47 | 46 |
| | AT4G24240 | | | |
| | Seq_ID_No_104_ | 47 | 100 | 61 |
| | GRMZM2G091331 | | | |
| | Seq_ID_No_106_ | 46 | 61 | 100 |
| | GRMZM2G102583 | | | |

### Trait Gene TF0097

As mentioned above the Populus tremula x tremuloides TF0097 gene, PttTF6497, is a transcription factor belonging to the basic helix-loop-helix (bHLH) protein superfamily of transcription factors. In the present disclosure a TF0097 gene is a nucleic acid encoding a TF0097 gene product that preferably has an amino acid sequence at least 50%, such as 60%, 70%, 75%, 80%, 85%, 90%, 95%, 99% or 100% identical to an amino acid sequence having the amino acid sequence according to Seq ID No.: 3 (PttTF0097). A summary of orthologous TF0097 genes and their peptide percent identities are found the Table 4, below.

The TF0097 gene product, Seq ID No: 3 is orthologous to two Populus trichocarpa UPA20-like polypeptides presented in WO2012117330. The amino acid sequence identity between PttTF0097 and the sequences is 97% and 96% respectively. However, WO2012117330 does not provide a description of the function or a phenotypical effect of a modified expression of the TF0097 gene or any genes closely related to TF0097.

TABLE 4a

| | | | Number | | |
|---|---|---|---|---|---|
| | | Amino acid | of | Nucleotide | |
| Gene name | Ortholog accession | sequence | amino | sequence | Number of |
| or Organism | number: | Seq ID No: | acids | Seq ID No: | nucleotides |
| PttTF0097 | | 3 | 568 | 23 | 1707 |
| | Potri.014G148900 | 64 | 572 | 65 | 1719 |
| | Potri.002G235400 | 66 | 567 | 67 | 1704 |
| EucTF0097 | Eucgr.H02628 | 68 | 531 | 69 | 1596 |
| | AT5G48560 | 70 | 498 | 71 | 1497 |
| | AT3G07340 | 72 | 456 | 73 | 1371 |
| Zea mays | GRMZM2G083504 | 108 | 350 | 109 | 1056 |
| | GRMZM2G180406 | 110 | 460 | 111 | 1383 |
| | GRMZM5G828396 | 112 | 1218 | 113 | 404 |

TABLE 4b
Peptide percent Identity Matrix—created by Clustal 2.1

| | Seq_ID_No_3_ | Seq_ID_No_64_ | Seq_ID_No_66_ | Seq_ID_No_68_ | Seq_ID_No_70_ |
|---|---|---|---|---|---|
| | PttTF0097 | Potri.014G148900 | Potri.002G235400 | Eucgr.H02628 | AT5G48560 |
| Seq_ID_No_3_ | 100 | 91 | 86 | 56 | 46 |
| PttTF0097 | | | | | |
| Seq_ID_No_64_ | 91 | 100 | 81 | 52 | 43 |
| Potri.014G148900 | | | | | |
| Seq_ID_No_66_ | 86 | 81 | 100 | 55 | 44 |
| Potri.002G235400 | | | | | |
| Seq_ID_No_68_ | 56 | 52 | 55 | 100 | 41 |
| Eucgr.H02628 | | | | | |
| Seq_ID_No_70_ | 46 | 43 | 44 | 41 | 100 |
| AT5G48560 | | | | | |
| Seq_ID_No_72_ | 46 | 43 | 45 | 42 | 65 |
| AT3G07340 | | | | | |
| Seq_ID_No_108_ | 35 | 34 | 35 | 34 | 31 |
| GRMZM2G083504 | | | | | |
| Seq_ID_No_110_ | 38 | 36 | 39 | 34 | 33 |
| GRMZM2G180406 | | | | | |
| Seq_ID_No_112_ | 36 | 35 | 37 | 32 | 32 |
| GRMZM5G828396 | | | | | |
| | | Seq_ID_No_72_ | Seq_ID_No_108_ | Seq_ID_No_110_ | Seq_ID_No_112_ |
| | | AT3G07340 | GRMZM2G083504 | GRMZM2G180406 | GRMZM5G828396 |
| | Seq_ID_No_3_ | 46 | 35 | 38 | 36 |
| | PttTF0097 | | | | |
| | Seq_ID_No_64_ | 43 | 34 | 36 | 35 |
| | Potri.014G148900 | | | | |
| | Seq_ID_No_66_ | 45 | 35 | 39 | 37 |
| | Potri.002G235400 | | | | |
| | Seq_ID_No_68_ | 42 | 34 | 34 | 32 |
| | Eucgr.H02628 | | | | |
| | Seq_ID_No_70_ | 65 | 31 | 33 | 32 |
| | AT5G48560 | | | | |
| | Seq_ID_No_72_ | 100 | 35 | 36 | 35 |
| | AT3G07340 | | | | |
| | Seq_ID_No_108_ | 35 | 100 | 48 | 60 |
| | GRMZM2G083504 | | | | |
| | Seq_ID_No_110_ | 36 | 48 | 100 | 49 |
| | GRMZM2G180406 | | | | |
| | Seq_ID_No_112_ | 35 | 60 | 49 | 100 |
| | GRMZM5G828396 | | | | |

### Trait Gene TF0132

The TF0132 gene is a WRKY transcription factor, belonging to WRKY subfamily lid according to the WRKY gene classification presented above. In the present disclosure a TF0132 gene is a nucleic acid encoding a TF0132 gene product that preferably has an amino acid sequence at least 50%, such as 60%, 70%, 75%, 80%, 85%, 90%, 95%, 99% or 100% identical to an amino acid sequence having the amino acid sequence according to Seq ID No.: 5 (PttTF0132). A summary of orthologous TF0132 genes and their peptide percent identities are found the Table 5, below.

TABLE 5a

| | | Amino | | | |
|---|---|---|---|---|---|
| | | acid | Number | | |
| | | sequence | of | Nucleotide | |
| | Ortholog accession | Seq ID | amino | sequence | Number of |
| Gene name: | number: | No: | acids | Seq ID No: | nucleotides |
| PttTF0132 | | 4 | 347 | 24 | 1044 |
| | Potri.005G219500 | 84 | 347 | 85 | 1044 |
| | Potri.002G043500 | 86 | 351 | 87 | 1056 |
| | Eucgr.B01503 | 88 | 343 | 89 | 1032 |
| AtWRKY21 | AT2G30590 | 90 | 380 | 91 | 1143 |
| Zea mays | GRMZM2G018487 | 114 | 397 | 115 | 1194 |
| | GRMZM2G130374 | 116 | 374 | 117 | 1125 |
| | GRMZM2G173680 | 118 | 367 | 119 | 1104 |
| | GRMZM5G880069 | 120 | 369 | 121 | 1110 |
| | GRMZM2G070211 | 122 | 354 | 123 | 1065 |
| | GRMZM2G147880 | 124 | 395 | 125 | 1188 |

TABLE 5b
Peptide percent Identity Matrix—created by Clustal 2.1

| | Seq_ID_No_4_ | Seq_ID_No_84_ | Seq_ID_No_86_ | Seq_ID_No_88_ | Seq_ID_No_90_ | Seq_ID_No_114_ |
|---|---|---|---|---|---|---|
| | PttTF0132 | Potri.005G219500 | Potri.002G043500 | Eucgr.B01503 | AT2G30590 | GRMZM2G018487 |
| Seq_ID_No_4_ | 100 | 97 | 93 | 80 | 65 | 51 |
| PttTF0132 | | | | | | |
| Seq_ID_No_84_ | 97 | 100 | 95 | 82 | 65 | 51 |
| Potri.005G219500 | | | | | | |
| Seq_ID_No_86_ | 93 | 95 | 100 | 81 | 64 | 53 |
| Potri.002G043500 | | | | | | |
| Seq_ID_No_88_ | 80 | 82 | 81 | 100 | 65 | 50 |
| Eucgr.B01503 | | | | | | |
| Seq_ID_No_90_ | 65 | 65 | 64 | 65 | 100 | 47 |
| AT2G30590 | | | | | | |
| Seq_ID_No_114_ | 51 | 51 | 53 | 50 | 47 | 100 |
| GRMZM2G018487 | | | | | | |
| Seq_ID_No_116_ | 49 | 50 | 51 | 50 | 47 | 46 |
| GRMZM2G130374 | | | | | | |
| Seq_ID_No_118_ | 51 | 51 | 51 | 51 | 48 | 47 |
| GRMZM2G173680 | | | | | | |
| Seq_ID_No_120_ | 51 | 51 | 51 | 50 | 44 | 47 |
| GRMZM5G880069 | | | | | | |
| Seq_ID_No_122_ | 51 | 51 | 51 | 52 | 45 | 49 |
| GRMZM2G070211 | | | | | | |
| Seq_ID_No_124_ | 50 | 50 | 51 | 50 | 48 | 90 |
| GRMZM2G147880 | | | | | | |
| | | Seq_ID_No_116_ | Seq_ID_No_118_ | Seq_ID_No_120_ | Seq_ID_No_122_ | Seq_ID_No_124_ |
| | | GRMZM2G130374 | GRMZM2G173680 | GRMZM5G880069 | GRMZM2G070211 | GRMZM2G147880 |
| | Seq_ID_No_4_ | 49 | 51 | 51 | 51 | 50 |
| | PttTF0132 | | | | | |
| | Seq_ID_No_84_ | 50 | 51 | 51 | 51 | 50 |
| | Potri.005G219500 | | | | | |
| | Seq_ID_No_86_ | 51 | 51 | 51 | 51 | 51 |
| | Potri.002G043500 | | | | | |
| | Seq_ID_No_88_ | 50 | 51 | 50 | 52 | 50 |
| | Eucgr.B01503 | | | | | |
| | Seq_ID_No_90_ | 47 | 48 | 44 | 45 | 48 |
| | AT2G30590 | | | | | |
| | Seq_ID_No_114_ | 46 | 47 | 47 | 49 | 90 |
| | GRMZM2G018487 | | | | | |
| | Seq_ID_No_116_ | 100 | 86 | 60 | 61 | 46 |
| | GRMZM2G130374 | | | | | |
| | Seq_ID_No_118_ | 86 | 100 | 60 | 62 | 47 |
| | GRMZM2G173680 | | | | | |
| | Seq_ID_No_120_ | 60 | 60 | 100 | 87 | 47 |
| | GRMZM5G880069 | | | | | |
| | Seq_ID_No_122_ | 61 | 62 | 87 | 100 | 48 |
| | GRMZM2G070211 | | | | | |
| | Seq_ID_No_124_ | 46 | 47 | 47 | 48 | 100 |
| | GRMZM2G147880 | | | | | |

### Trait Gene TF0109

The TF0109 gene is a WRKY transcription factor, belonging to WRKY subfamily lid according to the WRKY gene classification presented above. In the present disclosure a TF0109 gene is a nucleic acid encoding a TF0109 gene product that preferably has an amino acid sequence at least 50%, such as 60%, 70%, 75%, 80%, 85%, 90%, 95%, 99% or 100% identical to an amino acid sequence having the amino acid sequence according to Seq ID No.: 4 (PttTF0109). A summary of orthologous TF0109 genes and their peptide percent identities are found the Table 6, below.

TABLE 6

| | | Amino | | | |
|---|---|---|---|---|---|
| | | acid | Number | | |
| | | sequence | of | Nucleotide | |
| Gene name | Ortholog accession | Seq ID | amino | sequence | Number of |
| or Organism | number: | No: | acids | Seq ID No: | nucleotides |
| PttTF0109 | | 5 | 301 | 25 | 906 |
| | Potri.006G072400 | 74 | 301 | 75 | 906 |
| | Potri.018G139300 | 76 | 300 | 77 | 903 |
| | Eucgr.C04011 | 78 | 343 | 79 | 1032 |
| AtWRKY11 | AT4G31550 | 80 | 325 | 81 | 978 |
| AtWRKY17 | AT2G24570 | 82 | 321 | 83 | 966 |
| Zea mays | GRMZM2G102583 | 126 | 331 | 127 | 996 |
| | GRMZM2G091331 | 128 | 316 | 129 | 951 |
| | GRMZM2G071907 | 130 | 298 | 131 | 897 |

TABLE 6b
Peptide percent Identity Matrix—created by Clustal 2.1

| | Seq_ID_No_5_ | Seq_ID_No_74_ | Seq_ID_No_76_ | Seq_ID_No_78_ | Seq_ID_No_80_ |
|---|---|---|---|---|---|
| | PttTF0109 | Potri.006G072400 | Potri.018G139300 | Eucgr.C04011 | AT4G31550 |
| Seq_ID_No_5_ | 100 | 98 | 87 | 59 | 57 |
| PttTF0109 | | | | | |
| Seq_ID_No_74_ | 98 | 100 | 87 | 59 | 57 |
| Potri.006G072400 | | | | | |
| Seq_ID_No_76_ | 87 | 87 | 100 | 58 | 58 |
| Potri.018G139300 | | | | | |
| Seq_ID_No_78_ | 59 | 59 | 58 | 100 | 63 |
| Eucgr.C04011 | | | | | |
| Seq_ID_No_80_ | 57 | 57 | 58 | 63 | 100 |
| AT4G31550 | | | | | |
| Seq_ID_No_82_ | 59 | 59 | 57 | 66 | 76 |
| AT2G24570 | | | | | |
| Seq_ID_No_126_ | 55 | 55 | 57 | 55 | 57 |
| GRMZM2G102583 | | | | | |
| Seq_ID_No_128_ | 57 | 58 | 58 | 52 | 56 |
| GRMZM2G091331 | | | | | |
| Seq_ID_No_130_ | 57 | 58 | 60 | 53 | 56 |
| GRMZM2G071907 | | | | | |
| | | Seq_ID_No_82_ | Seq_ID_No_126_ | Seq_ID_No_128_ | Seq_ID_No_130_ |
| | | AT2G24570 | GRMZM2G102583 | GRMZM2G091331 | GRMZM2G071907 |
| | Seq_ID_No_5_ | 59 | 55 | 57 | 57 |
| | PttTF0109 | | | | |
| | Seq_ID_No_74_ | 59 | 55 | 58 | 58 |
| | Potri.006G072400 | | | | |
| | Seq_ID_No_76_ | 57 | 57 | 58 | 60 |
| | Potri.018G139300 | | | | |
| | Seq_ID_No_78_ | 66 | 55 | 52 | 53 |
| | Eucgr.C04011 | | | | |
| | Seq_ID_No_80_ | 76 | 57 | 56 | 56 |
| | AT4G31550 | | | | |
| | Seq_ID_No_82_ | 100 | 55 | 57 | 56 |
| | AT2G24570 | | | | |
| | Seq_ID_No_126_ | 55 | 100 | 61 | 62 |
| | GRMZM2G102583 | | | | |
| | Seq_ID_No_128_ | 57 | 61 | 100 | 90 |
| | GRMZM2G091331 | | | | |
| | Seq_ID_No_130_ | 56 | 62 | 90 | 100 |
| | GRMZM2G071907 | | | | |

### Plant Transformation

DNA constructs were transformed into Agrobacterium and subsequently into hybrid aspen, where Populus tremula x tremuloides clone T89, also called “poplar” in this application, was transformed and regenerated. Typically, 8 independent lines were generated for each construct. One such group of transgenic trees produced using the same DNA construct is hereafter called a “construction group”, that is different transgenic trees emanating from one construct.

Each transgenic line within each construction group derives from a different transformation event and has most probably the recombinant DNA inserted into a unique location in the plant genome. This makes the different transgenic lines within one construction group partly different. For example it is known that different transformation events will produce plants with different expression levels of the gene product. It is also known that different levels of expression of a gene will result in different levels of phenotypic effects.

Maize transformation can be performed according to Coussens et al., 2012 and WO2014195287.

### Plant Growth

The transgenic hybrid aspen lines were grown together with wild type control trees, in a greenhouse under a photoperiod of 18 h and a temperature of 22° C./15° C. (day/night). All transgenic lines were grown in three clonal replicates. The plants were grown for 8-9 weeks before harvest and fertilized weekly. During this time height and diameter were measured weekly. Wild type (typically 35-45 trees) and transgenic trees were grown in parallel in the greenhouse under the same conditions. All comparisons between wild type trees and the transgenic trees with a specific promoter-gene combination are made within the cultivation group.

Maize and Arabidopsis plants might be tested for biomass, leaf length width and yield, more details according to the following examples and in WO2014195287.

### Growth Analyses

To identify construction groups showing a significant difference compared to the wild type population, data from each construction group was subjected to a number of growth data analyses of growth/biomass and wood density measurements.

After 8 to 9 weeks growth in the greenhouse the trees were harvested and sampled. Two principal types of harvests were used; either a general setup designed for e.g. chemical analysis, wood morphology analysis, gene expression analysis, wood density analysis and metabolomics analysis, or a second setup designed for dry weight measurements of bark, wood, leaves and roots.

Measurements of plant height and diameter were recorded one to two times per week during the cultivation and before harvest of the plants. Final height and diameter measurements were subsequently used to identify construction groups with altered growth characteristics.

The volume of the stem of each individual plant was approximated from final height and final diameter measurements using the formula for volume of a cone.

Stem volume approximation:

$$V = \frac{π * {r}^{2} * h}{3}$$

where: V=Volume; h=height (Final height), r=radius (Final diameter/2)

Average final volumes of each construction group population and corresponding wild type population were subsequently calculated.

### Wood Density Analyses

Wood density is an important trait for increasing biomass production. An increase in wood density increases the energy content per cubic metre reduces the volume of a fixed amount of biomass and hence, e.g. the volume required to transport a fixed amount of biomass. Correspondingly, more biomass can be transported per volume. Therefore increased density is of interest, even if total biomass is not increased. Increased density could also be of benefit coupled to pulp and paper production.

A 5 cm long stem segment, sampled between 36 and 41 cm from the soil from each harvested plant and stored in a freezer after harvest, was used for density measurements. Samples to be analysed were thawed followed by removal of bark and pith. The weight (w) was measured using a balance and the volume (V) was determined using the principle of Archimedes, where wood samples were submerged (using a needle) into a beaker (placed on a balance) with water. The recorded increase in weight is equivalent to the weight of the water displaced by the wood sample. Since the density of water is 1 g/cm3 at ambient room temperature the recorded increase is also equivalent to the volume of the wood sample. The samples were subsequently dried in oven for >48 h at 60° C.

The dry weights (dw) were measured and the density (d) was calculated according to:

$$d = \frac{dw}{V}$$

Samples from each construction group were compared to wild type samples from the same cultivation.

### Analysis of Expression Levels

Real-time RT-PCR was used to compare construct gene expression levels of the construction group with corresponding wild type group. The expression level of 26S proteasome regulatory subunit S2 was used as a reference to which construct gene expression was normalized. The comparative CT method was used for calculation of relative construct gene expression level, where the ratio between construction and reference gene expression level is described by (1+E_{target})−CT_{target}/(1+E_{reference})−CT_{reference}, where E_{target} and E_{reference} are the efficiencies of construct and reference gene PCR amplification respectively and CT_{target} and CT_{reference} are the threshold cycles as calculated for construct and reference gene amplification respectively.

### Obtaining Plants

The present invention extends to any plant cell of the above genetically modified, or transgenic plants obtained by the methods described herein, and to all plant parts, including harvestable parts of a plant, seeds, somatic embryos and propagules thereof, and plant explant or plant tissue. The present invention also encompasses a plant, a part thereof, a plant cell or a plant progeny comprising a DNA construct according to the invention. The present invention extends further to encompass the progeny of a primary transformed or transfected cell, tissue, organ or whole plant that has been produced by any of the aforementioned methods, the only requirement being that progeny exhibit the same genotypic and/or phenotypic characteristic(s) as those produced in the parent by the methods according to the invention. It should be noted that embodiments and features described in the context of one of the aspects of the present invention also apply to the other aspects of the invention. Thus, definitions of one embodiment regard mutatis mutandis to all other embodiments comprising or relating to the one embodiment. When for example definitions are made regarding DNA constructs or sequences, such definitions also apply with respect to methods for producing a plant, vectors, plant cells, plants comprising the DNA construct and vice versa. A DNA construct described in relation to a plant also regards all other embodiments. Details about obtaining maize, soya and Arabidopsis can be found in WO2014195287, hereby included by reference.

### Methods for Enhancing the Productivity of a Plant by Genetic Modification

One or more of the constructs according to the invention may be introduced into a plant cell by transformation.

### Transformation of Plant Cells

In accordance with the present invention, the method comprises transforming regenerable cells of a plant with a nucleic acid construct or recombinant DNA construct and regenerating a transgenic plant from said transformed cell. Production of stable, fertile transgenic plants is now a routine method.

Various methods are known for transporting the construct into a cell to be transformed. Agrobacterium-mediated transformation is widely used by those skilled in the art to transform tree species, in particular hardwood species such as poplar and Eucalyptus. Other methods, such as microprojectile or particle bombardment, electroporation, microinjection, direct DNA uptake, liposome mediated DNA uptake, or the vortexing method may be used where Agrobacterium transformation is inefficient or ineffective, for example in some gymnosperm species.

A person of skill in the art will realize that a wide variety of host cells may be employed as recipients for the DNA constructs and vectors according to the invention. Non-limiting examples of host cells include cells in embryonic tissue, callus tissue type 1, 11, and Ill, hypocotyls, meristem, root tissue, tissues for expression in phloem, leaf discs, petioles and stem internodes. Once the DNA construct or vector is within the cell, integration into the endogenous genome can occur.

### Selection of Transformed Plant Cells and Regeneration of Plant or Woody Plants

Following transformation, transgenic plants are preferably selected using a dominant selectable marker incorporated into the transformation vector. Typically, such a marker will confer antibiotic or herbicide resistance on the transformed plants and selection of transformants can be accomplished by exposing the plants to appropriate concentrations of the antibiotic or herbicide. A selection marker using the D-form of amino acids and based on the fact that plants can only tolerate the L-form offers a fast, efficient and environmentally friendly selection system.

Subsequently, a plant may be regenerated, e.g. from single cells, callus tissue or leaf discs, as is standard in the art. Almost any plant can be entirely regenerated from cells, tissues and organs of the plant. After transformed plants are selected and they are grown to maturity and those plants showing altered growth properties phenotype are identified.

The promotors of genes G47, TF0002, TF0097, TF0132 and TF109 or their corresponding ortholog might be mutated using the methods for site-directed mutagenesis such as TALENs or CRISPR/Cas9 to modify the expression of these genes.

### Methods for Detecting Modified Expression of a Gene Encoding a Polypeptide in a Plant or Woody Plant of the Invention

Real-time RT-PCR can be used to compare gene expression, i.e. the mRNA expression levels, in a genetically modified (GM) plant or woody plant with the corresponding non-GM plant or woody plant. The amount of the polynucleotides disclosed herein can be determined using Northern blots, sequencing, RT-PCR or microarrays.

Western blots with immune detection or gel shift assays can be used to measure the expression levels or amounts of a polypeptide expressed in a GM plant or woody plant of the invention. Antibodies raised to the respective polypeptide may be used for specific immune-detection of the expressed polypeptide in tissue derived from a woody plant.

Eucalyptus plants are generated in a similar way, through transformation, regeneration and growth analysis.

The invention is further illustrated below by way of examples. The examples are not intended to restrict the scope of the invention, which is that of the appended claims.

### EXAMPLES

### Example 1 Cloning of Promoters

### Cloning of Eucalyptus Promoters:

The identification of novel Eucalyptus promoters is described in the detailed description above. All Eucalyptus promoters were cloned in the same way. The promoter DNA fragments were manufactured by DNA synthesis, using the DNA sequences of the identified promoter regions of the publically available Eucalyptus grandis genome as a template, thus creating identical copies of the corresponding Eucalyptus grandis promoter regions. The synthesized promoter fragments were flanked by Gateway recombination sites for sub-cloning purposes. All promoter fragments were sub-cloned using Gateway recombination into the pK7m24GW.3 vector (VIB, Rijvisschestraat 120, B-9052 Zwijnaarde, Belgium), where they were placed upstream of and thus controlling the expression of a trait gene. The novel combinations of promoters and genes are further described in Example 3, below.

The promoters were also cloned in front of the beta-glucuronidase (GUS) reporter gene, in order to verify their correct expression as further described in Example 2.

1.1 the Constitutive Promoter pECO1

The DNA sequence upstream of the Eucalyptus grandis gene with accession Eucgr.E00053 was thoroughly investigated as described (in the detailed description) above. A fragment of 1084 nucleotides immediately upstream, but not including, the start codon was selected to define the pECO1 promoter, Seq ID No: 6.

1.2 the Constitutive Promoter pECO2

The DNA sequence upstream of the Eucalyptus grandis gene with accession Eucgr.H04673 was thoroughly investigated as described (in the detailed description) above. A fragment of 2000 nucleotides immediately upstream, but not including, the start codon was selected to define the pECO2 promoter, Seq ID No: 7.

1.3 the Tissue-Specific Promoter pEA1

The DNA sequence upstream of the Eucalyptus grandis gene with accession Eucgr.C00732 was thoroughly investigated as described (in the detailed description) above. A fragment of 2000 nucleotides immediately upstream, but not including, the start codon was selected to define the pEA1 promoter, Seq ID No: 8.

1.4 the Tissue-Specific Promoter pEA2

The DNA sequence upstream of the Eucalyptus grandis gene with accession Eucgr.F02223 was thoroughly investigated as described (in the detailed description) above. A fragment of 2500 nucleotides immediately upstream, but not including, the start codon was selected to define the pEA2 promoter, Seq ID No: 9.

1.5 the Tissue-Specific Promoter pEA3

The DNA sequence upstream of the Eucalyptus grandis gene with accession Eucgr.K03130 was thoroughly investigated as described (in the detailed description) above. A fragment of 2700 nucleotides immediately upstream, but not including, the start codon was selected to define the pEA3 promoter, Seq ID No: 10.

1.6 the Tissue-Specific Promoter pEA4

The DNA sequence upstream of the Eucalyptus grandis gene with accession Eucgr.100802 was thoroughly investigated as described (in the detailed description) above. A fragment of 2500 nucleotides immediately upstream, but not including, the start codon was selected to define the pEA4 promoter, Seq ID No: 11.

1.7 the Tissue-Specific Promoter pEC1

The DNA sequence upstream of the Eucalyptus grandis gene with accession Eucgr.F02320 was thoroughly investigated as described (in the detailed description) above. A fragment of 2101 nucleotides immediately upstream, but not including, the start codon was selected to define the pEC1 promoter, Seq ID No: 12.

1.8 Cloning of the Tissue-Specific Hybrid Aspen Promoter pLMP1

The pLMP1 promoter was cloned as described in WO2004097024. The promoter was placed in a pPCV812 binary plasmid in front/upstream of a Gateway® cassette, consisting of a ccdB gene flanked by attR recombination sites to facilitate Gateway® recombination cloning. The pLMP1 promoter, similar to Genbank accession number DJ416318, consists of a 1486 base pair long fragment excluding the restriction sites used for cloning, Seq ID No: 13.

1.9 Cloning of the Tissue-Specific Hybrid Aspen Promoter pLMX5

The pLMX5 promoter was cloned as described in WO2004097024. The promoter was placed in a pPCV812 binary plasmid in front/upstream of a Gateway® cassette, consisting of a ccdB gene flanked by attR recombination sites to facilitate Gateway® recombination cloning. The pLMX5 promoter, similar to Genbank accession number DJ416317, consists of a 1780 base pair long fragment excluding the restriction sites used for cloning, Seq ID No: 14.

1.10 the Tissue-Specific Promoter pEX5

The DNA sequence upstream of the Eucalyptus grandis gene with accession Eucgr.B00045 was thoroughly investigated as described (in the detailed description) above. A fragment of 2001 nucleotides immediately upstream, but not including, the start codon was selected to define the pEX5 promoter, Seq ID No: 15.

1.11 the Tissue-Specific Promoters, pEL1.1 and pEL1.2

The DNA sequence upstream of the Eucalyptus grandis gene with accession Eucgr.K02223 was thoroughly investigated as described (in the detailed description) above. Based on these studies two promoter variants were selected; a shorter and a longer promoter fragment. Fragments of 600 and 1800 nucleotides immediately upstream, but not including, the start codon were selected to define the shorter pEL1.1 (Seq ID No: 16) and longer pEL1.2 (Seq ID No: 17) promoter variants respectively.

1.12 the Tissue-Specific Promoter pER1

The DNA sequence upstream of the Eucalyptus grandis gene with accession Eucgr.H00165 was thoroughly investigated as described (in the detailed description) above. A fragment of 2000 nucleotides immediately upstream, but not including, the start codon was selected to define the pER1 promoter, Seq ID No: 18.

1.13 the Tissue-Specific Promoter pER2

The DNA sequence upstream of the Eucalyptus grandis gene with accession Eucgr.D02090 was thoroughly investigated as described (in the detailed description) above. A fragment of 2000 nucleotides immediately upstream, but not including, the start codon was selected to define the pER2 promoter, Seq ID No: 19.

### Example 2 Verification of Expression Pattern of the Eucalyptus Promoters

The expression patterns of the Eucalyptus promoters were determined by histological studies of transgenic hybrid aspen plants harbouring the promoter-GUS construct. Promoter activity was assayed using the established histochemical GUS staining technique.

Samples were collected from young transgenic plants. Five to eight transgenic lines from each promoter-GUS construct were sampled and the following eight parts of the plant were stained for GUS expression; 1) Apex with leaf primordia and small young leaf; 2) Part of young leaf; 3) Young stem section, close to apex; 4) Part of petiole; 5) Axillary bud; 6) Part of old leaf; 7) Longitudinal stem section of old stem and 8) Root. The stained plant tissues were carefully studied under a light microscope.

### Results

The resolution of the GUS assay is sufficient to distinguish the tissue regions from which the product of GUS enzyme activity emanates, but not high enough to distinguish the specific cells from which the product of GUS enzyme activity emanates.

pECO1: Constitutive expression was confirmed in early stages of transgenic tissue formation. Faint expression observed in older plant tissues.

pECO2: Strong constitutive expression was confirmed.

pEA1: Tissue-specific expression in the regions of the meristematic tissue responsible for primary growth in the apex, axillary buds and in leaf primordia was confirmed.

pEA2: Tissue-specific expression in the regions of the actively dividing cells of the apex, in axillary buds and in the vascular tissues of young and older stem was confirmed.

pEA3: Very faint tissue-specific expression in the regions of the meristematic tissues responsible for primary growth in the apex and axillary buds was confirmed.

pEA4: Weak tissue-specific expression in the regions of meristematic tissues responsible for primary and secondary growth in the apex, cambium and root was confirmed.

pEC1: Low resolution GUS analysis confirmed expression in the vascular region, thus indicating the expected expression in the cambial region.

pLMP1: Expression is found in the cambium with the strongest expression in the phloem, Article 3 in Björklund, Doctoral thesis 2007:81, Faculty of Forest Sciences, Umen, Sweden.

pLMX5: Expression is found in the cambium with the strongest expression in the vascular cambium and early xylem, Article 3 in Björklund, Doctoral thesis 2007:81, Faculty of Forest Sciences, Umen, Sweden.

pEX5: Expression in the vascular tissues of young stem, older stem, root and leaf was confirmed. However, the resolution of the GUS assay is not high enough to distinguish the specific cells of the vascular tissue from which the product of GUS enzyme activity emanates.

pEL1.1: Strong green-tissue-specific expression, also in light-exposed root tissues was confirmed.

pER1: Tissue-specific expression in the root was confirmed, with strong expression in young root, root hairs and root tip. Expression in the regions of the meristematic tissues responsible for primary growth in the root as well as in the apex was also observed.

pER2: Tissue-specific expression in the root was confirmed, with strong expression in young root and root hairs. No expression was observed in root tip.

### Example 3 Construction of Novel Promoter-Gene Combinations

As described in Example 1 the Eucalyptus promoter DNA fragments were manufactured by DNA synthesis and flanked by Gateway recombination sites for sub-cloning purposes. All Eucalyptus promoter fragments were sub-cloned using Gateway recombination into the pK7m24GW,3 vector, where they were placed upstream of and thus controlling the expression of the gene of interest as described below.

3.1 Constructs with the Gene G47

### Construct Gro4

The G47 gene from Arabidopsis thaliana, AtG47, Seq ID No: 1, was operably linked with the 35S promoter, Seq ID No: 20, to create the recombinant DNA construct Gro4, p35S-AtG47. A construct comprising this promoter might be used to increase the level of ATG47 gene product in all tissues of the plant.

### Construct Gro5

The G47 gene from Arabidopsis thaliana, AtG47, Seq ID No: 1, was operably linked with the tissue-specific vascular promoter pLMX5, Seq ID No: 14 to create the recombinant DNA construct, Gro5, pLMX5-AtG47. A construct comprising this promoter might be used to increase the level of G47 gene product specifically in the vascular tissue.

### Construct Gro6

The G47 gene from Arabidopsis thaliana, AtG47, Seq ID No: 1, was operably linked with the tissue-specific vascular promoter pLMP1, Seq ID No: 13 to create the recombinant DNA construct, Gro6, pLMP1-AtG47. A construct comprising this promoter might be used to increase the level of G47 gene product specifically in the vascular tissue.

3.2 Constructs with the Gene TF0002

### Construct TF0002F1

The TF0002 gene from Populus tremula x tremuloides, PttTF0002, Seq ID No: 2, was operably linked with the 35S promoter, Seq ID No: 20, to create the recombinant DNA construct TF0002F1, p35S-PttTF0002. A construct comprising this promoter might be used to increase the level of TF0002 gene product specifically in all tissues of the plant.

### Construct LMX5-008

The TF0002 gene from Populus tremula x tremuloides, PttTF0002, Seq ID No: 2, was operably linked with the LMX5 promoter, Seq ID No: 14, to create the recombinant DNA construct LMX5-008, pLMX5-PttTF0002. A construct comprising this promoter might be used to increase the level of TF0002 gene product specifically in the vascular tissue.

Construct pLMP1-PttTF0002

The TF0002 gene from Populus tremula x tremuloides, PttTF0002, Seq ID No: 2, was operably linked with the LMP1 promoter, Seq ID No: 13, to create the recombinant DNA construct pLMP1-PttTF0002. A construct comprising this promoter might be used to increase the level of TF0002 gene product specifically in the vascular tissue.

3.3 Constructs with the Gene TF0097

### Construct F101

The PttTF0097 gene from Populus tremula x tremuloides, Seq ID No: 3, was operably linked with the 35S promoter, Seq ID No: 20, to create the recombinant DNA construct F101, 35S-PttTF0097. A construct comprising this promoter might be used to increase the level of TF0097 gene product in all tissues of the plant.

### Construct F102

The PttTF0097 gene from Populus tremula x tremuloides, Seq ID No: 3, was operably linked with the pECO1 promoter, Seq ID No: 6, to create the recombinant DNA construct F102, pECO1-PttTF0097. A construct comprising this promoter might be used to increase the level of TF0097 gene product specifically in all tissues of the plant.

### Construct F103

The PttTF0097 gene from Populus tremula x tremuloides, Seq ID No: 3, was operably linked with the pECO2 promoter, Seq ID No: 7, to create the recombinant DNA construct F103, pECO2-PttTF0097. A construct comprising this promoter might be used to increase the level of TF0097 gene product specifically in all tissues of the plant.

### Construct F104

The PttTF0097 gene from Populus tremula x tremuloides, Seq ID No: 3, was operably linked with the pEA1 promoter, Seq ID No: 8, to create the recombinant DNA construct F104, pEA1-PttTF0097. A construct comprising this promoter might be used to increase the level of TF0097 gene product specifically in the shoot apical meristem and organ primordia.

### Construct F105

The PttTF0097 gene from Populus tremula x tremuloides, Seq ID No: 3, was operably linked with the pEA3 promoter, Seq ID No: 10, to create the recombinant DNA construct F105, pEA3-PttTF0097. A construct comprising this promoter might be used to increase the level of TF0097 gene product specifically in the leaf primordia.

### Construct F106

The PttTF0097 gene from Populus tremula x tremuloides, Seq ID No: 3, was operably linked with the pEC1 promoter, Seq ID No: 12, to create the recombinant DNA construct F106, pEC1-PttTF0097. A construct comprising this promoter might be used to increase the level of TF0097 gene product specifically in the procambial/cambial stem cells.

### Construct F107

The PttTF0097 gene from Populus tremula x tremuloides, Seq ID No: 3, was operably linked with the pEX5 promoter, Seq ID No: 15, to create the recombinant DNA construct F107, pEX5-PttTF0097. A construct comprising this promoter might be used to increase the level of TF0097 gene product specifically in the vascular xylem tissue.

### Construct F108

The PttTF0097 gene from Populus tremula x tremuloides, Seq ID No: 3, was operably linked with the pEL1.1 promoter, Seq ID No: 16, to create the recombinant DNA construct F108, pEL1.1-PttTF0097. A construct comprising this promoter might be used to increase the level of TF0097 gene product specifically in all green tissues of the plant.

### Construct F109

The PttTF0097 gene from Populus tremula x tremuloides, Seq ID No: 3, was operably linked with the pER1 promoter, Seq ID No: 18, to create the recombinant DNA construct F109, pER1-PttTF0097. A construct comprising this promoter might be used to increase the level of TF0097 gene product specifically in the root, root tip and root hairs.

### Construct F110

The PttTF0097 gene from Populus tremula x tremuloides, Seq ID No: 3, was operably linked with the pER2 promoter, Seq ID No: 19, to create the recombinant DNA construct F110, pER2-PttTF0097. A construct comprising this promoter might be used to increase the level of TF0097 gene product specifically in the root and root hairs.

3.4 Constructs with the Gene TF0132

### Construct F111

The PttTF0132 gene from Populus tremula x tremuloides, Seq ID No: 4, was operably linked with the 35S promoter, Seq ID No: 20, to create the recombinant DNA construct F111, 35S-PttTF0132. A construct comprising this promoter might be used to increase the level of TF0132 gene product in all tissues of the plant.

### Construct F112

The PttTF0132 gene from Populus tremula x tremuloides, Seq ID No: 3, was operably linked with the pECO1 promoter, Seq ID No: 6, to create the recombinant DNA construct F112, pECO1-PttTF0132. A construct comprising this promoter might be used to increase the level of TF0132 gene product specifically in all tissues of the plant.

### Construct F113

The PttTF0132 gene from Populus tremula x tremuloides, Seq ID No: 4, was operably linked with the pECO2 promoter, Seq ID No: 7, to create the recombinant DNA construct F113, pECO2-PttTF0132. A construct comprising this promoter might be used to increase the level of TF0132 gene product specifically in all tissues of the plant.

### Construct F114

The PttTF0132 gene from Populus tremula x tremuloides, Seq ID No: 4, was operably linked with the pEA1 promoter, Seq ID No: 8, to create the recombinant DNA construct F114, pEA1-PttTF0132. A construct comprising this promoter might be used to increase the level of TF0132 gene product specifically in the shoot apical meristem and organ primordia.

### Construct F115

The PttTF0132 gene from Populus tremula x tremuloides, Seq ID No: 4, was operably linked with the pEA2 promoter, Seq ID No: 9, to create the recombinant DNA construct F115, pEA2-PttTF0132. A construct comprising this promoter might be used to increase the level of TF0132 gene product specifically in the actively dividing cells in the cambial region of the stem and the shoot apical meristem.

### Construct F116

The PttTF0132 gene from Populus tremula x tremuloides, Seq ID No: 4, was operably linked with the pEA3 promoter, Seq ID No: 10, to create the recombinant DNA construct F116, pEA3-PttTF0132. A construct comprising this promoter might be used to increase the level of TF0132 gene product specifically in the leaf forming tissues of the leaf primordia.

### Construct F117

The PttTF0132 gene from Populus tremula x tremuloides, Seq ID No: 4, was operably linked with the pEA4 promoter, Seq ID No: 11, to create the recombinant DNA construct F117, pEA4-PttTF0132. A construct comprising this promoter might be used to increase the level of TF0132 gene product specifically in the shoot apical meristem, leaf primordia and to some extent in younger leaves.

### Construct F118

The PttTF0132 gene from Populus tremula x tremuloides, Seq ID No: 4, was operably linked with the pEC1 promoter, Seq ID No: 12, to create the recombinant DNA construct F118, pEC1-PttTF0132. A construct comprising this promoter might be used to increase the level of TF0132 gene product specifically in the procambial/cambial stem cells.

### Construct F119

The PttTF0132 gene from Populus tremula x tremuloides, Seq ID No: 4, was operably linked with the pEL1.1 promoter, Seq ID No: 16, to create the recombinant DNA construct F119, pEL1.1-PttTF0132. A construct comprising this promoter might be used to increase the level of TF0132 gene product specifically in all green tissues of the plant.

3.5 Constructs with the Gene TF0109

### Construct F120

The PttTF0109 gene from Populus tremula x tremuloides, Seq ID No: 5, was operably linked with the 35S promoter, Seq ID No: 20, to create the recombinant DNA construct F120, 35S-PttTF0109. A construct comprising this promoter might be used to increase the level of TF0109 gene product specifically in all tissues of the plant.

### Construct F121

The PttTF0109 gene from Populus tremula x tremuloides, Seq ID No: 5, was operably linked with the pECO1 promoter, Seq ID No: 6, to create the recombinant DNA construct F121, pECO1-PttTF0109. A construct comprising this promoter might be used to increase the level of TF0109 gene product specifically in all tissues of the plant.

### Construct F122

The PttTF0109 gene from Populus tremula x tremuloides, Seq ID No: 5, was operably linked with the pECO2 promoter, Seq ID No: 7, to create the recombinant DNA construct F122, pECO2-PttTF0109. A construct comprising this promoter might be used to increase the level of TF0109 gene product specifically in all tissues of the plant.

### Construct F123

The PttTF0109 gene from Populus tremula x tremuloides, Seq ID No: 5, was operably linked with the pEA1 promoter, Seq ID No: 8, to create the recombinant DNA construct F123, pEA1-PttTF0109. A construct comprising this promoter might be used to increase the level of TF0109 gene product specifically in the shoot apical meristem and organ primordia.

### Construct F124

The PttTF0109 gene from Populus tremula x tremuloides, Seq ID No: 5, was operably linked with the pEA2 promoter, Seq ID No: 9, to create the recombinant DNA construct F124, pEA2-PttTF0109. A construct comprising this promoter might be used to increase the level of TF0109 gene product specifically in the actively dividing cells in the cambial region of the stem and the shoot apical meristem.

### Construct F125

The PttTF0109 gene from Populus tremula x tremuloides, Seq ID No: 5, was operably linked with the pEA3 promoter, Seq ID No: 10, to create the recombinant DNA construct F125, pEA3-PttTF0109. A construct comprising this promoter might be used to increase the level of TF0109 gene product specifically in the leaf forming tissues of the leaf primordia.

### Construct F126

The PttTF0109 gene from Populus tremula x tremuloides, Seq ID No: 5, was operably linked with the pEX5 promoter, Seq ID No: 15, to create the recombinant DNA construct F126, pEX5-PttTF0109. A construct comprising this promoter might be used to increase the level of TF0109 gene product specifically in the vascular xylem tissue.

### Example 4 Transformation of Hybrid Aspen

The DNA constructs described in Example 3 were transformed into hybrid aspen (Populus tremula x Populus tremuloides Michx., clone T89) by Agrobacterium-mediated transformation. The transformation and regeneration of transgenic plants were performed as described in the experimental part of WO2016108750. Typically, 8 independent transgenic lines were generated for each construct.

### Example 5 Growth Experiments and Analyses

### 5.1 Hybrid Aspen Greenhouse Experiments and Growth Analysis

For each promoter-gene construct, three transgenic hybrid aspen lines in three clonal replicates each were grown together with wild type reference trees in the greenhouse, as described in the experimental part of WO2016108750 and in the detailed description above.

After 8 weeks of growing in the greenhouse the hybrid aspen trees were measured, harvested and sampled for the following traits, plant height, width, stem volume, average internode length and wood density. For some trees the dry weight of stem and bark was measured.

### 5.2 Hybrid Aspen Field Trial Experiments and Growth Analysis

The same transgenic hybrid aspen lines that were studied in the greenhouse experiments were again propagated from tissue culture material for field trial experiments. In some filed trials a selection of transgenic hybrid aspen lines made before propagation. Wild type reference plants were propagated in parallel and treated exactly as the transgenic plants throughout the experiments. Plants were grown in vitro until ready for planting in soil. The plants were hardened during a period of five weeks; the first two weeks to establish rooting in soil in the greenhouse and then another three weeks in outdoor growth conditions. After this the plants were transported to the field site and kept in pots in outdoor conditions for 5 weeks before planting into the field. The height of the plants were measured at planting and in the end of the growing season, which was used for statistical analysis. Further measurements was done and will continue to be done during the field trail.

5.3 Statistical Analysis of Results from Greenhouse and in Field Grown Trees

Statistical analysis was used to determine phenotypical differences between transgenic and wild type trees. Firstly, the population of transgenic trees from each promoter-gene combination was compared to the wild type population of trees with the Student's t-test and a stringent p-value cut off of 0.01. Second, the population of transgenic trees from each promoter-gene combination was compared to the wild type population of trees using the established Dunnett's multiple comparison of means method and a stringent p-value cutoff of 0.01. Similarly, to identify the best performing transgenic lines, the population of trees from each transgenic line, that is, the three replicates, was compared to the wild type population of trees with the same statistical test and settings. The results of the statistical analyses are presented in the results tables as the percentage differences between averages of the compared populations of transgenic and wild type trees, wild type being the reference point. Percentage differences that are statistically significant according to the statistical criteria specified above are marked with an asterisk (*) in the results tables.

### Example 6 Promoter and Gene G47 Combinations

6.1 Greenhouse and Field Trial Experiments with the Promoter-Gene Construct p35S-G47 for Growth Improvements and/or Improved Water Stress Properties Such as Improved Water Use Efficiency.

Over-expression of a gene may elicit different phenotypical effects under two distinctly different growth conditions. With the recombinant DNA construct p35S-AtG47 the G47 gene from Arabidopsis thaliana, AtG47, Seq ID: 1, was constitutively over-expressed in hybrid aspen trees using the CaMV 35S promoter. This modification in the transgenic trees resulted in an average stem volume growth increase of 12% compared to wild type reference trees when grown in the greenhouse.

The same hybrid aspen trees were subsequently planted in an open field trial in the south of Sweden. However, it was discovered that the transgenic trees harbouring the promoter-gene construct p35S-AtG47 did not perform as well as expected in these field growth tests. The observed average stem volume increase of 12% in greenhouse tests was contrasted with a reduction of stem volume growth of 3% compared to the wild type reference trees grown at the same test site location, FIG. 1.

6.2 Greenhouse and Field Trial Experiments with the Promoter-Gene Construct pLMP1:G47 for Growth Improvements and/or Improved Water Stress Properties Such as Improved Water Use Efficiency.

When the AtG47 gene is specifically over-expressed by the pLMP1 promoter no considerable increase in plant growth is observed in the greenhouse. However, unlike the results of the constitutive p35S-G47 promoter-gene construct, a considerable significant increase in plant growth is observed when the transgenic pLMP1-G47 trees are grown in the field, under similar field trial conditions and on the same location as used for the p35S-G47 over-expressing trees. An average stem volume increase of the transgenic pLMP1-G47 trees of 15% compared to the wild type reference was observed in the field. Plant height also increased 8% in the field in the transgenic pLMP1-G47 trees compared to the wild type reference, FIG. 2. In the field the trees will be in conditions that includes reduced availability to water. The pLMP1-G47 trees have improved phenotypic properties coupled to drought response and water use efficiency which could be the reason for the improved growth in the pLMP1-G47 trees.

It is evident and unexpected from these results, that having a strong, constitutive expression of the G47 trait gene, as with the 35S-G47 promoter-gene construct, can have disadvantageous effects under some field trial conditions, whilst having a more specific expression pattern under the same conditions, as with the pLMP1-G47 promoter-gene construct, results in improved growth and improved drought response properties compared to wild type.

These results demonstrate the beneficial and inventive use of specific combinations of promoters and genes to tailor the expression pattern of the gene to the specific tissue and/or to the specific growth condition and to retain or further improve the positive phenotypical traits provided by the gene when growth conditions change.

6.3 Greenhouse Experiments with the Promoter-Gene Construct pLMX5-G47 for Growth Improvements

When the AtG47 gene is specifically over-expressed by the vascular cambium/early xylem pLMX5 promoter no considerable increase in plant growth is observed in the greenhouse. No field trail data is available for this promoter-gene combination.

6.4 Greenhouse Experiments with the Promoter-Gene G47 Constructs for Improved Drought Tolerance.

Transgenic hybrid aspen trees with the p35S-AtG47, pLMX5-AtG47 and pLMP1-AtG47 constructs were grown together with wild type reference trees in the greenhouse. The wild type and the transgenic plants were subjected to a period of limited water supply and the effects of drought stress on plant vigour and health were monitored.

Healthy plant cells are turgid and plants rely on turgidity to maintain rigidity. Turgor pressure pushes the plasma membrane against the cell wall of plant cells by osmotic flow of water from outside the cell into the cell's vacuole. Turgor pressure also plays a key role in plant cell growth where the cell wall undergoes irreversible expansion due to the force of turgor pressure.

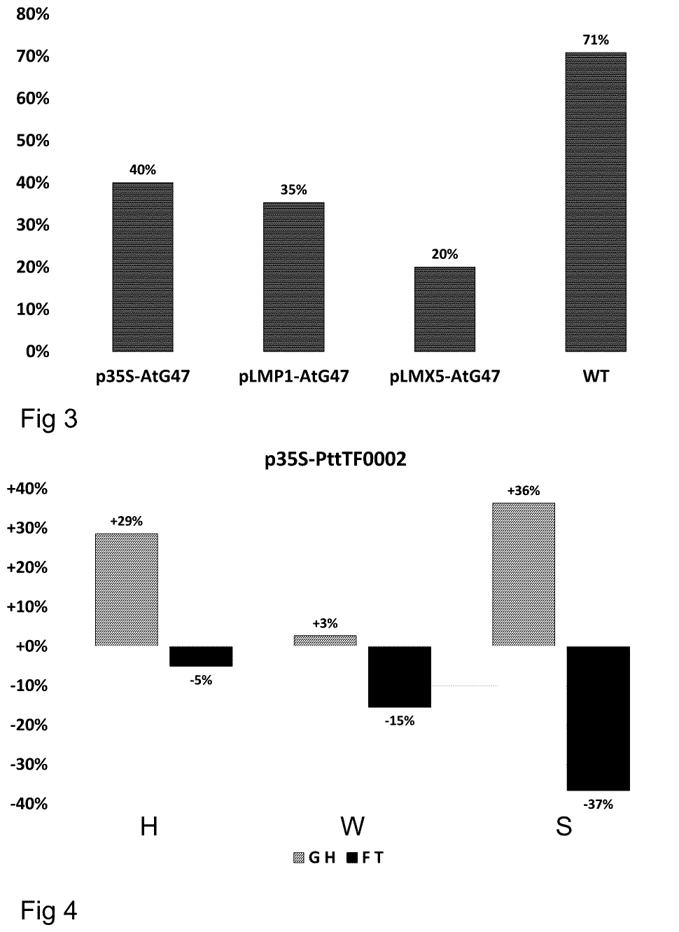

When water supply is reduced to test plant drought tolerance, 71% of wild type trees lose turgor pressure, which halts plant growth and causes the leaves to wilt. However, when the AtG47 gene is constitutively over-expressed by the 35S promoter a reduction in turgor loss of 44% compared to wild type trees is observed, as only 40% of the transgenic plants lose their turgor pressure, FIG. 3.

Furthermore, the pLMP1-AtG47 constructs, surprisingly showed an even more notable reduction in turgor loss of 50% compared to wild type trees is observed, as only 35% of the transgenic plants lose their turgor pressure, FIG. 3.

Moreover, the pLMX5-AtG47 construct showed a 72% reduction in turgor loss compared to wild type trees, as only 20% of the transgenic plants lose their turgor pressure, FIG. 3.

### 6.5 Water Use Efficiency (WUE)

A set of transgenic hybrid aspen trees grown in greenhouse containing constructs p35S-AtG47, pLMX5-AtG47 and pLMP1-AtG47 were tested according to the method presented in Farquhar, G. D., J. R. Ehleringer, and K. T. Hubick. 1989. Carbon isotope discrimination and photosynthesis. Ann. Rev. Plant Physiol. 40:503-537.

The ¹³C discrimination data from greenhouse experiments for constructs p35S-AtG47, pLMX5-AtG47 and pLMP1-AtG47 all showed reduced ¹³C discrimination and increased water use efficiency. One of the lines plant with the construct pLMP1-AtG47 from showed a reduced ¹³C discrimination of about 1 unit which is highly significant.

### 6.6 Conclusion: Promoter Gene G47 Combinations

As a summary the promoter-gene combinations pLMP1-G47 and pLMX5-G47 or these promoters in combinations with orthologues of the gene G47 can be used to tailor the expression pattern of the gene G47 or orthologues thereof to the specific growth condition for transgenic plants and trees harbouring such construct, resulting in an improved phenotypical effect such as plant height, width, stem volume, stem dry weight, bark dry weight, water use efficiency and improved drought tolerance compared to wild type plants and trees.

### Example 7 Promoter and Gene TF0002 Combinations

7.1 Greenhouse and Field Trial Experiments with the Promoter-Gene Construct p35S-PttTF0002

Over-expression of a gene may elicit different phenotypical effects under two distinctly different growth conditions. In WO2009084999, the recombinant DNA construct TF0002 was used to constitutively over-express the Populus tremula x tremuloides gene PttTF0002, Seq ID: 2, in hybrid aspen trees using the CaMV 35S promoter. This modification in the transgenic trees resulted in an average stem volume growth increase of 36% compared to wild type reference trees when grown in the greenhouse.

The same hybrid aspen trees were subsequently planted in an open field trial in the south of Sweden. After three years in the field the hybrid aspen trees were measured. It was discovered that the transgenic trees harbouring the promoter-gene construct p35S-PttTF0002 did not perform as well as expected in these field growth tests. The observed average stem volume increase of 36% in greenhouse tests was contrasted with a considerable reduction of stem volume growth of 37% compared to the wild type reference trees grown at the same test site location, FIG. 4. This result was much unexpected, since the CaMV 35S promoter construct consistently worked very well under greenhouse conditions. Constitutive over-expression may have disadvantageous effects on plant growth under field trial conditions.

7.2 Greenhouse and Field Trial Experiments with the Promoter-Gene Construct pLMX5-PttTF0002

The transgenic hybrid aspen trees with the promoter-gene construct pLMX5-PttTF0002 were grown in the greenhouse as well as in an open field trial in the south of Sweden.

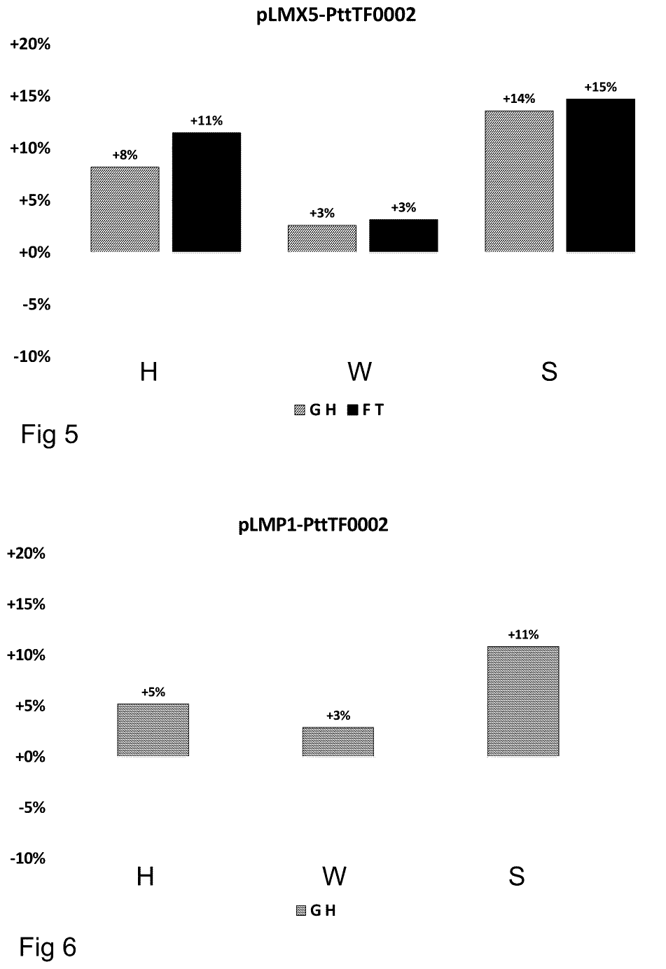

Surprisingly, transgenic hybrid aspen trees with the vascular/xylem specific promoter-gene construct pLMX5-PttTF0002 showed a considerable increase in plant growth both in the greenhouse and in the field, under similar field trial conditions and on the same location as used for the 35S over-expressing trees. Average stem volume increases of 14% and 15% compared to the wild type reference were observed in the greenhouse and the field respectively. Average plant height also increase 8% in the greenhouse and 11% the field in the transgenic trees, FIG. 5.

It is evident from these results, that having a strong, constitutive expression of a trait gene, as with the 35S promoter construct, can have disadvantageous effects under some field trial conditions, whilst having a more specific expression pattern under the same conditions still results in improved growth compared to wild type. These results demonstrate the beneficial and inventive use of specific combinations of promoters and genes to tailor the expression pattern to the specific growth condition and to retain or further improve the positive phenotypical traits provided by the gene when growth conditions change.

7.3 Greenhouse Experiments with the Promoter-Gene Construct pLMP1-PttTF0002

The PttTF0002 gene was over-expressed by the vascular/phloem specific pLMP1 promoter hybrid aspen trees, it was surprisingly noticed that plants grown in the greenhouse had an increase in an average stem volume increase of 11% compared to the wild type reference, FIG. 6. Albeit no field trail data is available for the pLMP-PttTF0002 promoter-gene combination, it is obvious that specifically expressing the TF0002 trait gene with different promoters have different phenotypical effects and can be used to tailor the expression pattern of the gene to the specific growth condition at hand.

### 7.4 Conclusion: Promoter Gene TF0002 Combinations

As a summary the promoter-gene combinations based on the promoter pLMP1 can be used to tailor the expression pattern of the gene TF0002 or orthologues of the gene TF0002 to the specific growth condition for transgenic plants or trees harbouring this construct, resulting in an improved phenotypical effect such as plant height, plant width, stem volume, stem dry weight, bark dry weight, wood density, and drought tolerance compared to wild type.

### Example 8 Promoter and Gene TF0097 Combinations

### 8.1 TF0097 Greenhouse Experiments

Eleven different novel promoter-gene constructs were tested in the greenhouse. A construct where the 35S promoter was used to drive TF0097 expression was included as a reference. These constructs were made as disclosed in Example 3 and plant material was transformed and trees generated as described in Example 4. Finally, transgenic tree growth experiments, measurements and statistics were performed as described in Example 5.

The growth characteristics of the transgenic trees of the different constructs in greenhouse, relative to the wild type reference trees, are summarized in Table 7 A, 7B, 7C, 7D and 7E, below.

TABLE 7A

| | | | | | Stem | Wood |
|---|---|---|---|---|---|---|
| Construct | Gene | Promoter | Height | Width | volume | density |
| TF0097 | PttTF0097 | p35S | +33% * | +11% * | +68% * | +11% * |
| LMP1-003 | PttTF0097 | pLMP1 | +5% | −14% * | −17% | +4% |
| LMX5-003 | PttTF0097 | pLMX5 | −1% | −1% | −2% | |

* Significant differences (p < 0.01) compared to wild type marked with an asterisk.

TABLE 7B

| | | | | | | Stem | Bark | Inter- | | Leaf |
|---|---|---|---|---|---|---|---|---|---|---|
| Con- | | Pro- | | | Stem | dry | dry | node | Wood | dry |
| struct | Gene | moter | Height | Width | volume | weight | weight | length | density | weight |
| F104 | PttTF0097 | pEA1 | +4% | +5% | +15% | +25% * | +13% | −2% | +3% | +24% * |
| F105 | PttTF0097 | pEA3 | +5% | +8% * | +23% * | +29% * | +15% * | +1% | +2% | +28% * |
| F106 | PttTF0097 | pEC1 | +6% * | +7% | +23% * | +26% * | +15% | +3% | −2% | +23% * |
| F107 | PttTF0097 | pEX5 | +2% | +9% * | +21% * | +31% * | +16% * | −5% | −2% | +35% * |
| F108 | PttTF0097 | pEL1.1 | +1% | +6% | +15% | +16% | +10% | −4% | −2% | +25% * |
| F109 | PttTF0097 | pER1 | +0% | +3% | +9% | +8% | −3% | −6% | +6% * | +11% |
| F110 | PttTF0097 | pER2 | −11% * | −5% | −19% * | −26% * | −22% * | −20% * | −4% | −21% * |
| F102 | PttTF0097 | pECO1 | +1% | +3% | +8% | +11% | +7% | −2% | −0% | +15% |
| F103 | PttTF0097 | pECO2 | −1% | +2% | +3% | +5% | +7% | −4% | +1% | +10% |

* Significant differences (p < 0.01) compared to wild type marked with an asterisk.

TABLE 7C
The results from each transgenic line presented individually.

| | | | | | | Stem | Bark | Inter- | | Leaf |
|---|---|---|---|---|---|---|---|---|---|---|
| | | Pro- | | | Stem | dry | dry | node | Wood | dry |
| Construct | Gene | moter | Height | Width | volume | weight | weight | length | density | weight |
| F104-Line1 | PttTF0097 | pEA1 | +8% | +4% | +16% | +30% * | +14% | +3% | +0% | +25% * |
| F104-Line2 | | | +4% | +5% | +13% | +18% | +17% | −5% | +2% | +17% |
| F104-Line3 | | | +0% | +8% | +15% | +26% * | +7% | −4% | +8% * | +29% * |
| F105-Line1 | PttTF0097 | pEA3 | +4% | +3% | +9% | +19% | +9% | −0% | +1% | +13% |
| F105-Line2 | | | +6% | +14% * | +38% * | +50% * | +27% * | −3% | +2% | +51% * |
| F105-Line3 | | | +5% | +7% | +21% | +20% | +8% | +5% | +2% | +19% |
| F106-Line1 | PttTF0097 | pEC1 | +5% | +12% * | +32% * | +44% * | +30% * | +4% | −2% | +31% * |
| F106-Line2 | | | +3% | +2% | +9% | +9% | +8% | +1% | +1% | +14% |
| F106-Line3 | | | +8% | +8% | +26% | +23% | +7% | +5% | −4% | +23% |
| F107-Line1 | PttTF0097 | pEX5 | +5% | +11% | +28% * | +46% * | +30% * | −5% | −2% | +43% * |
| F107-Line2 | | | +2% | +9% | +21% | +17% | +5% | −4% | −6% | +27% * |
| F107-Line3 | | | −1% | +8% | +15% | +31% * | +15% | −5% | +2% | +36% * |
| F108-Line1 | PttTF0097 | pEL1.1 | −1% | −1% | +0% | −4% | −7% | −6% | +1% | +1% |
| F108-Line2 | | | +1% | +10% | +21% | +22% | +10% | −6% | −3% | +36% * |
| F108-Line3 | | | +4% | +9% | +23% | +32% * | +27% * | +0% | −3% | +37% * |
| F109-Line1 | PttTF0097 | pER1 | −2% | −5% | −4% | −5% | −6% | −5% | +9% * | −4% |
| F109-Line2 | | | +7% | +13% * | +38% * | +32% * | +5% | −3% | +2% | +37% * |
| F109-Line3 | | | −4% | −1% | −7% | −4% | −8% | −9% | +8% * | −1% |
| F110-Line1 | PttTF0097 | pER2 | −2% | −4% | −10% | −21% | −12% | −21% * | +4% | −18% |
| F110-Line2 | | | −28% * | −6% | −36% * | −40% * | −41% * | −31% * | −15% * | −40% * |
| F110-Line3 | | | −2% | −5% | −12% | −18% | −14% | −9% * | −1% | −5% |
| F102-Line1 | PttTF0097 | pECO1 | +2% | −1% | +1% | −4% | −4% | −4% | −8% * | +1% |
| F102-Line2 | | | −4% | +3% | +5% | +11% | +10% | −3% | +6% | +20% |
| F102-Line3 | | | +5% | +5% | +16% | +20% | +11% | −0% | −0% | +20% |
| F103-Line1 | PttTF0097 | pECO2 | −1% | +6% | +10% | +1% | +2% | −7% | −0% | +5% |
| F103-Line2 | | | −1% | +0% | −0% | +12% | +11% | −1% | −4% | +16% |
| F103-Line3 | | | +0% | +1% | +1% | +1% | +8% | −3% | +6% | +9% |

* Significant differences (p < 0.01) compared to wild type marked with an asterisk.

TABLE 7D

| | | | | | | Stem | Bark | Inter- | | Leaf |
|---|---|---|---|---|---|---|---|---|---|---|
| | | | | | Stem | dry | dry | node | Wood | dry |
| Construct | Gene | Promoter | Height | Width | volume | weight | weight | length | density | weight |
| F101 | PttTF0097 | p35S | +19% * | +4% | +31% * | +26% * | +26% * | +12% * | +4% | −2% |

* Significant differences (p < 0.01) compared to wild type marked with an asterisk.

TABLE 7E
The results from each transgenic line presented individually.

| | | | | | | Stem | Bark | Inter- | | Leaf |
|---|---|---|---|---|---|---|---|---|---|---|
| | | | | | Stem | dry | dry | node | Wood | dry |
| Construct | Gene | Promoter | Height | Width | volume | weight | weight | length | density | weight |
| F101-Line1 | PttTF0097 | p35S | +28% * | +7% | +48% * | +27% | +25% * | +11% * | +3% | −3% |
| F101-Line2 | | | +6% | +1% | +8% | −0% | +11% | +5% | +8% * | −10% |
| F101-Line3 | | | +24% * | +5% | +35% * | +51% * | +42% * | +19% * | +2% | +8% |

* Significant differences (p < 0.01) compared to wild type marked with an asterisk.

### 8.2 TF0097 Field Trial Experiments

The transgenic hybrid aspen lines of six novel promoter-gene constructs, with the most improved growth characteristics in greenhouse compared to wild type, were selected for field trial testing. Transgenic hybrid aspen lines with the 35S promoter driving TF0097 gene expression was included as a reference. Trees were again propagated from tissue culture material for a field trial experiment, according to Example 5. Wild type reference plants were propagated in parallel and treated exactly as the transgenic plants throughout the experiment.

The growth characteristics of the transgenic trees of the different constructs in field, relative to the wild type reference trees, are summarized in Table 8A, 8B and 8C, below.

TABLE 8A
Significant differences (p < 0.05) compared to wild type
marked with an asterisk (*).

| Construct | Gene | Promoter | Height | Width | Stem volume |
|---|---|---|---|---|---|
| TF0097 | PttTF0097 | p35S | −22% * | −22% | −54% |

TABLE 8B

| Construct | Gene | Promoter | Height | |
|---|---|---|---|---|
| F101 | PttTF0097 | p35S | −12% | |
| F104 | PttTF0097 | pEA1 | +1% | |
| F105 | PttTF0097 | pEA3 | +8% | |
| F106 | PttTF0097 | pEC1 | +17% | |
| F107 | PttTF0097 | pEX5 | −0% | |
| F108 | PttTF0097 | pEL1.1 | +17% | * |
| F109 | PttTF0097 | pER1 | +3% | |

Significant differences according to Dunnett's test compared to wild type (p < 0.05) marked with an asterisk (*).

TABLE 8C
The results from each transgenic line presented individually.
Significant differences according to Dunnett's test compared
to wild type (p < 0.05) marked with an asterisk (*).

| Construct | Gene | Promoter | Height |
|---|---|---|---|
| F101-5A | PttTF0097 | p35S | −6% |
| F101-6B | | | −24% |
| F104-3A | PttTF0097 | pEA1 | +0% |
| F104-3B | | | +3% |
| F104-5B | | | +0% |
| F105-3B | PttTF0097 | pEA3 | +8% |
| F106-5A | PttTF0097 | pEC1 | +17% |
| F107-1A | PttTF0097 | pEX5 | −6% |
| F107-4A | | | −4% |
| F107-4B | | | +10% |
| F108-1A | PttTF0097 | pEL1.1 | +7% |
| F108-1B | | | +18% |
| F108-3B | | | +25% |
| F109-6B | PttTF0097 | PER1 | +3% |

### 8.3 CaMV 35S Over-Expression of TF0097

Although known to potentially increase the risk of gene silencing, constitutive over-expression was used to demonstrate the strong positive effect that TF0097 over-expression can have on plant growth under controlled greenhouse conditions. Transgenic hybrid aspen trees harbouring a recombinant DNA construct, wherewith the TF0097 gene from Populus tremula x tremuloides is over-expressed using the strong constitutive 35S promoter, grow significantly faster, becoming taller and wider as well as having an increased stem volume and dry weight compared to wild type trees, Table 8A, 8D and 8E. Average internode length and wood density are also positively affected by the modification.

A negative pleiotropic effect was observed in the 35S over-expressors. The trees grew tall but consistently had a spindly and slightly curved phenotype of the stem.

However, in the field trial experiment conducted with transgenic trees over-expressing the TF0097 gene using the 35S promoter, strong constitutive over-expression results in severely reduced growth, in contrast to the greenhouse results. These results demonstrate the need for new combinations of a promoter and a trait gene, such as TF0097, or like methods to modify the expression pattern of the trait gene, to achieve an increase in plant growth or biomass production under a given growth condition.

### 8.4 Tissue-Specific Over-Expression of TF0097

By using a number of tissue-specific promoters to control the expression of the TF0097 gene, the inventors are able to demonstrate that specific over-expression of TF0097 gene product does not necessarily lead to an increase in plant growth, Table 7A, 7B and 7C. Conversely, specific over-expression of TF0097 gene product will not generally have a significant effect on plant growth. This demonstrates the non-obvious and inventive use of the specific combinations of promoters and genes disclosed herein to increase plant growth.

Strong, constitutive over-expression of TF0097 is known to potentially have negative effects on growth, for example as described in section 8.2, Table 8A, and may also increase the risk of gene silencing. This risk of adverse effects can be reduced by using for example tissue-specific promoters, such as pEC1 or pEL1.1 to over-express the TF0097 gene.

When the constitutive 35S promoter is used to over-express the TF0097 gene product, leaf dry weight is not significantly affected while the dry weight of other plant parts increase compared to wild type. However, in several instances where a tissue-specific promoter is used to over-express the TF0097 gene product, both leaf dry weight and the dry weight of other plant parts are significantly increased compared to wild type. This again suggests a potentially negative pleotropic effect of strong, constitutive over-expression using the 35S promoter

When the pEA1 promoter is used to over-express the TF0097 gene product growth is significantly improved compared to wild type; looking at the average of all tested transgenic lines, including the three replicates of each, stem dry weight is increased by 25% and the total dry weight of all leaves is increased by 24%. If instead each transgenic line, including its three replicates, is compared to the wild type reference, increases in stem dry weight and total dry weight of all leaves by 30% and 25% respectively are observed in the most improved transgenic line; in the second most improved transgenic line increases in stem dry weight and total dry weight of all leaves by 26% and 29% respectively are observed, as well as an increase in wood density of 8%. Dry weight results confirm that the increase in growth also includes a considerable increase in biomass production in the transgenic trees compared to wild type. No adverse phenotypical effects of tissue-specific over-expression of the TF0097 gene using the pEA1 promoter, are observed.

When the pEA3 promoter is used to over-express the TF0097 gene product growth is significantly improved compared to wild type; looking at the average of all tested transgenic lines, including the three replicates of each, stem diameter is increased by 8% and stem volume is increased by 23%. Further, substantial increases in stem and bark dry weights as well as total dry weight of all leaves of in average 29%, 15% and 28% respectively, are observed. If instead each transgenic line, including its three replicates, is compared to the wild type reference, increases in stem and bark dry weight as well as total dry weight of all leaves by 50%, 27% and 51% respectively are observed in the most improved transgenic line; stem diameter and stem volume are increased by 14% and 38% respectively in the same line. Dry weight results confirm that the increase in growth also includes a considerable increase in biomass production in the transgenic trees compared to wild type. No adverse phenotypical effects of tissue-specific over-expression of the TF0097 gene using the pEA3 promoter, are observed.

When the pEC1 promoter is used to over-express the TF0097 gene product growth is significantly improved compared to wild type; looking at the average of all tested transgenic lines, including the three replicates of each, plant height is increased by 6% and stem volume is increased by 23%. Further, substantial increases in stem dry weight and total dry weight of all leaves of in average 26% and 23% respectively, are observed. If instead each transgenic line, including its three replicates, is compared to the wild type reference, increases in stem and bark dry weight as well as total dry weight of all leaves by 44%, 30% and 31% respectively are observed in the most improved transgenic line; stem diameter and stem volume are increased by 12% and 32% respectively in the same line. Dry weight results confirm that the increase in growth also includes a considerable increase in biomass production in the transgenic trees compared to wild type. No adverse phenotypical effects of tissue-specific over-expression of the TF0097 gene using the pEC1 promoter, are observed.

When the pEX5 promoter is used to over-express the TF0097 gene product growth is significantly improved

When the pEL1.1 promoter is used to over-express the TF0097 gene product growth is significantly improved compared to wild type; looking at the average of all tested transgenic lines, including the three replicates of each, the total dry weight of all leaves is increased by 25%. If instead each transgenic line, including its three replicates, is compared to the wild type reference, increases in stem and bark dry weight as well as total dry weight of all leaves by 32%, 27% and 37% respectively are observed in the most improved transgenic line; in the second most improved transgenic line an increase in total dry weight of all leaves by 36% is observed. Dry weight results confirm that the increase in growth also includes a considerable increase in biomass production in the transgenic trees compared to wild type. No adverse phenotypical effects of tissue-specific over-expression of the TF0097 gene using the pEL1.1 promoter, are observed. The strong promoter pEL1.1 has an expression level and a broad pattern of expression in all green tissues of the plant that make it, in some ways, comparable to the 35S promoter.

When the pER1 promoter is used to over-express the TF0097 gene product growth is significantly improved compared to wild type; looking at the average of all tested transgenic lines, including the three replicates of each, wood density is increased by 6%. If instead each transgenic line, including its three replicates, is compared to the wild type reference, increases in stem and bark dry weight as well as total dry weight of all leaves by 38%, 32% and 37% respectively are observed in the most improved transgenic line; stem diameter and stem volume are increased by 13% and 38% respectively in the same line. Dry weight results confirm that the increase in growth also includes a considerable increase in biomass production in the transgenic trees compared to wild type. No adverse phenotypical effects of tissue-specific over-expression of the TF0097 gene using the pER1 promoter, are observed.

When the pER2 promoter is used to over-express the TF0097 gene product growth is significantly reduced compared to wild type; looking at the average of all tested transgenic lines, including the three replicates of each, plant height, stem volume as well as the dry weight of stem, bark and leaves are significantly reduced. Furthermore, this construct showed narrower leaves in the transgenes compared to wild type leaves was also observed.

### 8.5 Constitutive Over-Expression of TF0097

No statistically significant positive phenotypical effect is observed in the greenhouse when over-expressing the TF0097 using the pECO1 or the pECO2 promoter. The constitutive promoters pECO1 and pECO2 are both weaker than the 35S promoter. The levels of gene over-expression conferred by the pECO1 or pECO2 promoters are too weak to significantly change the growth of the trees in this experiment, Table 7B. Wood density is slightly reduced in one of the transgenic lines when using pECO1 to drive the expression of the TF0097 gene, Table 7C. The risk of adverse effects of strong constitutive expression can be reduced by using weaker constitutive promoters, such as pECO1 or pECO2, to over-express the TF0097 gene.

### 8.3 Conclusions Promoter Gene TF0097 Combinations

Overexpressing the TF0097 gene with different tissue-specific promoters have different phenotypical effects which can be used to tailor the expression pattern of the gene to the specific growth condition at hand and to retain or further improve the positive phenotypical traits provided by the gene when growth conditions change.

Tissue-specific over-expression of the TF0097 gene product provides a more efficient use of resources for the tree compared to constitutive over-expression. When the TF0097 gene is strongly over-expressed in the majority of cells throughout the plant using, for example, the 35S promoter constructs, large quantities of TF0097 gene product are produced also in cells and tissues where there is little or no need of the TF0097 gene product. The impact on plant growth relative to the total amount of TF0097 gene product produced is therefore much higher in transgenic plants with a tissue-specific TF0097 gene over-expression driven by, for example, the pEC1, pEL1.1, pEA1, pEA3, pLMP1, pLMX5, pEX5 or pER1 promoter than in the 35S over-expressing plants. At the same time, specific over-expression will reduce the risk of adverse effects, such as those observed in the field trial described in section 8.2, Table 8A, where the TF0097 gene product was constitutively over-expressed at high levels. It is important to note that different specific promoters function differently and may minimize the risk for negatively growth effects.

It is obvious that specifically expressing the TF0097 gene with different promoters have different phenotypical effects and can be used to tailor the expression pattern of the gene to the specific growth condition at hand. Similarly, it is also obvious that transgenic trees harbouring the 35S promoter construct might still perform optimally for another set of field growth conditions, resulting in an improved phenotypical effect compared to wild type.

In an article by Noh et al 2015, “The poplar basic helix-loop-helix transcription factor BEE3-Like gene affects biomass production by enhancing proliferation of xylem cells in poplar” it is shown that a bHLH transcription factor has positive effect on biomass production. This bHLH protein have 39% identity when compared to Seq ID No.: 3.

### Example 9 Promoter and Gene TF0109 Combinations

### 9.1 TF0109 Greenhouse Experiments

Eight different novel promoter-gene constructs were tested in the greenhouse. A construct where the 35S promoter was used to drive TF0109 expression was included as a reference. These constructs were made as disclosed in Example 3 and plant material was transformed and trees generated as described in Example 4. Finally, transgenic tree growth experiments, measurements and statistics were performed as described in Example 5.

The growth characteristics of the transgenic trees relative to the wild type reference trees in greenhouse of the different constructs are summarized in Table 9A, 9B, 9C, 9D and 9E, below.

TABLE 9A

| | | | | | Stem | Wood |
|---|---|---|---|---|---|---|
| Construct | Gene | Promoter | Height | Width | volume | density |
| TF0109 | PttTF0109 | p35S | +22% * | +15% * | +58% * | +1% |
| LMP1-004 | PttTF0109 | pLMP1 | +5% | −9% | −16% | +1% |
| LMX5-004 | PttTF0109 | pLMX5 | +4% | +1% | +8% | |

* Significant differences (p < 0.01) compared to wild type marked with an asterisk.

TABLE 9B

| | | | | | | Stem | Bark | Inter | | Leaf |
|---|---|---|---|---|---|---|---|---|---|---|
| Con- | | Pro- | | | Stem | dry | dry | node | Wood | dry |
| struct | Gene | moter | Height | Width | volume | weight | weight | length | density | weight |
| F123 | PttTF0109 | pEA1 | +6% * | +4% | +15% * | +16% | +9% | −2% | +1% | +9% |
| F124 | PttTF0109 | pEA2 | +7% * | +7% * | +22% * | +22% * | +17% * | −4% | +3% | +15% * |
| F125 | PttTF0109 | pEA3 | +1% | +1% | +4% | +1% | −0% | −2% | −3% | −2% |
| F126 | PttTF0109 | pEX5 | −1% | −4% | −9% | −11% | −9% | +4% | +4% | −9% |
| F121 | PttTF0109 | pECO1 | +1% | +1% | +4% | +1% | −3% | +0% | +3% | −2% |
| F122 | PttTF0109 | pECO2 | +4% | +6% | +16% * | +20% * | +6% | −1% | +4% | +10% |

* Significant differences (p < 0.01) compared to wild type marked with an asterisk.

TABLE 9C
The results from each transgenic line presented individually.

| | | | | | | Stem | Bark | Inter- | | Leaf |
|---|---|---|---|---|---|---|---|---|---|---|
| | | Pro- | | | | dry | dry | node | Wood | dry |
| Construct | Gene | moter | Height | Width | Stem | weight | weight | length | density | weight |
| F123-2A | PttTF0109 | pEA1 | +4% | +9% | +24% * | +23% | +12% | −2% | +3% | +7% |
| F123-2B | | | +6% | +2% | +11% | +15% | +13% | −6% | −0% | +15% |
| F123-3A | | | +7% | +1% | +10% | +10% | +2% | +1% | −1% | +4% |
| F124-2B | PttTF0109 | pEA2 | +7% | +10% | +29% * | +30% * | +18% | −6% | +7% | +18% |
| F124-3B | | | +7% | +1% | +10% | +12% | +12% | −1% | +1% | +10% |
| F124-6A | | | +5% | +10% | +28% * | +23% | +21% | −5% | +1% | +18% |
| F125-2A | PttTF0109 | pEA3 | +3% | −0% | +2% | +1% | +1% | −3% | −1% | −5% |
| F125-4A | | | +1% | +0% | +1% | −2% | −7% | −1% | −3% | −3% |
| F125-4B | | | −1% | +4% | +8% | +6% | +5% | −2% | −4% | +2% |
| F126-4B | PttTF0109 | pEX5 | +4% | −4% | −4% | −7% | −9% | +3% | −2% | −7% |
| F126-5A | | | −5% | −4% | −14% | −23% | −18% | +14% * | −2% | −19% |
| F126-7B | | | −2% | −5% | −9% | −5% | +1% | −5% | +17% * | −3% |
| F121-2B | PttTF0109 | pECO1 | +3 | +0 | +4% | +1% | −5% | +3% | −2% | −3% |
| F121-3A | | | −1% | +2% | +2% | −2% | −4% | −5% | +5% | −5% |
| F121-3B | | | +1% | +2% | +5% | +4% | −2% | +3% | +7% | +2% |
| F122-2A | PttTF0109 | pECO2 | +4% | +3% | +10% | +11% | +4% | +0% | +4% | +2% |
| F122-3A | | | +2% | +6% | +15% | +17% | +8% | +2% | +3% | +9% |
| F122-4A | | | +6% | +7% | +23% | +31% * | +6% | −6% | +4% | +18% |

* Significant differences (p < 0.01) compared to wild type marked with an asterisk.

TABLE 9D

| | | | | | | Stem | Bark | Inter- | | Leaf |
|---|---|---|---|---|---|---|---|---|---|---|
| | | Pro- | | | Stem | dry | dry | node | Wood | dry |
| Construct | Gene | moter | Height | Width | volume | weight | weight | length | density | weight |
| F120 | PttTF0109 | p35S | +14% * | −5% | +2% | +10% | +0% | +7% * | +11% * | −18% * |

* Significant differences (p < 0.01) compared to wild type marked with an asterisk.

TABLE 9E
The results from each transgenic line presented individually.

| | | | | | | Stem | Bark | Inter- | | Leaf |
|---|---|---|---|---|---|---|---|---|---|---|
| | | | | | Stem | dry | dry | node | Wood | dry |
| Construct | Gene | Promoter | Height | Width | volume | weight | weight | length | density | weight |
| F120-1A | PttTF0109 | p35S | +16% * | −4% | +6% | +13% | +1% | +6% | +11% * | −28% * |
| F120-2A | | | +7% | −2% | +3% | +2% | +3% | −0% | +4% | +6% |
| F120-2B | | | +18% * | −9% | −1% | +15% | −3% | +16% * | +17% * | −33% * |

* Significant differences (p < 0.01) compared to wild type marked with an asterisk.

### 9.2 TF0109 Field Trial Experiments

The transgenic hybrid aspen lines of three novel promoter-gene constructs, with the most improved growth characteristics in greenhouse compared to wild type, were selected for field trial testing. Transgenic hybrid aspen lines with the 35S promoter driving TF0109 gene expression was included as a reference. Trees were again propagated from tissue culture material for a field trial experiment, according to Example 5. Wild type reference plants were propagated in parallel and treated exactly as the transgenic plants throughout the experiment.

The growth characteristics of the transgenic trees of the different constructs in field, relative to the wild type reference trees, are summarized in Table 9F and 9G, below.

TABLE 9F
Significant differences according to Dunnett's test compared
to wild type (p < 0.05) marked with an asterisk (*).

| Construct | Gene | Promoter | Height |
|---|---|---|---|
| F120 | PttTF0109 | p35S | −14% |
| F123 | PttTF0109 | pEA1 | −3% |
| F122 | PttTF0109 | pECO2 | +9% |

TABLE 9G
The results from each transgenic line presented individually.
Significant differences according to Dunnett's test compared
to wild type (p < 0.05) marked with an asterisk (*).

| Construct | Gene | Promoter | Height |
|---|---|---|---|
| F120-1A | PttTF0109 | p35S | −28% |
| F120-2B | | | −9% |
| F123-2A | PttTF0109 | pEA1 | −3% |
| F122-2A | PttTF0109 | pECO2 | +6% |
| F122-3A | | | +7% |
| F122-4A | | | +13% |

There is a variation in the observed level of phenotypical effect of the genetic modification between the different independent transgenic lines. This variation is anticipated for a person skilled in the art, since plants are living, multicellular organisms impossible to grow completely uniformly and since the point of integration of the recombinant DNA construct in the plant genome to a large extent is random and may affect the expression of the inserted genes.

### 9.3 CaMV 35S Over-Expression of TF0109

Although known to potentially increase the risk of gene silencing, constitutive over-expression was used to demonstrate the strong positive effect that TF0109 over-expression can have on plant growth under controlled greenhouse conditions. Transgenic hybrid aspen trees harbouring a recombinant DNA construct, wherewith the TF0109 gene from Populus tremula x tremuloides is over-expressed using the strong constitutive 35S promoter, grow significantly faster, becoming taller and wider with an increased stem volume as well as having an increased wood density and leaf dry weight compared to wild type trees, Table 9A, 9D and 9E. Average internode length can also be positively affected by the modification.

### 9.4 Tissue-Specific Over-Expression of TF0109

By using a number of tissue-specific promoters to control the expression of the TF0109 gene, the inventors are able to demonstrate that specific over-expression of TF0109 gene product does not necessarily lead to an increase in plant growth, Table 9A, 9B and 9C. Conversely, specific over-expression of TF0109 gene product will not generally have a significant effect on plant growth. This demonstrates the non-obvious and inventive use of the specific combinations of promoters and genes disclosed herein to increase plant growth.

Strong, constitutive over-expression of TF0109 is known to potentially have negative effects on growth and may also increase the risk of gene silencing. This risk of adverse effects can be reduced by using for example tissue-specific promoters, such as pEA1, pEA2 and pEC2 to over-express the TF0109 gene.

When the constitutive 35S promoter is used to over-express the TF0109 gene product, leaf dry weight is significantly reduced while the dry weight of other plant parts are not significantly affected compared to wild type. However, in other instances where a tissue-specific promoter is used to over-express the TF0109 gene product, leaf dry weight is not negatively affected while the dry weight of other plant parts are significantly increased compared to wild type. This again suggests a potentially negative pleotropic effect of strong, constitutive over-expression using the 35S promoter.

When the pEA1 promoter is used to over-express the TF0109 gene product growth is significantly improved compared to wild type; looking at the average of all tested transgenic lines, including the three replicates of each, plant height is increased by 6% and stem volume is increased by 15%. If instead each transgenic line, including its three replicates, is compared to the wild type reference, an increase in stem volume by 24% is observed in the most improved transgenic line. No adverse phenotypical effects of tissue-specific over-expression of the TF0109 gene using the pEA1 promoter, are observed.

When the pEA2 promoter is used to over-express the TF0109 gene product growth is significantly improved compared to wild type; looking at the average of all tested transgenic lines, including the three replicates of each, plant height and stem diameter increase by 7% each and stem volume increase by and 22%. Further, substantial increases in stem and bark dry weights as well as total dry weight of all leaves of in average 22%, 17% and 15% respectively, are observed. If instead each transgenic line, including its three replicates, is compared to the wild type reference, increases in stem volume and dry weight by 29% and 30% respectively are observed in the most improved transgenic line. Dry weight results confirm that the increase in growth also includes a considerable increase in biomass production in the transgenic trees compared to wild type. No adverse phenotypical effects of tissue-specific over-expression of the TF0109 gene using the pEA2 promoter, are observed.

No statistically significant positive phenotypical effect is observed in the greenhouse when over-expressing the TF0109 using the pEA3 promoter. No adverse phenotypical effects of tissue-specific over-expression of the TF0109 gene using the pEA3 promoter, are observed.

No statistically significant positive phenotypical effect is observed in the greenhouse when over-expressing the TF0109 using the pLMP1 promoter. No adverse phenotypical effects of tissue-specific over-expression of the TF0109 gene using the pLMP1 promoter, are observed.

The pEX5 and pLMX5 are orthologous promoters, as described in the detailed description of the invention. When the pEX5 promoter is used to over-express the TF0109 gene product wood density is significantly improved compared to wild type; if each transgenic line, including its three replicates, is compared to the wild type reference, an increase in wood density by 17% is observed in the most improved transgenic line. No statistically significant positive phenotypical effect is observed in the greenhouse when over-expressing the TF0109 using the pLMX5 promoter. Wood density was not measured in these transgenic trees. No adverse phenotypical effects of tissue-specific over-expression of the TF0109 gene using the either the pEX5 or the pLMX5 promoter, are observed.

### 9.5 Constitutive Over-Expression of TF0109

No statistically significant positive phenotypical effect is observed in the greenhouse when over-expressing the TF0109 using the pECO1 promoter. The constitutive promoters pECO1 and pECO2 are both weaker than the 35S promoter. The levels of gene over-expression conferred by the pECO1 promoter is too weak to significantly change the growth of the trees in this experiment, Table B. When the pECO2 promoter is used to over-express the TF0109 gene product growth is significantly improved compared to wild type; looking at the average of all tested transgenic lines, including the three replicates of each, stem volume and stem dry weight are increased by 16% and 20% respectively. If instead each transgenic line, including its three replicates, is compared to the wild type reference, an increase in stem dry weight by 31% is observed in the most improved transgenic line. Dry weight results confirm that the increase in growth also includes a considerable increase in biomass production in the transgenic trees compared to wild type. No adverse phenotypical effects of constitutive over-expression of the TF0109 gene using the either the pECO1 or the pECO2 promoter, are observed. The risk of adverse effects of strong constitutive expression might be reduced by using weaker constitutive promoters, such as pECO1 or pECO2, to over-express the TF0109 gene.

### 9.6 Conclusions Promoter Gene TF0109 Combinations

Overexpressing the TF0109 gene with different tissue-specific promoters have different phenotypical effects which can be used to tailor the expression pattern of the gene to the specific growth condition at hand and to retain or further improve the positive phenotypical traits provided by the gene when growth conditions change.

Tissue-specific over-expression of the TF0109 gene product provides a more efficient use of resources for the tree compared to constitutive over-expression. When the TF0109 gene is strongly over-expressed in the majority of cells throughout the plant using, for example, the 35S promoter constructs, large quantities of TF0109 gene product are produced also in cells and tissues where there is little or no substrate to process. The impact on plant growth relative to the total amount of TF0109 gene product produced is therefore much higher in transgenic plants with a tissue-specific TF0109 gene over-expression driven by, for example, the pEA1, pEA2, or pEC2 promoter than in the 35S over-expressing plants. At the same time, specific over-expression will reduce the risk of adverse effects, such as those observed in the field trial.

It is obvious that specifically expressing the TF0109 gene with different promoters have different phenotypical effects and can be used to tailor the expression pattern of the gene to the specific growth condition at hand. Similarly, it is also obvious that transgenic trees harbouring the 35S promoter construct might still perform optimally for another set of field growth conditions, resulting in an improved phenotypical effect compared to wild type.

### Example 10 Promoter and Gene TF0132 Combinations

### 10.1 TF0132 Greenhouse Experiments

Ten different novel promoter-gene constructs were tested in the greenhouse. A construct where the 35S promoter was used to drive TF0132 expression was included as a reference. These constructs were made as disclosed in Example 3 and plant material was transformed and trees generated as described in Example 4. Finally, transgenic tree growth experiments, measurements and statistics were performed as described in Example 5.

The growth characteristics of the transgenic trees relative to the wild type reference trees in greenhouse of the different constructs are summarized in Table 10A, B, C, D and E, below.

TABLE 10A

| | | | | | Stem | Wood |
|---|---|---|---|---|---|---|
| Construct | Gene | Promoter | Height | Width | volume | density |
| TF0132 | PttTF0132 | p35S | +27% * | +12% * | +65% * | +7% |
| LMP1-005 | PttTF0132 | pLMP1 | +6% | +5% | +17% | −1% |
| LMX5-005 | PttTF0132 | pLMX5 | +3% | −5% | −7% | |

* Significant differences (p < 0.01) compared to wild type marked with an asterisk.

TABLE 10B

| | | | | | | Stem | Bark | Inter- | | Leaf |
|---|---|---|---|---|---|---|---|---|---|---|
| Con- | | | | | Stem | dry | dry | node | Wood | dry |
| struct | Gene | Promoter | Height | Width | volume | weight | weight | length | density | weight |
| F114 | PttTF0132 | pEA1 | +6% * | +2% | +11% | +12% | +9% | +3% | +3% | +4% |
| F115 | PttTF0132 | pEA2 | +1% | +1% | +3% | +10% | +5% | −5% | +5% | −0% |
| F116 | PttTF0132 | pEA3 | +1% | +1% | +4% | +12% | +8% | −3% | +2% | +9% |
| F117 | PttTF0132 | pEA4 | +1% | +1% | +3% | +9% | +8% | +0% | +2% | +7% |
| F118 | PttTF0132 | pEC1 | +8% * | +4% | +15% * | +22% * | +20% * | +0% | +3% | +16% * |
| F119 | PttTF0132 | pEL1.1 | −3% | −4% | −10% | −6% | −7% | +2% | −2% | −5% |
| F112 | PttTF0132 | pECO1 | +2% | +5% | +13% | +21% * | +18% * | −1% | +8% * | +12% |
| F113 | PttTF0132 | pECO2 | +2% | +2% | +5% | +16% * | +13% | +3% | +9% * | +12% |

* Significant differences (p < 0.01) compared to wild type marked with an asterisk.

TABLE 10C
The results from each transgenic line presented individually.

| | | | | | | Stem | Bark | Inter- | | Leaf |
|---|---|---|---|---|---|---|---|---|---|---|
| | | | | | Stem | dry | dry | node | Wood | dry |
| Construct | Gene | Promoter | Height | Width | volume | weight | weight | length | density | weight |
| F114-1A | PttTF0132 | pEA1 | +4% | −5% | −6% | −4% | −5% | +4% | +2% | −12% |
| F114-3B | | | +11% * | +7% | +26% * | +26% * | +19% | +8% | +7% | +11% |
| F114-4A | | | +2% | +5% | +13% | +14% | +13% | −3% | +0% | +13% |
| F115-3A | PttTF0132 | pEA2 | +4% | +3% | +10% | +18% | +8% | −1% | +5% | +8% |
| F115-4A | | | −3% | −0% | −3% | −3% | −7% | −11% | +1% | −8% |
| F115-4B | | | +1% | −0% | +1% | +15% | +13% | −3% | +9% | −1% |
| F116-2B | PttTF0132 | pEA3 | +2% | +4% | +10% | +18% | +6% | +2% | +2% | +17% |
| F116-3A | | | −1% | +3% | +4% | +11% | +13% | −11% | +3% | +11% |
| F116-4A | | | +3% | −3% | −3% | +5% | +5% | +1% | +1% | −3% |
| F117-2B | PttTF0132 | pEA4 | −9% * | +1% | −7% | −12% | −12% | +2% | +1% | −1% |
| F117-4B | | | +8% * | +0% | +9% | +20% | +18% | +2% | +3% | +10% |
| F117-5B | | | +4% | +2% | +7% | +21% * | +19% | −5% | +2% | +12% |
| F118-1A | PttTF0132 | pEC1 | +13% * | +8% | +31% * | +33% * | +23% | +2% | −0% | +19% |
| F118-2A | | | +7% | −1% | +5% | +20% | +23% * | +5% | +5% | +12% |
| F118-3A | | | +4% | +5% | +15% | +18% | +14% | −5% | +3% | +18% |
| F119-1B | PttTF0132 | pEL1.1 | −0% | −0% | −1% | +14% | +12% | −5% | +4% | +10% |
| F119-2A | | | −14% * | −5% | −23% * | −32% * | −31% * | +12% | −9% | −21% * |
| F119-3A | | | +6% | −5% | −5% | +1% | −1% | −0% | −1% | −5% |
| F112-1A | PttTF0132 | pECO1 | −5% | −0% | −5% | −5% | +1% | +7% | +10% * | −6% |
| F112-3B | | | +5% | +7% | +20% | +35% * | +25% * | −7% | +5% | +20% * |
| F112-6B | | | +7% | +8% | +24% * | +33% * | +27% * | −0% | +8% | +22% * |
| F113-1A | PttTF0132 | pECO2 | +1% | +9% | +21% | +31% * | +23% * | +1% | +11% * | +25% * |
| F113-2A | | | +4% | −2% | −0% | +14% | +10% | +4% | +6% | +12% |
| F113-2B | | | −0% | −3% | −5% | +3% | +5% | +5% | +10% * | −2% |

* Significant differences (p < 0.01) compared to wild type marked with an asterisk.

TABLE 10D

| | | | | | | Stem | Bark | Inter- | | Leaf |
|---|---|---|---|---|---|---|---|---|---|---|
| | | | | | Stem | dry | dry | node | Wood | dry |
| Construct | Gene | Promoter | Height | Width | volume | weight | weight | length | density | weight |
| F111 | PttTF0132 | p35S | +30% * | −0% | +28% * | +46% * | +30% * | +19% * | +14% * | +8% |

* Significant differences (p < 0.01) compared to wild type marked with an asterisk.

TABLE 10E
The results from each transgenic line presented individually.

| | | | | | | Stem | Bark | Inter- | | Leaf |
|---|---|---|---|---|---|---|---|---|---|---|
| | | Pro- | | | Stem | dry | dry | node | Wood | dry |
| Construct | Gene | moter | Height | Width | volume | weight | weight | length | density | weight |
| F111-2A | PttTF0132 | p35S | +31% * | −3% | +22% * | +47% * | +33% * | +18% * | +23% * | +4% |
| F111-3A | | | +41% * | −3% | +33% * | +65% * | +47% * | +20% * | +22% * | +18% |
| F111-4A | | | +18% * | +5% | +29% * | +26% * | +12% | +20% * | −2% | +3% |

* Significant differences (p < 0.01) compared to wild type marked with an asterisk.

### 10.2 TF0132 Field Trial Experiments

The transgenic hybrid aspen lines of four novel promoter-gene constructs, with the most improved growth characteristics in greenhouse compared to wild type, were selected for field trial testing. Transgenic hybrid aspen lines with the 35S promoter driving TF0132 gene expression was included as a reference. Trees were again propagated from tissue culture material for a field trial experiment, according to Example 5. Wild type reference plants were propagated in parallel and treated exactly as the transgenic plants throughout the experiment.

The growth characteristics of the transgenic trees of the different constructs in field, relative to the wild type reference trees, are summarized in Table 1 OF, 10G and 10H, below.

TABLE 10F
Significant differences (p < 0.05) compared
to wild type marked with an asterisk (*).

| Construct | Gene | Promoter | Height | Width | Stem volume |
|---|---|---|---|---|---|
| TF0132 | PttTF0132 | p35S | +4% | −14% | −21% |

TABLE 10G
Significant differences according to Dunnett's test compared
to wild type (p < 0.05) marked with an asterisk (*).

| Construct | Gene | Promoter | Height |
|---|---|---|---|
| F111 | PttTF0132 | p35S | −14% |
| F114 | PttTF0132 | pEA1 | −4% |
| F118 | PttTF0132 | pEC1 | +3% |
| F112 | PttTF0132 | pECO1 | −3% |
| F113 | PttTF0132 | pECO2 | +3% |

TABLE 10H
The results from each transgenic line presented individually.
Significant differences according to Dunnett's test compared
to wild type (p < 0.05) marked with an asterisk (*).

| Construct | Gene | Promoter | Height |
|---|---|---|---|
| F111-2A | PttTF0132 | p35S | −21% |
| F111-3A | | | −10% |
| F111-4A | | | −11% |
| F114-3B | PttTF0132 | pEA1 | −4% |
| F118-1A | PttTF0132 | pEC1 | +6% |
| F118-2A | | | +12% |
| F118-3A | | | −10% |
| F112-1A | PttTF0132 | pECO1 | −9% |
| F112-3B | | | −1% |
| F112-6B | | | +4% |
| F113-1A | PttTF0132 | pECO2 | +3% |

### 10.3 Tissue-Specific Over-Expression of TF0132

By using a number of tissue-specific promoters to control the expression of the TF0132 gene, the inventors are able to demonstrate that specific over-expression of TF0132 gene product does not necessarily lead to an increase in plant growth, Table 10 A, B and C. Conversely, specific over-expression of TF0132 gene product will not generally have a significant effect on plant growth. This demonstrates the non-obvious and inventive use of the specific combinations of promoters and genes disclosed herein to increase plant growth.

### 10.4 CaMV 35S Over-Expression of TF0132

Although known to potentially increase the risk of gene silencing, constitutive over-expression was used to demonstrate the strong positive effect that TF0132 over-expression can have on plant growth under controlled greenhouse conditions. Transgenic hybrid aspen trees harbouring a recombinant DNA construct, wherewith the TF0132 gene from Populus tremula x tremuloides is over-expressed using the strong constitutive 35S promoter, grow significantly faster, becoming taller and wider with an increased stem volume and dry weight as well as having an increased wood density and average internode length compared to wild type trees, Table 10 A, D and E.

However, in the field trial experiment conducted with transgenic trees over-expressing the TF0132 gene using the 35S promoter, strong constitutive over-expression does not result in significantly increased growth, in contrast to the greenhouse results. These results demonstrate the need for new combinations of a promoter and a trait gene, such as TF0132, or like methods to modify the expression pattern of the trait gene, to achieve an increase in plant growth or biomass production under a given growth condition.

Strong, constitutive over-expression of TF0132 is known to potentially have negative effects on growth and may also increase the risk of gene silencing. This risk of adverse effects can be reduced by using for example tissue-specific promoters, such as pEC1 and/or pEA1, to over-express the TF0132 gene.

When the pEA1 promoter is used to over-express the TF0132 gene product growth is significantly improved compared to wild type; looking at the average of all tested transgenic lines, including the three replicates of each, plant height is increased by 6%. If instead each transgenic line, including its three replicates, is compared to the wild type reference, increases in plant height by 11% and in stem volume and dry weight by 26% each are observed in the most improved transgenic line. Dry weight results confirm that the increase in growth also includes a considerable increase in biomass production in the transgenic trees compared to wild type. No adverse phenotypical effects of tissue-specific over-expression of the TF0132 gene using the pEA1 promoter, are observed.

No statistically significant positive phenotypical effect is observed in the greenhouse when over-expressing the TF0132 using the pEA2 promoter. No adverse phenotypical effects of tissue-specific over-expression of the TF0132 gene using the pEA2 promoter, are observed.

No statistically significant positive phenotypical effect is observed in the greenhouse when over-expressing the TF0132 using the pEA3 promoter. No adverse phenotypical effects of tissue-specific over-expression of the TF0132 gene using the pEA3 promoter, are observed.

When the pEA4 promoter is used to over-express the TF0132 gene product growth is significantly improved compared to wild type; if each transgenic line, including its three replicates, is compared to the wild type reference, an increase in stem dry weight by 21% is observed in the most improved transgenic line. No adverse phenotypical effects of tissue-specific over-expression of the TF0132 gene using the pEA4 promoter, are observed.

When the pEC1 promoter is used to over-express the TF0132 gene product growth is significantly improved compared to wild type; looking at the average of all tested transgenic lines, including the three replicates of each, plant height is increased by 8% and stem volume is increased by 15%. Further, substantial increases in stem and bark dry weight as well as total dry weight of all leaves of in average 22%, 20% and 16% respectively, are observed. If instead each transgenic line, including its three replicates, is compared to the wild type reference, an increase in stem dry weight by 33% is observed in the most improved transgenic line; plant height and stem volume are increased by 13% and 31% respectively in the same line. Dry weight results confirm that the increase in growth also includes a considerable increase in biomass production in the transgenic trees compared to wild type. No adverse phenotypical effects of tissue-specific over-expression of the TF0132 gene using the pEC1 promoter, are observed.

No statistically significant positive phenotypical effect is observed in the greenhouse when over-expressing the TF0132 using the pLMP1 promoter. No adverse phenotypical effects of tissue-specific over-expression of the TF0132 gene using the pLMP1 promoter, are observed.

No statistically significant positive phenotypical effect is observed in the greenhouse when over-expressing the TF0132 using the pLMX5 promoter. No adverse phenotypical effects of tissue-specific over-expression of the TF0132 gene using the pLMX5 promoter, are observed.

When the pEL1.1 promoter is used to over-express the TF0132 gene product growth is significantly reduced compared to wild type; if each transgenic line, including its three replicates, is compared to the wild type reference, a reduction in plant height by 14% is observed in one of the transgenic lines; stem volume is reduced by 23% in the same line. Further, substantial reductions in stem and bark dry weight as well as total dry weight of all leaves of 32%, 31% and 21% respectively, are observed in this transgenic line.

### 10.5 Constitutive Over-Expression of TF0132

The constitutive promoters pECO1 and pECO2 are both weaker than the 35S promoter. When the pECO1 promoter is used to over-express the TF0132 gene product growth is significantly improved compared to wild type; looking at the average of all tested transgenic lines, including the three replicates of each, stem and bark dry weight are increased by 21% and 18% respectively and an increase in wood density of 8% is observed. If instead each transgenic line, including its three replicates, is compared to the wild type reference, stem and bark dry weight as well as total dry weight of all leaves increase by 33%, 27% and 22% respectively, in the most improved transgenic line. Stem volume is increased by 24% in the same line. When the pECO2 promoter is used to over-express the TF0132 gene product growth is significantly improved compared to wild type; looking at the average of all tested transgenic lines, including the three replicates of each, stem dry weight is increased by 16% and an increase in wood density of 9% is observed. If instead each transgenic line, including its three replicates, is compared to the wild type reference, stem and bark dry weight as well as total dry weight of all leaves increase by 31%, 23% and 25% respectively, in the most improved transgenic line. Wood density is increased by 11% in the same line. Dry weight results confirm that the increase in growth also includes a considerable increase in biomass production in the transgenic trees compared to wild type. No adverse phenotypical effects of constitutive over-expression of the TF0132 gene using the either the pECO1 or the pECO2 promoter, are observed. The risk of adverse effects of strong constitutive expression can be reduced by using weaker constitutive promoters, such as pECO1 or pECO2, to over-express the TF0132 gene.

### 10.6 Conclusions Promoter Gene TF0132 Combinations

Overexpressing the TF0132 gene with different tissue-specific promoters have different phenotypical effects which can be used to tailor the expression pattern of the gene to the specific growth condition at hand and to retain or further improve the positive phenotypical traits provided by the gene when growth conditions change.

Tissue-specific over-expression of the TF0132 gene product provides a more efficient use of resources for the tree compared to constitutive over-expression. When the TF0132 gene is strongly over-expressed in the majority of cells throughout the plant using, for example, the 35S promoter constructs, large quantities of TF0132 gene product are produced also in cells and tissues where there is little or no substrate to process. The impact on plant growth relative to the total amount of TF0132 gene product produced is therefore much higher in transgenic plants with a tissue-specific TF0132 gene over-expression driven by, for example, the pEA1, pEA2, pEA3, pEA4, pEC1, pLMP1, pLMX5 or pEL1.1 promoter than in the 35S over-expressing plants. The most preferred promoters are pEC1 and pEA1. At the same time, specific over-expression will reduce the risk of adverse effects, such as those observed in the field trial described in section 8.2, Table F, where the TF0097 gene product was constitutively over-expressed at high levels.

It is obvious that specifically expressing the TF0132 gene with different promoters have different phenotypical effects and can be used to tailor the expression pattern of the gene to the specific growth condition at hand. Similarly, it is also obvious that transgenic trees harbouring the 35S promoter construct might still perform optimally for another set of field growth conditions, resulting in an improved phenotypical effect compared to wild type.

### Example 11 Construction of Novel Promoter-Gene Combinations for Expression in Eucalyptus

As described in Example 1 the Eucalyptus promoter DNA fragments were manufactured by DNA synthesis and flanked by Gateway recombination sites for sub-cloning purposes. All Eucalyptus promoter fragments were cloned upstream of the gene of interest using the pSTT0111 vector or modification of it, and thus controlling the expression of the gene of interest as described below.

11.1 Constructs with the Gene G47

### Construct E0011

The promoter pLMP1, Seq ID No: 13, with expression in the cambium and in the phloem was combined with the Arabidopsis thaliana, AtG47 gene, Seq ID No: 1, in the pSTT0111 vector to create the recombinant DNA construct E0011, pLPM1-AtG47. The construct is used to produce transgenic Eucalyptus trees.

11.2 Constructs with the Gene TF0097

### Construct E0012

In this construct the stem/cambium specific promoter pEC1, Seq ID No: 12, was combined with the Populus tremula x tremuloides, PttTF0097 gene, Seq ID No: 23, in the pSTT0118 vector to create the recombinant DNA construct E0012, pEC1-PttTF0097. This construct is used to produce transgenic Eucalyptus trees.

### Construct E0017

The strong constitutive promoter p35S, Seq ID No: 20, was combined with the Populus tremula x tremuloides, PttTF0097 gene, Seq ID No: 23, in the pSTT0117 vector to create the recombinant DNA construct E0017, p35S-PttTF0097. The construct is used to produce transgenic Eucalyptus trees.

### Construct E0018

The strong constitutive promoter p35S, Seq ID No: 20, was combined with the Eucalyptus grandis x urophylla, EucTF0097 gene, Seq ID No: 55, in the pSTT0117 vector to create the recombinant DNA construct E0018, p35S-EucTF0097. The construct is used to produce transgenic Eucalyptus trees.

### Construct E0019

The root and the meristematic tissues promoter pER1, Seq ID No: 18, was combined with the Eucalyptus grandis x urophylla, EucTF0097 gene, Seq ID No: 55, in the pSTT0117 vector to create the recombinant DNA construct E0019, pER1-EucTF0132. The construct is used to produce transgenic Eucalyptus trees.

11.3 Constructs with the Gene TF0132

### Construct E0025

The strong constitutive promoter p35S, Seq ID No: 20, was combined with the Populus tremula x tremuloides, PttTF0132 gene, Seq ID No: 24 in the pSTT0117 vector to create the recombinant DNA construct E0025, p35S-PttTF0132. The construct is used to produce transgenic Eucalyptus trees.

### Construct E0026

The constitutive promoter pECO1, Seq ID No: 6, was combined with the Populus tremula x tremuloides, PttTF0132 gene, Seq ID No: 24 in the pSTT0117 vector to create the recombinant DNA construct E0026, pECO1-PttTF0132. The construct is used to produce transgenic Eucalyptus trees.

### Construct E0027

The constitutive promoter pEC1, Seq ID No: 12, was combined with the Populus tremula x tremuloides, PttTF0132 gene, Seq ID No: 24 in the pSTT0117 vector to create the recombinant DNA construct E0027, pEC1-PttTF0132. The construct is used to produce transgenic Eucalyptus trees.

### Construct E0028

The constitutive promoter pEA1, Seq ID No: 8, was combined with the Populus tremula x tremuloides, PttTF0132 gene, Seq ID No: 24 in the pSTT0117 vector to create the recombinant DNA construct E0028, pEA1-PttTF0132. The construct is used to produce transgenic Eucalyptus trees.

### Example 12 Eucalyptus Transformation

A new transformation vector is constructed for expression of a trait gene in Eucalyptus. The vector backbone is based on the established plasmid-PZP (pPZP) vector system, a small, versatile pPZP family of Agrobacterium binary vectors for plant transformation, Hajdukiewicz et al. 1994, Plant Mol. Biol. 25 (6), 989-994. The T-DNA cassette is designed to contain the desired genetic elements, a selectable marker cassette and a trait gene expression cassette. The genetic elements are separated by linker sequences containing unique restriction sites to facilitate cloning. The selectable marker is kanamycin for both bacterial selection (plasmid selection) and selection of transgenic plants during the transformation process. The method of transformation of Eucalyptus may be Agrobacterium mediated transformation using a standard protocol and kanamycin selection essentially as described by Tournier et al. Transgenic Research, 2003, Volume 12, Issue 4, pp 403-411, or by Ho et al., Plant Cell Reports, 1998, Volume 17, Issue 9, pp 675-680.

### Example 13 Regeneration and Growth of Eucalyptus Plants

The transformed tissue generated in Example 12 is further treated under conditions for plant formation and root formation to get a transgenic Eucalyptus plant. The regeneration may be essentially done according to the protocol presented by Tournier et al. Transgenic Research, 2003, Volume 12, Issue 4, pp 403-411, or by Ho et al., Plant Cell Reports, 1998, Volume 17, Issue 9, pp 675-680.

1-27. (canceled)

28. A genetically modified plant comprising a heterologous nucleic acid construct comprising a promoter operably linked to a coding sequence encoding a TF0132 gene, comprising:
(i) a nucleotide sequence as set forth in SEQ ID NO: 24, 85, 87, 89, 91, 115, 117, 119, 121, 123 or 125, or an ortholog thereof; or
(ii) a nucleotide sequence encoding a polypeptide comprising an amino acid sequence as set forth in SEQ ID NO: 4, 84, 86, 88, 90, 114, 116, 118, 120, 122, 124, or an ortholog thereof,
wherein the promoter is selected from the group consisting of: a promoter preferentially or specifically expressed in phloem tissue of the plant, a promoter preferentially or specifically expressed in meristematic tissue of the plant, a promoter preferentially or specifically expressed in xylem tissue of the plant, and a promoter preferentially or specifically expressed in root tissue of the plant.

29. The genetically modified plant of claim 28, wherein the promoter comprises a nucleotide sequence selected from the group consisting of: a nucleotide sequence of pEC1 (SEQ ID NO: 12, 32 or 39), a nucleotide sequence of pEL1.1 (SEQ ID NO: 16, 34 or 40), a nucleotide sequence of pEA1 (SEQ ID NO: 8 or 28), a nucleotide sequence of pEA2 (SEQ ID NO: 9 or 29), a nucleotide sequence of pECO2 (SEQ ID NO: 7 or 27), pEA3 (SEQ ID NO: 10 or 30), a nucleotide sequence of pEA4 (SEQ ID NO: 11, 31 or 38), a nucleotide sequence of pECO1 (SEQ ID NO: 6 or 26), and a nucleotide sequence of promoter that has the same, or essentially the same, capability of initiating transcription of a coding sequence when operably linked to the coding sequence.

30. The genetically modified plant of claim 28, wherein the promoter preferentially or specifically expressed in meristematic tissue of the plant is preferentially or specifically expressed in at least one of cambium, vascular meristematic tissue, and shoot meristem tissue of the plant.

31. The genetically modified plant of claim 30, wherein the promoter is not significantly expressed in at least one of mature xylem, stem phloem, whole leaves, whole roots, and bark of the plant.

32. The genetically modified plant of claim 28, wherein the TF0132 gene is from Eucalyptus grandis, Zea Mays, Populus trichocarpa, or Arabidopsis thaliana.

33. The genetically modified plant of claim 28, comprising a modified trait as compared to a wild-type plant of the same species, wherein the modified trait is plant yield, plant height, plant width, stem volume, stem dry weight, bark dry weight, wood density, leaf dry weight, average internode length, or number of internodes.

34. The genetically modified plant of claim 33, wherein the modified trait is increased as compared to a wild-type plant of the same species when the plants are grown under identical field conditions for a period of at least one year.

35. The genetically modified plant of claim 28, wherein the plant is a woody plant.

36. The genetically modified plant of claim 28, wherein the plant is a hardwood plant.

37. The genetically modified plant of claim 28, wherein the plant is of the genus Eucalyptus or Populus.

38. The genetically modified plant of claim 28, wherein the plant is a crop plant.

39. The genetically modified plant of claim 28, wherein the coding sequence encodes the gene product TF0132, wherein the promoter is pEC1, pEL1.1, pEA1, pEA2, pEA3, pEA4, pECO1, or pECO2, and wherein the modified trait is at least one of plant yield, plant height, plant width, stem volume, stem dry weight, bark dry weight, wood density, leaf dry weight.

40. A method of making the genetically modified plant of claim 28, the method comprising:
a) providing a suitable part of a plant;
b) providing a heterologous nucleic acid construct comprising a promoter operably linked to a coding sequence encoding a TF0132 gene,
wherein the promoter is pEC1, pEL1.1, pEA1, pEA2, pECO2, pEA3, pEA4, pECO1, or a promoter that has the same, or essentially the same, capability of initiating transcription of a coding sequence when operably linked to the coding sequence;
c) introducing the heterologous nucleic acid construct into the suitable part of the plant; and
d) regenerating the genetically modified plant from the suitable part of the plant.

41. A genetically modified woody plant comprising a heterologous nucleic acid construct comprising a promoter operably linked to a coding sequence encoding a TF0132 gene, comprising:
(i) the nucleotide sequence of SEQ ID NO: 24, 85, 87, 89, 91, 115, 117, 119, 121, 123, or 125, or an ortholog thereof, or
(ii) a nucleotide sequence encoding a polypeptide comprising the amino acid sequence of SEQ ID NO: 4, 84, 86, 88, 90, 114, 116, 118, 120, 122, 124, or an ortholog thereof,
wherein the promoter comprises a nucleotide sequence selected from the group consisting of: a nucleotide sequence of pEC1 (SEQ ID NO: 12, 32 or 39), a nucleotide sequence of pEL1.1 (SEQ ID NO: 16, 34 or 40), a nucleotide sequence of pEA1 (SEQ ID NO: 8 or 28), a nucleotide sequence of pEA2 (SEQ ID NO: 9 or 29), pECO2 (SEQ ID NO: 7 or 27), a nucleotide sequence of pEA3 (SEQ ID NO: 10 or 30), a nucleotide sequence of pEA4 (SEQ ID NO: 11, 31 or 38), a nucleotide sequence of pECO1 (SEQ ID NO: 6 or 26), and a nucleotide sequence of a promoter that has the same, or essentially the same, capability of initiating transcription of a coding sequence when operably linked to the coding sequence,
wherein the promoter is preferentially or specifically expressed in at least one of cambium, vascular meristematic tissue, and shoot meristem tissue of the plant,
wherein the modified plant has a modified trait as compared to a wild-type plant of the same species,
wherein the modified trait is selected from plant yield, plant height, plant width, stem volume, stem dry weight, bark dry weight, wood density, leaf dry weight, average internode length, and number of internodes, and
wherein the modified trait is increased as compared to a wild-type plant of the same species when the plants are grown under identical field conditions for a period of at least one year.
